(19) United States
(12) Patent Application Publication
(10) Pub. No.: US 2022/0152223 A1
(43) Pub. Date: May 19, 2022

(54) VECTOR AND METHOD FOR TREATING ANGELMAN SYNDROME

(71) Applicants: PTC THERAPEUTICS, INC., South Plainfield, NJ (US); UNIVERSITY OF SOUTH FLORIDA, Tampa, FL (US)

(72) Inventors: Antonio ARULANANDAM, Winchester, MA (US); Kevin NASH, Temple Terrace, FL (US); Edwin WEEBER, Apollo Beach, FL (US); Liangxian CAO, East Brunswick, NJ (US); Min Jung KIM, Westfield, NJ (US)

(21) Appl. No.: 17/439,140

(22) PCT Filed: Mar. 20, 2020
(86) PCT No.: PCT/US2020/024030

§ 371 Date: Sep. 14, 2021

Related U.S. Application Data
(60) Provisional application No. 62/821,442, filed on Mar. 21, 2019.

(51) Int. Cl. A61K 48/00; C12N 15/86; A61P 25/00

(52) U.S. Cl. CPC A61K 48/005; C12N 15/86; C12N 2750/14143; A61P 25/00; A61K 48/0075

(57) ABSTRACT

One aspect described herein relates to a recombinant adeno-associated virus (rAAV) vector and a method for use thereof or treating Angelman Syndrome. Another aspect described herein is a UBE3A rAAV vector and method for use thereof for treating a UBE3A deficiency, e.g. Angelman syndrome, in humans.

### CROSS-REFERENCE TO RELATED APPLICATIONS

This application claims the benefit of U.S. Provisional Patent Application 62/821,442, filed Mar. 21, 2019, the content of which is incorporated by reference herein in its entirety.

### FIELD

One aspect described herein relates to a mutated recombinant adeno-associated virus (mrAAV) vector and a method for use thereof for treating Angelman Syndrome. Another aspect described herein is a UBE3A mrAAV vector and method for use thereof for treating Angelman syndrome.

### BACKGROUND

Angelman Syndrome (AS) is a neurodegenerative genetic disorder that is estimated to affect about one in every 10-15,000 births showing no population preference and worldwide expression. However, the actual number of diagnosed AS cases is likely greater due to misdiagnosis. AS manifests as a delay in reaching major milestones of normal development within the first year of life. The AS phenotypic characteristics include significant motor dysfunction, severe cognitive disruption, speech and communication impairments, and often seizures.

The ubiquitin protein ligase E3A gene (also referred to herein as “UBE3A”) is located on chromosome 15q11-13 and, due to its unique imprinting regulation, is only transcribed from the maternal copy in neurons while the paternal is silenced. UBE3A expression is otherwise bi-allelic expression in all non-CNS tissues. Thus, disruption of the maternal gene results in loss of protein in neurons. AS is considered a monogenic disorder resulting from mutation, unipaternal disomy, or methyl-transferase disorder; however, disruption of the UBE3A allele can also occur from large chromosomal deletions effecting multiple genes (Kishino, et al., UBE3A/E6AP mutations cause Angelman syndrome; Nat Gen.; 1997 Jan. 15. 15(1):70-3, the content of which is incorporated herein in its entirety). Specifically, loss of UBE3A expression in the hippocampus and cerebellum is implicated in the etiology of Angelman Syndrome. AS can result from single loss-of-function mutation or from the disruption of the UBE3A allele as a result of large chromosomal deletions affecting multiple genes.

The published International Application number WO2019/006107 describes a recombinant adeno-associated virus (rAAV) serotype 4 vector comprising a sequence encoding a variation of a UBE3A protein sequence, a cell uptake sequence, and a secretion sequence and plasmid vectors comprising such sequences for use in the treatment of UBE3A deficiency diseases, including Angelman Syndrome. The secretion sequence of those vectors encodes for a secretion signaling peptide that promotes the secretion of UBE3A from cells. Unfortunately, WO2019/006107 only reported on localized UBE3A protein expression within on a small region of the brain. Accordingly, there remains an ongoing need for gene therapy that can produce broad UBE3A gene expression throughout the entire brain of an Angelman Syndrome patient.

### SUMMARY

One aspect described herein is a UBE3A vector comprising, a nucleic acid component and protein component. The nucleic acid comprising:
i) a 5′ inverted terminal repeat (ITR) sequence;
ii) a promoter downstream of the 5′ ITR sequence;
iii) a UBE3A nucleotide sequence encoding a human UBE3A protein isoform operably linked downstream of the promoter sequence; and
iv) a 3′ ITR sequence downstream of the UBE3A nucleotide sequence;
   The protein component comprises:

an adeno-associated virus serotype 9 (AAV9) capsid,

wherein the polynucleotide is in the AAV9 capsid, and

wherein the polynucleotide does not include a secretion sequence.

In another aspect, the 5′ and 3′ ITR sequences are independently selected from the group consisting of adeno-associated virus serotype 1 (AAV1) ITRs, serotype 2 (AAV2) ITRs, serotype 3 (AAV3) ITRs, serotype 4 (AAV4) ITRs, serotype 5 (AAV5) ITRs, serotype 6 (AAV6) ITRs, serotype 7 (AAV7) ITRs, serotype 8 (AAV8) ITRs and serotype 9 (AAV9) ITRs. In another aspect, the 5′ and 3′ ITR sequences are independently from the group consisting of AAV1 ITRs, AAV2 ITRs, AAV4 ITRs, and AAV9 ITRs.

In another aspect, the 5′ and 3′ ITR sequences are both serotype 2 (AAV2) ITRs.

In certain aspects, the AAV9 capsid has an amino acid sequence of SEQ ID NO: 32 or SEQ ID NO: 27.

In another aspect, the 5′ and/or 3′ ITR sequence comprises a nucleotide sequence of SEQ ID NO: 22.

In another aspect, the AAV9 capsid is a mutant AAV9 (mAAV9) capsid selected from the group consisting of mAAV9.v1 having the amino acid sequence of SEQ ID NO: 32 and, mAAV9.v2 having the amino acid sequence of SEQ ID NO: 27.

In another aspect, the promoter sequence is a cytomegalovirus chicken-beta actin hybrid promoter, or human Ubiquitin ligase C promoter.

In another aspect, the promoter sequence is a human Ubiquitin ligase C promoter.

In another aspect, the UBE3A nucleotide sequence encodes human UBE3A isoform 1 having the amino acid sequence of SEQ ID NO: 4. In another aspect, the UBE3Av1 cDNA nucleotide sequence that encodes human UBE3A isoform 1 is SEQ ID NO:25.

In one aspect described herein, a method of delivering to a nerve cell in a brain of a living subject in need thereof comprising administering a therapeutically effective amount of a UBE3A vector via intracranial injection.

In another aspect, the therapeutically effective amount of the UBE3A vector is in a range from about 5×10⁶ viral genomes per gram (vg/g) to about 2.86×10¹² vg/g of brain mass, from about 4×10⁷ vg/g to about 2.86×10¹² vg/g of brain mass, or from about 1×10⁸ to about 2.86×10¹² vg/g of brain mass.

In another aspect, intracranial administration comprises bilateral injection.

In another aspect, administration via intracranial injection includes intrahippocampal or intracerebroventricular injection (ICV).

In another aspect, the administration is via intracerebroventricular injection.

In another aspect, the human UBE3A vector is transduced into at least two of hippocampus, auditory cortex, prefrontal cortex, stratum, thalamus, and cerebellum.

In another aspect, the subject treated according to a method of the invention has a UBE3A deficiency.

In another aspect, the UBE3A deficiency is Angelman Syndrome.

In another aspect, ICV injection of the human UBE3A vector restores UBE3A expression to wild type levels in at least two of the hippocampus, auditory cortex, prefrontal cortex and stratum.

In another aspect, ICV injection of the therapeutically effective amount of the UBE3A vector treats at least one symptom of Angelman Syndrome. In another aspect, the symptom of Angelman Syndrome treated comprises learning and memory deficits.

In another aspect, the method treats Angelman Syndrome by correcting a UBE3A protein deficiency in a subject in need thereof, the method comprising, administering a therapeutically effective amount of the UBE3A vector via intracranial injection to the subject.

One aspect described herein is a human UBE3A vector comprising:
a nucleic acid having
   i) a 5′ inverted terminal repeat (ITR) sequence;
   ii) a promoter downstream of the 5′ ITR sequence;
   iii) a UBE3A nucleotide sequence encoding a human UBE3A protein isoform 1 having SEQ ID NO: 4 operably linked downstream of the promoter; and,
   iv) a 3′ ITR sequence downstream of the UBE3A sequence; and
an adeno-associated virus serotype 5 (AAV5) capsid,
wherein the nucleic acid is packaged in the AAV5 capsid, and
wherein the nucleic acid does not include a secretion sequence. In another aspect, the UBE3A nucleotide sequence has SEQ ID NO: 24.

### BRIEF DESCRIPTION OF THE DRAWINGS

FIG. 1A shows a map of two versions of a UphUbe plasmid comprising a human ubiquitin ligase C promoter, a nucleotide sequence encoding a human UBE3A isoform 1 protein, a bovine growth hormone regulatory element with a poly A signal flanked by AAV2 ITRs, wherein the remaining elements are part of the plasmid backbone. The backbone includes an antibiotic resistance gene and a bacterial origin of replication. In the pTR-UphUbe plasmid of FIG. 1A(i) the antibiotic resistance gene is an ampicillin resistance gene, while in the pUphUbe/kan plasmid of FIG. 1A(ii) the antibiotic resistance gene is a Kanamycin resistance gene.

FIG. 1B shows the nucleotide sequence of the pTR-UphUbe plasmid (SEQ ID NO: 1) depicted in FIG. 1A(i).

FIG. 1C(i) shows the ITR-ITR nucleotide sequence (SEQ ID NO: 2) of the pTR-UphUbe plasmid depicted in FIG. 1A(i).

FIG. 1C(ii) shows the ITR-ITR nucleotide sequence (SEQ ID NO: 44) of the pUphUbe/kan plasmid of FIG. 1A(ii).

FIG. 1D shows the UBE3A genomic sequence of SEQ ID NO: 3.

FIG. 1E shows the nucleotide sequence of the UBE3Av1 cDNA (SEQ ID NO: 5) and the open reading frame (ORF) encoding the UBE3A Isoform 1 having an amino acid sequence of SEQ ID NO: 4.

FIG. 1F shows the nucleotide sequence of the UBE3Av1 coding region (SEQ ID NO: 25) having an open reading frame (ORF) encoding the UBE3A Isoform 1 polypeptide having an amino acid sequence of SEQ ID NO: 4.

FIG. 1G shows the nucleotide sequence of the UBE3Av2 cDNA (SEQ ID NO: 6) and the open reading frame (ORF) encoding the UBE3A Isoform 2 having an amino acid sequence of SEQ ID NO: 7.

FIG. 1H shows the nucleotide sequence of the UBE3Av3 cDNA (SEQ ID NO: 8) and the open reading frame (ORF) encoding the UBE3A Isoform 3 having an amino acid sequence of SEQ ID NO: 9.

FIG. 1I shows a comparison of the amino acid sequences of UBE3A isoforms 1, 2 and 3.

FIG. 1J shows the nucleotide sequences of AAV1-8 inverted terminal repeats (ITRs) (SEQ ID Nos: 14-21 respectively) identified from AAV1-8 genomic sequences reported in Genbank (Accession Nos. NC_002077.1, NC_001401.2, JB292182.1, NC_001829.1, NC_006152, AF028704.1, NC_006260.1 and NC_006261.1 respectfully) and scientific literature (Earley, L. F., et al. Hum Gene Ther (2020) 31(3-4): 151-162; Grimm D et al. J Virol (2006) 80:426-439; Chiorini et al., J. Virol. (1999) 73:1309-1319; Chiorini, J. A. et al. J. Virol. (1997) 71:6823-6833; Rutledge, E. A. et al. J. Virol. (1998) 72:309-319 and Xiao, W., N. et al. J. Virol. (1998) 73:3994-4003, the contents of which are incorporated by reference herein in their entireties). Shaded sequences show identity with AAV2 ITR sequence (SEQ ID NO: 15).

FIG. 1K shows the nucleotide sequence of SEQ ID NO: 30 that encodes the AAV9.1 capsid protein having an amino acid sequence of SEQ ID NO: 32.

FIG. 1L shows the nucleotide sequence of SEQ ID NO: 33 that encodes the AAV9.2 capsid protein having an amino acid sequence of SEQ ID NO: 27.

FIG. 1M shows an alignment of the amino acid sequences of wt AAV-9 capsid protein (SEQ ID NO: 28) with the amino acid sequence of mAAV9.2 capsid protein (SEQ ID NO: 27) and wt AAV9 capsid protein (SEQ ID NO: 28).

FIG. 1N shows the nucleotide sequence of SEQ ID NO: 35 that encodes UBE3A's AZUL domain having the amino acid sequence of SEQ ID NO: 36.

FIGS. 2A and B are graphs of the results of a quantitative polymerase chain reaction (qPCR), as described in Example 8, comparing copy numbers in the Hippocampus (HPC), Auditory Cortex (ACX), Prefrontal Cortex (PCX), Striatum (STR), Thalamus (THL) and Cerebellum (CER) of a nucleotide sequence encoding hUBE3A protein delivered by rAAV5 (FIG. 2A) and mrAAV9 (FIG. 2B) vectors in an Angelman Syndrome rat model dosed via intracerebroventricular (ICV) delivery with 10 μL, wherein the mrAAV9 vector includes a mutated adeno-associated serotype 9 (mAAV9.2) capsid with an amino acid sequence with two tyrosine mutations (SEQ ID NO: 28) and the rAAV5 vector includes an adeno-associated serotype 5 (AAV5) capsid.

FIG. 3A shows intensity of UBE3A protein distribution in the cortex normalized to actin in an Angelman Syndrome (AS) rat model dosed with 10 μL of the mrAAV9 vector described above compared to dosing AS rat models dosed with 10 μL of the rAAV5 vector and normal wild-type (wt) rat UBE3A protein expression levels, as described in Example 8. FIG. 3B shows percent (%) density in the cortex of the mrAAV9.2 vector compared to the rAAV5 vector and normalized to wt UBE3A expression levels. FIG. 3C shows intensity of hUBE3A protein distribution in the hippocampus normalized to actin in the Angelman Syndrome rat model. FIG. 3D shows percent (%) density in the hippocampus of the mrAAV9.2 vector compared to the rAAV5 vector and normalized to wt UBE3A expression levels.

FIG. 4 shows copy numbers of the nucleotide sequence encoding hUBE3A found in brain regions in an Angelman Syndrome rat model dosed via ICV with 50 μL of the mrAAV9.2 vector compared to the rAAV5 vector, as determined by qPCR.

FIG. 5A shows E6AP protein expression as a percent of wild type expression as measured in brain regions in the AS rat model after treatment with the mrAAV9.2 vector, rAAV5 vector and vehicle compared to wild type E6AP expression levels. FIG. 5B shows E6AP protein expression as a percent of wild type expression as measured in the cerebral spinal fluid in the AS rat model after treatment with the mrAAV9.2 vector, rAAV5 vector and vehicle compared to wild type E6AP expression levels.

FIG. 6 shows E6AP protein expression as a percent of wild type expression as measured in brain regions in the AS rat model after treatment with the mrAAV9.2 vector (v9) and vehicle compared to wild type E6AP expression levels.

FIG. 7A shows Western blot results of protein expression in the hippocampus and cortex regions in the AS rat model after treatment with the mrAAV9.2 vector. FIG. 7B shows Western blot results of protein expression in the prefrontal cortex and striatum regions in the AS rat model after treatment with the mrAAV9.2 vector. FIG. 7C shows Western blot results of protein expression in the thalamus and midbrain/brainstem regions in the AS rat model after treatment with the mrAAV9.2 vector. FIG. 7D shows Western blot results of protein expression in the cerebellum region in the AS rat model after treatment with the mrAAV9.2 vector.

FIG. 8 shows rAAV5 containing the human UBE3A gene can increase E6AP expression in the AS mouse. (A) Insertion of hUBE3A variant included a CBA promotor for mRNA transcription and flanked by AAV2 terminal repeats. (B-D) Immuno-staining of ICV injected animals showed an increase in E6AP expression in AAV5-hUBE3A injected AS mice (C) compared to AAV5-GFP injected AS animals (B). Scale bar set at 700 microns. (E) E6AP protein was detectable by Western blotting in the hippocampus, striatum, prefrontal cortex, and cerebellum of AS mice injected with AAV5-hUBE3A (AAV5-hUBE3A n=4 per region, sham injected WT n=4 per region). AAV5-GFP injected mice showed no measurable levels of E6AP and therefore are not listed. (F) Injection of AAV5-hUBE3A by ICV markedly increased protein expression in the hippocampus compared to sham injected WT controls (n=4 per group). (G) Representative Western blot of E6AP and actin in the hippocampus showed increased E6AP protein. (H) Representative Western blot for E6AP and actin in the cortex showed detectable E6AP protein. HPC: Hippocampus, STR: Striatum, PFC: Prefrontal cortex, CTX: Cortex, CER: Cerebellum.

FIG. 9 shows reduced movement and compulsive behaviors in AS. (A) Distance traveled in the open field test showed a significant increase in sham injected WT mice compared to both AS groups (*p<0.0001). (B) No change in anxiety was observed as measured by immobility in the center region of the open field. (C) No anxiety behavior was detected with time spent in the open arms of the elevated plus maze. (D) Marble burying showed a significant increase in compulsive behavior with number of marbles buried in sham injected WT mice only (*p<0.0001).

FIG. 10 shows motor coordination did not change with injection of AAV5-hUBE3A. (A) Training of mice on a 4-40 rpm Rotorod showed a significant difference in latency to fall between sham injected WT and both AS mice treatments in trials 4-8 (2-way ANOVA p<0.05). (B) Significant increase in time spent on rod is seen from trial 1 to trial 8 in all groups tested (p<0.05 between trial 1 to 8). (C) Correlating weight with average time spent on rod for trial 8 indicated that regardless of treatment, AS mice are heavier and spend less time on rod.

FIG. 11 shows ICV injection of AAV5-hUBE3A in AS mice improved spatial memory in the hidden platform water maze task. (A) Latency to locate escape platform during 5 days of training improved over time. (B) Swim speed (cm/s) during training indicated sham injected WT mice swam faster (2-way ANOVA). (C) Number of platform crosses in each platform location during a probe trial taken 72 hours after last training session showed AAV5-hUBE3A injected AS mice performed significantly better than AAV5-GFP injected AS mice (*p<0.05). (D) No differences were seen between treatments in time spent in each quadrant during the probe trial. (E) Sham injected WT mice swam a longer distance (m) than both AS groups during the probe trial (*p<0.05). (F) Sham injected WT mice swam faster (cm/sec) than AS mice during the probe trial (*p<0.05). (G) Representative occupancy plots of AAV5-hUBE3A AS mice, AAV5-GFP AS mice, and sham injected WT mice during the probe trial. T: Location of target platform for training.

FIG. 12 shows the recovery of synaptic plasticity deficits after AAV5-hUBE3A ICV injection. (A) Synaptic response was measured through an input-output curve (change in fiber-volley amplitude versus slope of the fEPSP; AAV5-hUBE3A n=11, AAV5-GFP n=41, sham injected WT n=31). (B) Paired-pulse facilitation measured by percent change in the fEPSP slopes between 2 stimulations given at increasing time points (AAV5-hUBE3A n=22, AAV5-GFP n=55, sham injected WT n=37). (C) Stabile baseline recordings were obtained before initiating tbs (θ: tbs). Changes in slopes of the fEPSP recordings indicated synaptic plasticity changes between AAV treatments (AAV5-hUBE3A n=15, AAV5-GFP n=46, sham injected WT n=44). (D) Average of the last 10 minutes of recording indicated a significant decrease in AAV5-GFP AS mice to all other groups (p<0.0001). (E) Representative traces of all three groups. Grey line: baseline trace; black line: trace at 60 minutes post tbs. Scale bar 2 mV/2 ms.

### DETAILED DESCRIPTION

One aspect described herein is a UBE3A vector comprising, a nucleic acid comprising:

i) a 5′ inverted terminal repeat (ITR) sequence;

ii) a promoter downstream of the 5′ ITR sequence;

iii) a UBE3A nucleotide sequence encoding a hUBE3A protein isoform operably linked downstream of the promoter; and,

iv) a 3′ ITR sequence downstream of the UBE3A sequence; and

an AAV9 capsid,

wherein the nucleic acid is packaged in the AAV9 capsid, and wherein the nucleic acid does not include a secretion sequence.

In another aspect, the 5′ and 3′ ITR sequences are independently selected from the group consisting of AAV1 ITRs, AAV2 ITRs, AAV3 ITRs, AAV4 ITRs and AAV9 ITRs.

In another aspect, the 5′ and 3′ ITR sequences are both AAV2 ITRs.

In another aspect, the 5′ and/or 3′ ITR sequence comprises a nucleotide sequence of SEQ ID NO: 22.

In another aspect, the AAV9 capsid is a mutant AAV9 capsid selected from the group consisting of mAAV9.v1 having the amino acid sequence SEQ ID NO: 32; and mAAV9.v2 having the amino acid sequence SEQ ID NO: 27.

In another aspect, the promoter sequence is a cytomegalovirus chicken-beta actin hybrid promoter, or human ubiquitin ligase C promoter.

In another aspect, the promoter sequence is a human ubiquitin ligase C promoter.

In another aspect, the UBE3A nucleotide sequence encodes hUBE3A isoform 1 having the amino acid sequence of SEQ ID NO: 4.

One aspect described herein is a method of delivering to a nerve cell in a brain of a living subject in need thereof comprising, administering a therapeutically effective amount of the UBE3A vector of the disclosure via intracranial injection to the subject.

In another aspect, the therapeutically effective amount of the UBE3A vector can range between about 5×10⁶ viral genomes per gram (vg/g) to about 2.86×10¹² vg/g of brain mass, from about 4×10⁷ vg/g to about 2.86×10¹² vg/g of brain mass, or from about 1×10⁸ to about 2.86×10¹² vg/g of brain mass.

In another aspect, intracranial administration comprises bilateral injection.

In another aspect, administration via intracranial injection includes intrahippocampal or intracerebroventricular injection. In another aspect, administration is via intracerebroventricular injection.

In another aspect, the administration is via intracerebroventricular injection.

In another aspect, the human UBE3A vector is transduced into at least two of hippocampus, auditory cortex, prefrontal cortex, stratum, thalamus, and cerebellum.

In another aspect, the subject treated according to a method of the invention has a UBE3A deficiency.

In another aspect, the UBE3A deficiency is Angelman Syndrome.

In another aspect, ICV injection of the human UBE3A vector restores UBE3A expression to wild type levels in at least two of the hippocampus, auditory cortex, prefrontal cortex and stratum.

In another aspect, ICV injection of the therapeutically effective amount of the UBE3A vector treats at least one symptom of Angelman Syndrome. In another aspect, the symptom of Angelman Syndrome treated comprises learning and memory deficits.

In another aspect, the method treats Angelman Syndrome by correcting a UBE3A protein deficiency in a subject in need thereof comprising, administering a therapeutically effective amount of the UBE3A vector via intracranial injection to the subject.

### Definitions

As used herein, all numerical designations, such as pH, temperature, time, concentration, molecular weight, dosage amounts, including ranges, are approximations which may be varied up or down by increments of 1.0 or 0.1, as appropriate. It is to be understood, even if it is not always explicitly stated that all numerical designations are preceded by the term “about”. It is also to be understood, even if it is not always explicitly stated, that the reagents described herein are merely exemplary and that equivalents of such are known in the art and can be substituted for the reagents explicitly stated herein.

As used herein, the term “about” means a numerical value that is approximately or nearly the same as the value to which it refers or within a range of such value to the degree that the value may be in the range of ±15% of the stated value.

As used herein, the singular forms “a,” “an” and “the” include, without limitation, plural forms of the aspects described herein unless usage clearly dictates otherwise. Thus, for example, reference to “a polypeptide,” “a vector,” “a plasmid” and the like may include at least one or more of the aspects described.

A “subject” is a mammal (e.g., a non-human mammal), more preferably a primate and still more preferably a human. Mammals include, but are not limited to, primates, humans, farm animals, rodents, sport animals, and pets.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one aspect, to at least one, optionally including more than one, A, with no B present (and optionally including elements other than B); in another aspect, to at least one, optionally including more than one, B, with no A present (and optionally including elements other than A); in yet another aspect, to at least one, optionally including more than one, A, and at least one, optionally including more than one, B (and optionally including other elements); etc.

When a range of values is listed herein, it is intended to encompass each value and sub-range within that range. For example, “1-5 ng” is intended to encompass 1 ng, 2 ng, 3 ng, 4 ng, 5 ng, 1-2 ng, 1-3 ng, 1-4 ng, 1-5 ng, 2-3 ng, 2-4 ng, 2-5 ng, 3-4 ng, 3-5 ng, and 4-5 ng.

As used herein, the term “promoter” refers generally to proximal promoters found in the 5′ flanking region of protein-coding genes that facilitates the binding of transcription factors required for their transcription by RNA polymerase II. In certain aspects, the promoter may further comprise an enhancer and other position independent cis-acting regulatory elements that enhance transcription from the proximal promoter such as scaffold/matrix attachment region (S/MAR) element. In certain aspects, genes transcribed by RNA polymerase III can have their promoter located within the gene itself, i.e. downstream of the transcription start site.

In certain aspects, the transgene may comprise a protein-coding region operably linked to either a constitutive, inducible or tissue-specific promoter.

As used herein, the term “expression” includes transcription and translation.

As used herein, the term “gene” refers to a DNA sequence that encodes through its template or messenger RNA a sequence of amino acids characteristic of a specific peptide, polypeptide, or protein. The term “gene” also refers to a DNA sequence that encodes a non-coding RNA product. The term gene as used herein with reference to genomic DNA includes intervening, non-coding regions as well as regulatory regions and can include 5′ and 3′ ends.

As used herein, the term “transcription regulatory sequence” refers to a DNA sequence that controls and regulates the transcription and/or translation of another DNA sequence. In eukaryotes, transcription regulatory sequences include, but are not limited to, promoters, enhancers, polyadenylation signals and silencers.

As used herein, the term “endogenous” refers to nucleic acid and/or amino acid sequence naturally occurring in the cell of interest.

As used herein, the term “exogenous” refers to a heterologous nucleic acid and/or amino acid sequence that is not normally found in the cell of interest. For example, a transgene refers to a heterologous nucleic acid sequence that is introduced into a cell of interest by transfection.

As used herein the term “a secretion sequence” (sometimes referred to as signal sequence, signal peptide, targeting signal, localization signal, localization sequence, transit peptide, leader sequence, leader peptide, secretion signal peptide) refers to a N terminal short peptide (usually 16-30 amino acids long) in newly synthesized proteins that are destined towards the secretory pathway. The secretion sequence is comprised of a hydrophilic, usually positively charged N-terminal region, a central hydrophobic domain and a C-terminal region that is cleaved by signal peptidase. Besides these common characteristics, signal sequences do not share sequence similarity, and some are more than 50 amino acid residues long.

As used herein, a secretion sequence is an added nucleotide sequence encoding a signal peptide that is ligated in frame to the UBE3A nucleotide sequence.

In one aspect, the secretion sequence is an added nucleotide sequence encoding a signal peptide that is ligated in frame to the 5′ end of the UBE3A nucleotide sequence (corresponding to the N terminus of the UBE3A polypeptide).

Exemplary secretion sequences include:

the secretion sequence of the glial cell derived
neurotrophic factor (GDNF) gene:
(SEQ ID No: 41)
ATGAAGTTATGGGATGTCGTGGCTGTCTGCCTGGTGCTGCTCCACACCGCG
TCCGC,
the secretion sequence of the insulin protein:
(SEQ ID No: 42)
ATGGCCCTGTGGATGCGCCTCCTGCCCCTGCTGGCGCTGCTGGCCCTCTGG
GGACCTG ACCCAGCCGCAGCC (AH002844.2),
or
the secretion sequence of the IgK;
(SEQ ID No: 43)
ATGGAGACAGACACACTCCTGCTATGGGTACTGCTGCTCTGGGTTCCAGG
TTCCACTGGT (NG 000834.1).

In one aspect, the UBE3A nucleotide sequence does not contain a secretion sequence.

As used herein, the term “transfection” refers to the introduction of an exogenous nucleotide sequence, such as DNA vectors in the case of mammalian target cells, into a target cell whether or not any coding sequences are ultimately expressed. Numerous methods of transfection are known to those skilled in the art, such as: chemical methods (e.g., calcium-phosphate transfection), physical methods (e.g., electroporation, microinjection, and particle bombardment), fusion (e.g., liposomes), receptor-mediated endocytosis (e.g., DNA-protein complexes, viral envelope/capsid-DNA complexes), nanoparticles or by transduction with recombinant viruses.

As used herein, the term “construct” refers to a recombinant genetic molecule having one or more isolated polynucleotide sequences. Genetic constructs used for transgene expression in a host organism include in the 5′-3′ direction, a promoter sequence; a sequence encoding a gene of interest; and a polyadenylation sequence. The construct may also include selectable marker gene(s) and other regulatory elements for expression.

As used herein, the term “UBE3A vector” refers to a nucleic acid which includes a UBE3A nucleotide sequence encoding a hUBE3A protein isoform and flanking ITR sequences encapsulated in an AAV capsid. In one aspect, an AAV capsid is selected from rAAV2, rAAV3, rAAV4, rAAV5, rAAV5, rAAV6, rAAV7, rAAV8, rAAV10, rAAV11, rAAV12, mrAAV2, mrAAV5 rAAV9, having the SEQ ID NO: 28, mrAAV9.1 having the amino acid sequence of SEQ ID NO: 32; or mrAAV9.2 having the amino acid sequence of SEQ ID NO: 27. In one aspect, the nucleic acid is packaged in an AAV9 capsid. In another aspect, the AAV9 capsid is a mAAV9 capsid selected from the group consisting of mAAV9.v1 having the amino acid sequence of SEQ ID NO: 32, and, mAAV9.v2 having the amino acid sequence of SEQ ID NO: 27. In another aspect, the nucleic acid is packaged in an AAV5 capsid.

As used herein, the term “adeno-associated virus (AAV) capsid” refers to an AAV capsid that is engineered for specific functionality, tissue penetration or tissue permeability for use in a gene therapy. In one aspect, the AAV capsid can be obtained from a recombinant adeno-associated virus (rAAV) plasmid. In another aspect, the AAV capsid can be obtained from a mutated adeno-associated virus (mrAAV) plasmid, wherein one or more amino acids within the wild type amino acid sequence are each replaced with a non-endogenous amino acid to enhance specific functionality, tissue penetration or tissue permeability for use in a gene therapy.

In one aspect, the capsid amino acid sequence comprises a mutation, wherein one or more tyrosine (Tyr) amino acids are each mutated to a phenylalanine (Phe) amino acid.

In one aspect, the AAV capsid for use herein includes, but is not limited to, an AAV2, AAV5 or AAV9 capsid. In another aspect, an AAV9 capsid is described for use herein. In another aspect, a mutated AAV9 capsid is described for use herein.

In another aspect, the wild-type AAV2 capsid is mutated, wherein one or more Tyr amino acids are mutated to a Phe amino acid. In another aspect, the AAV2 capsid amino acid sequence is mutated, wherein certain Tyr amino acids are each mutated to a Phe amino acid.

In another aspect, the wild-type AAV5 capsid is mutated, wherein one or more Tyr amino acids are mutated to a Phe amino acid. In another aspect, the AAV5 capsid sequence is mutated, wherein certain Tyr amino acids are each mutated to a Phe amino acid.

In another aspect, the wild-type AAV9 capsid is mutated, wherein one or more Tyr amino acids are mutated to a Phe amino acid. In another aspect, the AAV9 capsid sequence is mutated, wherein certain Tyr amino acids are each mutated to a Phe amino acid. In another aspect, the AAV9 capsid sequence is mutated, wherein the Tyr cDNA at position 445 is mutated to encode a Phe amino acid. In another aspect, the AAV9 capsid sequence is mutated, wherein the Tyr amino acid at each of positions 445 and 731 is mutated to encode a Phe amino acid.

As used herein, the term “administration” or “administering” describes the process in which an UBE3A vector described herein, alone or in combination with another therapy, is delivered to a patient. In one aspect, the UBE3A vector may be administered to a nerve cell in a brain of a subject in need thereof via intracranial injection to the subject including, but not limited to, by intrastriatal, intrahippocampal, ventral tegmental area (VTA) injection, intracerebral, intracerebellar, intramedullary, intranigral, intracerebroventricular, intracisternal, intracranial or intraparenchymal injection. In another aspect, administration via intracranial injection is selected from intrahippocampal or intracerebroventricular injection. In another aspect, intracranial administration includes bilateral injection.

As used herein, the terms “treatment” or “treating” refer to any effect of alleviation, amelioration, elimination, stabilization or delay in progression of Angelman Syndrome or a symptom thereof resulting from administration of the UBE3A vector described herein to a subject in need thereof. In one aspect, “treatment” of Angelman Syndrome may include any one or more of the following: amelioration and/or elimination of one or more symptoms associated with Angelman Syndrome, reduction of one or more symptoms of Angelman Syndrome, stabilization of symptoms of Angelman Syndrome, or delay in progression of one or more symptoms of Angelman Syndrome.

As used herein, the terms “prevention” or “preventing” refer to any effect of halting the progression of Angelman Syndrome, reducing the effects of Angelman Syndrome, reducing the incidence of Angelman Syndrome, reducing the development of Angelman Syndrome, delaying the onset of symptoms of Angelman Syndrome, increasing the time to onset of symptoms of Angelman Syndrome, and reducing the risk of development of Angelman Syndrome.

As used herein, the term “animal” refers to a multicellular, eukaryotic organism classified in the kingdom Animalia or Metazoa. The term includes, but is not limited to, mammals. Non-limiting examples include rodents, mammals, aquatic mammals, domestic animals such as dogs and cats, farm animals such as sheep, pigs, cows and horses, and humans. Wherein the terms “animal” or the plural “animals” are used, it is contemplated that it also applies to any animals.

As used herein, the term “therapeutically effective amount” refers to that amount of a therapy (e.g., a therapeutic agent or vector) sufficient to result in the treatment, prevention or amelioration of Angelman syndrome or other UBE3A-related disorder or one or more symptoms thereof, prevent advancement of Angelman syndrome or other UBE3A-related disorder, or cause regression of Angelman syndrome or other UBE3A-related disorder. In one aspect, a dose that prevents or alleviates (i.e., reduces or eliminates) a symptom in a patient when administered one or more times over a suitable time period may be considered a therapeutically effective amount.

The dosing of the vector described herein to obtain a therapeutic or prophylactic effect is determined by the circumstances of the patient, as known in the art. The dosing of a patient herein may be accomplished through individual or unit doses of the vector described herein or by a combined or prepackaged or pre-formulated dose of the vector described herein. An average 40 g mouse has a brain weighing 0.416 g; therefore, a 160 g mouse has a brain weighing 1.02 g, and a 250 g mouse has a brain weighing 1.802 g. An average human brain weighs 1508 g, which can be used to direct the amount of therapeutic needed or useful to accomplish the treatment described herein.

The vector described herein may be administered individually, or in combination with or concurrently with one or more other therapeutics for neurodegenerative disorders, specifically UBE3A protein deficiency disorders.

As used herein “patient” is used to describe an animal, preferably a human, to whom treatment is administered, including prophylactic treatment with the vector described herein.

“Neurodegenerative disorder” or “neurodegenerative disease” as used herein refers to any abnormal physical or mental behavior or experience where the death or dysfunction of neuronal cells is involved in the etiology of the disorder. Further, the term “neurodegenerative disease” as used herein describes “neurodegenerative diseases” which are associated with UBE3A deficiencies resulting in Angelman Syndrome.

The term “UBE3A deficiency” as used herein can refer to a deficiency in UBEA protein due to a mutation or deletion in the UBE3A gene sequence.

The term “normal” or “control” as used herein refers to a sample or cells or patient which are assessed as not having Angelman syndrome or any other neurodegenerative disease or any other UBE3A deficient neurological disorder.

### Recombinant AAV Vector

The nucleic acid component of the human UBE3A vector disclosed herein is a recombinant AAV vector. Recombinant AAV (rAAV) vectors are typically composed of, at a minimum, a transgene and its regulatory sequences, and 5′ and 3′ AAV inverted terminal repeats (ITRs). It is this recombinant AAV vector which is packaged into a capsid protein and delivered to a selected target cell. In some aspects, the transgene is a nucleic acid sequence, heterologous to the vector sequences, which encodes a polypeptide, protein, functional RNA molecule (e.g., miRNA, miRNA inhibitor) or other gene product, of interest. The nucleic acid coding sequence is operatively linked to regulatory components in a manner which permits transgene transcription, translation, and/or expression in a cell of a target tissue.

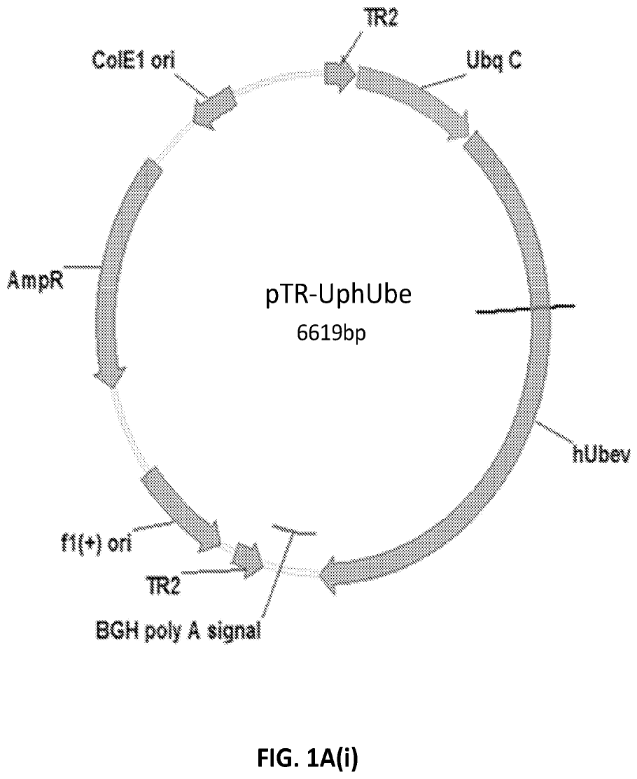

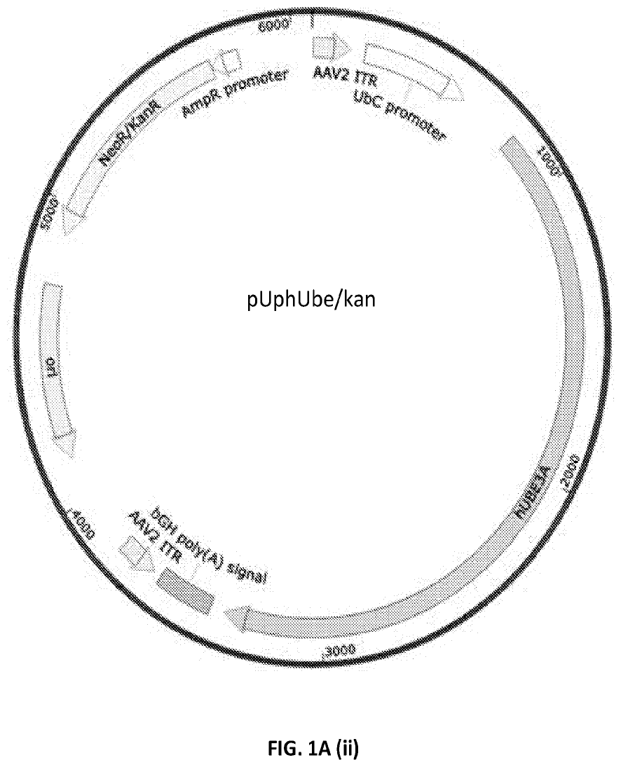

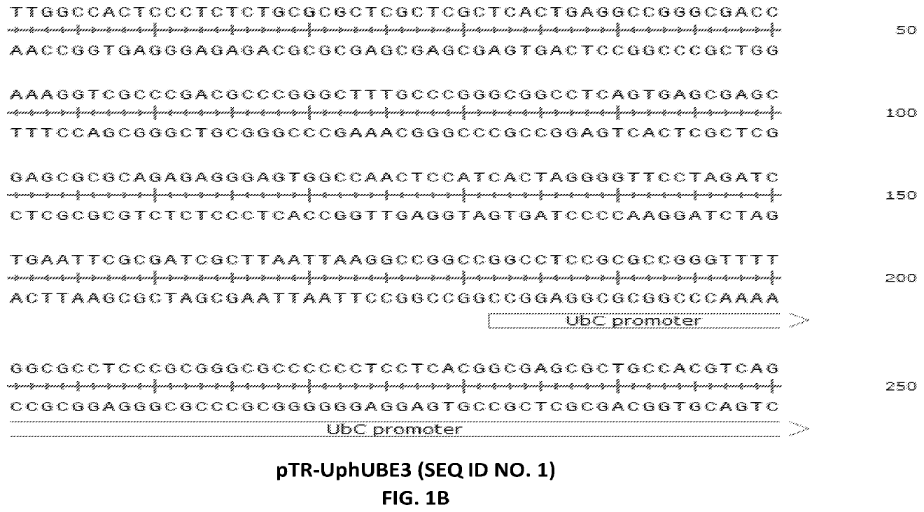

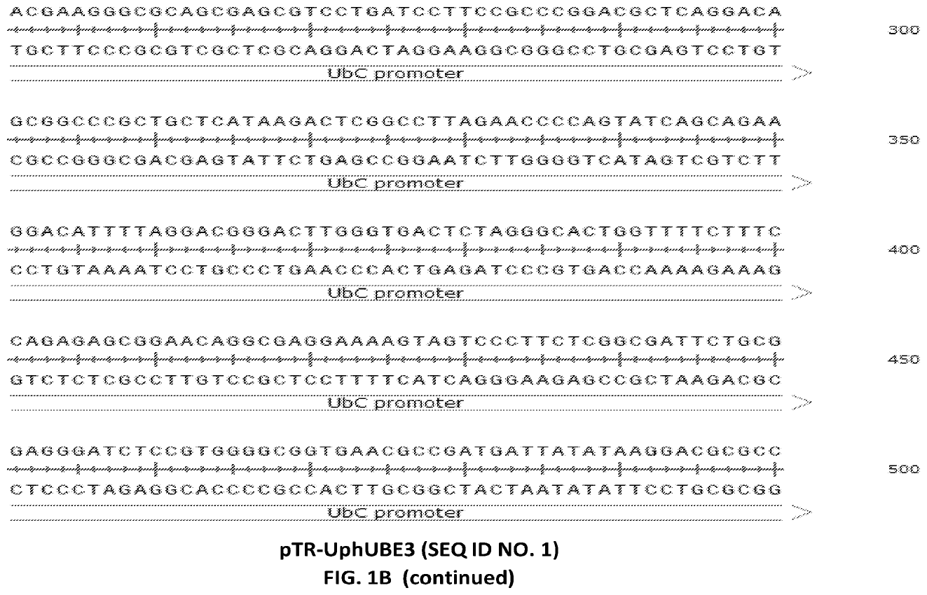

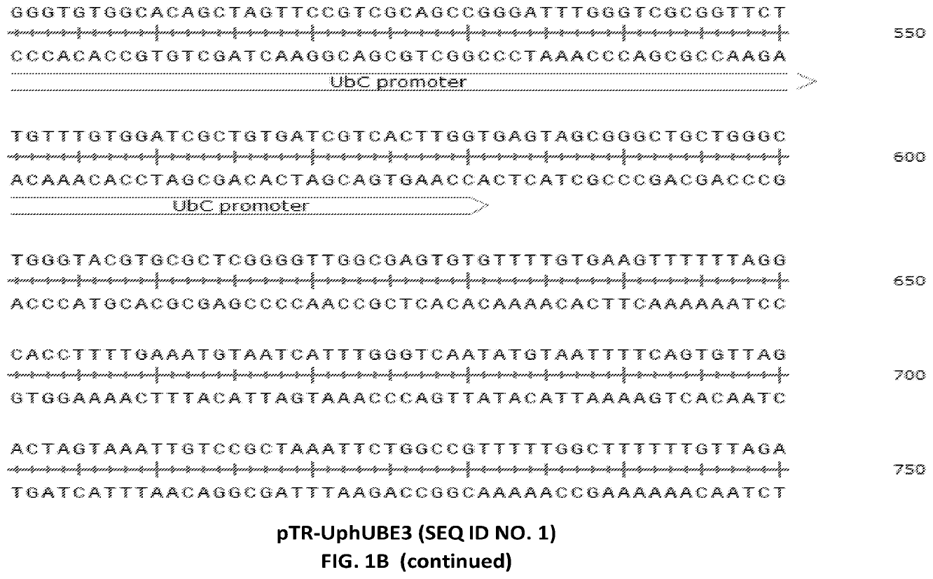

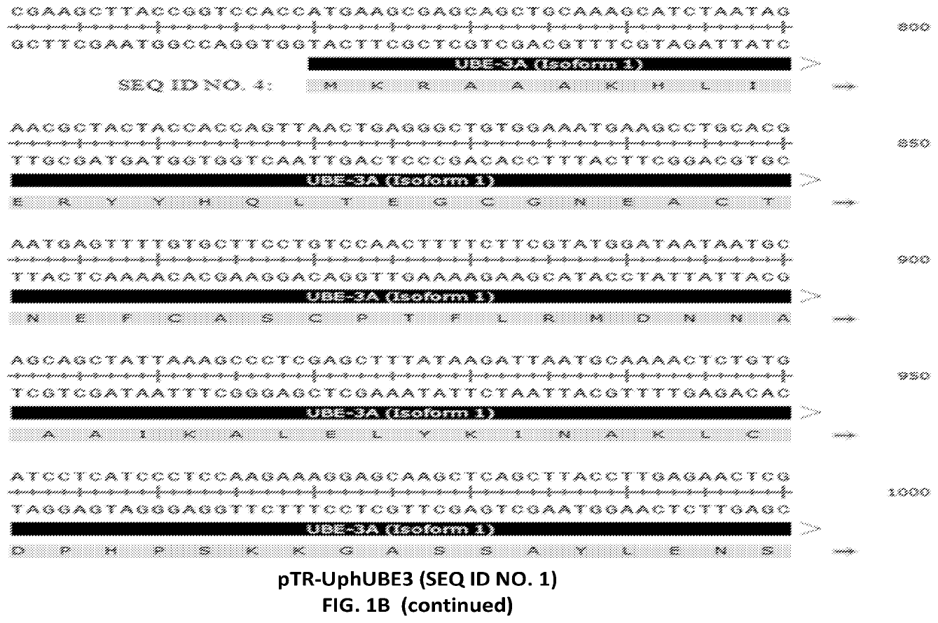

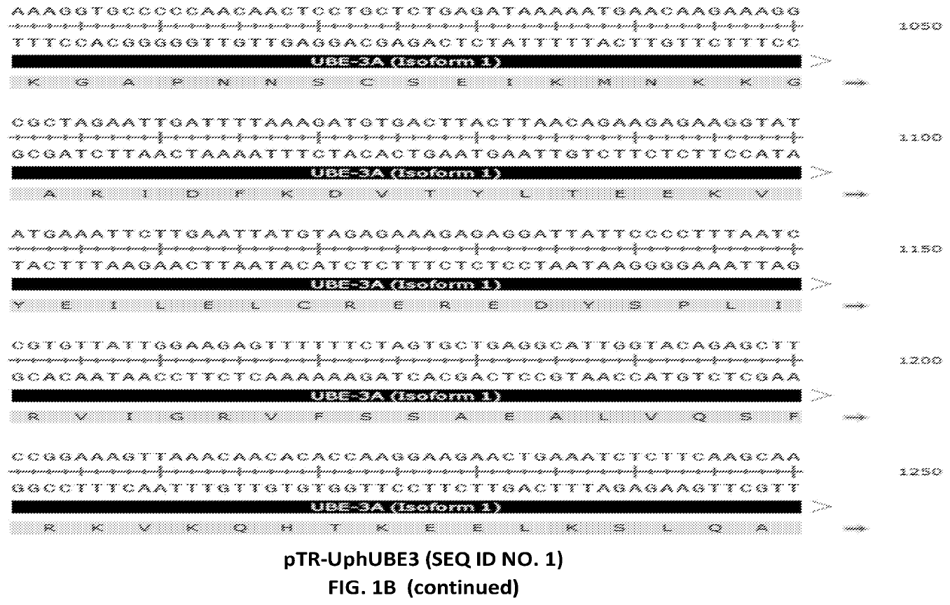

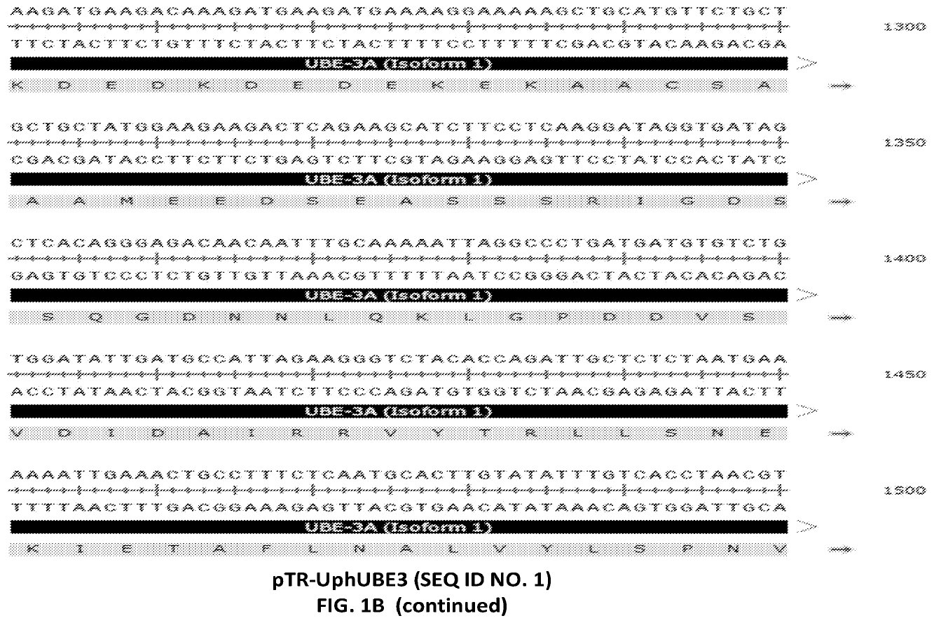

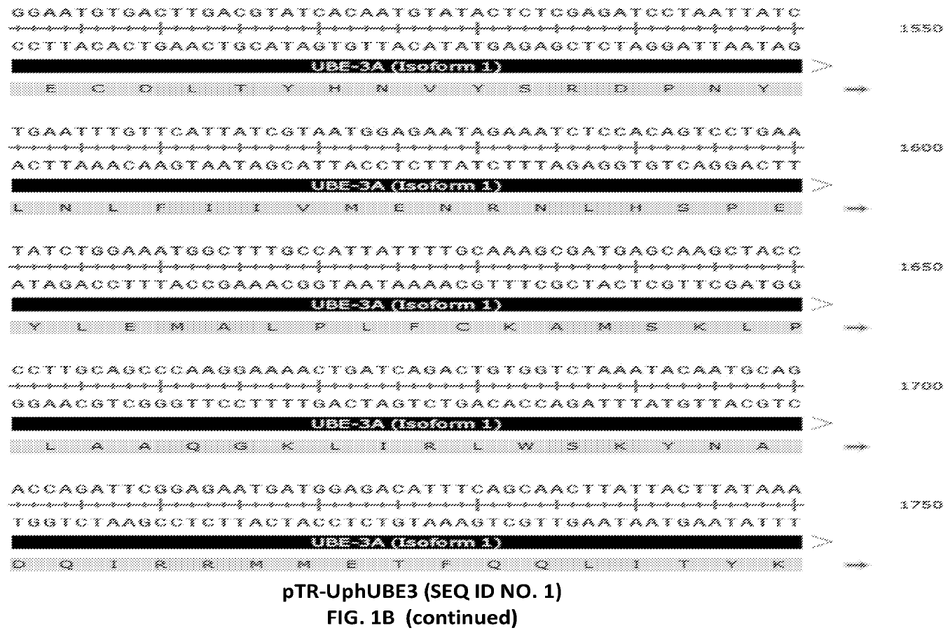

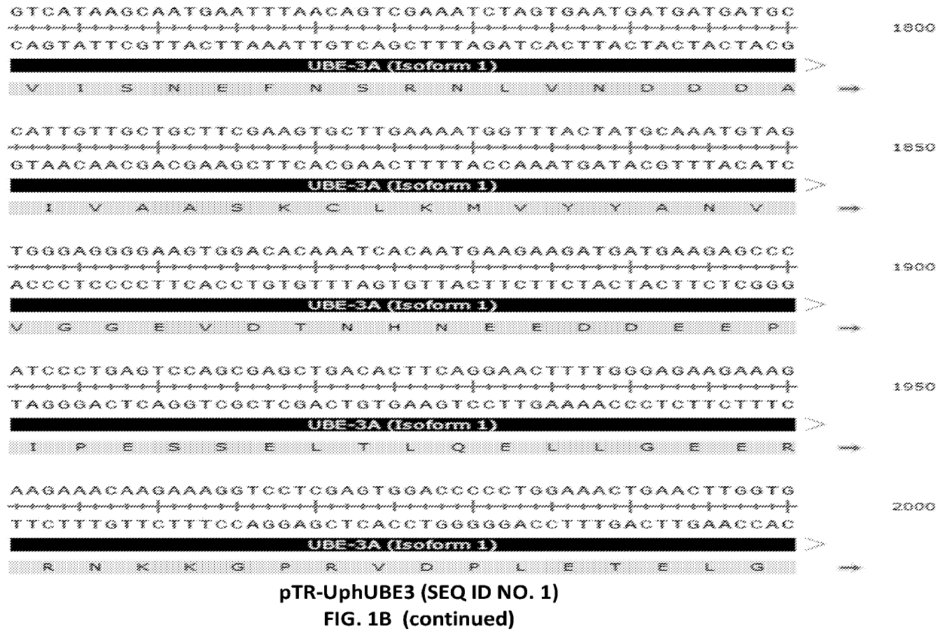

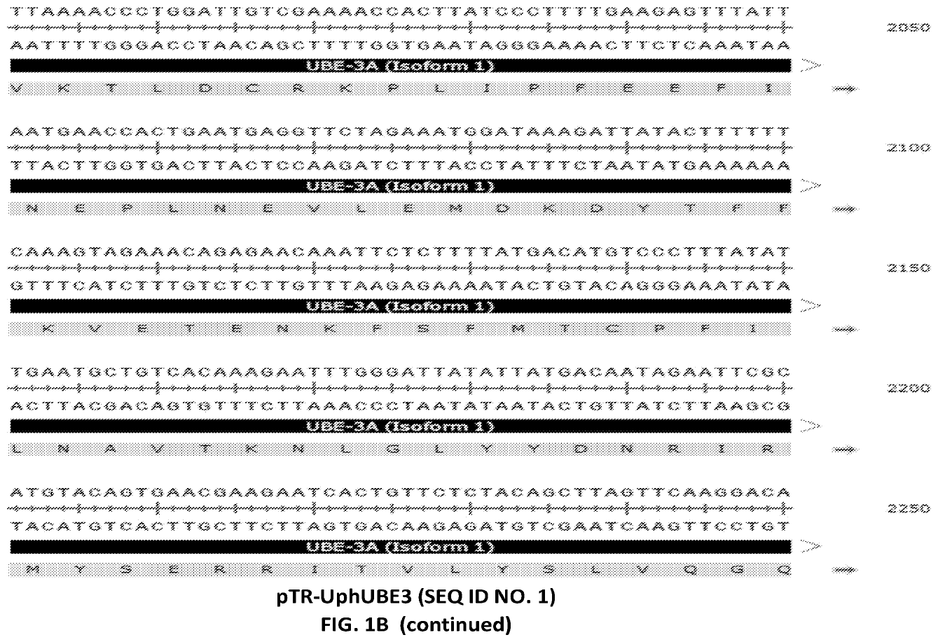

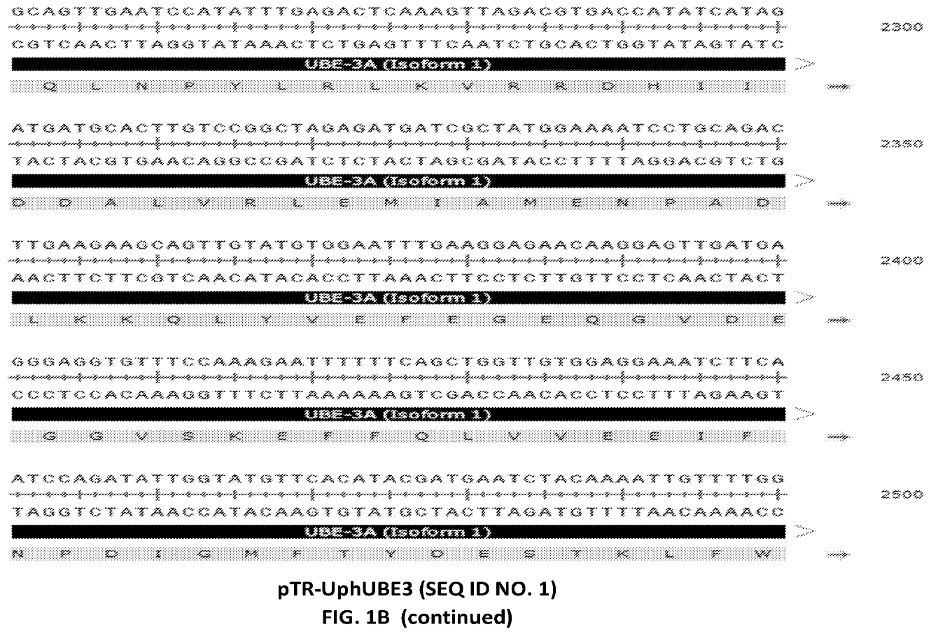

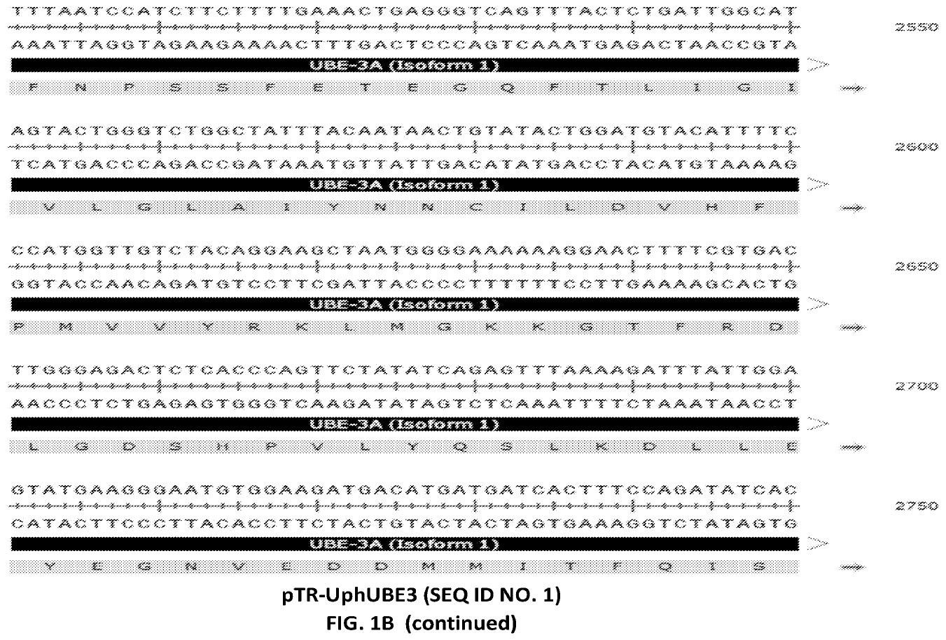

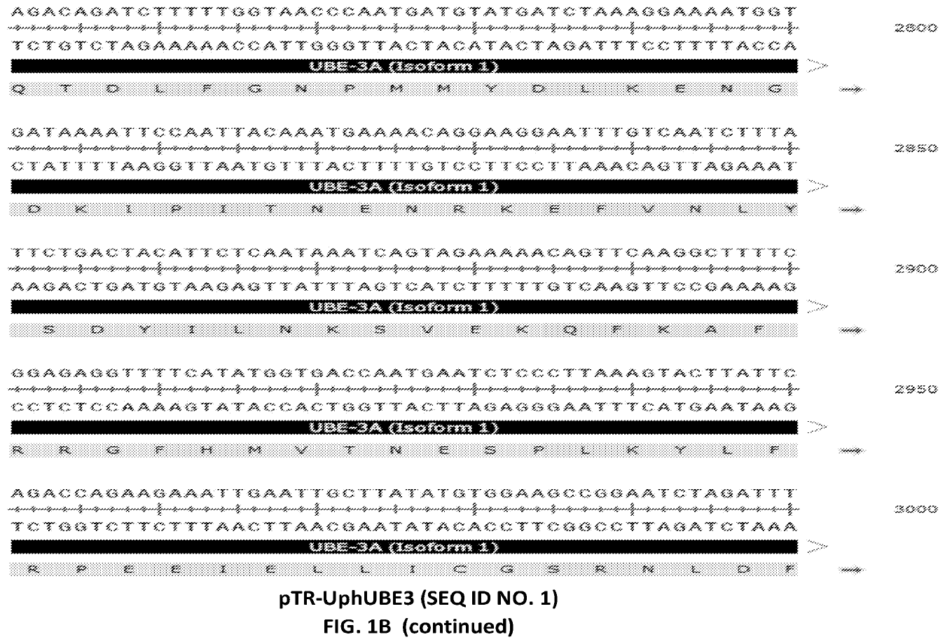

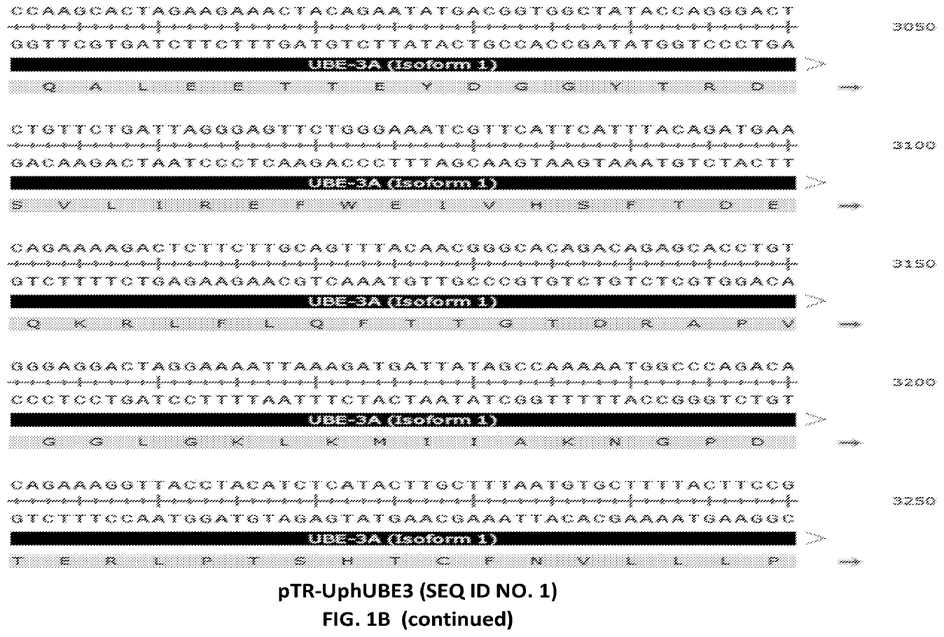

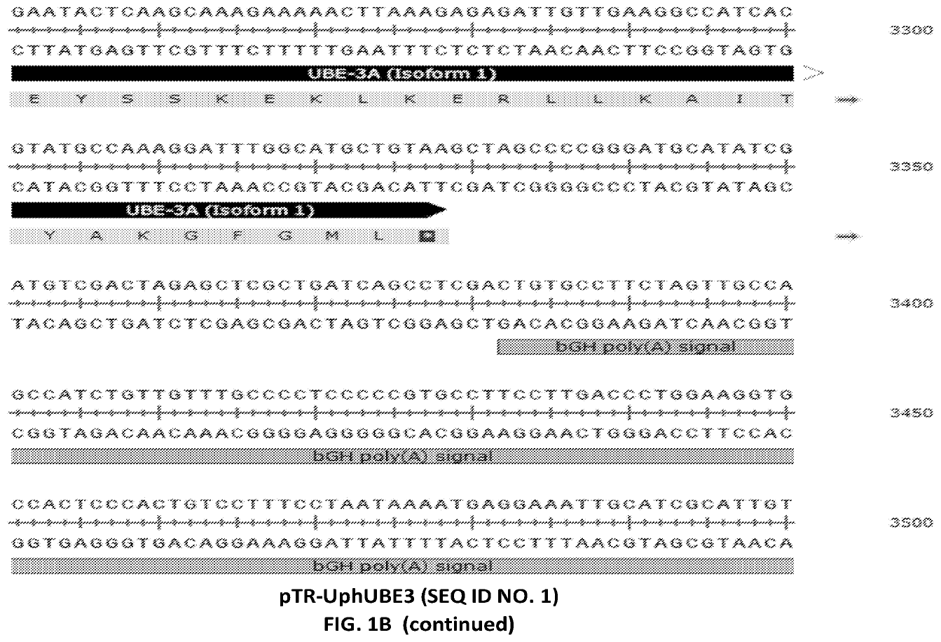

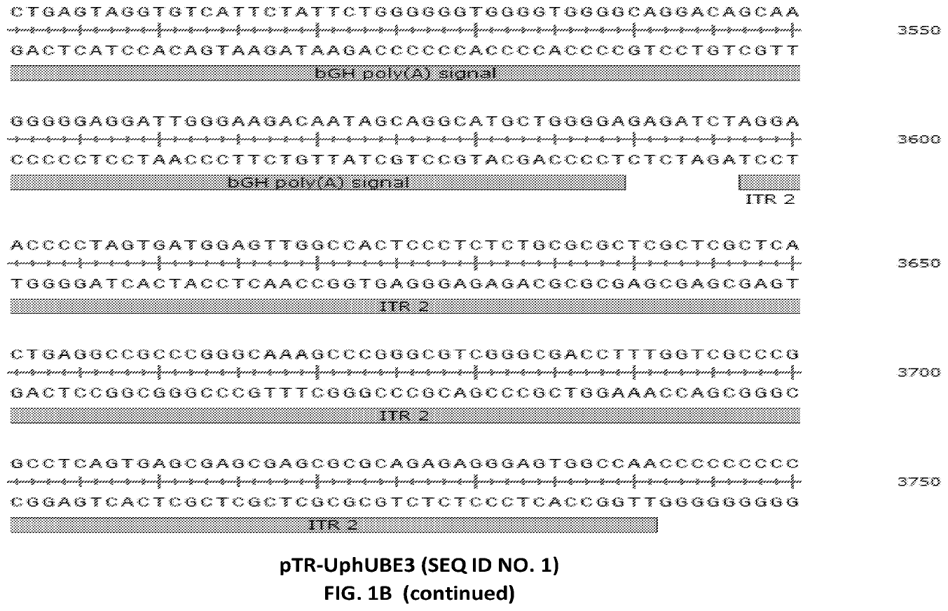

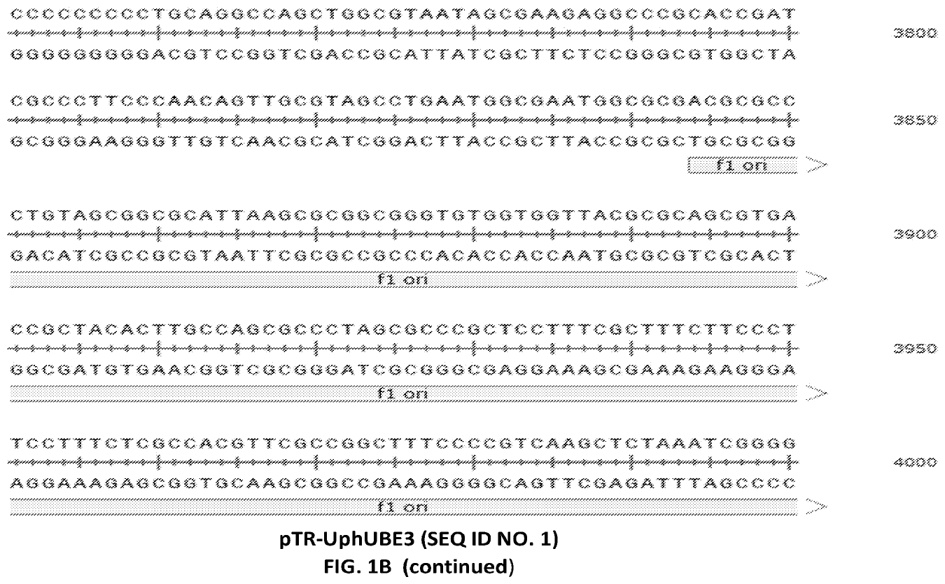

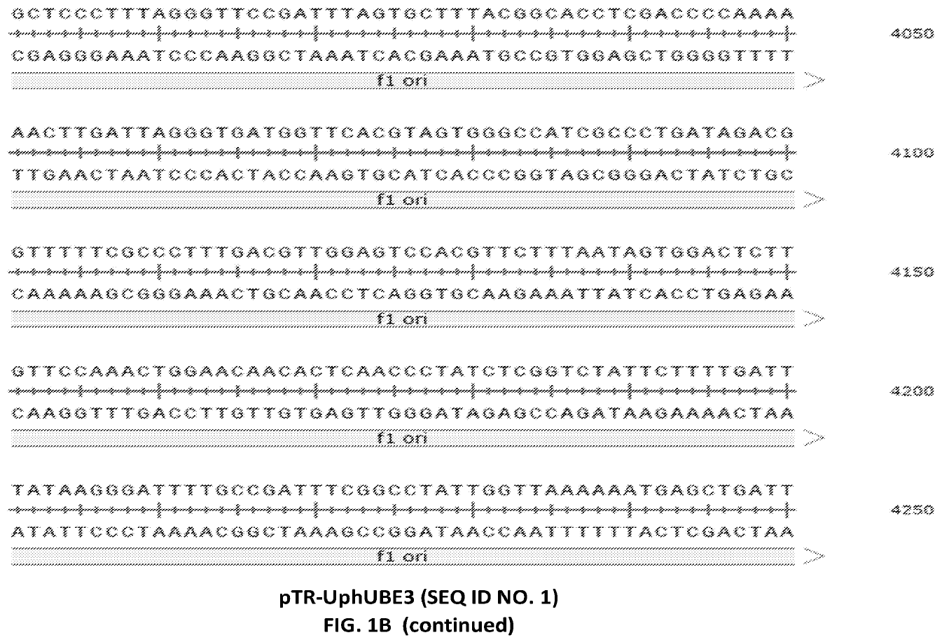

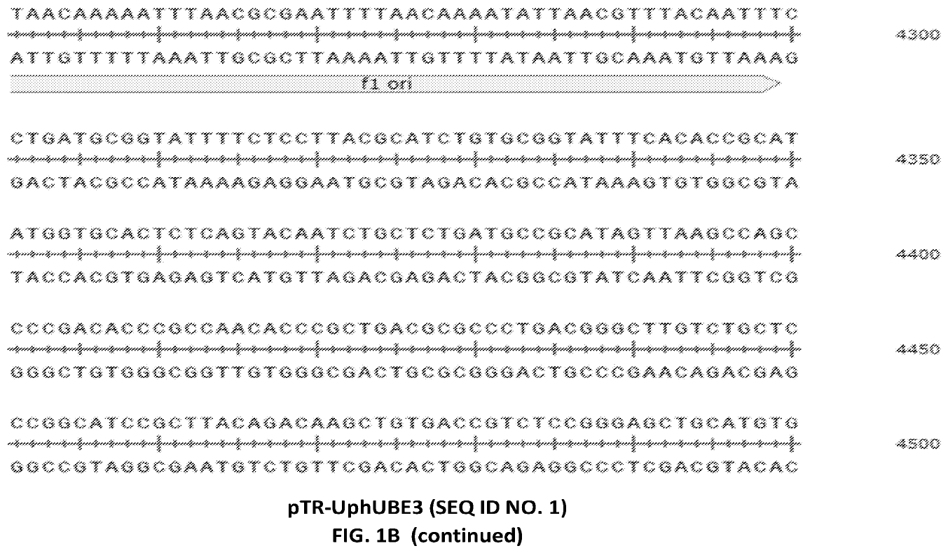

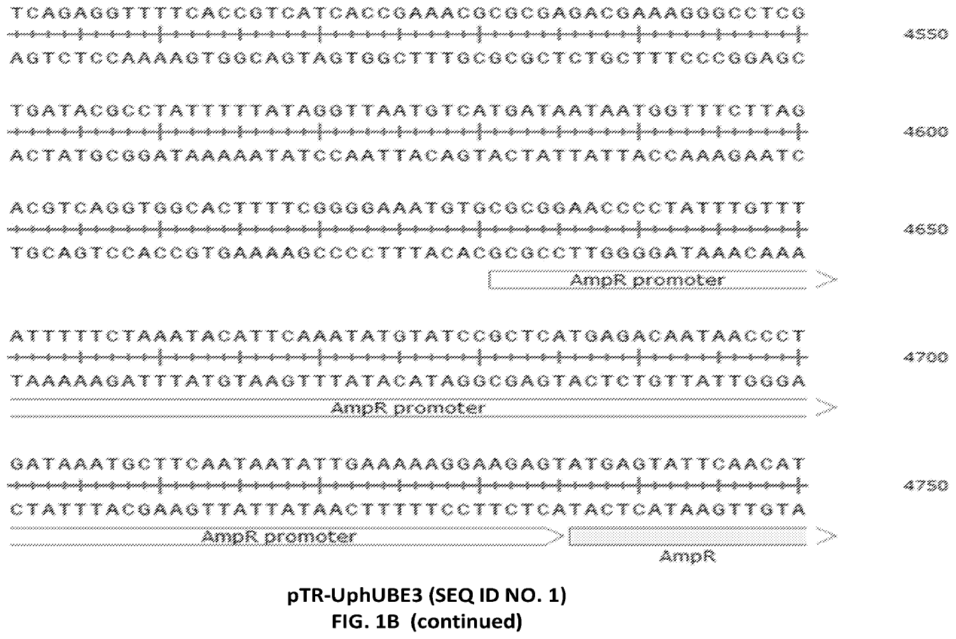

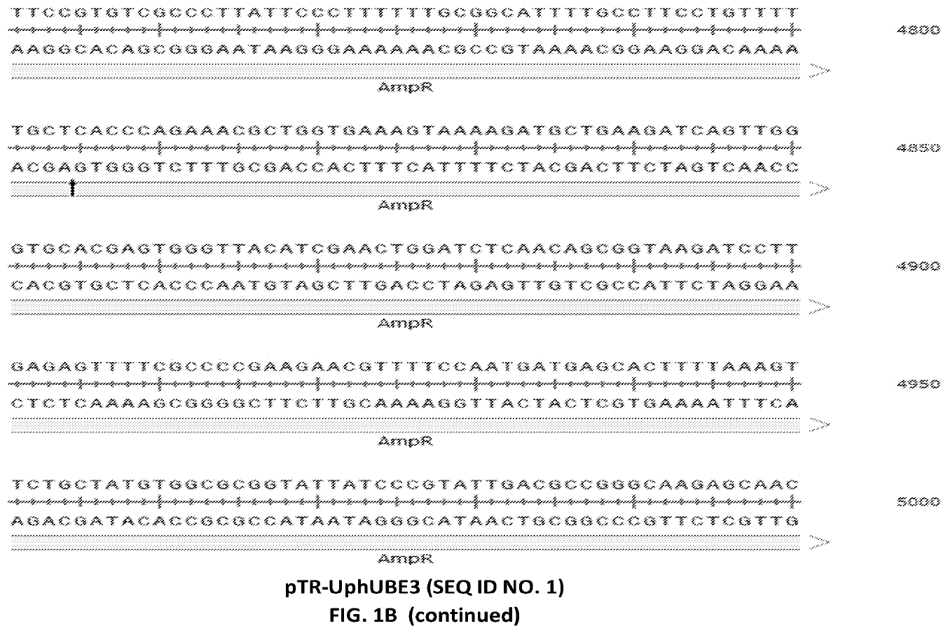

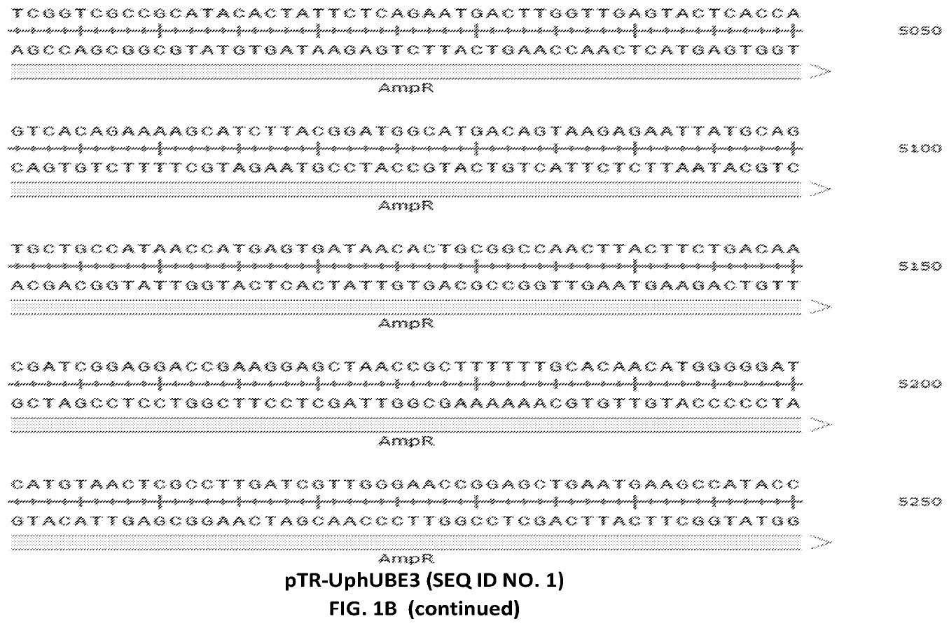

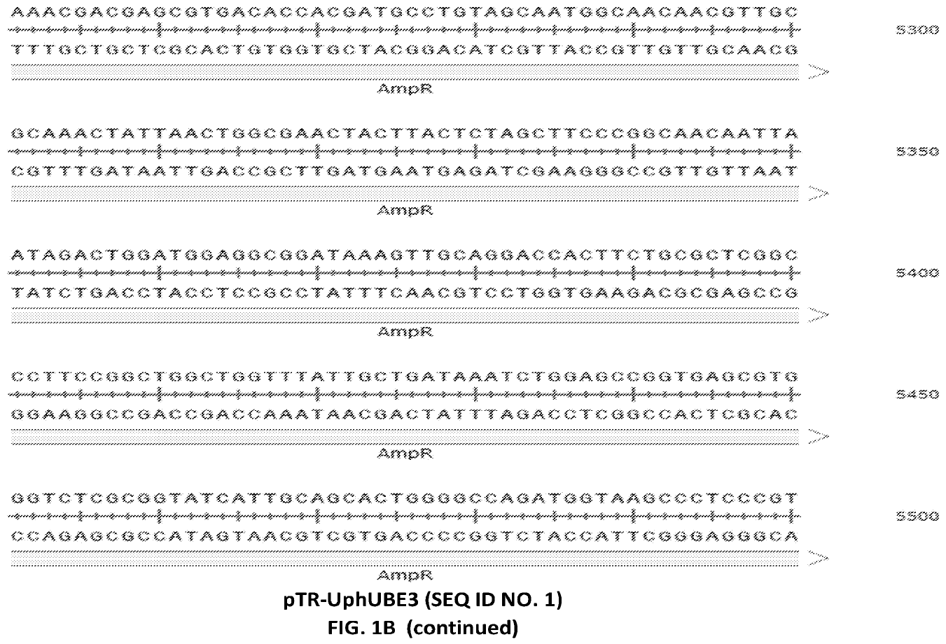

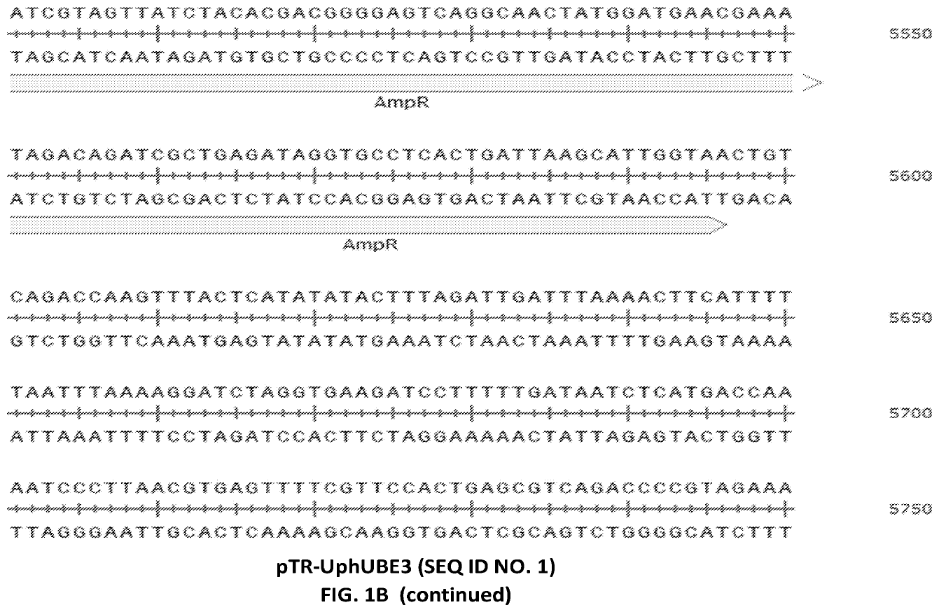

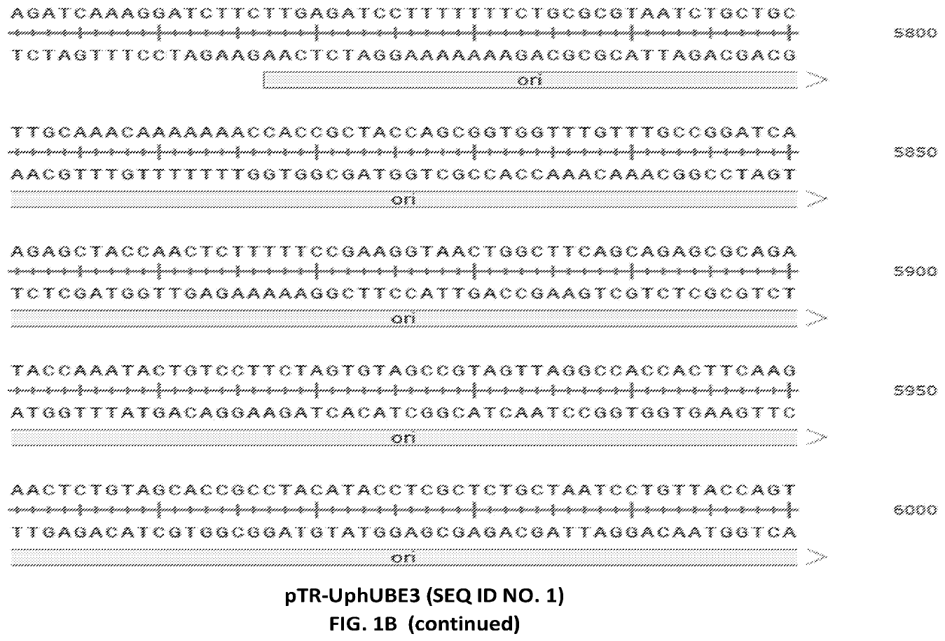

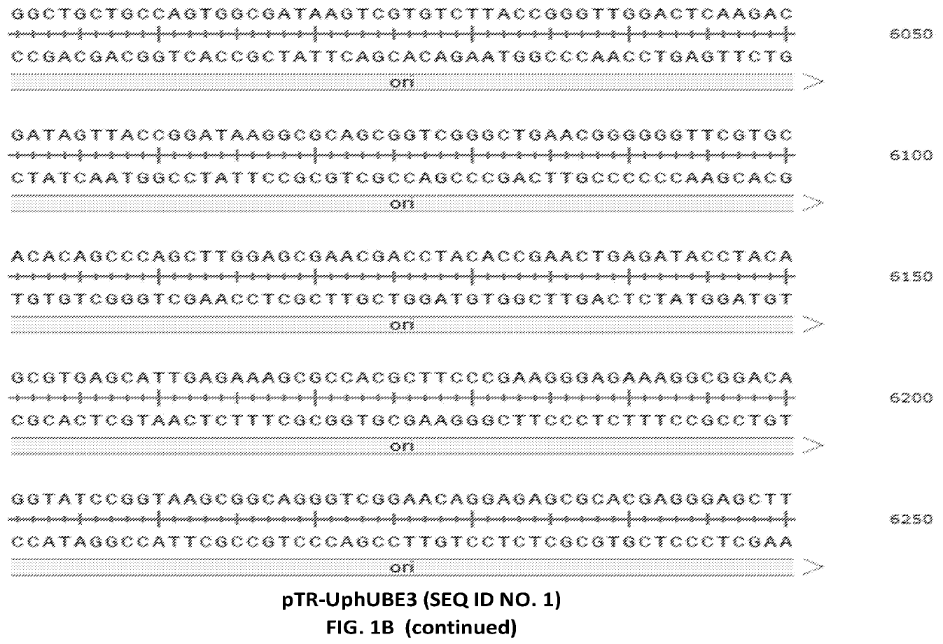

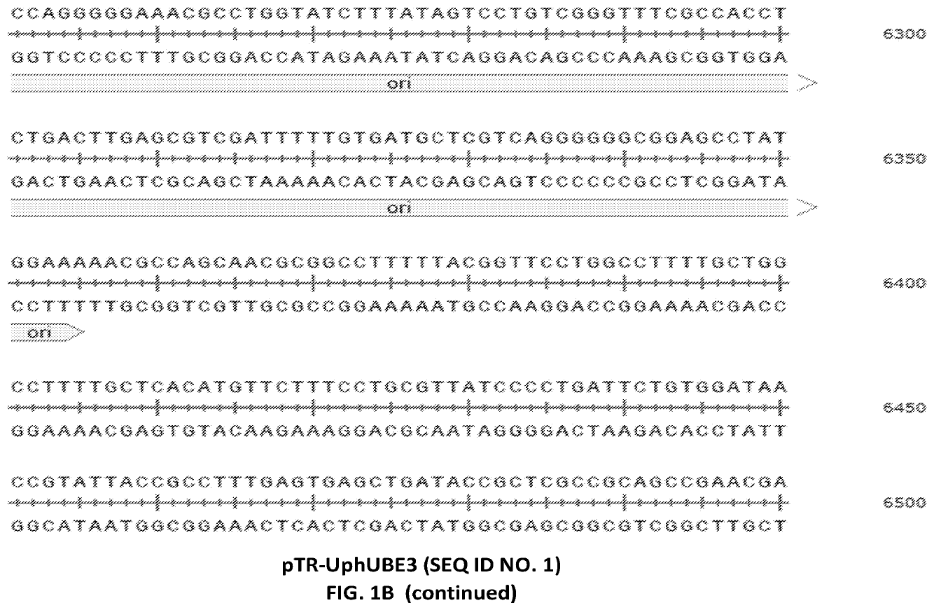

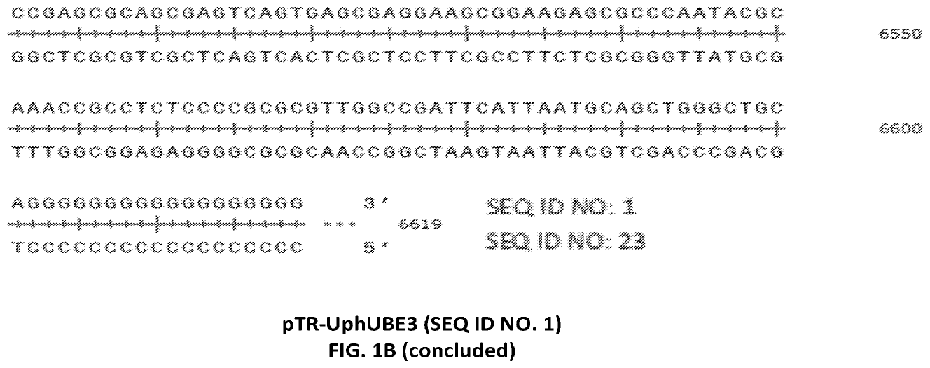

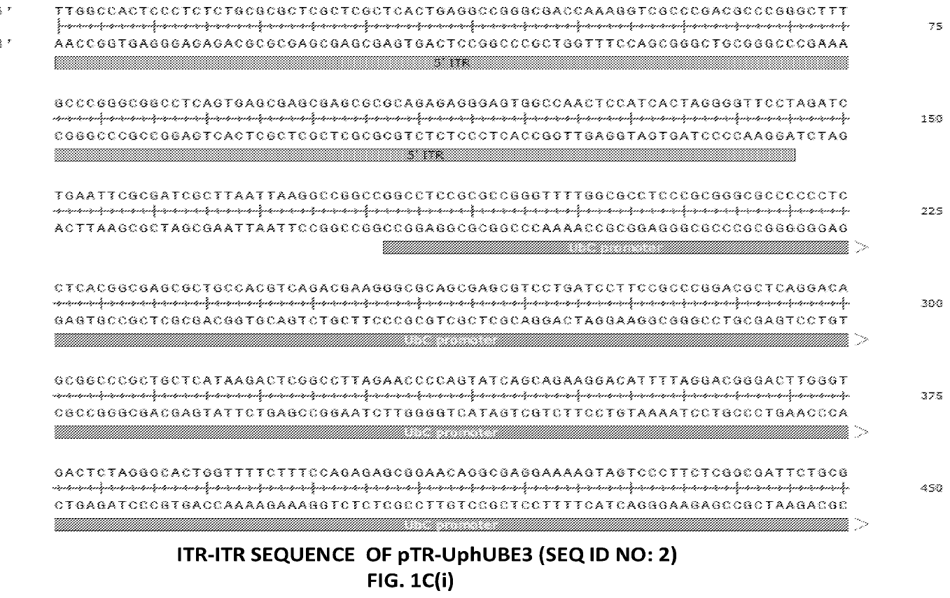

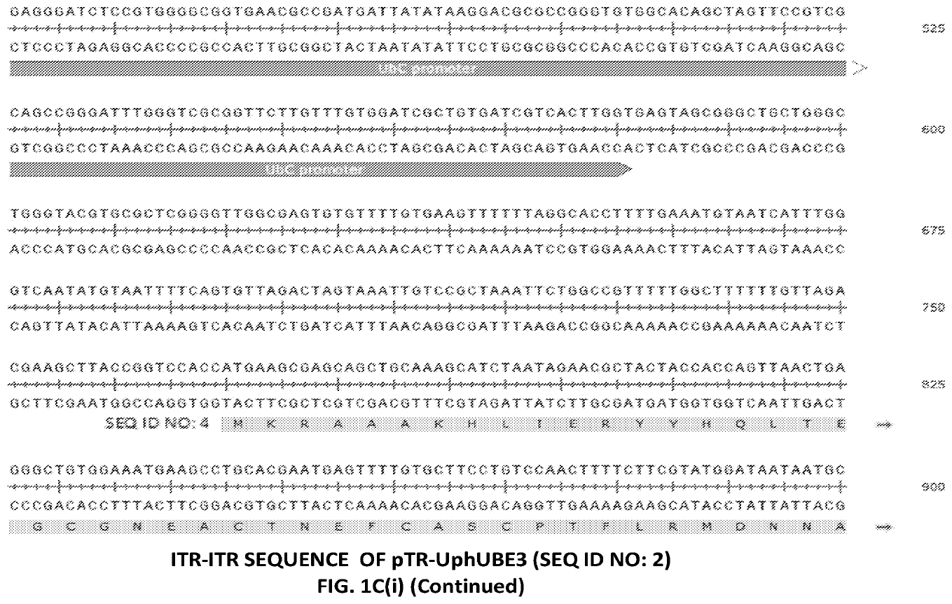

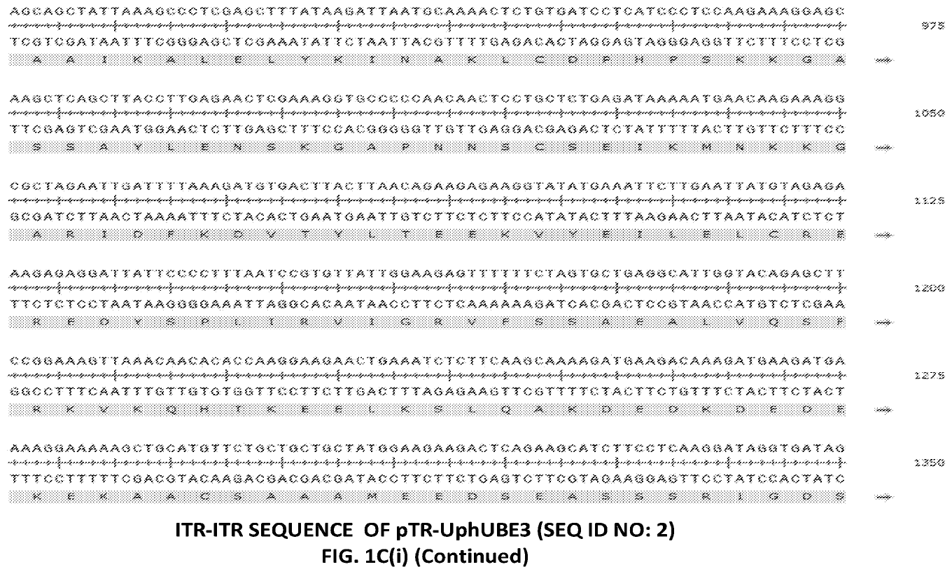

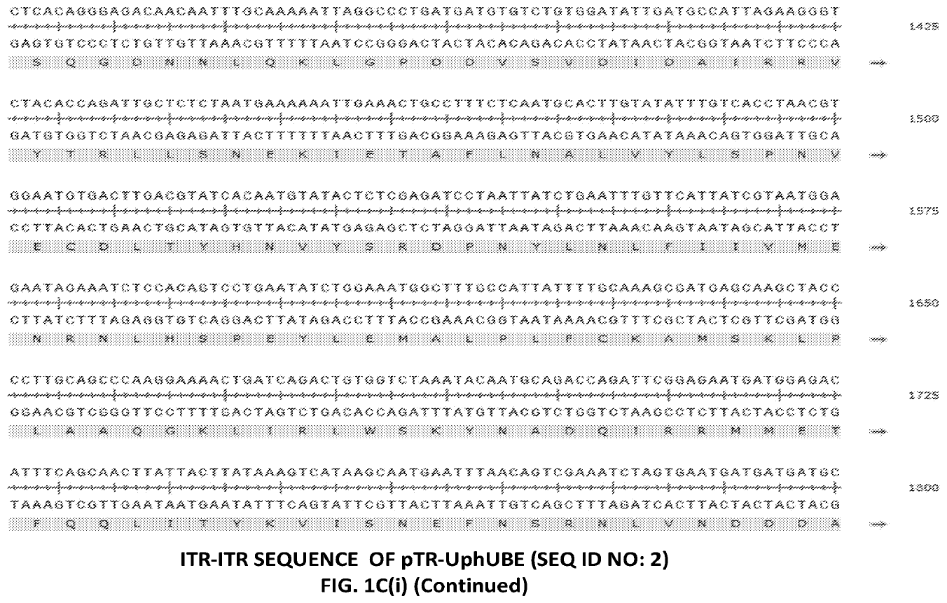

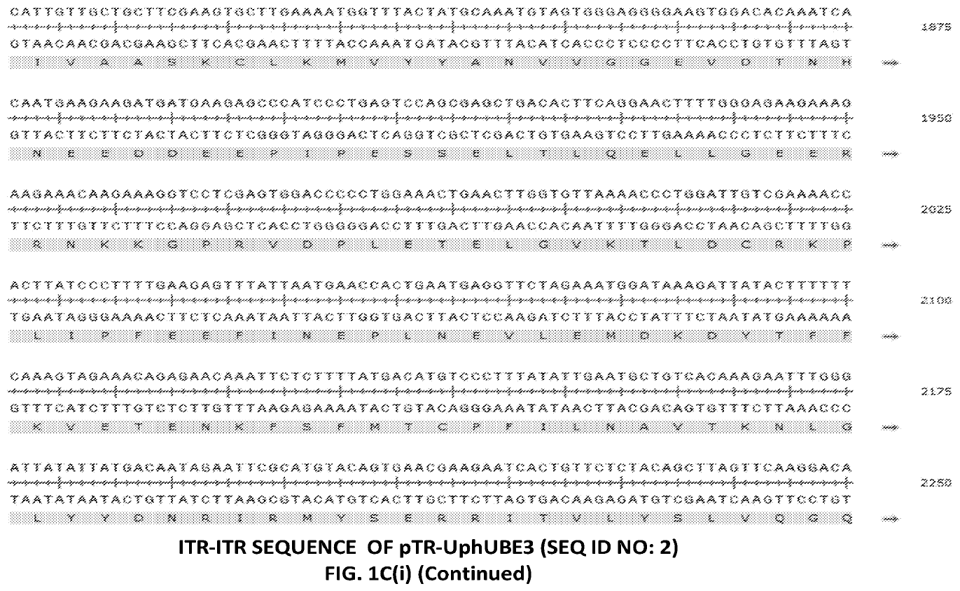

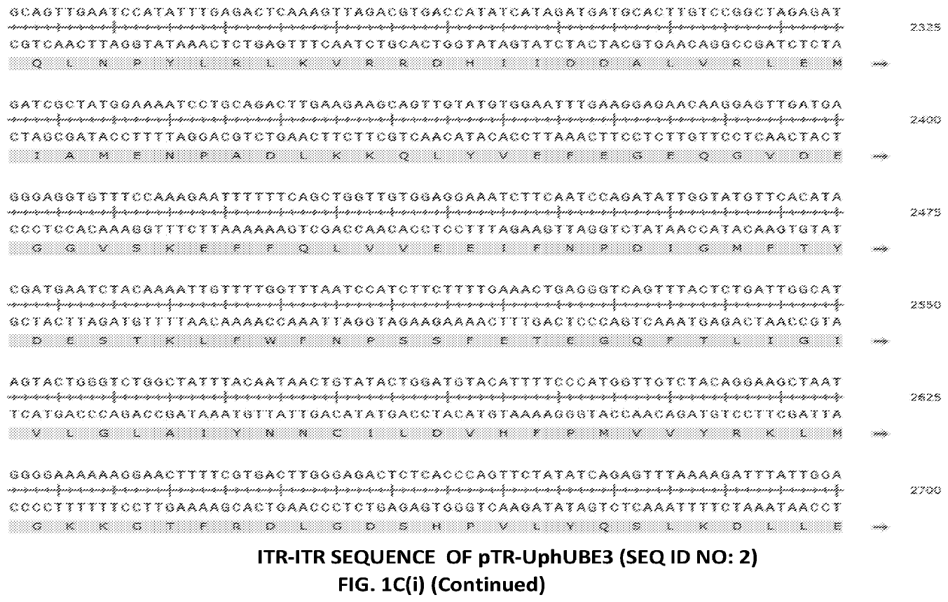

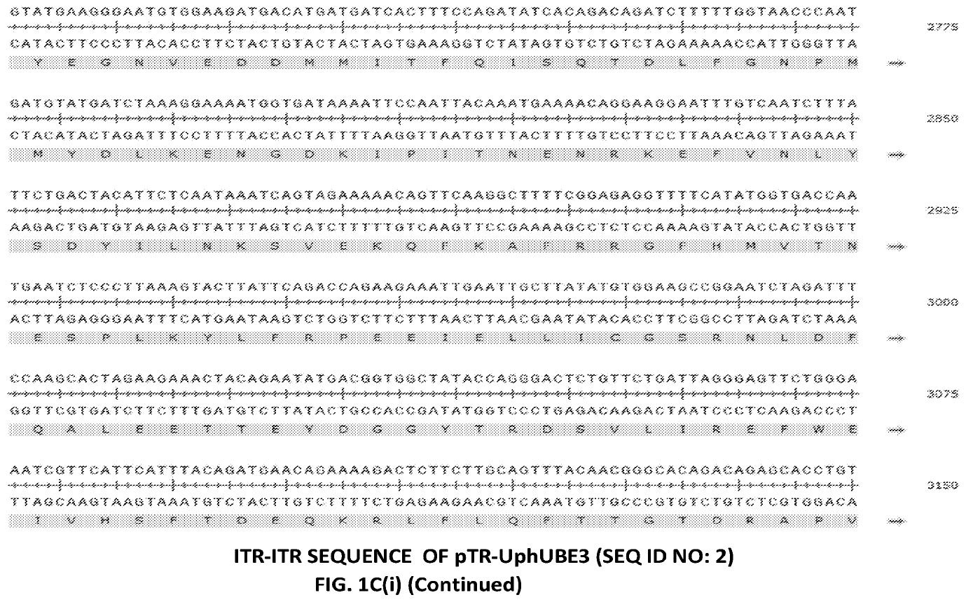

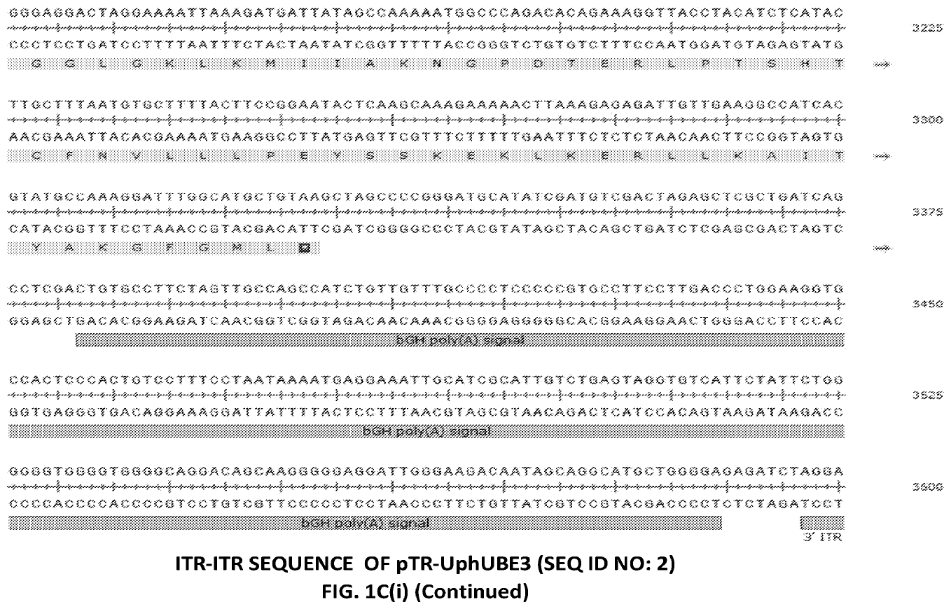

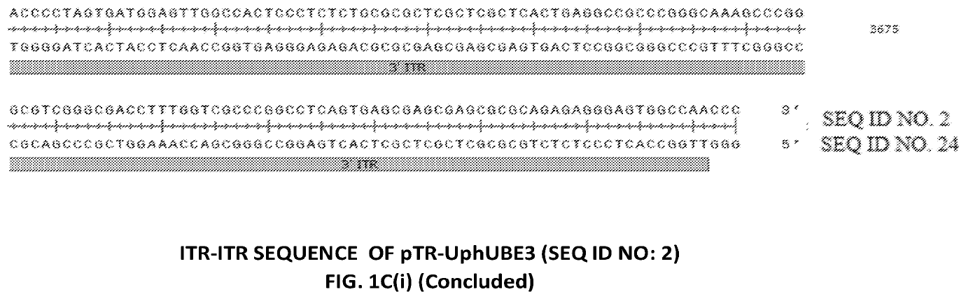

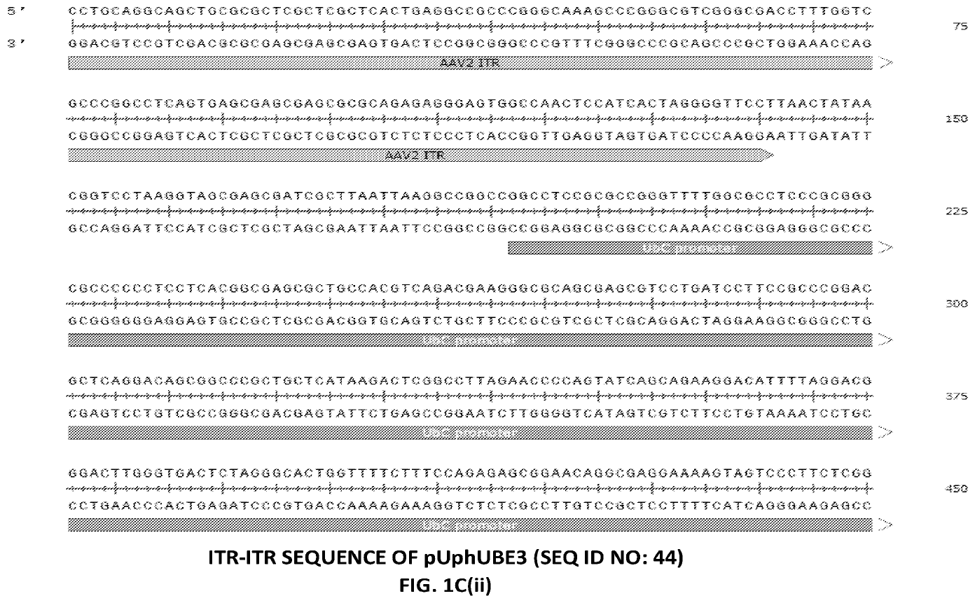

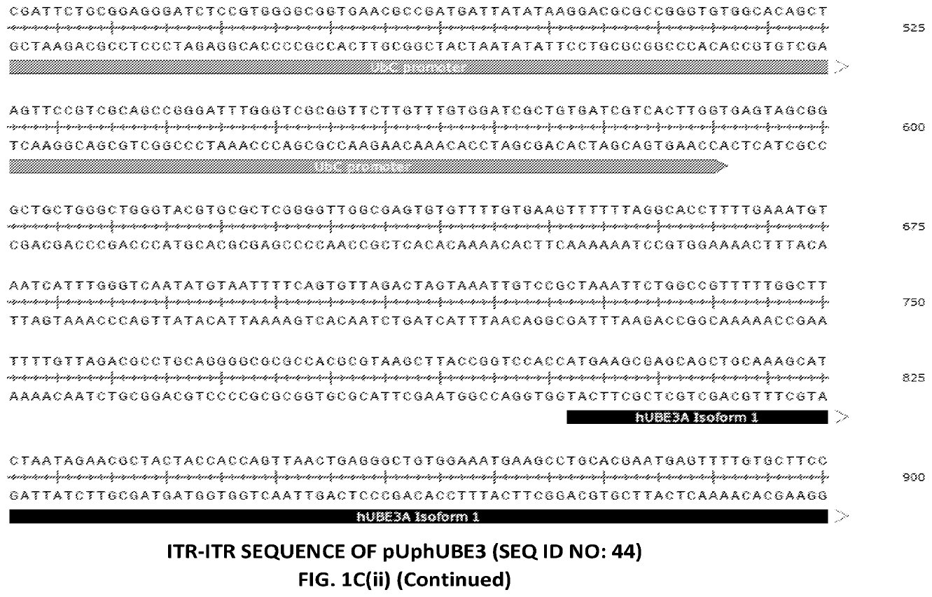

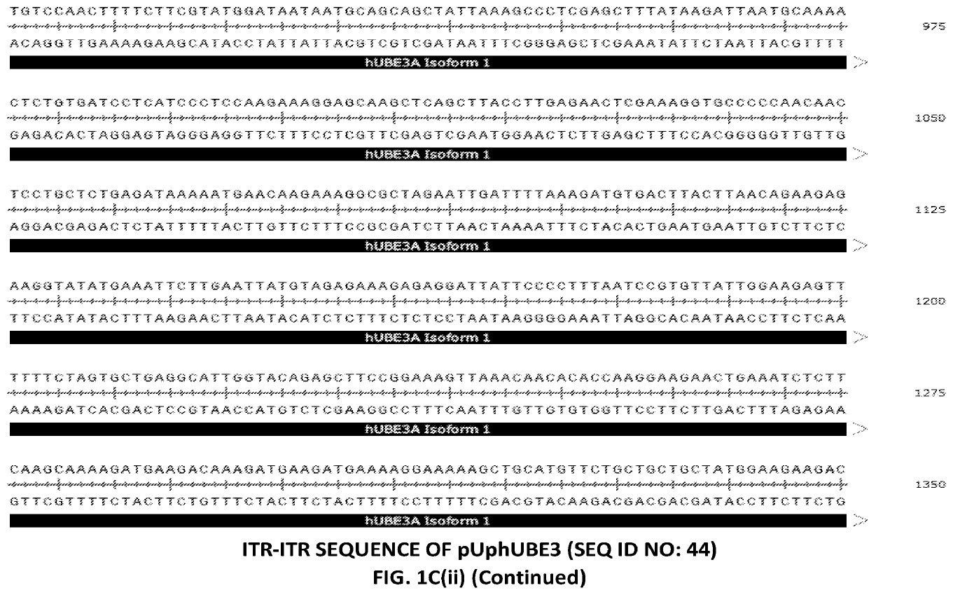

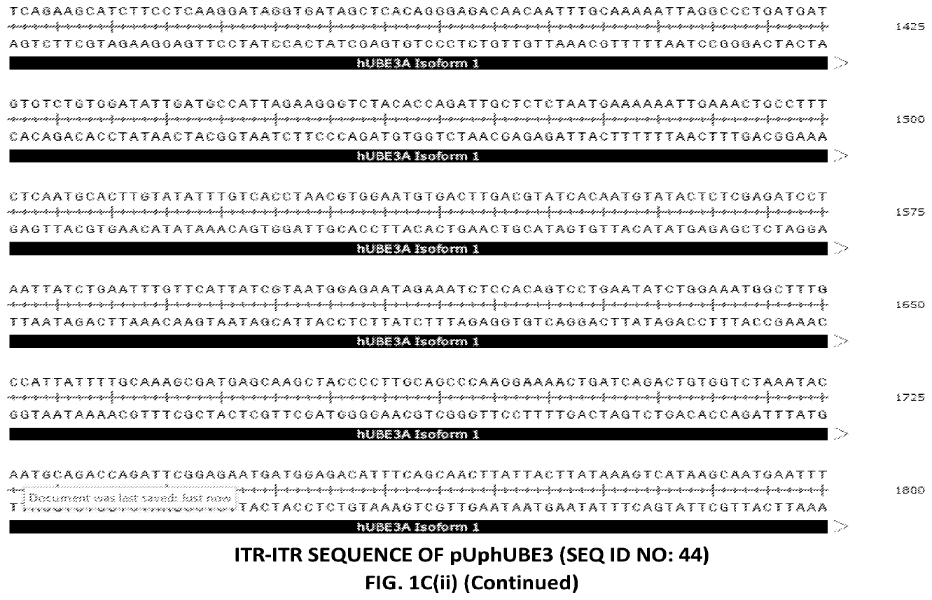

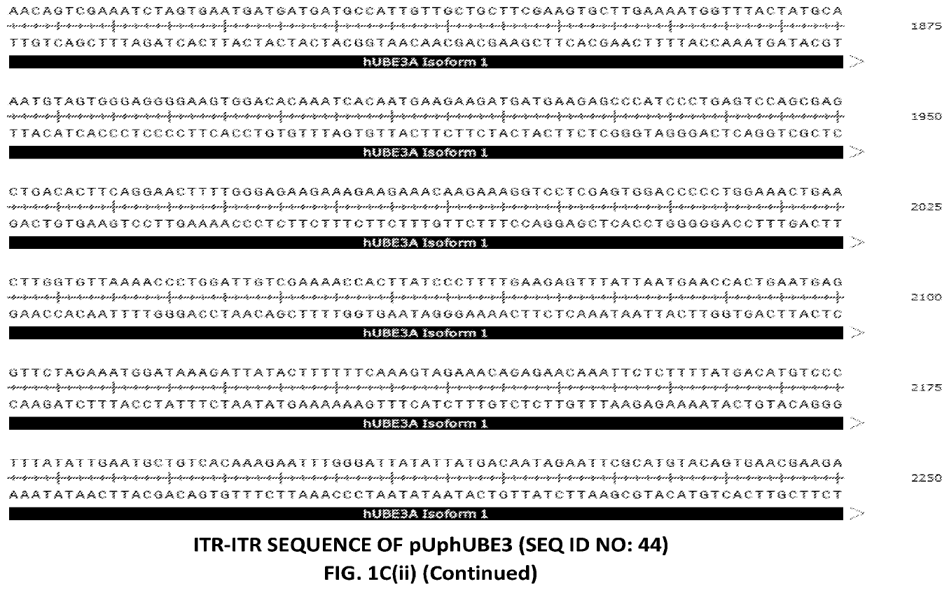

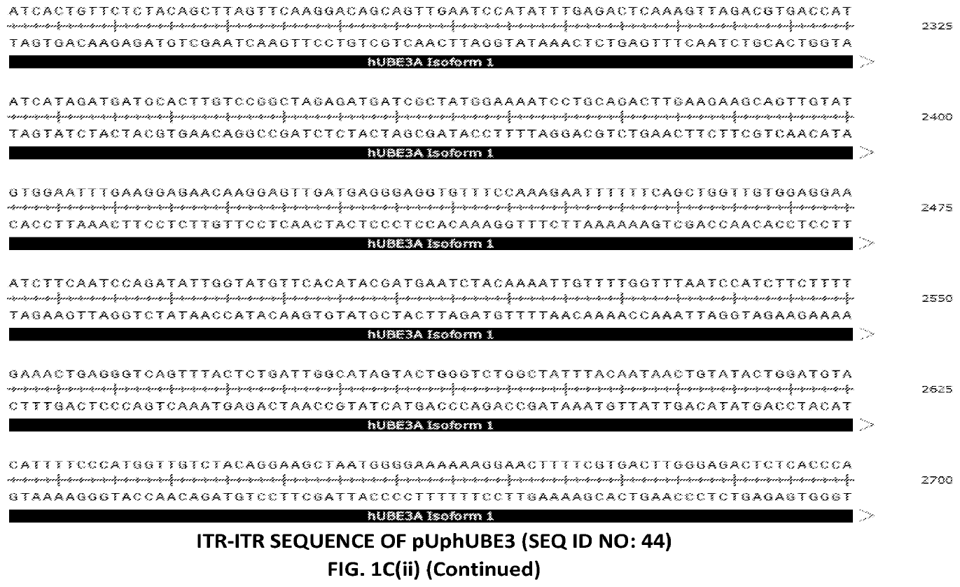

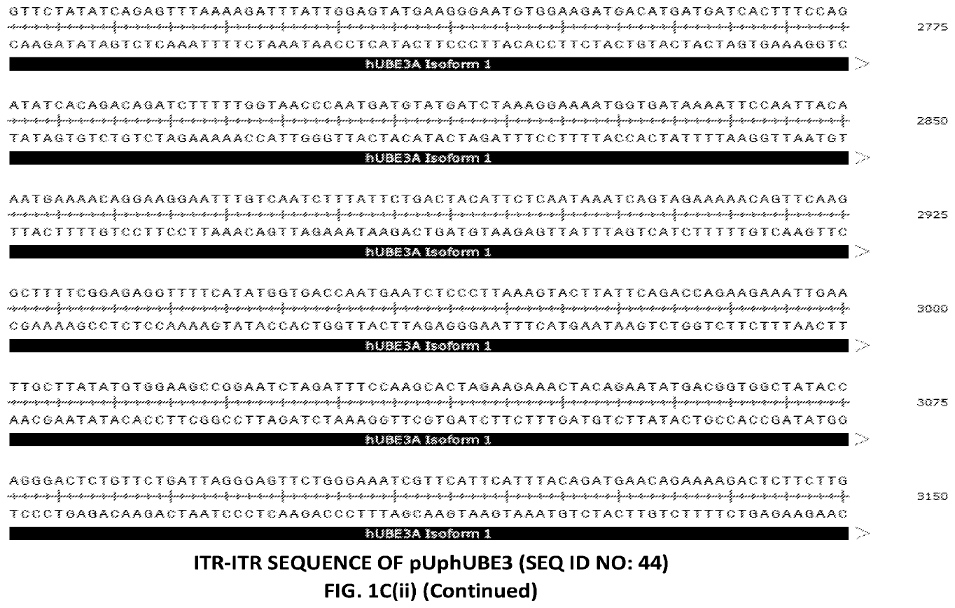

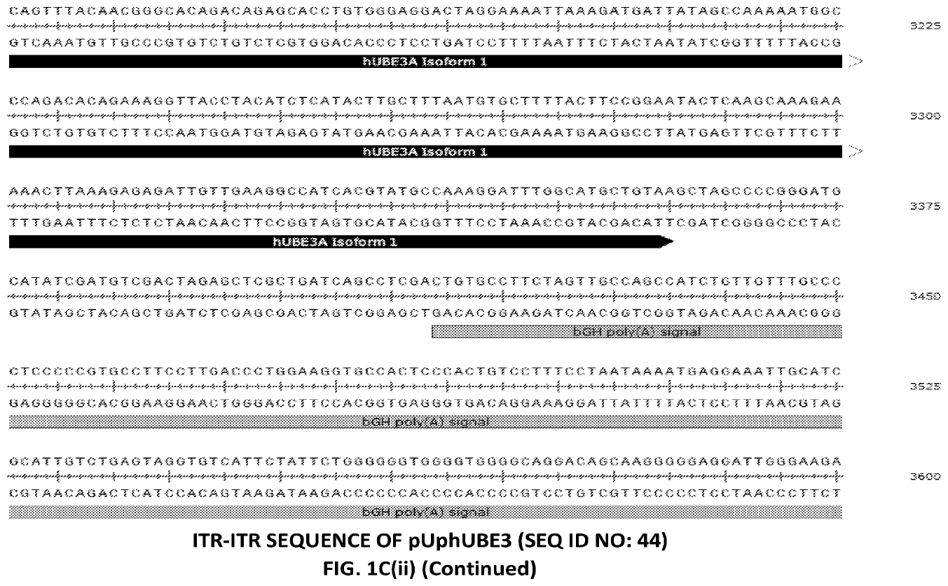

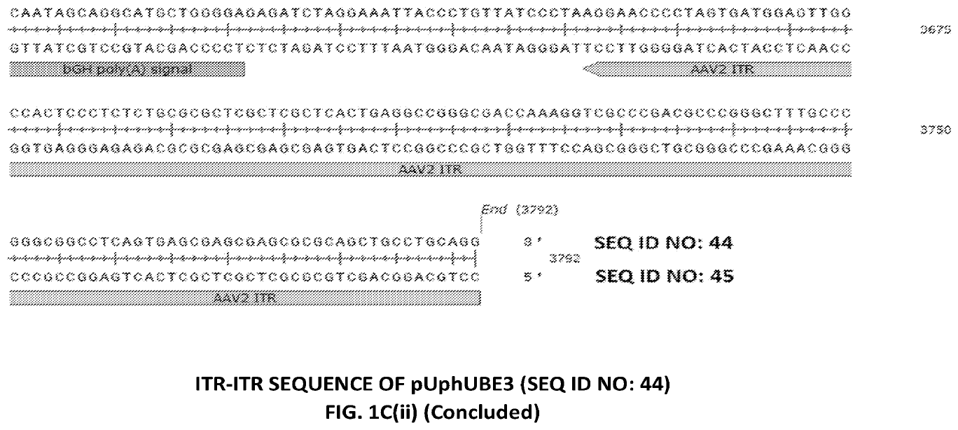

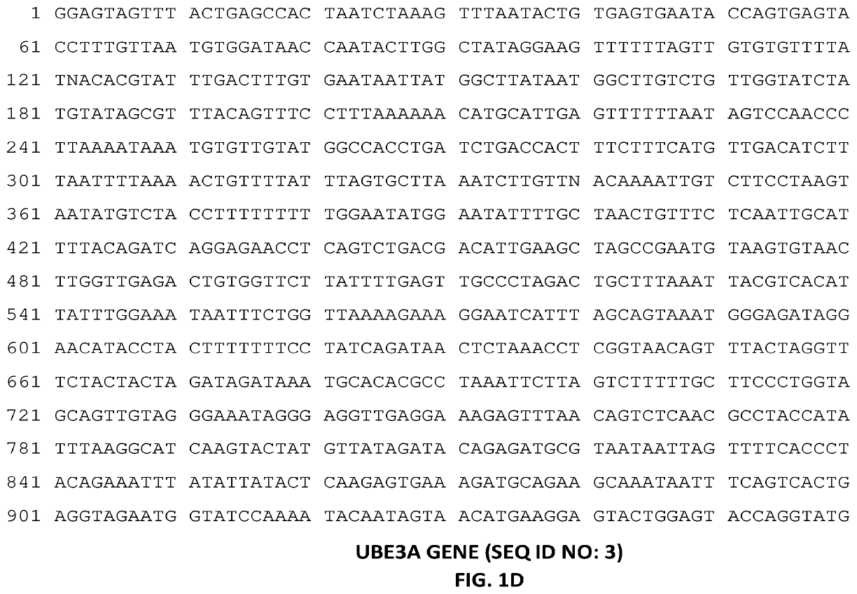

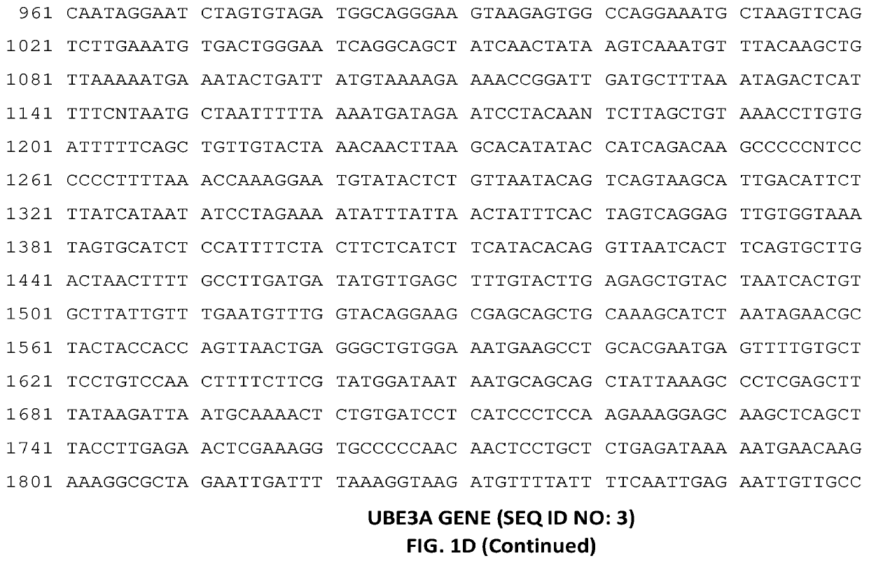

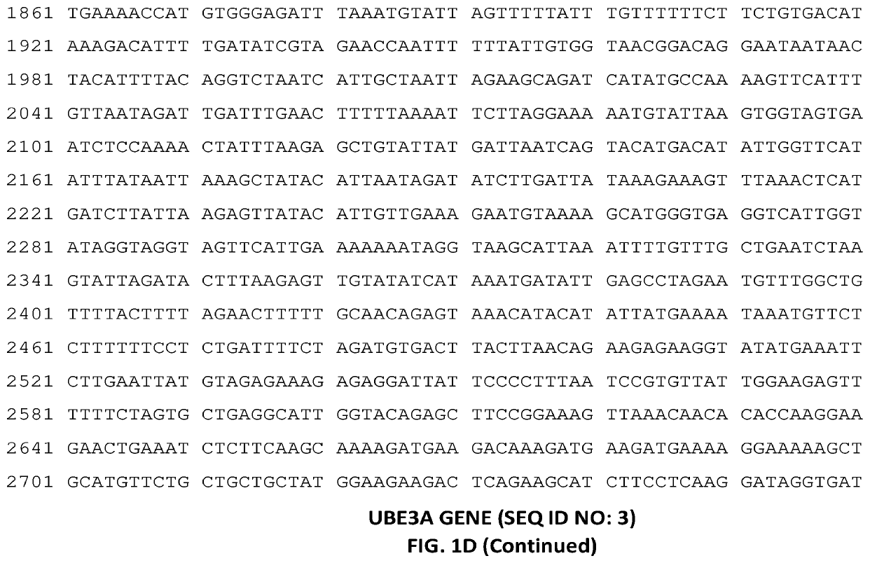

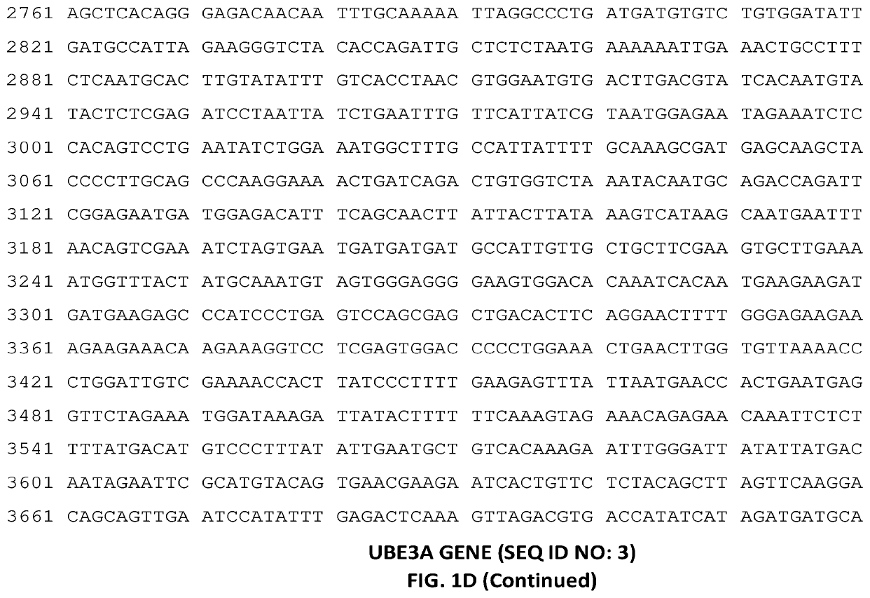

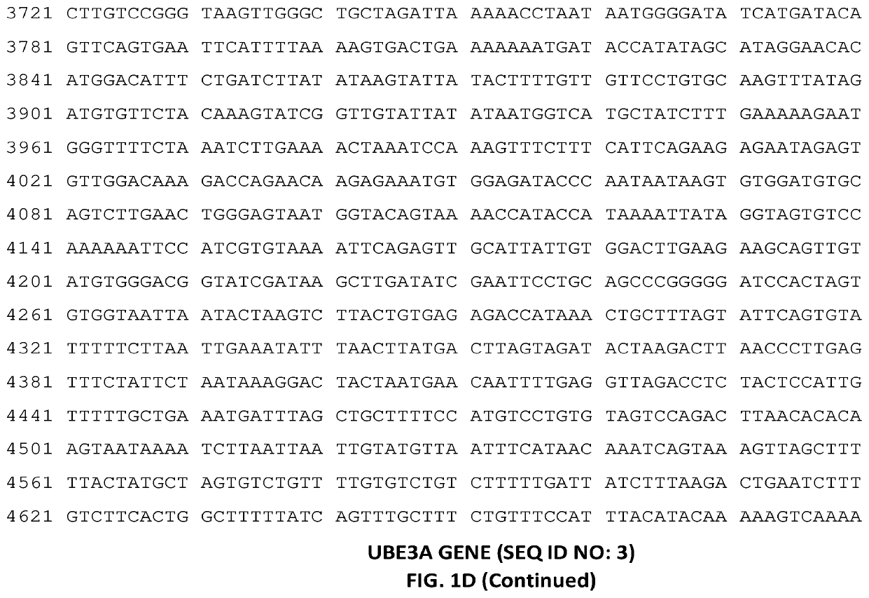

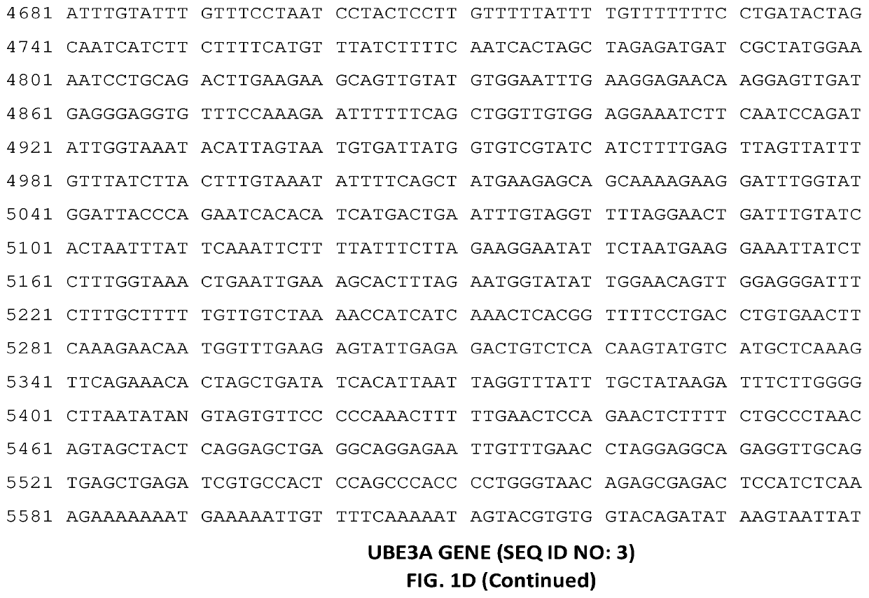

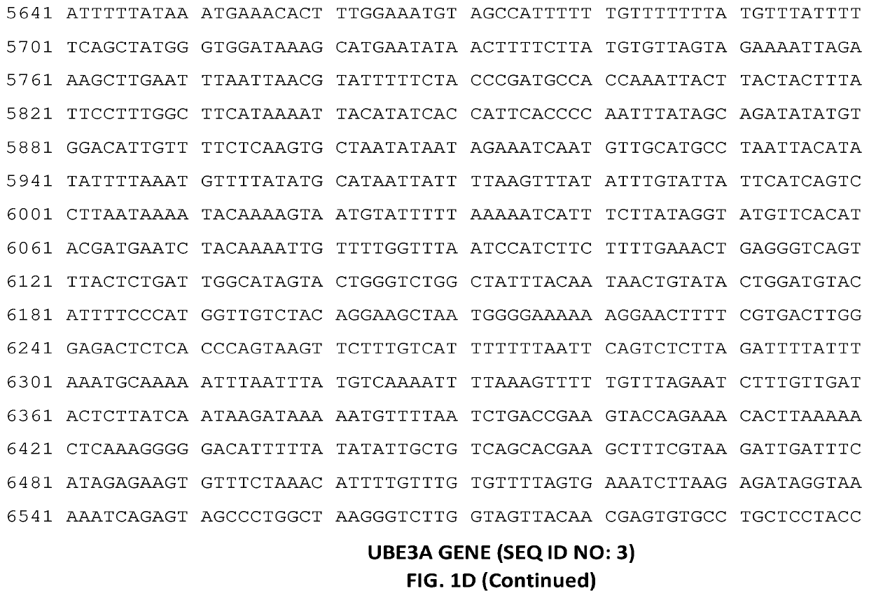

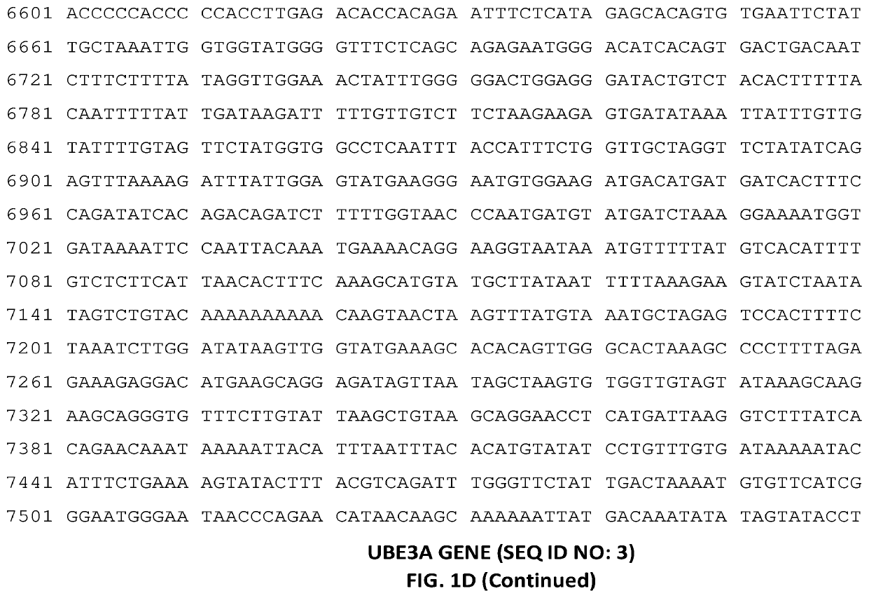

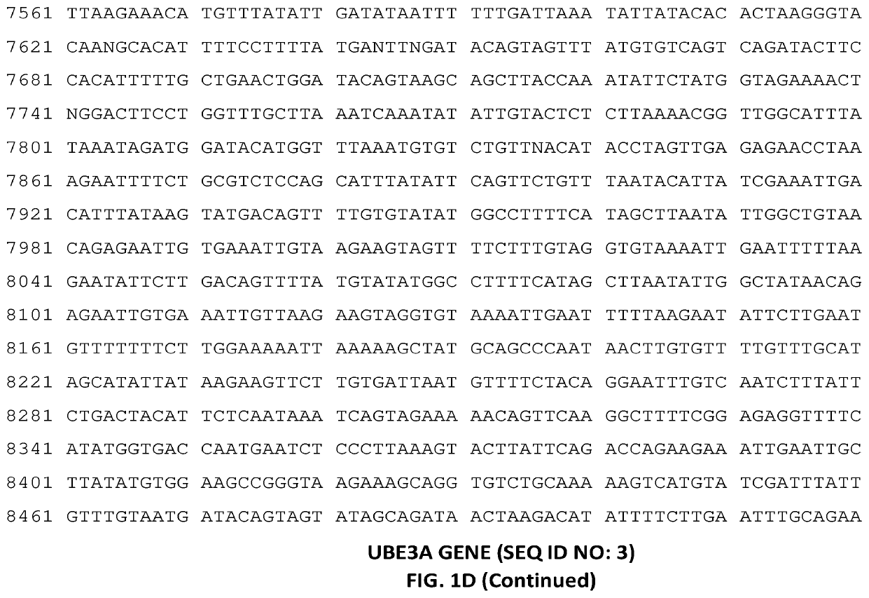

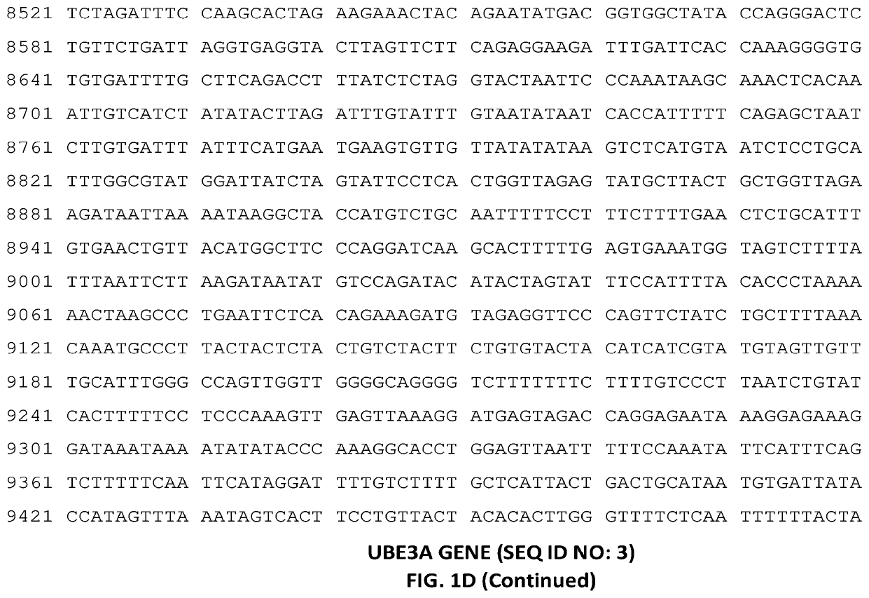

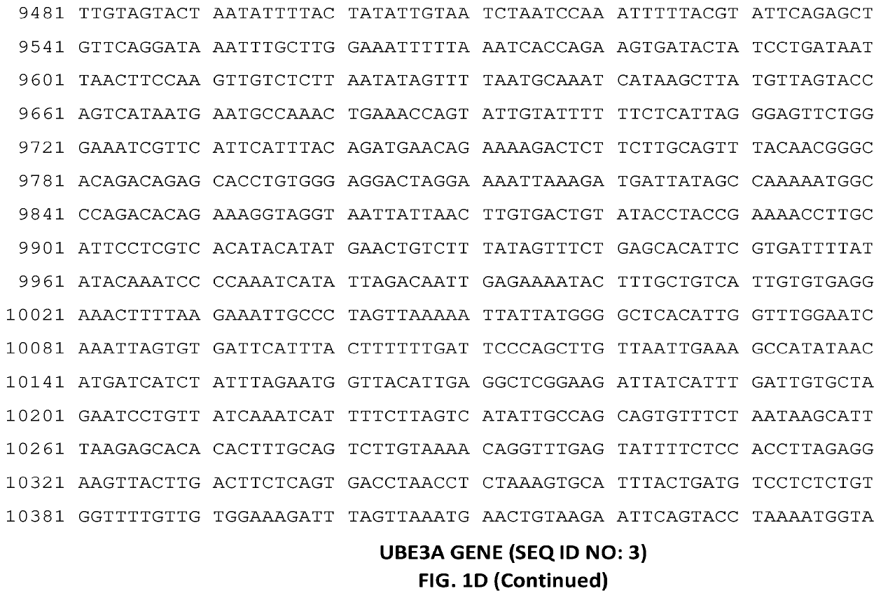

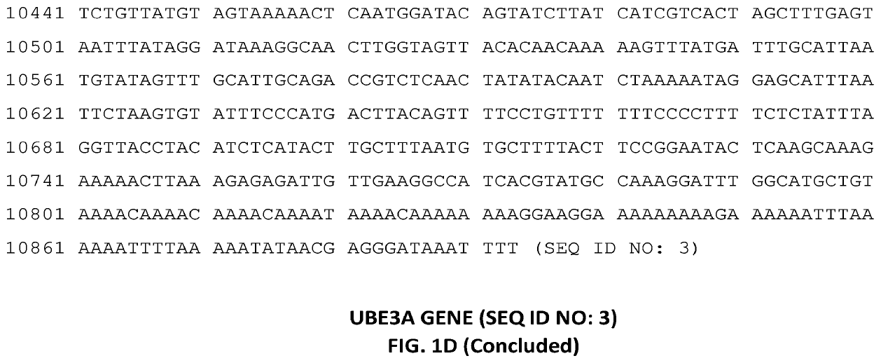

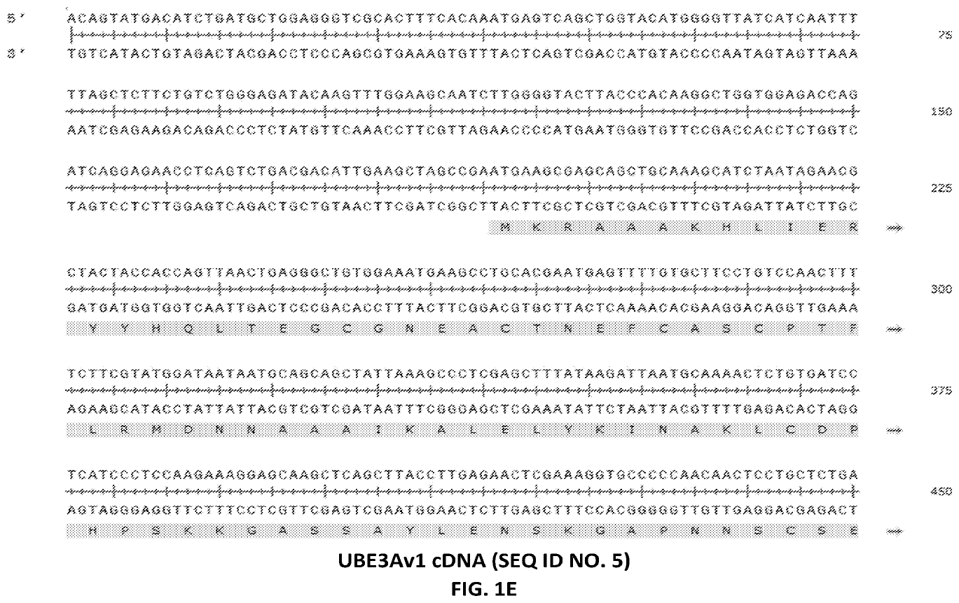

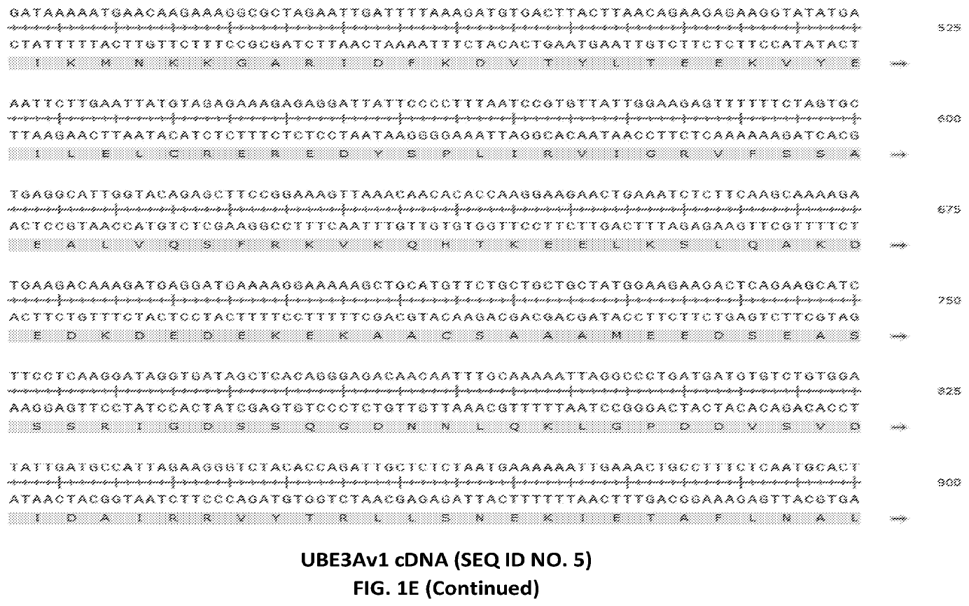

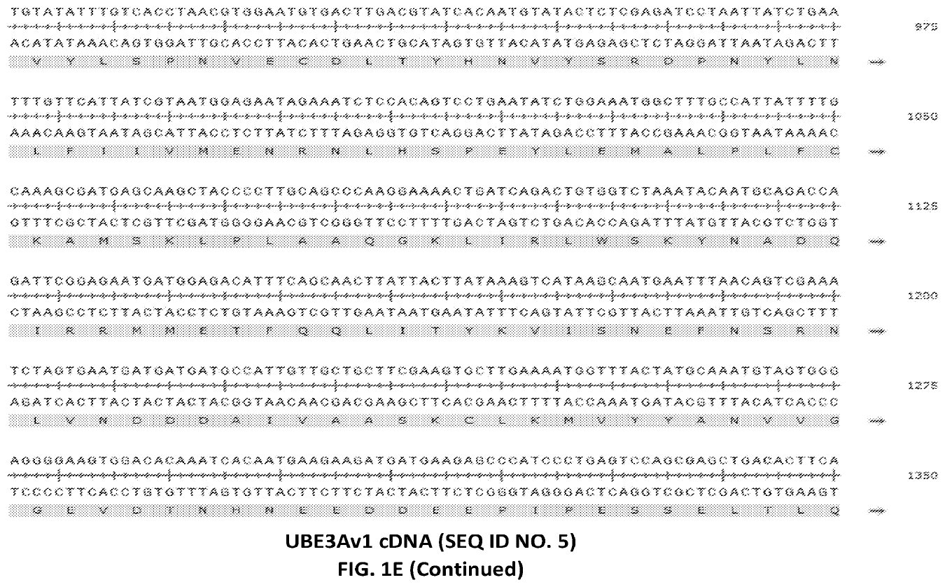

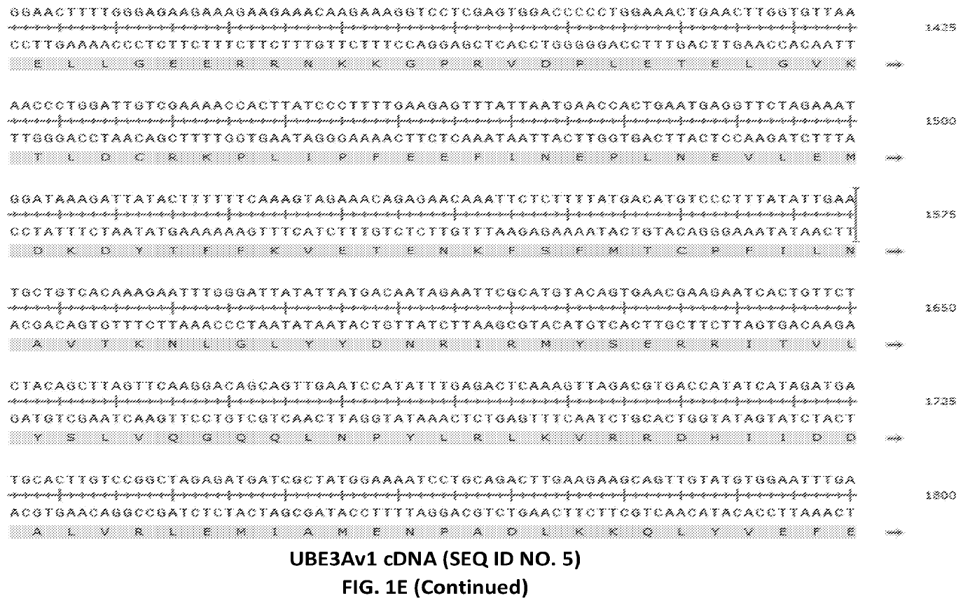

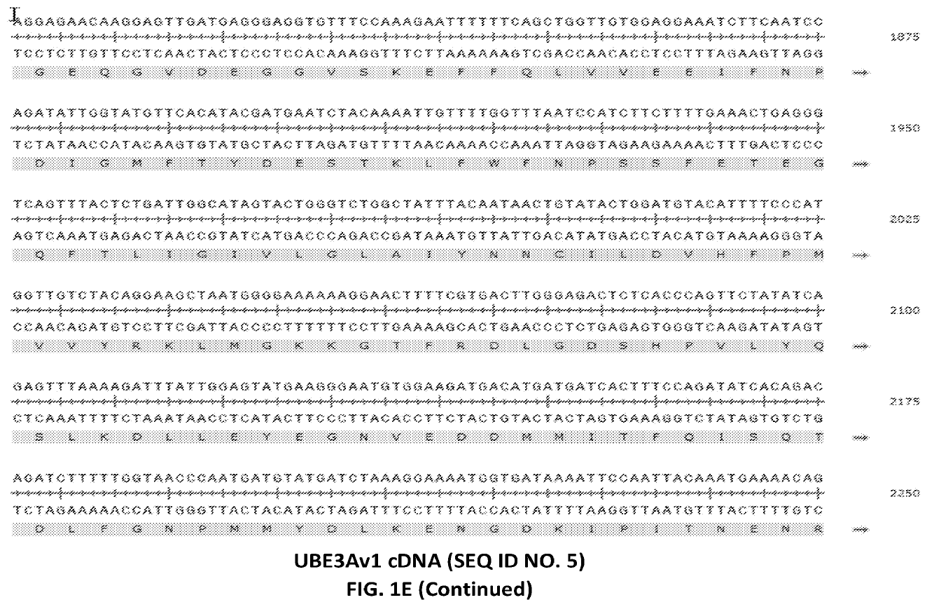

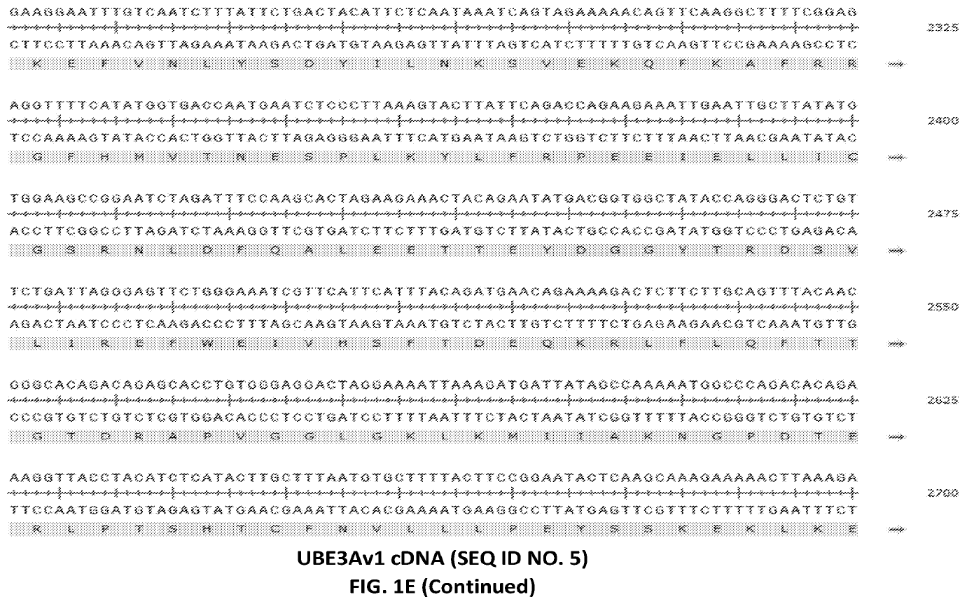

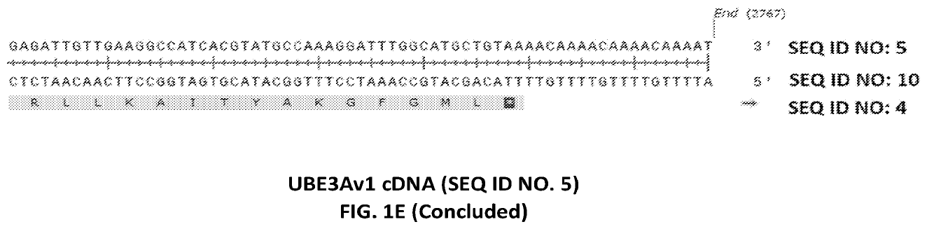

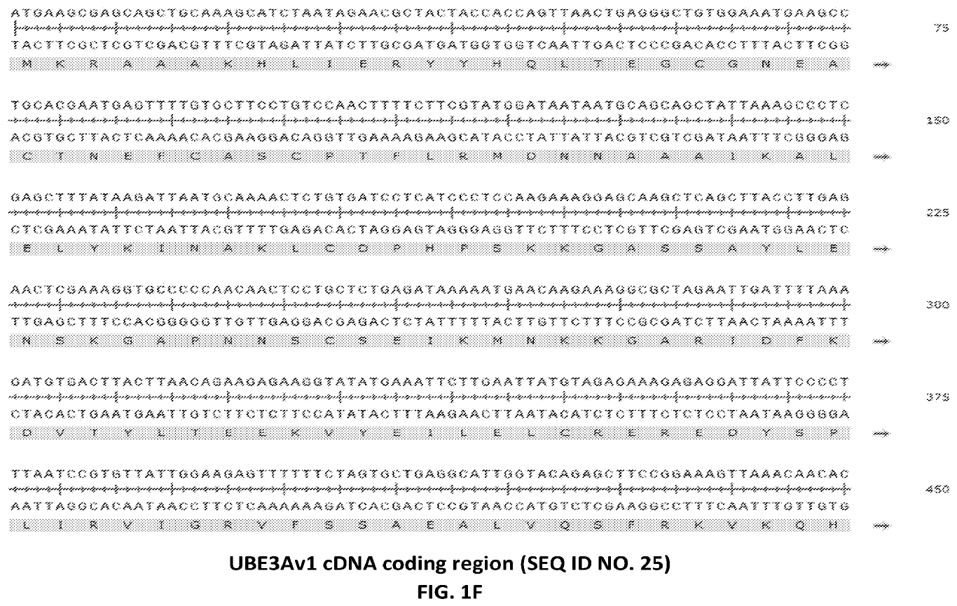

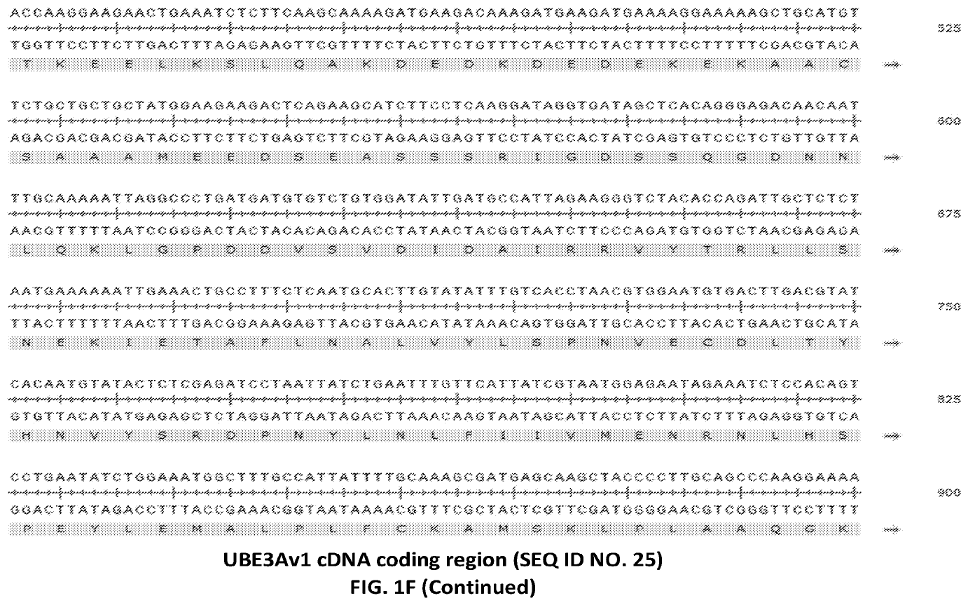

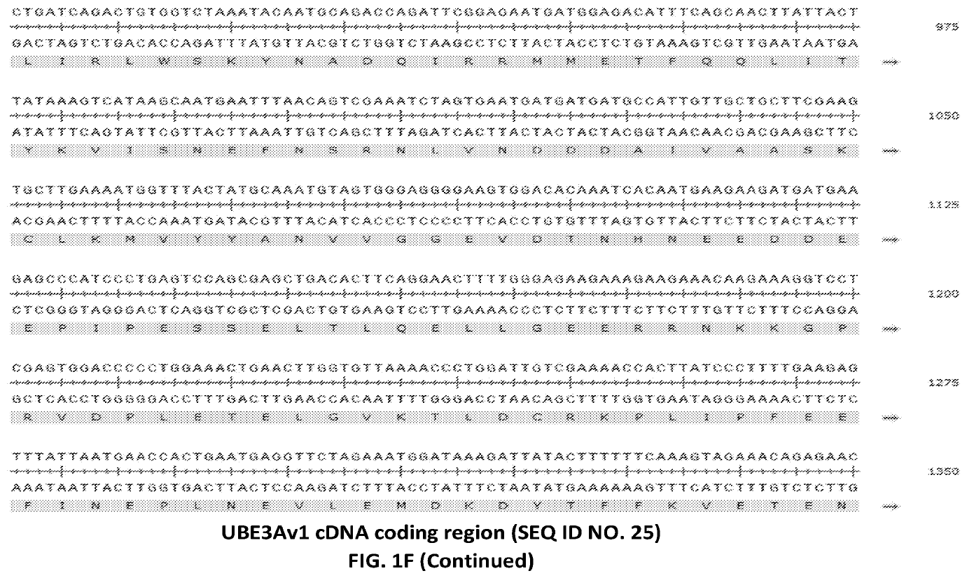

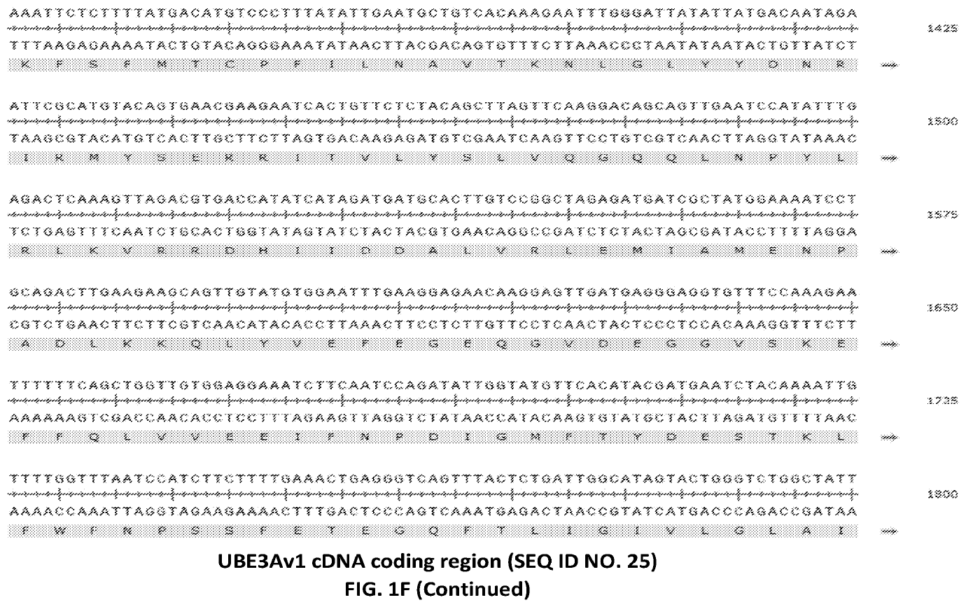

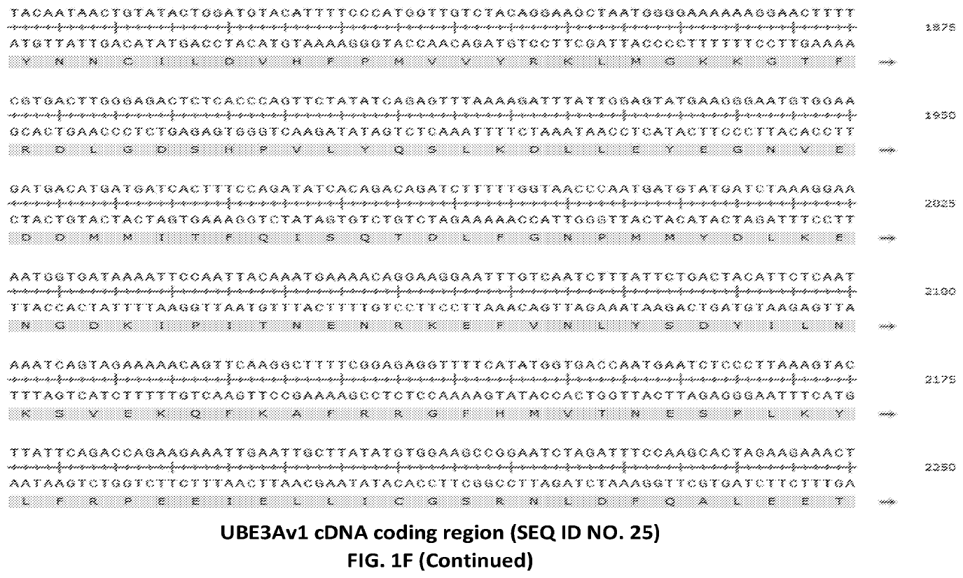

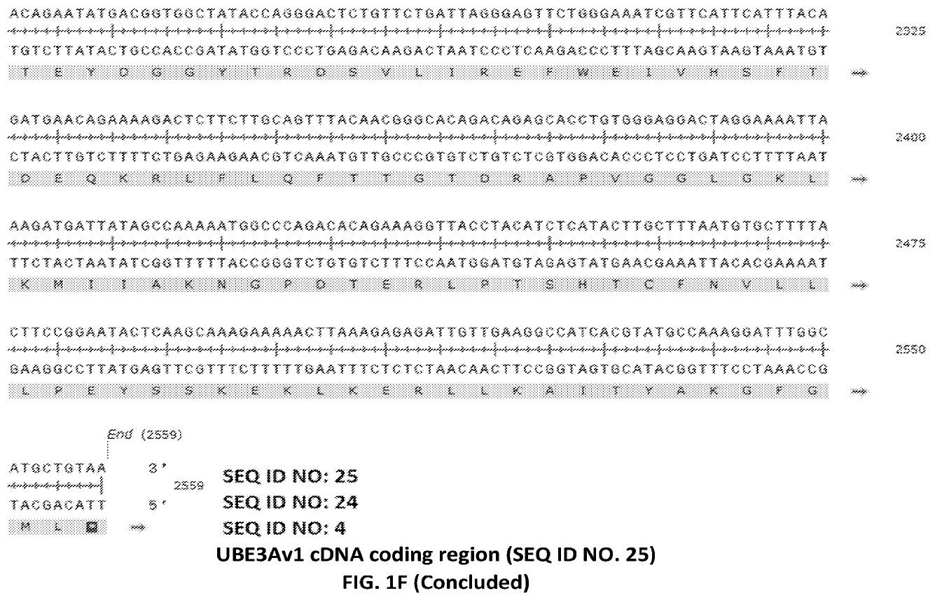

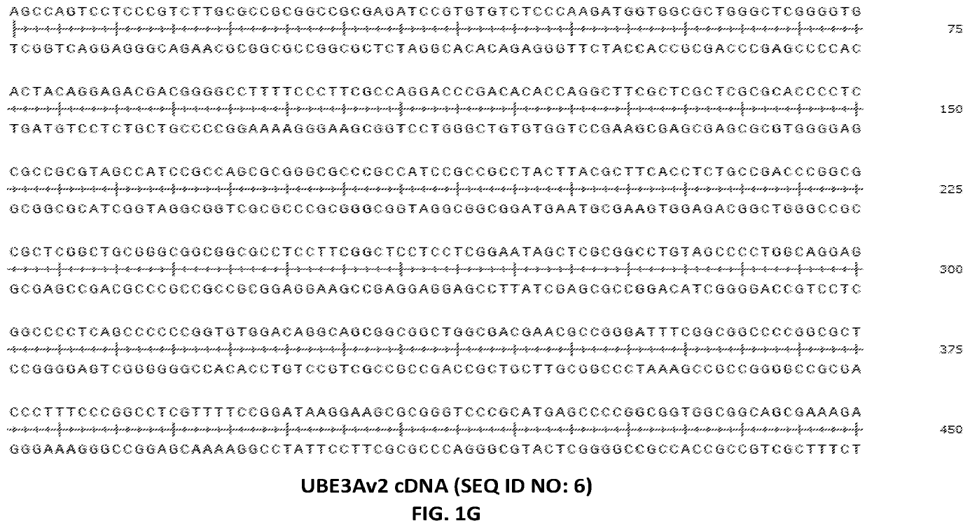

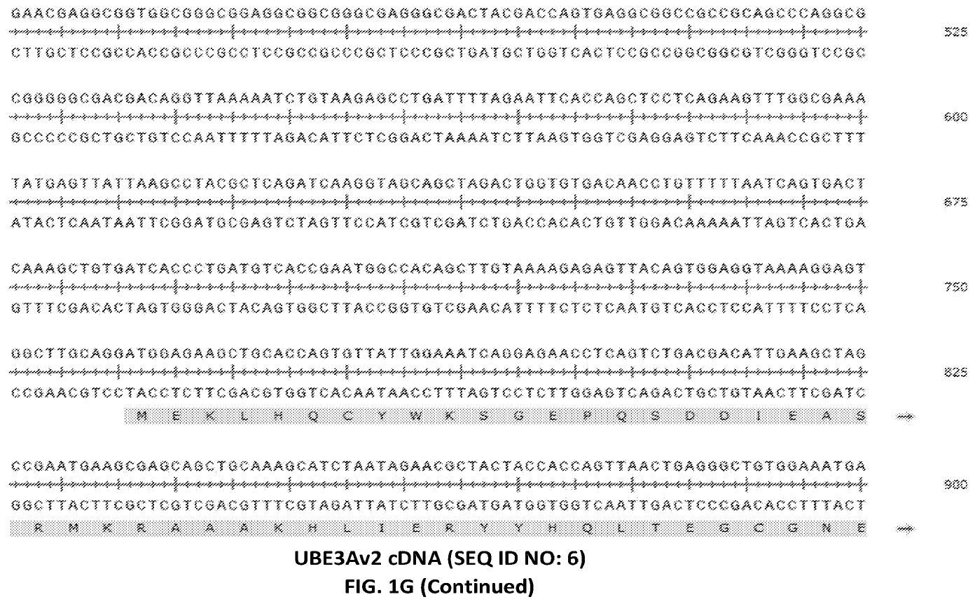

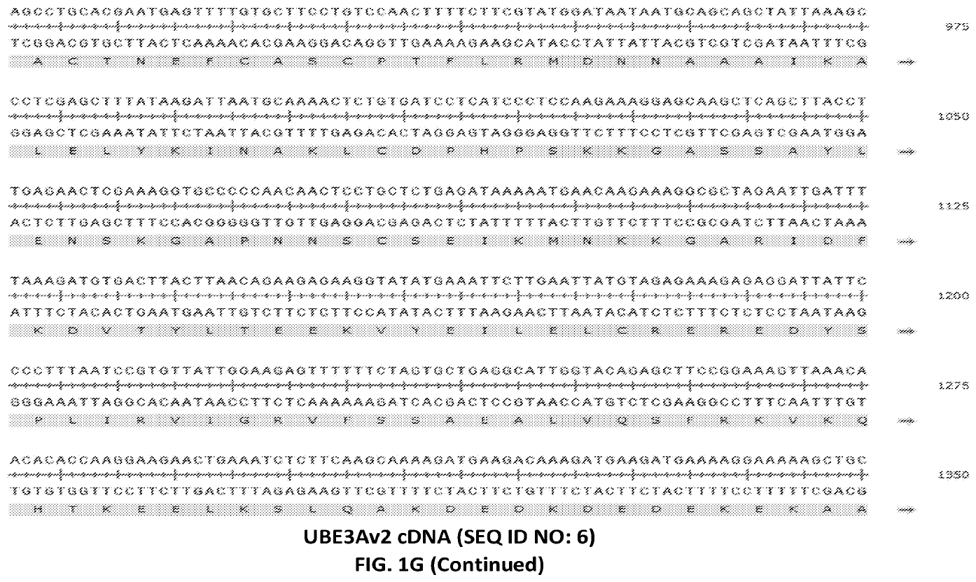

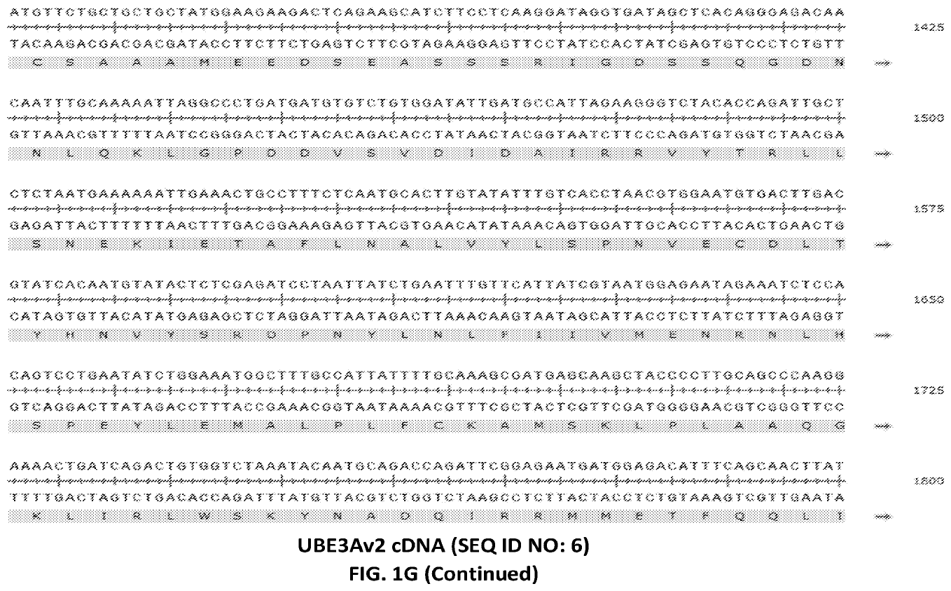

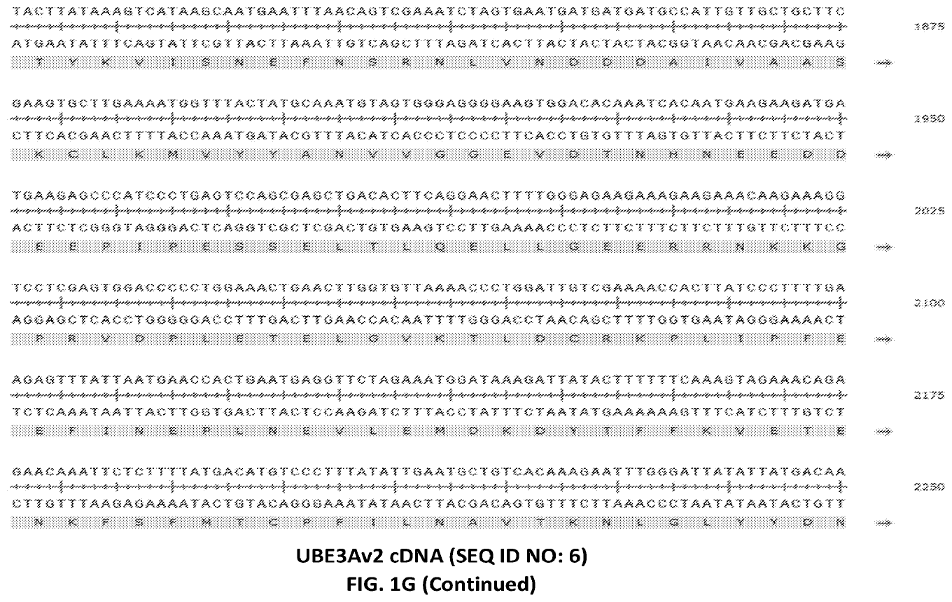

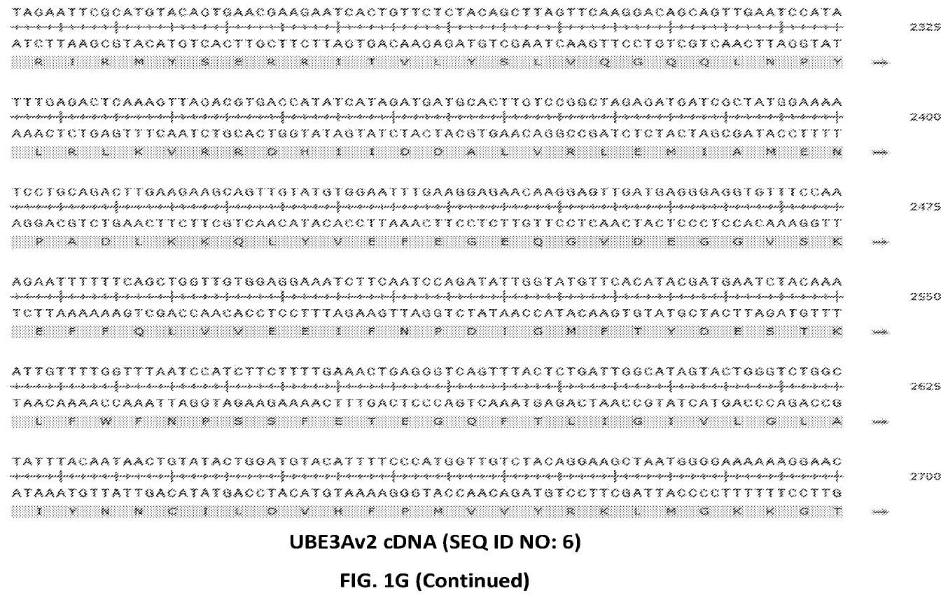

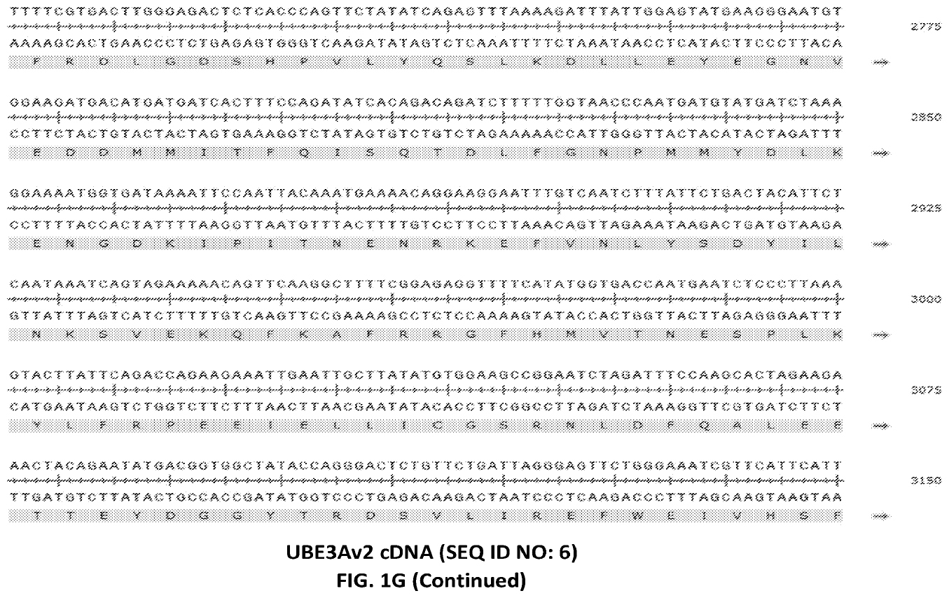

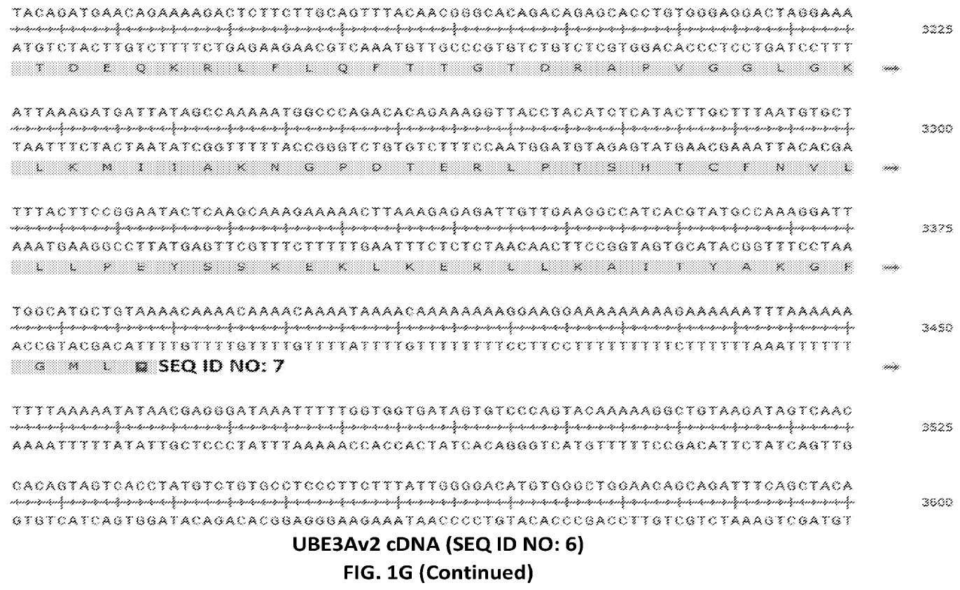

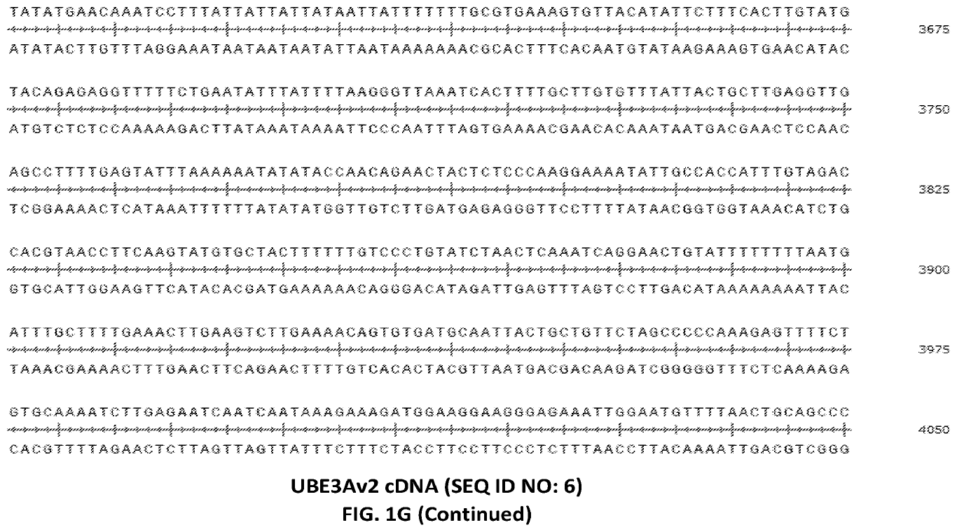

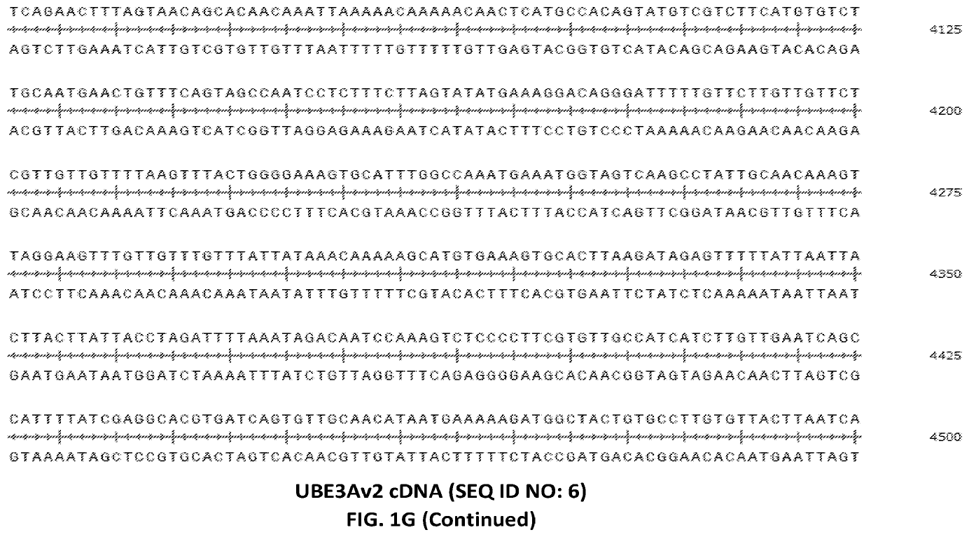

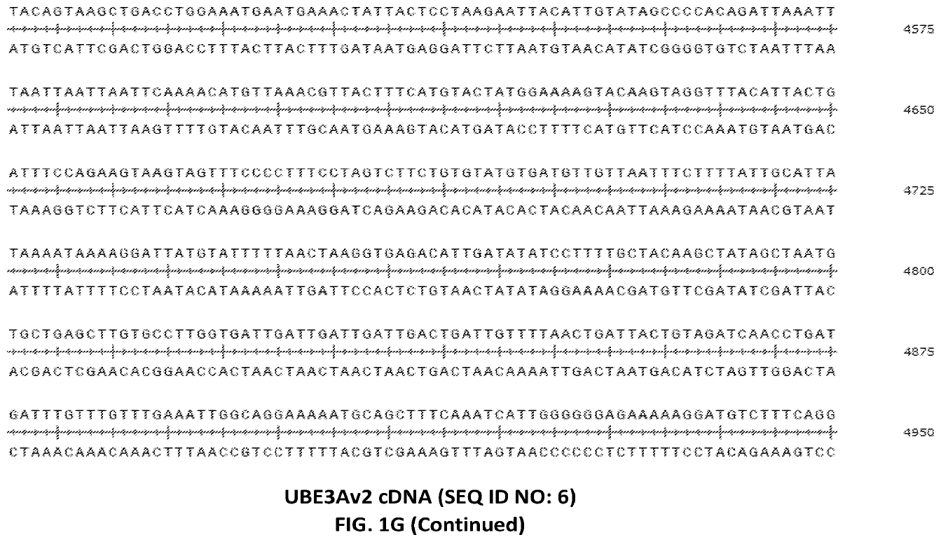

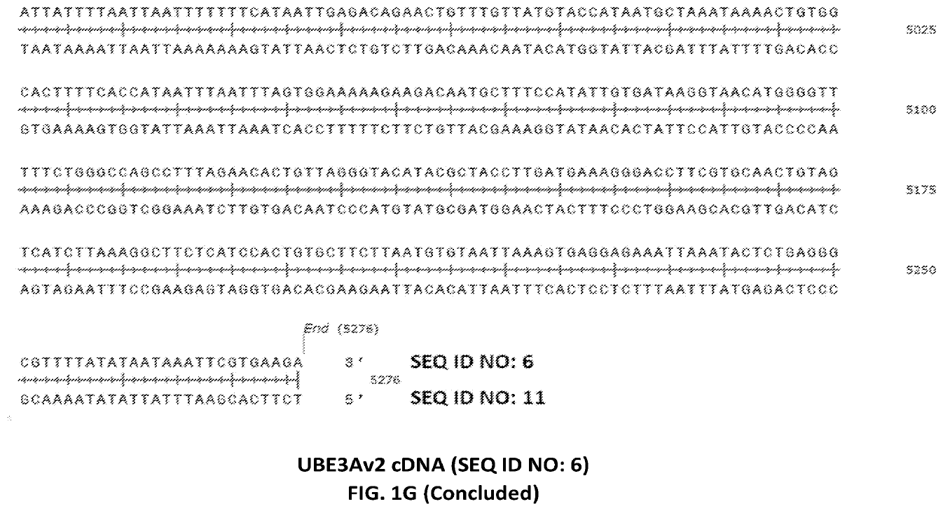

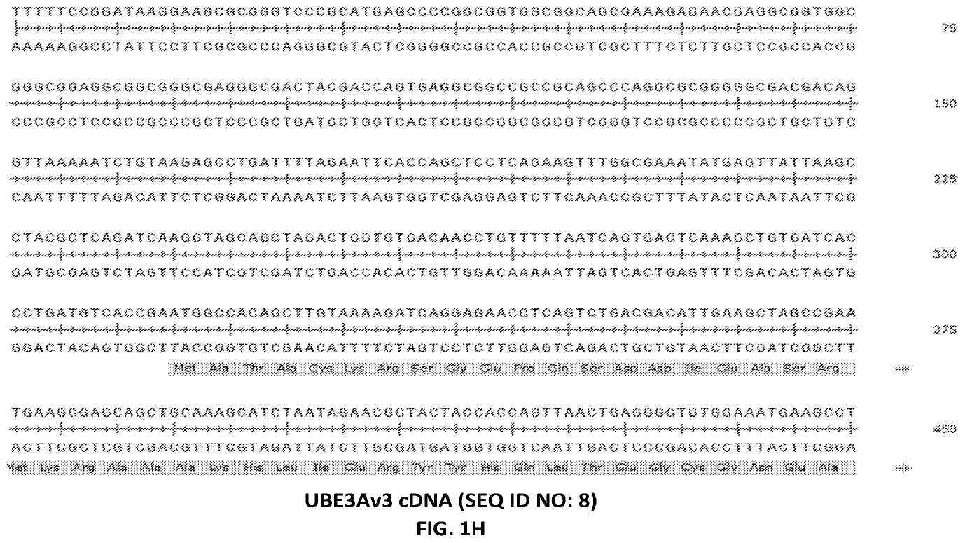

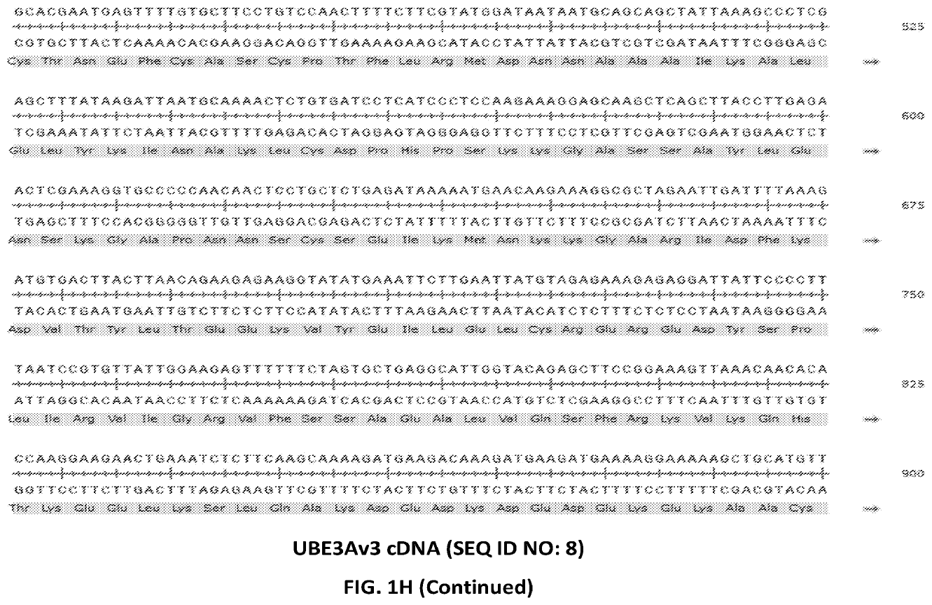

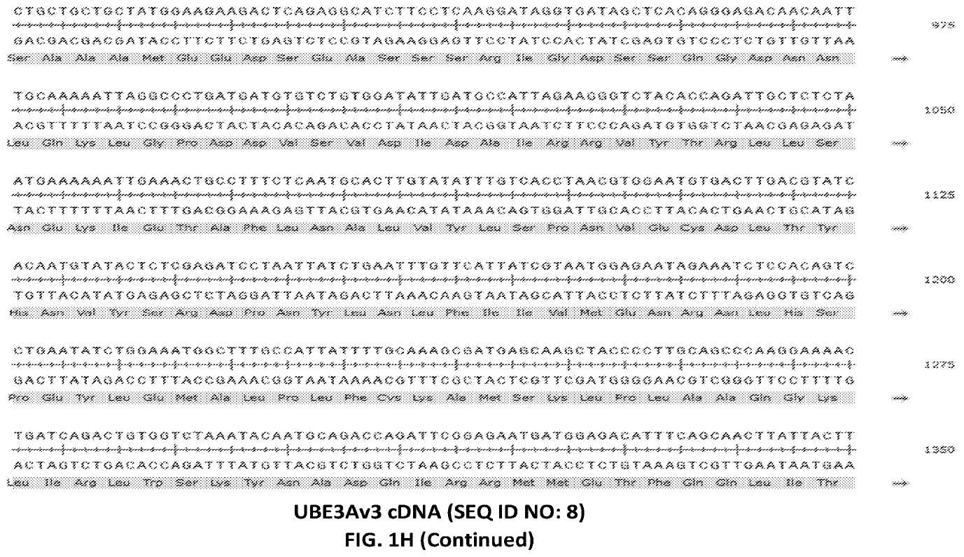

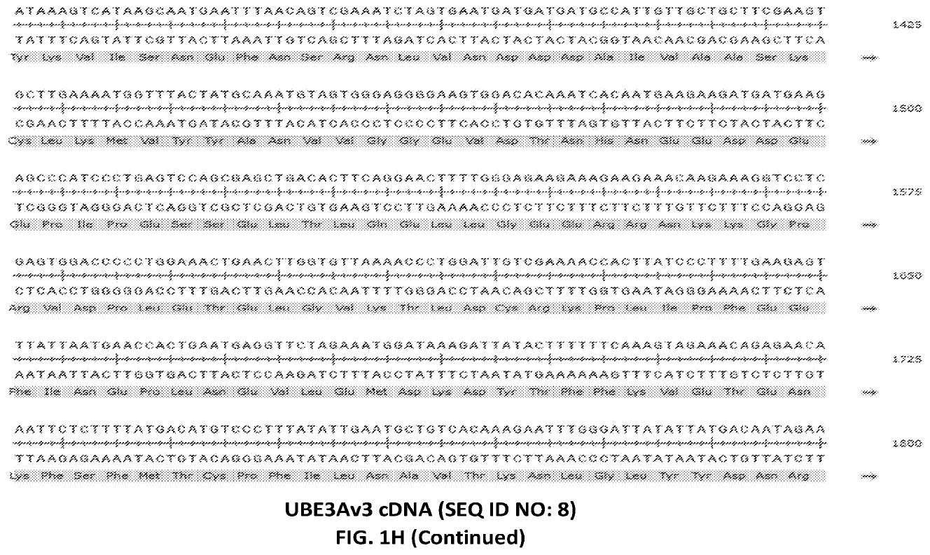

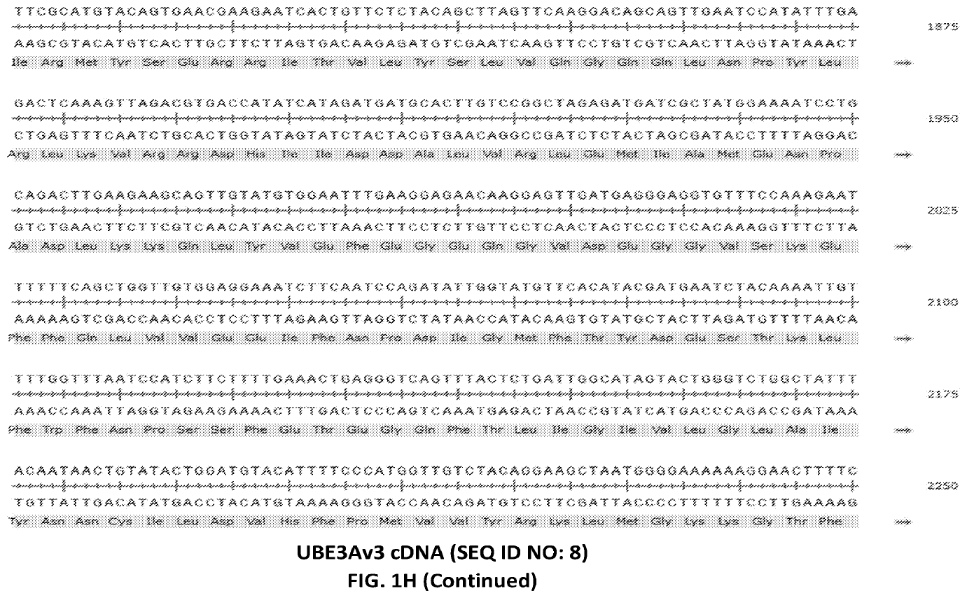

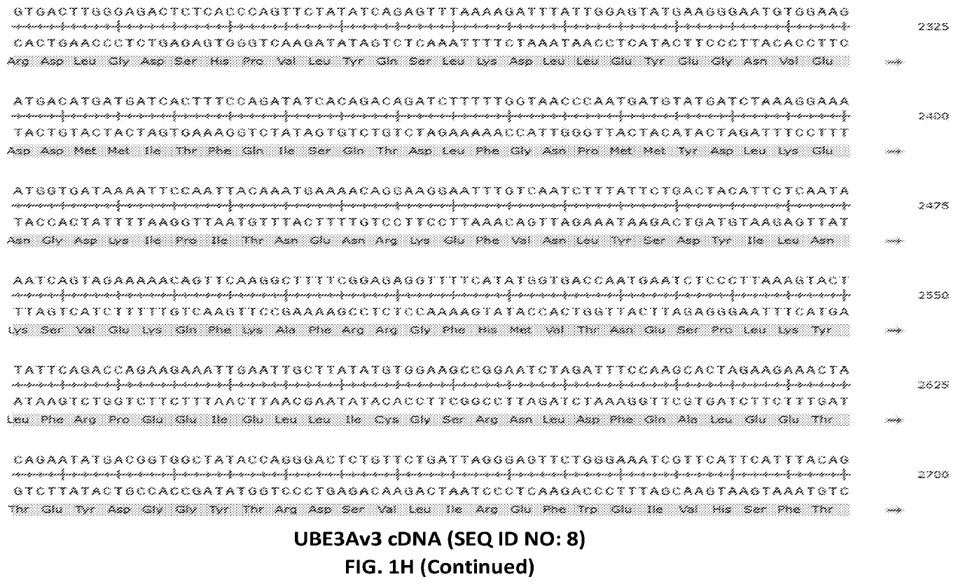

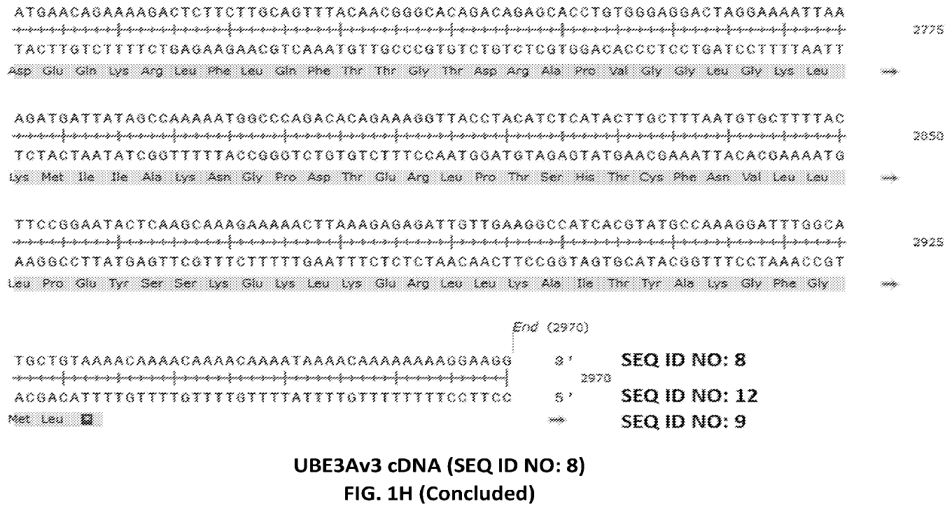

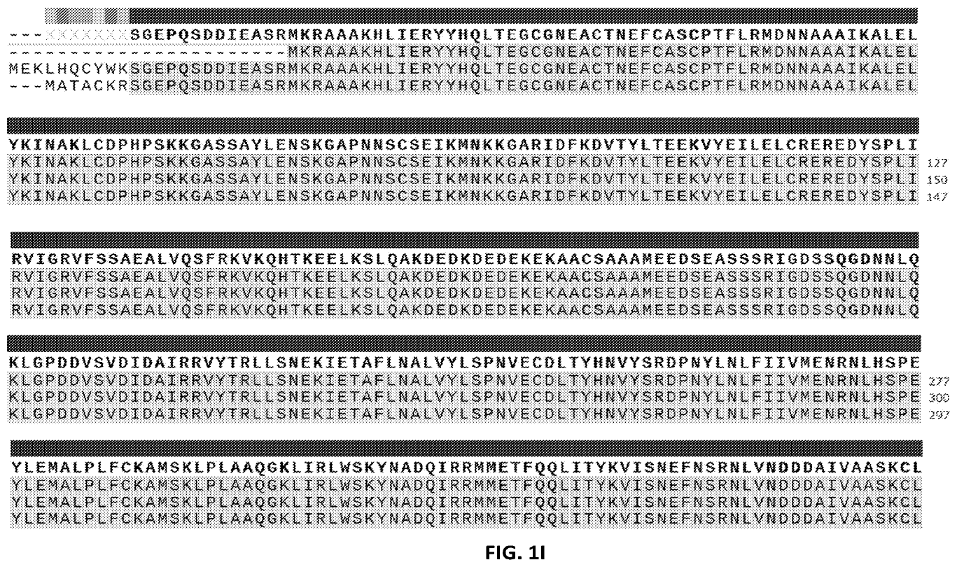

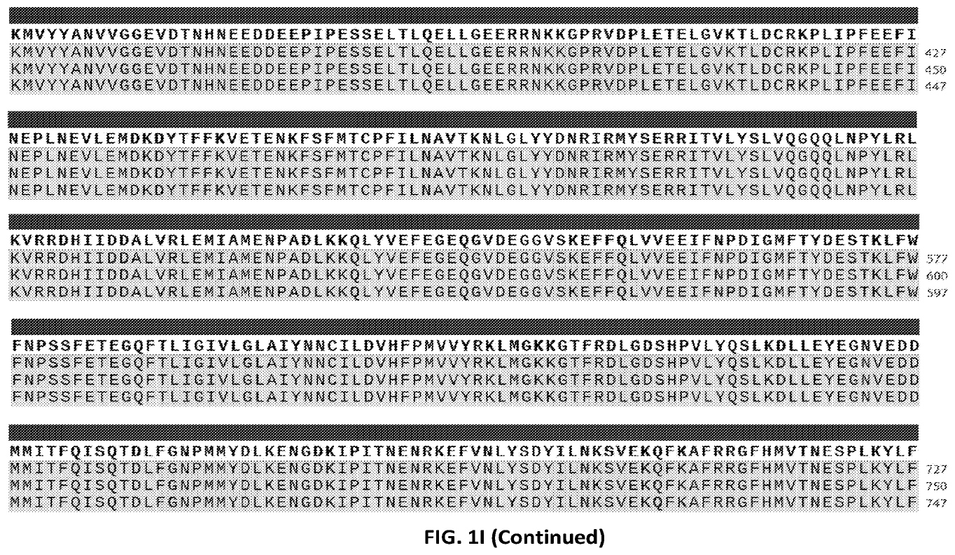

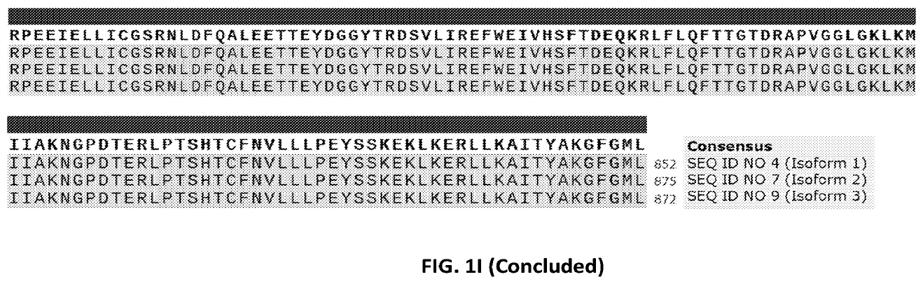

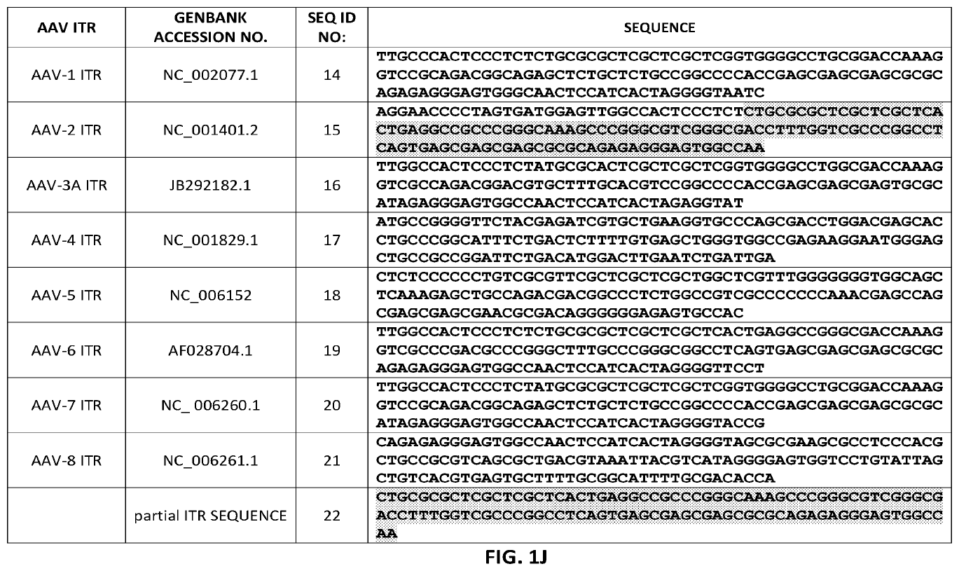

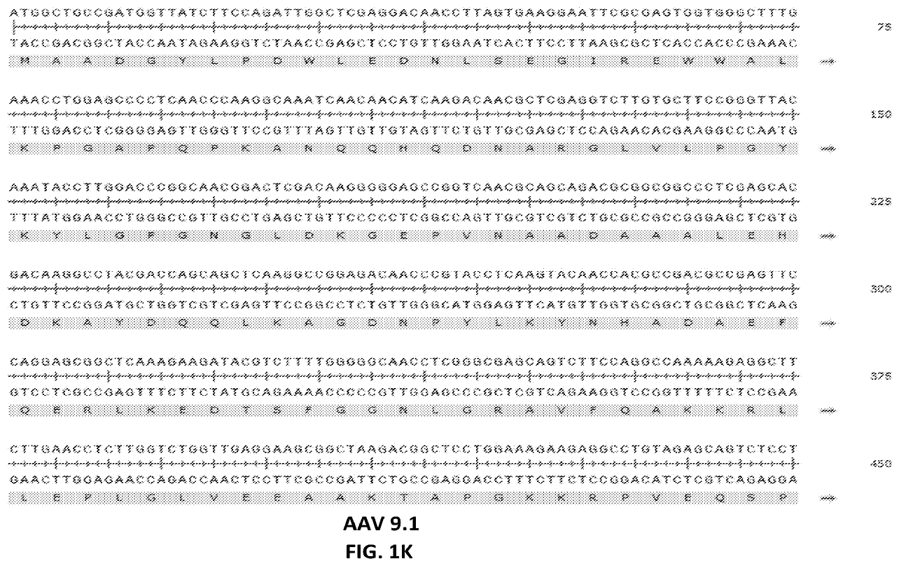

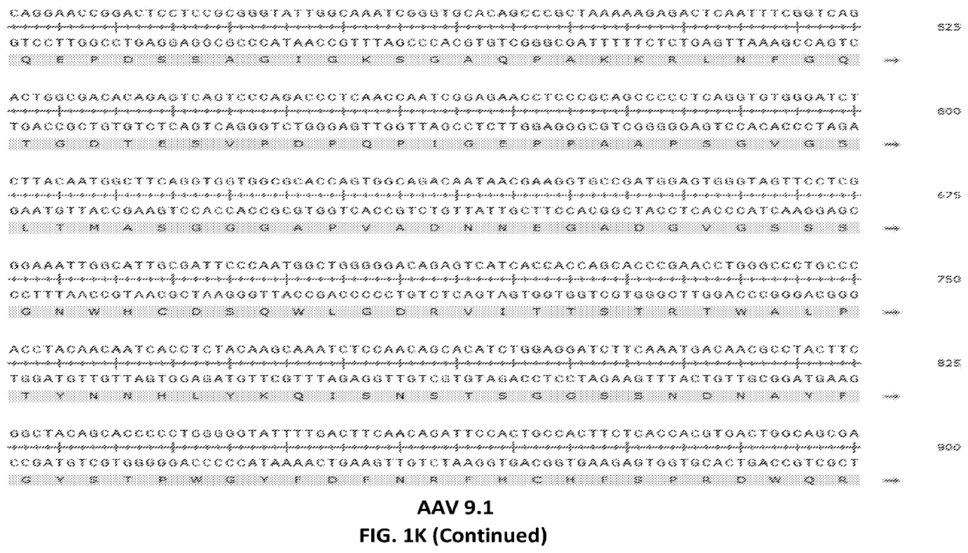

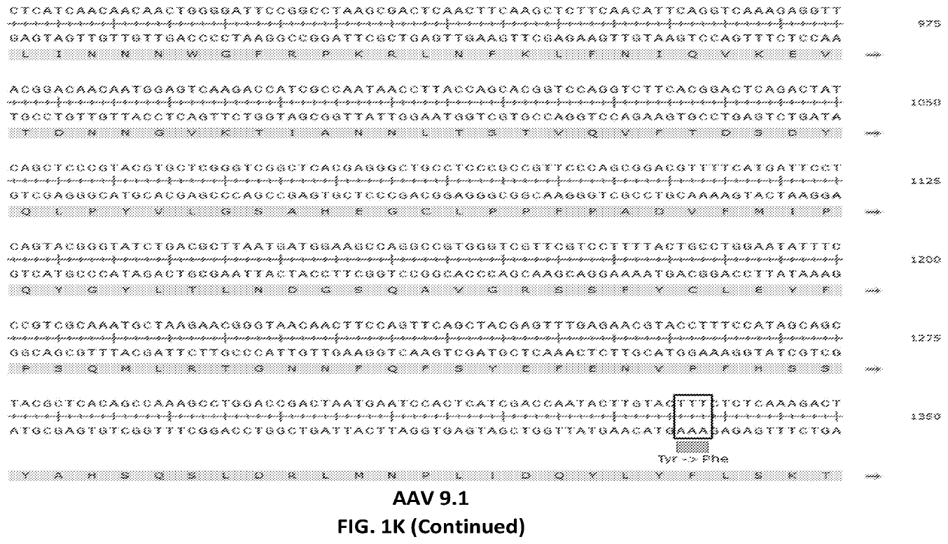

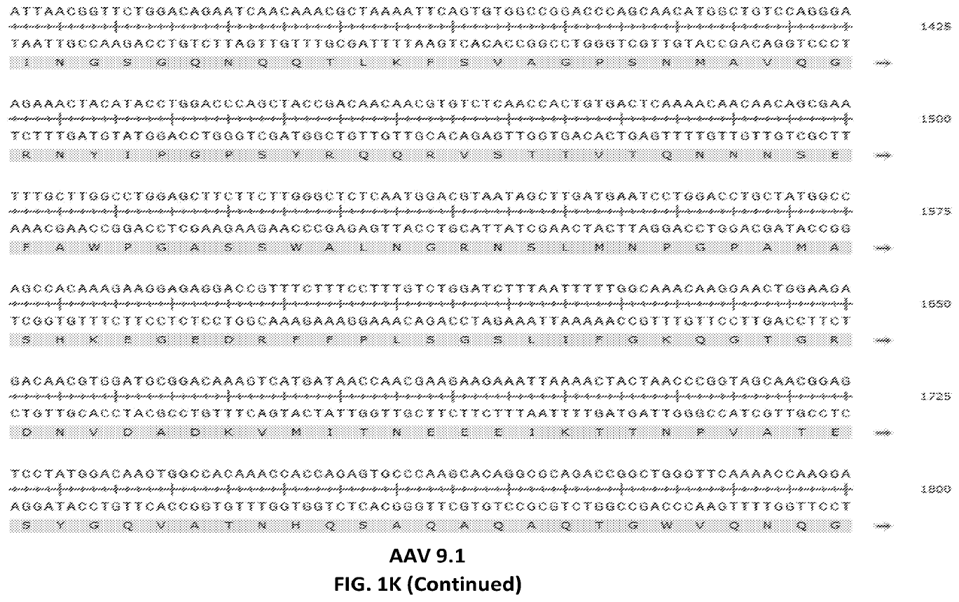

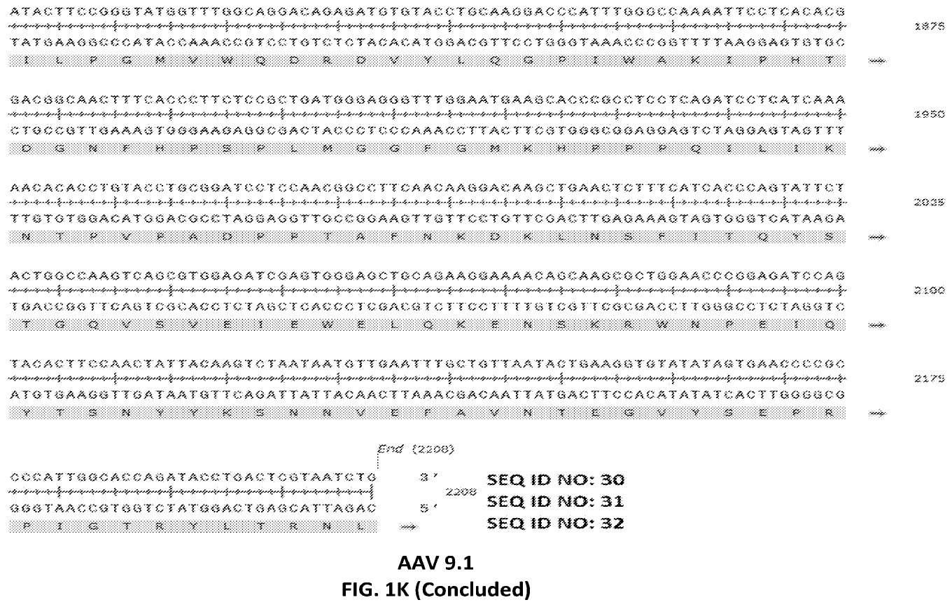

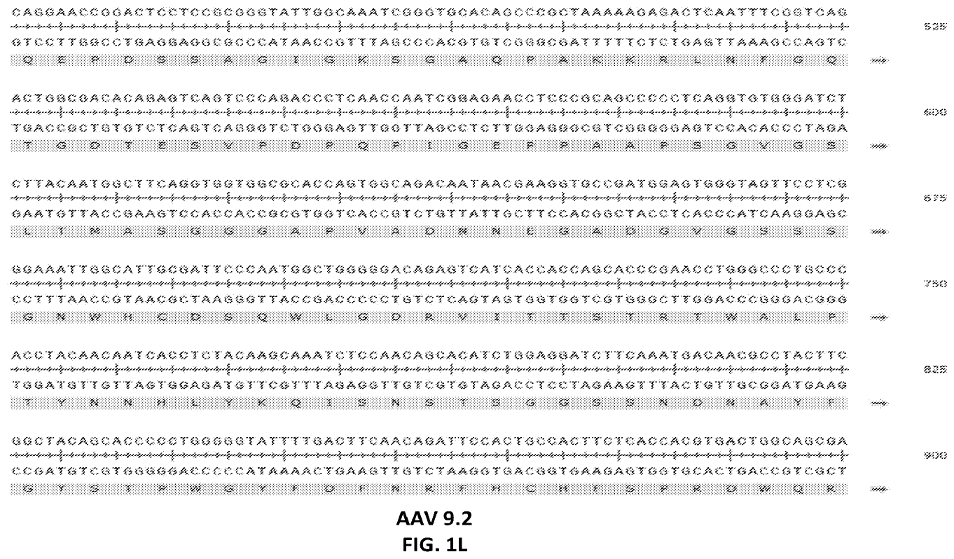

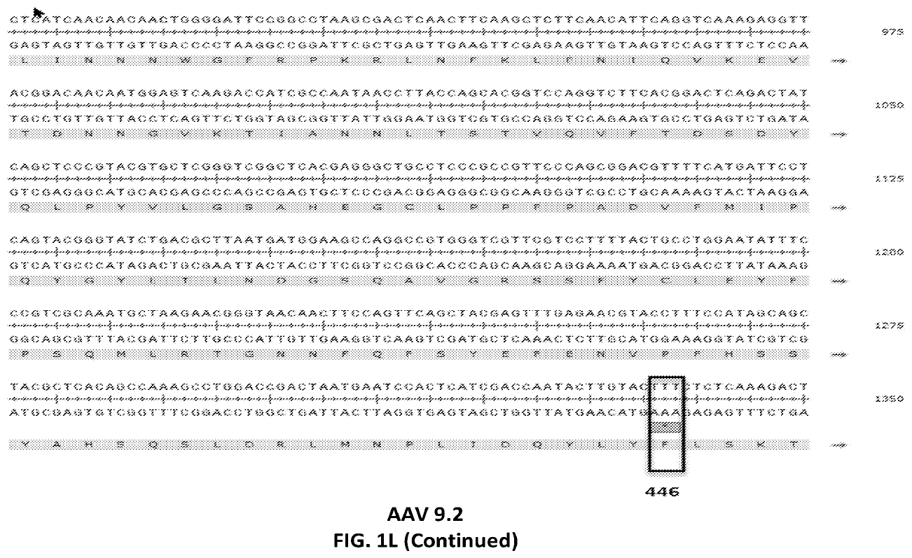

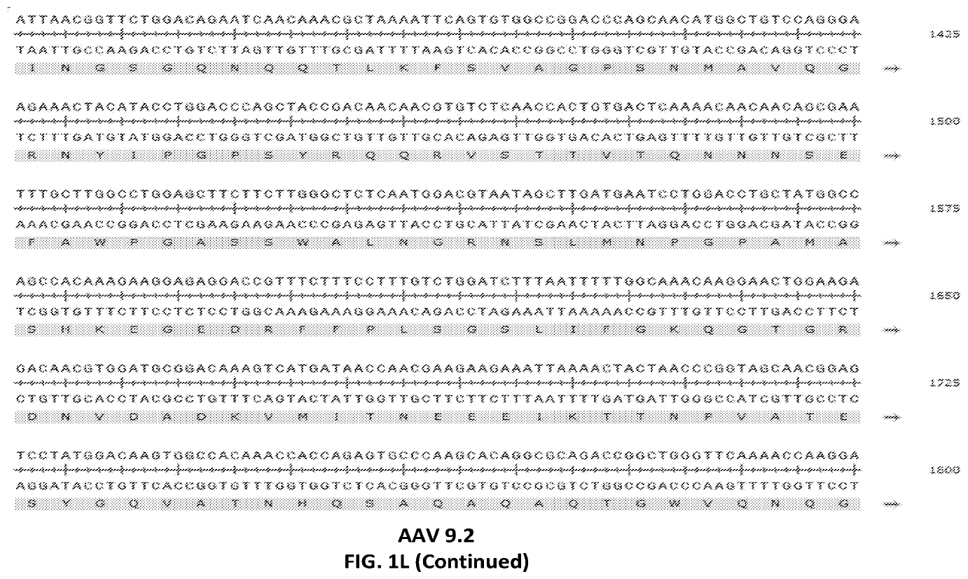

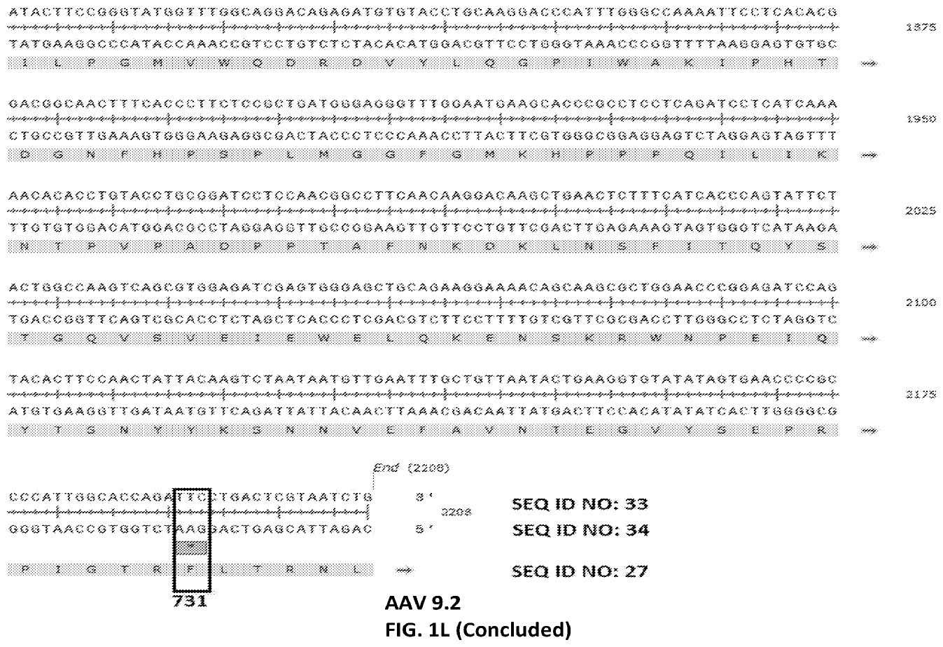

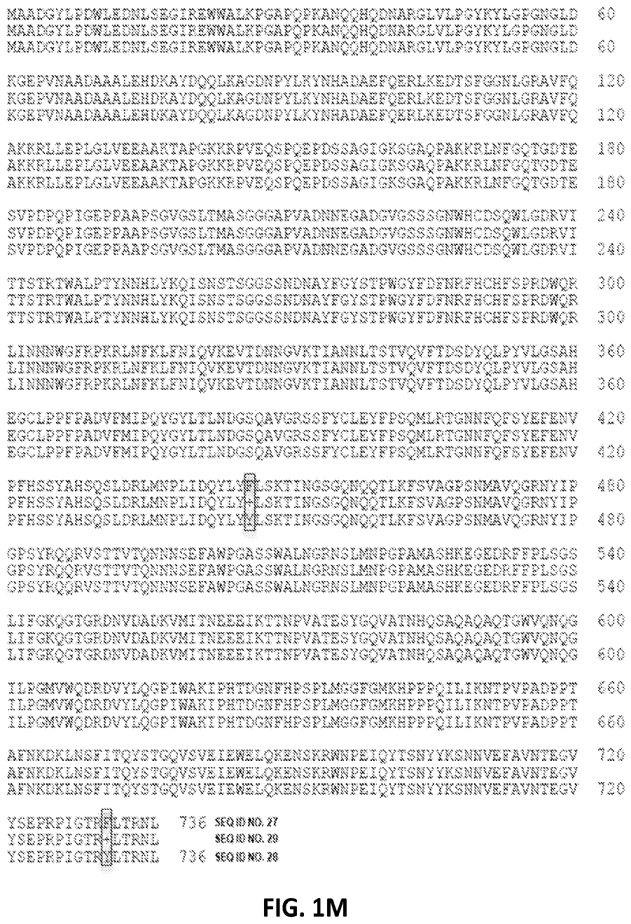

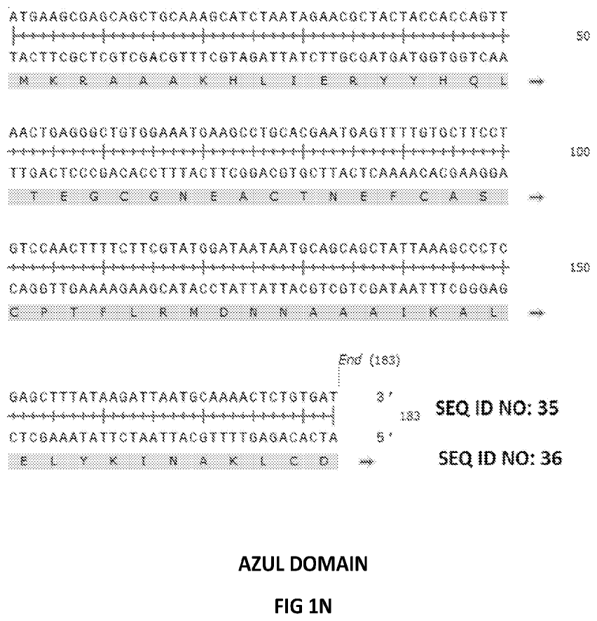

The AAV sequences of the vector typically comprise the cis-acting 5′ and 3′ inverted terminal repeat sequences (See, e.g., B. J. Carter, in “Handbook of Parvoviruses”, ed., P. Tijsser, CRC Press, pp. 155-168 (1990)). Preferably, substantially the entire sequences encoding the ITRs are used in the molecule, although some degree of minor modification of these sequences is permissible. The ability to modify these ITR sequences is within the skill of the art. (See, e.g., texts such as Green and Sambrook, “Molecular Cloning. A Laboratory Manual”, 4^{th} ed., Cold Spring Harbor Laboratory, New York (2014); and K. Fisher et al., J Virol., 70:520 532 (1996)). An example of such a molecule employed in the present disclosure is a “cis-acting” plasmid containing the transgene, in which the selected transgene sequence and associated regulatory elements are flanked by the 5′ and 3′ AAV ITR sequences. The AAV ITR sequences may be obtained from any known AAV, including presently identified mammalian AAV types (see, e.g. FIG. 1J).

In addition to the major elements identified above for the recombinant AAV vector, the vector also includes conventional control elements which are operably linked to the transgene in a manner which permits its transcription, translation and/or expression in a cell transfected with the plasmid vector or infected with the virus produced by the disclosure. As used herein, “operably linked” sequences include both expression control sequences that are contiguous with the gene of interest and expression control sequences that act in trans or at a distance to control the gene of interest. Expression control sequences include appropriate transcription initiation, termination, promoter and enhancer sequences; efficient RNA processing signals such as splicing and polyadenylation (polyA) signals; sequences that stabilize cytoplasmic mRNA; sequences that enhance translation efficiency (i.e., Kozak consensus sequence); sequences that enhance protein stability. A great number of expression control sequences, including promoters which are native, constitutive, inducible and/or tissue-specific, are known in the art and may be utilized.

For nucleic acids encoding proteins, a polyadenylation sequence generally is inserted following the transgene sequences and before the 3′ AAV ITR sequence. A rAAV construct useful in the present disclosure may also contain an intron, desirably located between the promoter/enhancer sequence and the transgene. One possible intron sequence is derived from SV40 and is referred to as the SV40 T intron sequence. Another vector element that may be used is an internal ribosome entry site (IRES). An IRES sequence is used to produce more than one polypeptide from a single gene transcript. An IRES sequence would be used to produce a protein that contain more than one polypeptide chains. Selection of these and other common vector elements are conventional, and many such sequences are available [see, e.g., Sambrook et al, and references cited therein at, for example, pages 3.18 3.26 and 16.17 16.27 and Ausubel et al., Current Protocols in Molecular Biology, John Wiley & Sons, New York, 1989]. In some aspects, a Foot and Mouth Disease Virus 2A sequence is included in polyprotein; this is a small peptide (approximately 18 amino acids in length) that has been shown to mediate the cleavage of polyproteins (Ryan, M D et al., EMBO, 1994; 4: 928-933; Mattion, N M et al., J Virology, November 1996; p. 8124-8127; Furler, S et al., Gene Therapy, 2001; 8: 864-873; and Halpin, C et al., The Plant Journal, 1999; 4: 453-459). The cleavage activity of the 2A sequence has previously been demonstrated in artificial systems including plasmids and gene therapy vectors (AAV and retroviruses) (Ryan, M D et al., EMBO, 1994; 4: 928-933; Mattion, N M et al., J Virology, November 1996; p. 8124-8127; Furler, S et al., Gene Therapy, 2001; 8: 864-873; and Halpin, C et al., The Plant Journal, 1999; 4: 453-459; de Felipe, P et al., Gene Therapy, 1999; 6: 198-208; de Felipe, P et al., Human Gene Therapy, 2000; 11: 1921-1931.; and Klump, H et al., Gene Therapy, 2001; 8: 811-817).

In some aspects, the nucleic acid in the UBE3A vector comprises an ITR of AAV1, AAV2, AAV3, AAV4, AAV5, AA6, AAV7, AAV8, AAV9, AAVrh.8, AAVrh.10, AAV11, AAV12 or the like.

Unless otherwise specified, the AAV ITRs, and other selected AAV components described herein, may be readily selected from among any AAV serotype, including without limitation, AAV1, AAV2, AAV3, AAV4, AAV5, AAV6, AAV7, AAV8 and AAV9. These ITRs or other AAV components may be readily isolated using techniques available to those of skill in the art from an AAV serotype. AAVs may be isolated or obtained from academic, commercial, or public sources (e.g., the American Type Culture Collection, Manassas, Va.). Alternatively, the AAV sequences may be obtained through synthetic or other suitable means by reference to published sequences such as are available in the literature or in databases such as, e.g., GenBank, PubMed, or the like (see for example, the ITR sequences shown in FIG. 1G).

The precise nature of the regulatory sequences needed for gene expression in host cells may vary between species, tissues or cell types, but shall in general include, as necessary, 5′ non-transcribed and 5′ non-translated sequences involved with the initiation of transcription and translation respectively, such as a TATA box, capping sequence, CAAT sequence, enhancer elements, and the like. Especially, such 5′ non-transcribed regulatory sequences will include a promoter region that includes a promoter sequence for transcriptional control of the operably joined gene. Regulatory sequences may also include enhancer sequences or upstream activator sequences as desired. The vectors of the disclosure may optionally include 5′ leader or signal sequences. The choice and design of an appropriate vector is within the ability and discretion of one of ordinary skill in the art. In some aspects of the disclosure, the vector does not comprise an extraneous signal sequence.

Examples of constitutive promoters include, but are not limited to, the retroviral Rous sarcoma virus (RSV) LTR promoter, optionally with the RSV enhancer, the cytomegalovirus immediate-early promoter (CMV), optionally with the CMV enhancer (see, e.g., Boshart et al, Cell, 41:521-530 (1985)), the simian virus 40 early promoter (SV40), the human elongation factor 1α promoter (EF1A), the dihydrofolate reductase promoter, the mouse phosphoglycerate kinase 1 promoter (PGK) promoter, the human Ubiquitin C (UBC) promoter and the chicken β-Actin promoter coupled with CMV early enhancer (CAGG).

Inducible promoters allow regulation of gene expression and can be regulated by exogenously supplied compounds, environmental factors such as temperature, or the presence of a specific physiological state, e.g., acute phase, a particular differentiation state of the cell, or in replicating cells only.

Examples of inducible expression systems include but are not limited to: a tetracycline (Tet) inducible system (see e.g., Gossen et al. (1992) Proc. Natl. Acad. Sci. USA 89:5547 5551; Gossen et al. (1995) Science 268:1766 1769; and Harvey et al., Curr. Opin. Chem. Biol., 2:512-518 (1998)) which are incorporated by reference herein in their entireties); a FK506/rapamycin inducible system (see e.g., Spencer et al. (1993) Science 262:1019 1024; Belshaw et al. (1996) Proc. Natl. Acad. Sci. USA 93:4604 4607 and Magari et al, J. Clin. Invest., 100:2865-2872 (1997), which are incorporated by reference herein in their entireties); a RU486/mifepristone inducible system (Wang et al., Proc. Natl. Acad. Sci. USA (1994) 91(17):8180-4, Wang et al, Nat. Biotech., 15:239-243 (1997) and Wang et al, Gene Ther., 4:432-441 (1997)), which are incorporated by reference herein in their entireties); a cumate inducible system (Mullick et al. BMC Biotechnol. 2006 3; 6:43, which is incorporated by reference herein in its entirety), an ecdysone inducible system (for review, see Rossi et al. (1989) Curr. Op. Biotech. 9:451 456, which is incorporated by reference herein in its entirety), a zinc-inducible sheep metallothionine (MT) promoter, a dexamethasone (Dex)-inducible mouse mammary tumor virus (MMTV) promoter, the T7 polymerase promoter system (WO 98/10088) or an ecdysone-inducible insect promoter (No et al, Proc. Natl. Acad. Sci. USA, 93:3346-3351 (1996)). Many constitutive, tissue-specific and inducible promoters are commercially available from vendors such as Origene, Promega, Invitrogen, System Biosciences and Invivogen.

In certain aspects, the term “inducible” means the transcription of a protein-coding sequence can be regulated by an inducer or repressor molecule acting on one or more transcription factors binding to its promoter. For example, removal of the inducer down-regulates transgene expression whereas the presence of the inducer up-regulates transgene expression. Conversely, removal of a repressor up-regulates transgene expression whereas the presence of the repressor down-regulates transgene expression.

In other aspects, the expression of a protein-coding sequence can be down-regulated by site-specific recombinase mediated excision of the transgene or a portion thereof.

In certain aspects, the transgenes disclosed herein can be fused in frame to sequences encoding destabilizing domains (DD), e.g., FK506- and rapamycin-binding protein (FKBP12) that destabilize the resulting fusion proteins. The level of the fusion protein can then be regulated through the addition of the small-molecule rapamycin. In the absence of the small molecule the fusion protein is destabilized and degraded. Expression of the fusion protein can then be regulated by the small molecule in a dose-dependent manner. Small-Molecule Modulation of Protein Homeostasis is reviewed by Burslem and Crews Chem. Rev. (2017) 117, 11269-11301, the content of which is incorporated by reference herein in its entirety.

In another aspect, the native promoter, or fragment thereof, for the transgene can be used to drive transgene expression. The native promoter may be preferred when it is desired that expression of the transgene should mimic the native expression. The native promoter may be used when expression of the transgene must be regulated temporally or developmentally, or in a tissue-specific manner, or in response to specific transcriptional stimuli. In a further aspect, other native expression control elements, such as enhancer elements, polyadenylation sites or Kozak consensus sequences may also be used to mimic the native expression.

In some aspects, the regulatory sequences impart tissue-specific gene expression capabilities. In some cases, the tissue-specific regulatory sequences bind tissue-specific transcription factors that induce transcription in a tissue specific manner. Such tissue-specific regulatory sequences (e.g., promoters, enhancers, etc.) are well known in the art. Exemplary tissue-specific regulatory sequences include but are not limited to the following tissue specific promoters: neuronal such as neuron-specific enolase (NSE) promoter (Andersen et al., Cell. Mol. Neurobiol., 13:503-15 (1993)), neurofilament light-chain gene promoter (Piccioli et al., Proc. Natl. Acad. Sci. USA, 88:5611-5 (1991)), and the neuron-specific vgf gene promoter (Piccioli et al., Neuron, 15:373-84 (1995)). In some aspects, the tissue-specific promoter is a promoter of a gene selected from: neuronal nuclei (NeuN), glial fibrillary acidic protein (GFAP), adenomatous polyposis coli (APC), and ionized calcium-binding adapter molecule 1 (Iba-1). Other appropriate tissue specific promoters will be apparent to the skilled artisan.

In some aspects, one or more bindings sites for one or more of miRNAs are incorporated into a transgene of a rAAV vector, to inhibit the expression of the transgene in one or more tissues of a subject harboring the transgenes, e.g., non-CNS tissues. The skilled artisan will appreciate that binding sites may be selected to control the expression of a transgene in a tissue-specific manner. For example, expression of a transgene may be inhibited by incorporating a binding site for miR-122 such that mRNA expressed from the transgene binds to and inhibits in the liver. Expression of a transgene in the heart may be inhibited by incorporating a binding site for miR-133a or miR-1, such that mRNA expressed from the transgene binds to and is inhibited by miR-133a or miR-1 in the heart. The miRNA target sites in the mRNA may be in the 5′ UTR, the 3′ UTR or in the coding region. Typically, the target site is in the 3′ UTR of the mRNA. Furthermore, the transgene may be designed such that multiple miRNAs regulate the mRNA by recognizing the same or multiple sites. The presence of multiple miRNA binding sites may result in the cooperative action of multiple RNA-induced silencing complexes (RISCs) and provide highly efficient inhibition of expression. The target site sequence may comprise a total of 5-100, 10-60, or more nucleotides. The target site sequence may comprise at least 5 nucleotides of the sequence of a target gene binding site.

### UBE3A Transgenes

In some aspects, the disclosure provides rAAV vectors for use in methods of preventing or treating Angelman's Syndrome (AS) in a mammal by rescuing a UBE3A gene defect that results in a deficiency in the expression of functional UBE3A polypeptide within a cerebral tissue of a subject having or suspected of having such a disorder.

The UBE3A gene encodes E3 ubiquitin-protein ligase is part of the ubiquitin protein degradation system. This imprinted gene is maternally expressed in brain and biallelically expressed in other tissues. Maternally inherited deletion of this gene is implicated in the etiology of Angelman Syndrome, characterized by severe motor and intellectual retardation, ataxia, hypotonia, epilepsy, absence of speech, and characteristic facies.

In humans, the E6AP ubiquitin-protein ligase (UBE3A) gene is located within the q11-q13 region on chromosome 15 and has the nucleotide sequence of SEQ ID NO. 3 (see FIG. 1D; Accession No: AH005553). Alternative splicing of this gene results in three transcript variants encoding three isoforms with different N-termini (Yamamoto, Y., et al. (1997) Genomics 41(2): 263-266; the content of which is incorporated by reference herein in its entirety). A sequence alignment of UBE3A isoforms 1, 2, and 3 is depicted in FIG. 1I.

The hUBE3A.v1 (variant 1) cDNA sequence (SEQ ID NO: 5; see FIG. E) comprises the nucleotide sequence of SEQ ID NO: 25 that encodes UBE3A protein isoform 1 having the amino acid sequence SEQ. ID. NO. 4 (see FIG. 1F).

The hUBE3A Variant 2 (hUBE3a.v2) cDNA having the nucleotide sequence of SEQ ID NO: 6 comprises an open reading frame (ORF) that encodes the hUBEA3 Isoform 2 having the amino acid sequence of SEQ ID NO. 7 (see FIG. 1G).

The hUBE3A Variant 3 (hUBE3a.v3) cDNA having the nucleotide sequence of SEQ ID NO: 8 comprises an open reading frame (ORF) that encodes the hUBE3A Isoform 3 having the amino acid sequence SEQ ID NO. 9 (see FIG. 1H).

The disclosed AAV therapy for the treatment of Angelman Syndrome aims to rescue defective UBE3A gene expression in brain cells using UBE3A AAV vectors, that when transduced into the affected neural cells, drive the episomal expression of a functional UBE3A transgene.

The nucleic acid packaged in the AAV capsid in the human UBE3A vector of the present disclosure includes a UBE3A transgene, specifically, a UBE3A nucleotide sequence encoding a human UBE3A protein.

In some aspects, the UBE3A transgene can be UBE3A Isoform 1.

In some aspects, the UBE3A transgene can be UBE3A Isoform 2.

In some aspects, the UBE3A transgene can be UBE3A Isoform 3.

In one aspect, the UBE3A transgene encodes a polypeptide comprising a functional fragment of any one of the hUBE3A isoforms.

In some aspects, the UBE3A transgene comprises a nucleotide sequence encoding an ‘Homologous to the E6AP Carboxyl Terminus' (HECT) domain (see Huibregtse et al., (1995) Proc. Natl. Acad. Sci. U.S.A. 92 (7): 2563-7, the content of which is incorporated herein in its entirety).

In another aspect, the UBE3A transgene comprises a nucleotide sequence of SEQ ID NO: 35 that encodes the AZUL Zn finger domain having an amino acid sequence of SEQ ID NO: 36 (see FIG. 1N; Trezza et al. Nat Neurosci. 22, 1235-1247 (2019); see FIG. 1N)

In another aspect, the UBE3A transgene can be a DNA sequence encoding a chimeric polypeptide formed by the fusion of a polypeptide with any one of the hUBE3A isoforms or functional fragments thereof.

In one aspect, the nucleotide sequence encoding the UBE3A isoforms can be codon optimized.

In some aspects, the cloning capacity of the recombinant AAV vector may be limited if they exceed about 4.8 kilobases in length. The skilled artisan will appreciate that options are available in the art for overcoming a limited coding capacity. For example, the AAV ITRs of two genomes can anneal to form head to tail concatemers, almost doubling the capacity of the vector. Insertion of splice sites allows for the removal of the ITRs from the transcript. Other options for overcoming a limited cloning capacity will be apparent to the skilled artisan.

### Recombinant AAVs

In some aspects, the disclosure provides isolated AAVs. As used herein with respect to AAVs, the term “isolated” refers to an AAV that has been isolated from its natural environment (e.g., from a host cell, tissue, or subject) or artificially produced. Isolated AAVs may be produced using recombinant methods. Such AAVs are referred to herein as “recombinant AAVs”. Recombinant AAVs (rAAVs) preferably have tissue-specific targeting capabilities, such that a transgene of the rAAV will be delivered specifically to one or more predetermined tissue(s).

AAV capsid proteins self-assemble to form an icosahedral capsid with a T=1 symmetry, about 22 nm in diameter, and consisting of 60 copies of three size variants of the capsid protein VP1, VP2 and VP3 which differ in their N-terminus. The capsid encapsulates the UBE3A recombinant AAV (rAAV) vector. Without being bound by any theory, the capsid binds to host cell heparan sulfate and uses host ITGA5-ITGB1 as coreceptor on the cell surface to provide virion attachment to target cell. This attachment induces virion internalization predominantly through clathrin-dependent endocytosis. Binding to the host receptor also induces capsid rearrangements leading to surface exposure of VP1 N-terminus, specifically its phospholipase A2-like region and putative nuclear localization signal(s). Without being bound by any theory, the VP1 N-terminus might serve as a lipolytic enzyme to breach the endosomal membrane during entry into host cell and might contribute to virus transport to the nucleus.

In one aspect, the UBE3A vector may comprise a capsid of any AAV serotype. Exemplary AAV serotypes can be found in WO2019222441, the content of which is incorporated by reference herein in its entirety.

In one aspect, the UBE3A recombinant vector is episomal i.e. it does not integrate into the genome.

The AAV capsid, e.g. AAV VP1, is an important element in determining tissue-specific targeting capabilities.

In one aspect, the VP1 capsid for the transduction of neural tissue can be the AAV9 capsid of SEQ ID NO: 28.

In other aspects, the VP1 capsid can be a mutated AAV9.1 capsid having the amino acid of SEQ ID NO: 32.

In other aspects, the VP1 capsid can be a mutated AAV9.1 capsid having the amino acid of SEQ ID NO: 27.

### AAV Packaging

Methods for obtaining recombinant AAVs having a desired capsid protein are well known in the art (See, for example, US 2003/0138772, the contents of which are incorporated herein by reference in their entirety). AAVs capsid protein that may be used in the rAAVs of the disclosure include, for example, those disclosed in G. Gao, et al., J. Virol, 78(12):6381-6388 (June 2004); G. Gao, et al, Proc Natl Acad Sci USA, 100(10):6081-6086 (May 13, 2003); US 2003-0138772, US 2007/0036760, US 2009/0197338, and U.S. provisional application Ser. No. 61/182,084, filed May 28, 2009, the contents of which relating to AAVs capsid proteins and associated nucleotide and amino acid sequences are incorporated herein by reference.

Methods of AAV packaging involve culturing a host cell which contains a nucleic acid sequence encoding an AAV capsid protein or fragment thereof; a functional rep gene; a recombinant AAV vector composed of, AAV inverted terminal repeats (ITRs) and a transgene; and sufficient helper functions to permit packaging of the recombinant AAV vector into the AAV capsid proteins.

The components to be cultured in the host cell to package a rAAV vector in an AAV capsid may be provided to the host cell in trans. Alternatively, any one or more of the required components (e.g., recombinant AAV vector, rep sequences, cap sequences, and/or helper functions) may be provided by a stable host cell which has been engineered to contain one or more of the required components using methods known to those of skill in the art. Most suitably, such a stable host cell will contain the required component(s) under the control of an inducible promoter. However, the required component(s) may be under the control of a constitutive promoter. Examples of suitable inducible and constitutive promoters are provided herein, in the discussion of regulatory elements suitable for use with the transgene. In still another alternative, a selected stable host cell may contain selected component(s) under the control of a constitutive promoter and other selected component(s) under the control of one or more inducible promoters. For example, a stable host cell may be generated which is derived from 293 cells (which contain E1 helper functions under the control of a constitutive promoter), but which contain the rep and/or cap proteins under the control of inducible promoters. Still other stable host cells may be generated by one of skill in the art.

The recombinant AAV vector, rep sequences, cap sequences, and helper functions required for producing the rAAV of the invention may be delivered to the packaging host cell using any appropriate genetic element (vector). The selected genetic element may be delivered by any suitable method, including those described herein. The methods used to construct any aspect of this invention are known to those with skill in nucleic acid manipulation and include genetic engineering, recombinant engineering, and synthetic techniques. See, e.g., Sambrook et al, Molecular Cloning: A Laboratory Manual, Cold Spring Harbor Press, Cold Spring Harbor, N.Y. Similarly, methods of generating rAAV virions are well known and the selection of a suitable method is not a limitation on the present disclosure. See, e.g., K. Fisher et al, J. Virol., 70:520-532 (1993) and U.S. Pat. No. 5,478,745, the contents of which are incorporated by reference herein in their entireties.

In some aspects, recombinant AAVs may be produced using the triple transfection method (e.g., as described in detail in U.S. Pat. No. 6,001,650, the contents of which relating to the triple transfection method are incorporated herein by reference). Typically, the recombinant AAVs are produced by transfecting a host cell with a recombinant AAV vector (comprising a transgene) to be packaged into AAV particles, an AAV helper function vector, and an accessory function vector. An AAV helper function vector encodes the “AAV helper function” sequences (i.e., rep and cap), which function in trans for productive AAV replication and encapsidation. Preferably, the AAV helper function vector supports efficient AAV vector production without generating any detectable wild-type AAV virions (i.e., AAV virions containing functional rep and cap genes). Non-limiting examples of vectors suitable for use with the present disclosure include pHLP19, described in U.S. Pat. No. 6,001,650 and pRep6cap6 vector, described in U.S. Pat. No. 6,156,303, the entirety of both incorporated by reference herein. The accessory function vector encodes nucleotide sequences for non-AAV derived viral and/or cellular functions upon which AAV is dependent for replication (i.e., “accessory functions”). The accessory functions include those functions required for AAV replication, including, without limitation, those moieties involved in activation of AAV gene transcription, stage specific AAV mRNA splicing, AAV DNA replication, synthesis of cap expression products, and AAV capsid assembly. In one aspect, a UBE3A plasmid is formed using a transcription initiation sequence, and a UBE3A gene construct disposed downstream of the transcription initiation sequence.

In one aspect, a UBE3A expression plasmid is formed from cDNA cloned from a Homo sapiens UBE3A gene to form a UBE3A gene, Version 1 (UBE3A.v1) gene with a promoter, such as a human Ubiquitin ligase C promoter (see, e.g. FIGS. 1A and 1B).

In another aspect, methods for preparing a UBE3A expression plasmid may be found, for example, in International Publication Numbers WO2016/179584 and WO2019/006107, which are incorporated by reference herein in their entireties.

An rAAV vector with the UBE3A transgene transcribed from the UBE3A expression plasmid (the ITR to ITR sequence) is then packaged according to methods that are well known in the art. Exemplary methods of preparing UBE3A rAAV are disclosed in U.S. Pat. Nos. 10,557,149, the published U.S. patent application No. 2018/0327722 and the International Patent Application Nos. WO2020/041773, WO2019/217483, and WO2019/210267.

### Animal Models of Angelman Syndrome

The efficacy of a recombinant UBE3A AAV vector in treating Angelman's Syndrome can be tested in an appropriate animal model of the disease. Angelman Syndrome in humans is caused by a disruption to the maternal UBE3A allele. This includes uniparental disomy, deletion, and mutation (Fang P et al., Human Molecular Genetics, 1999, 8(1):129-135; the content of which is incorporated by reference herein in its entirety). Each of these naturally occurring situations can be replicated in an animal (see, e.g., the published U.S. patent application 2019/0208752, the content of which is incorporated by reference herein in its entirety). The UBE3A-deficient animals may be produced using any technique that results in the deletion or inactivation of the UBE3A gene. In one aspect, clustered regularly interspaced short palindromic repeats (CRISPR) may be used at the germline level to recreate animals where the gene is changed or it may be targeted at non-germline cells, such as brain cells (van Erp P B et al., Current Opinion in Virology, 2015, 12:85-90; Maggio I et al., Trends in Biotechnology, 2015, 33(5):280-291; Rath D et al., Biochimi, 2015, 117:119-128; and Freedman B S et al., Nature Communications, 2015, 6:8715, the contents of which are hereby incorporated by reference herein in their entireties).

### Administration of the Human UBE3A Vector

Non-limiting examples of methods of administration include intravenous administration, infusion, intracranial administration, intrathecal administration, intraganglionic administration, intraspinal administration, cisterna magna administration and intraneural administration. In some cases, administration can involve injection of a liquid formulation of the vector. In other cases, a vector can be intravenously, intrathecally, intrecranially, intraneurally, intraganglionicly, intraspinally, or intracerebroventricularly administered to a subject in order to introduce the vector into one or more neuronal cells.

The intrathecal (IT) route delivers AAV to the cerebrospinal fluid (CSF). This route of administration may be suitable for the treatment of e.g., chronic pain or other peripheral nervous system (PNS) or central nervous system (CNS) indications. In animals, IT administration has been achieved by inserting an IT catheter through the cisterna magna and advancing it caudally to the lumbar level. In humans, IT delivery can be easily performed by lumbar puncture (LP), a routine bedside procedure with excellent safety profile.

In yet another particular case, a vector may be administered to the subject by intracranial administration (i.e., directly into the brain). In non-limiting examples of intracranial administration, a vector of the disclosure may be delivered into the cortex of the brain.

A vector dose may be expressed as the number of vector genome units delivered to a subject. A “vector genome unit” as used herein refers to the number of individual vector genomes administered in a dose. The size of an individual vector genome will generally depend on the type of viral vector used. Vector genomes of the disclosure may be from about 1.0 kilobase, 1.5 kilobases, 2.0 kilobases, 2.5 kilobases, 3.0 kilobases, 3.5 kilobases, 4.0 kilobases, 4.5 kilobases, 5.0 kilobases, 5.5 kilobases, 6.0 kilobases, 6.5 kilobases, 7.0 kilobases, 7.5 kilobases, 8.0 kilobases, 8.5 kilobases, 9.0 kilobases, 9.5 kilobases, 10.0 kilobases, to more than 10.0 kilobases. Therefore, a single vector genome may include up to or greater than 10,000 base pairs of nucleotides. In some cases, a vector dose may be about 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 3×10¹¹, 4×10¹¹, 5×10¹¹, 6×10¹¹, 7×10¹¹, 8×10¹¹, 9×10¹¹, 1×10¹², 2×10¹², 3×10¹², 4×10¹², 5×10¹², 6×10¹², 7×10¹², 8×10¹², 9×10¹², 1×10¹³, 2×10¹³, 3×10¹³, 4×10¹³, 5×10¹³, 6×10¹³, 7×10¹³, 8×10¹³, 9×10¹³, 1×10¹⁴, 2×10¹⁴, 3×10¹⁴, 4×10¹⁴, 5×10¹⁴, 6×10¹⁴, 7×10¹⁴, 8×10¹⁴, 9×10¹⁴, 1×10¹⁵, 2×10¹⁵, 3×10¹⁵, 4×10¹⁵, 5×10¹⁵, 6×10¹⁵, 7×10¹⁵, 8×10¹⁵, 9×10¹⁵, 1×10¹⁶, 2×10¹⁶, 3×10¹⁶, 4×10¹⁶, 5×10¹⁶, 6×10¹⁶, 7×10¹⁶, 8×10¹⁶, 9×10¹⁶, 1×10¹⁷, 2×10¹⁷, 3×10¹⁷, 4×10¹⁷, 5×10¹⁷, 6×10¹⁷, 7×10¹⁷, 8×10¹⁷, 9×10¹⁷, 1×10¹⁸, 2×10¹⁸, 3×10¹⁸, 4×10¹⁸, 5×10¹⁸, 6×10¹⁸, 7×10¹⁸, 8×10¹⁸, 9×10¹⁸, 1×10¹⁹, 2×10¹⁹, 3×10¹⁹, 4×10¹⁹, 5×10¹⁹, 6×10¹⁹, 7×10¹⁹, 8×10¹⁹, 9×10¹⁹, 1×10²⁰, 2×10²⁰, 3×10²⁰, 4×10²⁰, 5×10²⁰, 6×10²⁰, 7×10²⁰, 8×10²⁰, 9×10²⁰ or more vector genome units.

In one aspect, a vector contemplated herein is administered to a subject at a titer of at least about 1×10⁹ genome particles/mL, at least about 1×10¹⁰ genome particles/mL, at least about 5×10¹⁰ genome particles/mL, at least about 1×10¹¹ genome particles/mL, at least about 5×10¹¹ genome particles/mL, at least about 1×10¹² genome particles/mL, at least about 5×10¹² genome particles/mL, at least about 6×10¹² genome particles/mL, at least about 7×10¹² genome particles/mL, at least about 8×10¹² genome particles/mL, at least about 9×10¹² genome particles/mL, at least about 10×10¹² genome particles/mL, at least about 15×10¹² genome particles/mL, at least about 20×10¹² genome particles/mL, at least about 25×10¹² genome particles/mL, at least about 50×10¹² genome particles/mL, or at least about 100×10¹² genome particles/mL. The terms “genome particles (gp),” or “genome equivalents,” or “genome copies” (gc) as used in reference to a viral titer, refer to the number of virions containing the recombinant UBE3A AAV DNA genome, regardless of infectivity or functionality. The number of genome particles in a vector preparation can be measured by procedures such as described in the Examples herein, or for example, in Clark et al. (1999) Hum. Gene Ther., 10: 1031-1039; Veldwijk et al. (2002) Mol. Ther., 6:272-278, the content of which is incorporated by reference herein in its entirety.

A vector of the disclosure may be administered in a volume of fluid. In some cases, a vector may be administered in a volume of about 0.1 mL, 0.2 mL, 0.3 mL, 0.4 mL, 0.5 mL, 0.6 mL, 0.7 mL, 0.8 mL, 0.9 mL, 1.0 mL, 2.0 mL, 3.0 mL, 4.0 mL, 5.0 mL, 6.0 mL, 7.0 mL, 8.0 mL, 9.0 mL, 10.0 mL, 11.0 mL, 12.0 mL, 13.0 mL, 14.0 mL, 15.0 mL, 16.0 mL, 17.0 mL, 18.0 mL, 19.0 mL, 20.0 mL or greater than 20.0 mL. In some cases, a vector dose may be expressed as a concentration or titer of vector administered to a subject. In this case, a vector dose may be expressed as the number of vector genome units per volume (i.e., genome units/volume).

In one aspect, a vector contemplated herein is administered to a subject at a titer of at least about 5×10⁹ infectious units/mL, at least about 6×10⁹ infectious units/mL, at least about 7×10⁹ infectious units/mL, at least about 8×10⁹ infectious units/mL, at least about 9×10⁹ infectious units/mL, at least about 10×10⁹ infectious units/mL, at least about 15×10⁹ infectious units/mL, at least about 20×10⁹ infectious units/mL, at least about 25×10⁹ infectious units/mL, at least about 50×10⁹ infectious units/mL, or at least about 100×10⁹ infectious units/mL. The terms “infection unit (iu),” “infectious particle,” or “replication unit,” as used in reference to a viral titer, refer to the number of infectious and replication-competent recombinant AAV vector particles as measured by the infectious center assay, also known as replication center assay, as described, for example, in McLaughlin et al. (1988) J. Virol., 62: 1963-1973, the content of which is incorporated by reference herein in its entirety.

In one aspect, a vector contemplated herein is administered to a subject at a titer of at least about 5×10¹⁰ transducing units/mL, at least about 6×10¹⁰ transducing units/mL, at least about 7×10¹⁰ transducing units/mL, at least about 8×10¹⁰ transducing units/mL, at least about 9×10¹⁰ transducing units/mL, at least about 10×10¹⁰ transducing units/mL, at least about 15×10¹⁰ transducing units/mL, at least about 20×10¹⁰ transducing units/mL, at least about 25×10¹⁰ transducing units/mL, at least about 50×10¹⁰ transducing units/mL, or at least about 100×10¹⁰ transducing units/mL. The term “transducing unit (tu)” as used in reference to a viral titer, refers to the number of infectious recombinant AAV vector particles that result in the production of a functional transgene product as measured in functional assays such as described in Examples herein, or for example, in Xiao et al. (1997) Exp. Neurobiol., 144: 113-124; or in Fisher et al. (1996) J. Virol., 70:520-532 (LFU assay).

In one aspect, a vector contemplated herein is administered to a subject at a titer of 1×10⁶ vg/g of brain mass to about 2.86×10¹² vg/g of brain mass, 2×10⁶ vg/g of brain mass to about 2.86×10¹² vg/g of brain mass, 3×10⁶ vg/g of brain mass to about 2.86×10¹² vg/g of brain mass, 4×10⁶ vg/g of brain mass to about 2.86×10¹² vg/g of brain mass, 5×10⁶ vg/g of brain mass to about 2.86×10¹² vg/g of brain mass, 6×10⁶ vg/g of brain mass to about 2.86×10¹² vg/g of brain mass, 7×10⁶ vg/g of brain mass to about 2.86×10¹² vg/g of brain mass, 8×10⁶ vg/g of brain mass to about 2.86×10¹² vg/g of brain mass, 9×10⁶ vg/g of brain mass to about 2.86×10¹² vg/g of brain mass, 1×10⁷ vg/g of brain mass to about 2.86×10¹² vg/g of brain mass, 2×10⁷ vg/g of brain mass to about 2.86×10¹² vg/g of brain mass, 3×10⁷ vg/g of brain mass to about 2.86×10¹² vg/g of brain mass, 4×10⁷ vg/g of brain mass to about 2.86×10¹² vg/g of brain mass, 5×10⁷ vg/g of brain mass to about 2.86×10¹² vg/g of brain mass, 6×10⁷ vg/g of brain mass to about 2.86×10¹² vg/g of brain mass, 7×10⁷ vg/g of brain mass to about 2.86×10¹² vg/g of brain mass, 8×10⁷ vg/g of brain mass to about 2.86×10¹² vg/g of brain mass, 9×10⁷ vg/g of brain mass to about 2.86×10¹² vg/g of brain mass, 1×10⁸ vg/g of brain mass to about 2.86×10¹² vg/g of brain mass, 2×10⁸ vg/g of brain mass to about 2.86×10¹² vg/g of brain mass, 3×10⁸ vg/g of brain mass to about 2.86×10¹² vg/g of brain mass, 4×10⁸ vg/g of brain mass to about 2.86×10¹² vg/g of brain mass, 5×10⁸ vg/g of brain mass to about 2.86×10¹² vg/g of brain mass, 6×10⁸ vg/g of brain mass to about 2.86×10¹² vg/g of brain mass, 7×10⁸ vg/g of brain mass to about 2.86×10¹² vg/g of brain mass, 8×10⁸ vg/g of brain mass to about 2.86×10¹² vg/g of brain mass, 9×10⁸ vg/g of brain mass to about 2.86×10¹² vg/g of brain mass, 1×10⁹ vg/g of brain mass to about 2.86×10¹² vg/g of brain mass, 2×10⁹ vg/g of brain mass to about 2.86×10¹² vg/g of brain mass, 3×10⁹ vg/g of brain mass to about 2.86×10¹² vg/g of brain mass, 4×10⁹ vg/g of brain mass to about 2.86×10¹² vg/g of brain mass, 5×10⁹ vg/g of brain mass to about 2.86×10¹² vg/g of brain mass, 6×10⁹ vg/g of brain mass to about 2.86×10¹² vg/g of brain mass, 7×10⁹ vg/g of brain mass to about 2.86×10¹² vg/g of brain mass, 8×10⁹ vg/g of brain mass to about 2.86×10¹² vg/g of brain mass, 9×10⁹ vg/g of brain mass to about 2.86×10¹² vg/g of brain mass, 1×10¹⁰ vg/g of brain mass to about 2.86×10¹² vg/g of brain mass, 2×10¹⁰ vg/g of brain mass to about 2.86×10¹² vg/g of brain mass, 3×10¹⁰ vg/g of brain mass to about 2.86×10¹² vg/g of brain mass, 4×10¹⁰ vg/g of brain mass to about 2.86×10¹² vg/g of brain mass, 5×10¹⁰ vg/g of brain mass to about 2.86×10¹² vg/g of brain mass, 6×10¹⁰ vg/g of brain mass to about 2.86×10¹² vg/g of brain mass, 7×10¹⁰ vg/g of brain mass to about 2.86×10¹² vg/g of brain mass, 8×10¹⁰ vg/g of brain mass to about 2.86×10¹² vg/g of brain mass, 9×10¹⁰ vg/g of brain mass to about 2.86×10¹² vg/g of brain mass, 1×10¹¹ vg/g of brain mass to about 2.86×10¹² vg/g of brain mass, 2×10¹¹, 3×10¹¹ vg/g of brain mass to about 2.86×10¹² vg/g of brain mass, 4×10¹¹ vg/g of brain mass to about 2.86×10¹² vg/g of brain mass, 5×10¹¹ vg/g of brain mass to about 2.86×10¹² vg/g of brain mass, 6×10¹¹ vg/g of brain mass to about 2.86×10¹² vg/g of brain mass, 7×10¹¹ vg/g of brain mass to about 2.86×10¹² vg/g of brain mass, 8×10¹¹ vg/g of brain mass to about 2.86×10¹² vg/g of brain mass, 9×10¹¹ vg/g of brain mass to about 2.86×10¹² vg/g of brain mass or 1×10¹² vg/g of brain mass to about 2.86×10¹² vg/g of brain mass.

In one aspect, a vector contemplated herein is administered to a subject at a titer of 1×10⁶ vg/g of brain mass to about 2×10⁶ vg/g of brain mass, 1×10⁶ vg/g of brain mass to about 3×10⁶ vg/g of brain mass, 1×10⁶ vg/g of brain mass to about 4×10⁶ vg/g of brain mass, 1×10⁶ vg/g of brain mass to about 5×10⁶, 1×10⁶ vg/g of brain mass to about 6×10⁶ vg/g of brain mass, 10⁶ vg/g of brain mass to about 7×10⁶ vg/g of brain mass, 1×10⁶ vg/g of brain mass to about 8×10⁶ vg/g of brain mass, 10⁶ vg/g of brain mass to about 9×10⁶, vg/g of brain mass, 10⁶ vg/g of brain mass to about 1×10⁷ vg/g of brain mass, 10⁶ vg/g of brain mass to about 2×10⁷ vg/g of brain mass, 1×10⁶ vg/g of brain mass to about 3×10⁷ vg/g of brain mass, 1×10⁶ vg/g of brain mass to about 4×10⁷ vg/g of brain mass, about 1×10⁶ vg/g of brain mass to about 5×10⁷ vg/g of brain mass, 1×10⁶ vg/g of brain mass to about 6×10⁷ vg/g of brain mass, 1×10⁶ vg/g of brain mass to about 7×10⁷ vg/g of brain mass, about 1×10⁶ vg/g of brain mass to about 8×10⁷ vg/g of brain mass, about 1×10⁶ vg/g of brain mass to about 9×10⁷ vg/g of brain mass, about 1×10⁶ vg/g of brain mass to about 1×10⁸ vg/g of brain mass, about 1×10⁶ vg/g of brain mass to about 2×10⁸ vg/g of brain mass, about 1×10⁶ vg/g of brain mass to about 3×10⁷ vg/g of brain mass, about 1×10⁶ vg/g of brain mass to about 4×10⁸ vg/g of brain mass, about 1×10⁶ vg/g of brain mass to about 5×10⁸ vg/g of brain mass, about 1×10⁶ vg/g of brain mass to about 6×10⁸ vg/g of brain mass, about 1×10⁶ vg/g of brain mass to about 7×10⁸ vg/g of brain mass, about 1×10⁶ vg/g of brain mass to about 8×10⁸ vg/g of brain mass, about 1×10⁶ vg/g of brain mass to about 9×10⁸ vg/g of brain mass, about 1×10⁶ vg/g of brain mass to about 1×10⁹ vg/g of brain mass, about 1×10⁶ vg/g of brain mass to about 2×10⁹ vg/g of brain mass, about 1×10⁶ vg/g of brain mass to about 3×10⁹ vg/g of brain mass, about 1×10⁶ vg/g of brain mass to about 4×10⁹ vg/g of brain mass, about 1×10⁶ vg/g of brain mass to about 5×10⁹ vg/g of brain mass, about 1×10⁶ vg/g of brain mass to about 6×10⁹ vg/g of brain mass, about 1×10⁶ vg/g of brain mass to about 7×10⁹ vg/g of brain mass, about 1×10⁶ vg/g of brain mass to about 8×10⁹ vg/g of brain mass, about 1×10⁶ vg/g of brain mass to about 9×10⁹ vg/g of brain mass, about 1×10⁶ vg/g of brain mass to about 1×10¹⁰ vg/g of brain mass, about 1×10⁶ vg/g of brain mass to about 2×10¹⁰ vg/g of brain mass, about 1×10⁶ vg/g of brain mass to about 3×10¹⁰ vg/g of brain mass, about 1×10⁶ vg/g of brain mass to about 4×10¹⁰ vg/g of brain mass, about 1×10⁶ vg/g of brain mass to about 5×10¹⁰ vg/g of brain mass, about 1×10⁶ vg/g of brain mass to about 6×10¹⁰ vg/g of brain mass, about 1×10⁶ vg/g of brain mass to about 7×10¹⁰ vg/g of brain mass, about 1×10⁶ vg/g of brain mass to about 8×10¹⁰ vg/g of brain mass, about 1×10⁶ vg/g of brain mass to about 9×10¹⁰ vg/g of brain mass, about 1×10⁶ vg/g of brain mass to about 1×10¹¹ vg/g of brain mass, about 1×10⁶ vg/g of brain mass to about 2×10¹¹ vg/g of brain mass, about 1×10⁶ vg/g of brain mass to about 3×10¹¹ vg/g of brain mass, about 1×10⁶ vg/g of brain mass to about 4×10¹¹ vg/g of brain mass, about 1×10⁶ vg/g of brain mass to about 5×10¹¹ vg/g of brain mass, about 1×10⁶ vg/g of brain mass to about 6×10¹¹ vg/g of brain mass, about 1×10⁶ vg/g of brain mass to about 7×10¹¹ vg/g of brain mass, about 1×10⁶ vg/g of brain mass to about 8×10¹¹ vg/g of brain mass, about 1×10⁶ vg/g of brain mass to about 9×10¹¹ vg/g of brain mass, about 1×10⁶ vg/g of brain mass to about 1×10¹² vg/g of brain mass, about 1×10⁶ vg/g of brain mass to about 2×10¹² vg/g of brain mass, 1×10⁶ vg/g of brain mass to about 3×10¹² or about 1×10⁶ vg/g of brain mass to about 4×10¹² vg/g of brain mass.

In one case, a vector is delivered to a subject by infusion. A vector dose delivered to a subject by infusion can be measured as a vector infusion rate. Non-limiting examples of vector infusion rates include: 1-10 μL/min for intra-ganglionic, intraspinal, intracranial or intraneural administration; and 10-1000 μL/min for intrathecal or cisterna magna administration. In some cases, the vector is delivered to a subject by MRI-guided Convection Enhanced Delivery (CED). This technique enables increased viral spread and transduction distributed throughout large volumes of the brain, as well as reduces reflux of the vector along the needle path.

In one aspect, a therapeutically effective dose of vector can be administered to a patient as a gene therapy for treating Angelman syndrome or another neurological disorder having UBE3A deficiency. The vector may be administered via injection into the hippocampus or ventricles, in some cases, bilaterally. Exemplary dosages of the therapeutic can range between about 5.55×10¹¹ to about 2.86×10¹² vector genome units/g brain mass.

### Kits and Related Compositions

The agents described herein may, in some aspects, be assembled into pharmaceutical or diagnostic or research kits to facilitate their use in therapeutic, diagnostic or research applications. A kit may include one or more containers housing the components of the invention and instructions for use. Specifically, such kits may include one or more agents described herein, along with instructions describing the intended application and the proper use of these agents. In certain aspects agents in a kit may be in a pharmaceutical formulation and dosage suitable for a particular application and for a method of administration of the agents. Kits for research purposes may contain the components in appropriate concentrations or quantities for running various experiments.

The kit may be designed to facilitate use of the methods described herein by researchers and can take many forms. Each of the compositions of the kit, where applicable, may be provided in liquid form (e.g., in solution), or in solid form, (e.g., a dry powder). In certain cases, some of the compositions may be constitutable or otherwise processable (e.g., to an active form), for example, by the addition of a suitable solvent or other species (for example, water or a cell culture medium), which may or may not be provided with the kit. As used herein, “instructions” can define a component of instruction and/or promotion, and typically involve written instructions on or associated with packaging of the invention. Instructions also can include any oral or electronic instructions provided in any manner such that a user will clearly recognize that the instructions are to be associated with the kit, for example, audiovisual (e.g., videotape, DVD, etc.), Internet, and/or web-based communications, etc. The written instructions may be in a form prescribed by a governmental agency regulating the manufacture, use or sale of pharmaceuticals or biological products, which instructions can also reflect approval by the agency of manufacture, use or sale for animal administration.

The kit may contain any one or more of the components described herein in one or more containers. As an example, in one aspect, the kit may include instructions for mixing one or more components of the kit and/or isolating and mixing a sample and applying to a subject. The kit may include a container housing agent described herein. The agents may be in the form of a liquid, gel or solid (powder). The agents may be prepared sterilely, packaged in syringe and shipped refrigerated. Alternatively, it may be housed in a vial or other container for storage. A second container may have other agents prepared sterilely. Alternatively, the kit may include the active agents premixed and shipped in a syringe, vial, tube, or other container. The kit may have one or more or all the components required to administer the agents to a subject.

### EXAMPLES

Examples have been set forth below for the purpose of illustration and to describe certain specific aspects of the disclosure. However, the scope of the claims is not to be in any way limited by the examples set forth herein. Various changes and modifications to the disclosed aspects will be apparent to those skilled in the art and such changes and modifications including, without limitation, those relating to the packaging vectors, cell lines and/or methods of the disclosure may be made without departing from the spirit of the disclosure and the scope of the appended claims.

The practice of the invention employs, unless otherwise indicated, conventional molecular biological and immunological techniques within the skill of the art. Such techniques are well known to the skilled worker and are explained fully in the literature. See, Current Protocols in Molecular Biology, John Wiley & Sons, Inc., NY, N.Y. (1987-2015), including all supplements; Green and Sambrook, Molecular Cloning: A Laboratory Manual, 4^{th} Edition, Cold Spring Harbor, N.Y. (2014); and Harlow and Lane, Antibodies, a Laboratory Manual, Cold Spring Harbor, N.Y. (1989), all the contents of which are incorporated by reference herein in their entireties.

### Example 1: Production of pTR-UphUBE Plasmid with Human UBE3A Isoform 1

In one aspect described herein, a hUBE3A plasmid, pTR-UphUbe, was generated by inserting a Homo sapiens UBE3A gene (hUBE3A) into a pTR plasmid backbone between a UBC promoter and a bovine growth hormone regulatory element (a poly A sequence). As shown in FIG. 1A(i), the UBC promoter is operably linked to the downstream hUBE3A gene in order to drive the hUBE3A gene transcription in vivo. ITR sequences (labeled “TR” in FIG. 1A(i)) were inserted upstream of the UBC promoter and downstream of the bovine growth hormone polyadenylation site. The backbone further included an antibiotic resistance gene, an ampicillin resistance gene, and a bacterial origin of replication.

The nucleotide sequence (SEQ ID NO: 1) of the hUBE3a plasmid, pTR-UphUbe, formed as described above, is depicted in FIG. 1B.

The pTR-UphUbe construct therefore includes a UphUbe3A transgene ITR to ITR nucleic acid sequence of SEQ ID NO: 2 (see FIG. 1C(i)).

The Homo sapiens chromosome 15 E6AP ubiquitin-protein ligase (UBE3A) gene sequence is disclosed in FIG. 1D (Accession No: AH005553; Matsuura et al. Nat. Genet. (1997)15 (1), 74-77, the content of which is incorporated herein in its entirety).

As disclosed in the ITR to ITR sequence (SEQ ID NO: 2; FIG. 1C) and pTR-UphUbe construct (SEQ ID NO: 1; FIG. 1B), the hUBE3A.v1 (variant 1) cDNA sequence (SEQ ID NO: 5) comprises the coding region of the human UBE3A variant 1 cDNA having a nucleotide sequence of SEQ ID NO: 25 that encodes hUBE3A protein isoform 1 with the amino acid sequence SEQ. ID. NO. 4 (FIG. 1F).

The region of SEQ ID NO: 5 that encodes for the amino acid sequence of hUBE3A isoform protein 1 (SEQ ID NO: 4) has the nucleic acid sequence of SEQ ID NO: 11.

Variations to the ITR to ITR region of the pTR construct described in Example 1 above, can be made using a different nucleotide sequence, e.g. codon optimized cDNA sequence, that codes for the same hUBE3A isoform 1 protein sequence described above (SEQ ID No: 4).

In other aspects, the UBE3A transgene within the ITR to ITR region of the UphUbe construct in Example 1 can be replaced with UBE3A cDNAs encoding alternate UBE3A isoforms.

For example, the UBE3A transgene can be replaced with the Homo sapiens UBE3A Variant 2 (hUBE3a.v2) cDNA having the nucleotide sequence of SEQ ID NO: 6 comprising an open reading frame (ORF) that encodes the hUBEA3 Isoform 2 having the amino acid sequence of SEQ ID NO. 7 (see FIG. 1G).

In another example, the UBE3A transgene can be replaced with the Homo sapiens UBE3A Variant 3 (hUBE3a.v3) cDNA nucleotide sequence of SEQ ID NO: 8 comprising an open reading frame (ORF) that encodes the hUBEA3 Isoform 3 having the amino acid sequence SEQ ID NO. 9 (see FIG. 1H).

### Example 2: mAAV9 Vector

Mutant AAV9 vectors were produced incorporating the ITR to ITR sequence of Example 1, above.

In one aspect, vectors derived from wt AAV9 include, and are not limited to, a mutant AAV9 vector having a mutated AAV9 capsid protein in which a tyrosine (Tyr) amino acid residue at position 501 in wt AAV9 (residue 500 in AAV2) mutated to phenylalanine (Phe).

In one aspect, vectors derived from wt AAV9 include, and are not limited to, a mutated recombinant (mrAAV9) vector having an AAV9 capsid protein tyrosine (Tyr) amino acid residues at positions 446 and 731 in wt AAV9 mutated to phenylalanine (Phe) (see, Iida A., et al. “Systemic Delivery of Tyrosine-Mutant AAV Vectors Results in Robust Transduction of Neurons in Adult Mice,” BioMed Res. Internat. 2013).

The amino acid sequence of a mutant form of AAV9 capsid protein (AAV9.1) having a tyrosine (Tyr) amino acid residue at position 446 in WT AAV9 mutated to phenylalanine (Phe) is SEQ ID NO: 32, shown with a corresponding nucleic acid sequence (SEQ ID NO: 30) in FIG. 1K.

The amino acid sequence encoding for a mutant form of AAV9 capsid protein (AAV9.2) having tyrosine (Tyr) amino acid residues at positions 446 and 731 in WT AAV9, respectively, mutated to phenylalanine (Phe) is SEQ ID NO: 10, shown with a corresponding nucleotide sequence (SEQ ID NO:33) in FIG. 1L.

In the first instance, differences between the nucleic acid sequence encoding the wt AAV9 capsid protein (not shown) and the nucleic acid encoding the AAV9.1 capsid protein (SEQ ID NO:30) is a single point mutation of an adenosine (a) nucleotide to a thymidine (t) at position 1337, corresponding to a codon change of “tat” to “ttt” (see FIG. 1K). In the second instance, differences between the nucleotide acid sequence encoding wtAAV9 capsid protein and the nucleotide sequence encoding AAV9.2 capsid protein (SEQ. ID. NO. 33) include the same adenosine to thymidine mutation at position 1337 and a second adenosine to thymidine mutation at position 2192-2193, corresponding to a codon change of “tat” to “ttc” resulting in changes in amino acid residues 446 and 731 from tyrosine (Tyr) to phenylalanine (Phe), respectively. Both mutations in amino acid and nucleic acid sequence are described in Ida et al (Id.), where it is noted that neither mutation leads to any sequence changes in the potential assembly activating protein (AAP) gene and the mutant capsids package the gene plasmid with titers similar to those of the wild-type capsids.

### Example 3: Human UBE3A AAV Vector

A Human UBE3a AAV9.2 vector was produced by transient transfection of HEK293 cells with the pTR-UphUbe plasmid described in Example 1, a plasmid encoding a helper rep gene sequence and an mrAAV9 capsid. The rep gene and adenoviral helper plasmids were transfected into HEK293 cells separately.

### Example 4: In Vivo Administration of the Human UBE3 AAV Vector

The hUBE3 AAV vector produced as described in Example 3 was suspended in 0.1 M Phosphate Buffered Saline (PBS) at a concentration of ˜1.2×10¹³ vg/ml.

Animal subjects were weighed before surgery and anesthetized using isoflurane. Surgery was performed using a stereotaxic apparatus (Digital Mice Stereotaxic Instrument, World Precision Instruments). The skin was cut (1-2 cm) with a scalpel and the cranium was exposed by an incision along the midsagittal plane. Two burr holes were drilled through the cranium using a Dremel and Dental drill bit (SSW HP-3, SSWhite Burs Inc) using bregma to ascertain and serve as the fiduciary to calculate positions of injection location, as listed in Tables 1 and 2 below. The mrAAV9 vector dose was injected using a syringe pump at 2.5 μL/min. The surgical incision was closed with nylon (Ethilon® or an equivalent product) sutures.

A Hamilton microsyringe was lowered, and viral vector (hUBE3a mrAAV9 vector) was dispensed at the following unilateral doses per hemisphere: Study #1 Rats 5 μL (1.2×10¹³ vg/mL); Study #2 Rats 25 μL (4.8×10¹² vg/mL); and, Study #3 Mice 5 μL (1.2×10¹³ vg/mL). The total bilateral dose for each study: Study #1 Rats 1.2×10¹¹ vgs; Study #2 Rats 2.4×10¹¹ vgs; and, Study #3 Mice 1.2×10¹¹ vgs.

hUBE3A mrAAV9 vector was dispensed bilaterally into the lateral ventricle as shown in Tables 1 and 2 using a convection enhanced method. The incision was cleaned and closed with surgical sutures. Control injected animals received injections of 0.1 M sterile PBS based upon dosing experiment (Study #1 Rats 5 μL; Study #2 Rats 25 μL; Study #3 Mice 5 μL), as shown in Tables 1 and 2:

TABLE 1
Mouse Lateral Ventricle

| Hemisphere: | LLV | RLV |
|---|---|---|
| Injection LAT (X): | −1.0 | +1.0 |
| AP (Y): | −0.4 | −0.4 |
| DV (Z): | −2.4 | −2.4 |

TABLE 2
Rat Lateral Ventricle

| Hemisphere: | LLV | RLV |
|---|---|---|
| Injection LAT (X): | −1.5 | +1.5 |
| AP (Y): | −0.5 | −0.5 |
| DV (Z): | −4.3 | −4.3 |

### Example 5: Isolation of Genomic DNA

Genomic DNA was isolated from the animals treated as described in Example 4 using DNeasy® Blood & Tissue kit (Qiagen, Germantown, Md.) using a protocol for the animal tissue. Briefly, 25-30 mg of samples were immersed in 180 μL Buffer ATL+20 μl Proteinase K, mixed thoroughly, and incubated at 56° C. for 4 hours, vortexing intermittently. 200 μL Buffer AL and 200 μL absolute EtOH were added and mixed thoroughly. The mixture was applied to a Mini-spin column and centrifuged. The column was washed twice and eluted in 100 μL Buffer AE. The quality and the concentration of the eluent was determined using Nanodrop machine.

### Example 6: Analysis of Copy Number of pTR-UphUBE Plasmids by Quantitative PCR for Use as Reference Standard

The copy numbers for pTR-UphUBE1 plasmid was calculated per reaction mix and serially diluted to generate a standard curve. The qPCR primers, length (mer) and base pairs (bp), in Table 3 were used to capture promoter and hUBEVI sequence amplicons for specificity.

TABLE 3
qPCR Primers

| No. | Name | Primer | Pair | mer | bp |
|---|---|---|---|---|---|
| 1 | UphUbe-718F | TAAATTCTGGCCGTTTTTGG | 1 | 20 | 122 |
| | | (SEQ ID NO: 37) | | | |
| 2 | UphUbe-839R | CATTTCCACAGCCCTCAGTT | 1 | 20 | |
| | | (SEQ ID NO: 38) | | | |
| 3 | UphUbe-718F | TAAATTCTGGCCGTTTTTGG | 2 | 29 | 136 |
| | | (SEQ ID NO: 39) | | | |
| 4 | UphUbe-853R | ATTCGTGCAGGCTTCATTTC | 2 | 20 | |
| | | (SEQ ID NO: 40) | | | |

Primer Pairs shown in Table 3 have been demonstrated to be ˜100% efficient, having a standard curve (R²=0.99) for both pairs and a dynamic range between from about 10⁸ to about 10¹² plasmid copies. Additionally, a single distinct melt curve peak for each primer pair indicates no primer-dimer or off-target amplification product, confirming specificity of the amplicons.

Quantitative PCR was done using SsoAdvanced™ universal SYBR Green supermix and CFX96 instrument [Bio-Rad] using filter-tips to avoid contamination. 20 μL mix was prepared by adding supermix and gDNA (100 ng) or titration plasmid and Primer Pair 1 (250 nM each) in water. CFX96 was programmed to run for 95° C. for 150s, 40 cycles (95° C. for 15s+60° C. for 30s), and a melt curve default cycle.

The data was imported into Bio-Rad's CFX manager software (version 3.1) for further analysis. The standard curve was generated for each experiment and the copy numbers were determined by extrapolation. The summary statistics were done using either GraphPad Prism 7 or JMP Pro 13.

### Example 7: Western Blotting and Analysis

For Western blotting, samples were dissected and homogenized in mammalian protein extraction reagent (M-PER, Pierce) and protease and phosphatase inhibitor cocktail: Sigma; 1× phosphatase inhibitors I and II, 1× complete protease inhibitors, 1× phenylmethylsulfonyl flouride. Protein concentration was standardized to 2 μg/μL after biquinoline acid assay (Pierce) and mixed with equal parts Laemmli buffer. Samples were loaded into a 4-15% gradient gels (Bio-Rad). Transferred PVDF (Immobilion-P) membranes were blocked with 5% non-fat milk and 1× Tris-buffered saline (TBS) for 1 hour before incubating with anti-E6AP (for mice: 1:1000, MyBioSource, for rats: 1:1000, Sigma-Aldrich) or anti-beta actin (1:5000, Cell Signaling Technology) overnight at 4° C. The anti-E6AP antibody refers to an anti-rabbit secondary antibody (1:2000; Bethyl Labs). The membranes were rinsed three times, for 10 minutes each, with TBS and Tween-20. The secondary antibody was subsequently applied and allowed to incubate for 90 minutes at room temperature. The membranes were washed 3 additional times before exposed by enhanced chemiluminescence method (Thermo Scientific).

### Example 8: Composition and Methods for Increasing Expression of a UBE3A Gene Therapy Vector for Angelman Syndrome

Herein we describe the composition and methods of use of a hUBE3A gene therapy vector via intracerebroventricular dosing for increased DNA and transgene expression in Angelman syndrome. The hUBE3A gene therapy vector is comprised of a hUBE3A transgene flanked by AAV2-ITR's, human ubiquitin ligase c promoter and 3′ bovine growth hormone regulatory elements that are encapsulated by a double tyrosine mutated (Y/F 446 and Y/F 731) AAV9 capsid. Mutations of surface exposed tyrosine residues to phenylalanine are known to reduce tyrosine phosphorylation and ubiquitination of capsid proteins thus salvaging them from the proteasome degradation pathway and improving intracellular trafficking to the nucleus. The increased trafficking of the mrAAV9 vector to the nucleus results in increased DNA and transgene expression. The hUBE3A vector used in this Example was produced as described in Example 3.

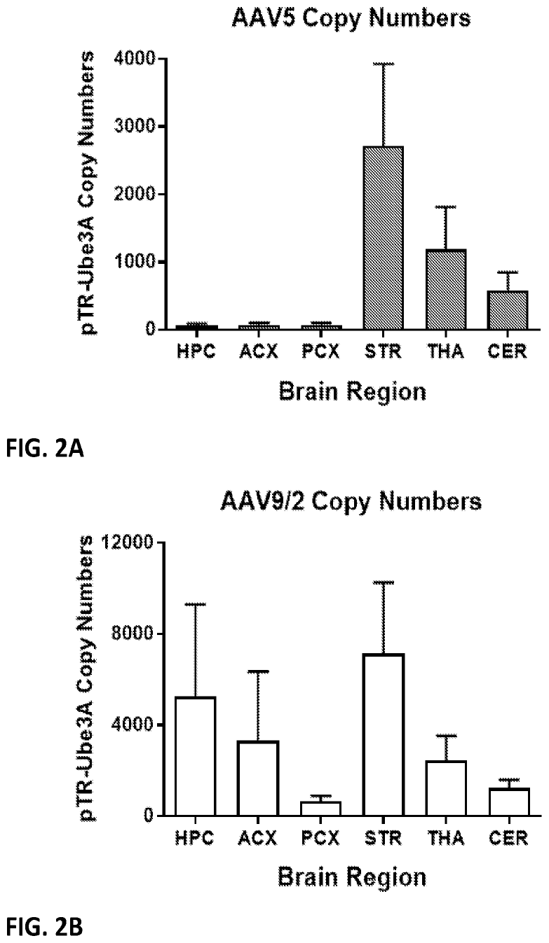

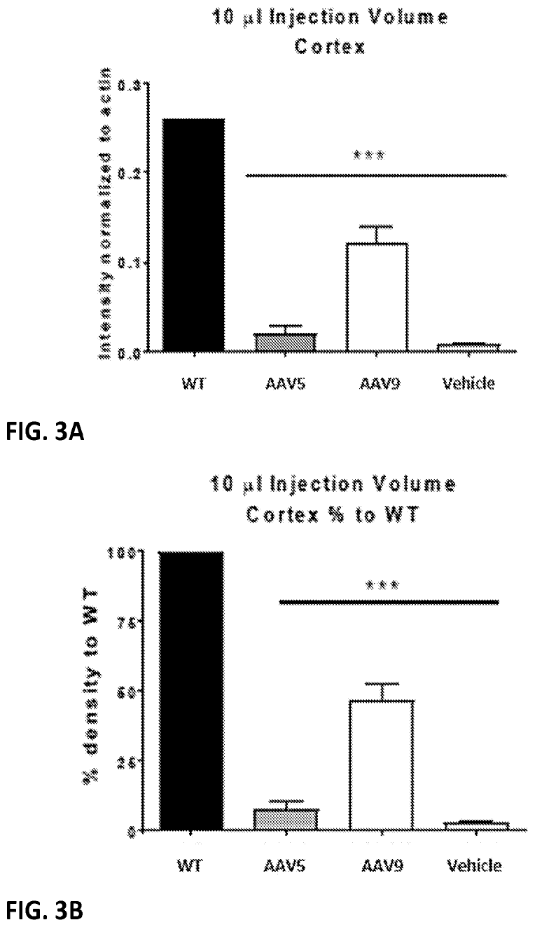

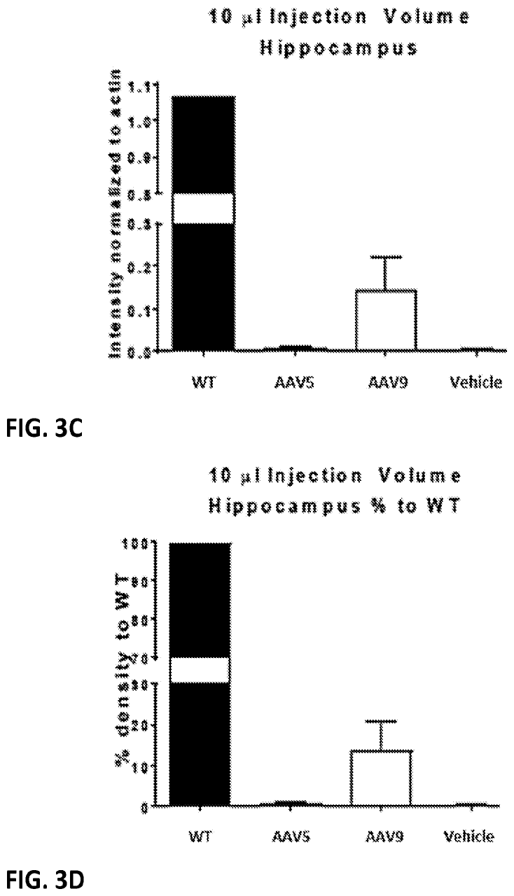

FIGS. 2A and B and FIGS. 3 A-D show expression of E6AP protein in AS rats dosed bilaterally in the lateral ventricle with unilateral doses of 5 μL (1.2×10¹³ vg/mL) per side of hUBE3a mrAAV9 vector and AAV5 vectors compared to WT. FIG. 2A shows hUBE3a plasmid copies in the brain of AS rats administered the hUBE3a rAAV5 vector. FIG. 2B shows hUBE3a plasmid copies in the brain of rats administered the hUBE3a mrAAV9 vector.

Both show distribution of vector DNA in the hippocampus (HPC), anterior cortex (ACX), posterior cortex (PCX), striatum (STR), thalamus (THA), and cerebellum (CER). The figures show increased vector DNA biodistribution to the brain of animals dosed with the hUBE3a mrAAV9 vector compared to the rAAV5 vector.

FIG. 3A-D compares hUBE3A protein biodistribution in the cortex and hippocampus of AS and wild type rats from Study 1. FIG. 3A shows the intensity normalized to actin in the cortex. FIG. 3B shows the intensity normalized to actin in the hippocampus. FIG. 3C shows the results expressed as percent density compared to wild type in the cortex, while FIG. 3D shows the same type of results from the hippocampus. The results show increased hUBE3a protein expression and biodistribution in the brain of animals dosed with the mrAAV9 tyrosine mutated vector compared to the rAAV5 vector.

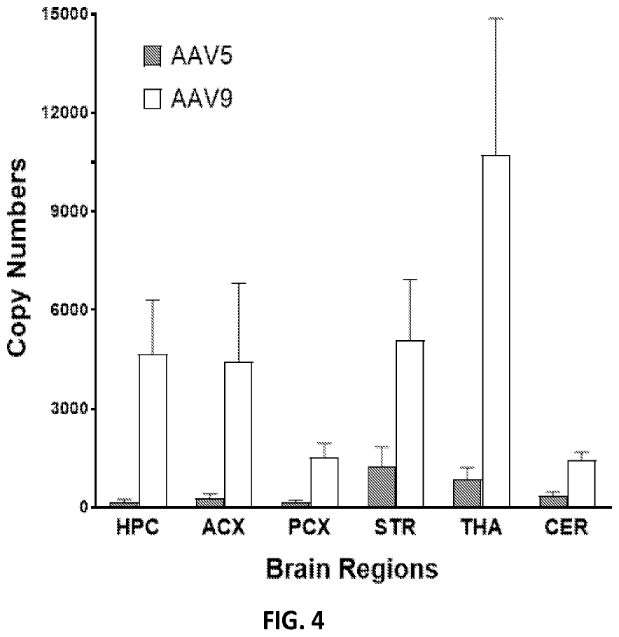

FIG. 4 shows hUBE3a vector DNA biodistribution in the brain of AS rats dosed bilaterally in the lateral ventricle with unilateral doses of 25 μL (4.8×10¹² vg/ml) of hUBE3a AAV vectors from either rAAV5 or mrAAV9 per side. Distribution results from the hippocampus (HPC), anterior cortex (ACX), posterior cortex (PCX), striatum (STR), thalamus (THA), and cerebellum (CER) are shown, with results from administration of vector from rAAV5 (shaded) and from mrAAV9 (clear). The results show increased vector DNA biodistribution in the brain of animals dosed with the mrAAV9 tyrosine mutated vector compared to the rAAV5 vector.

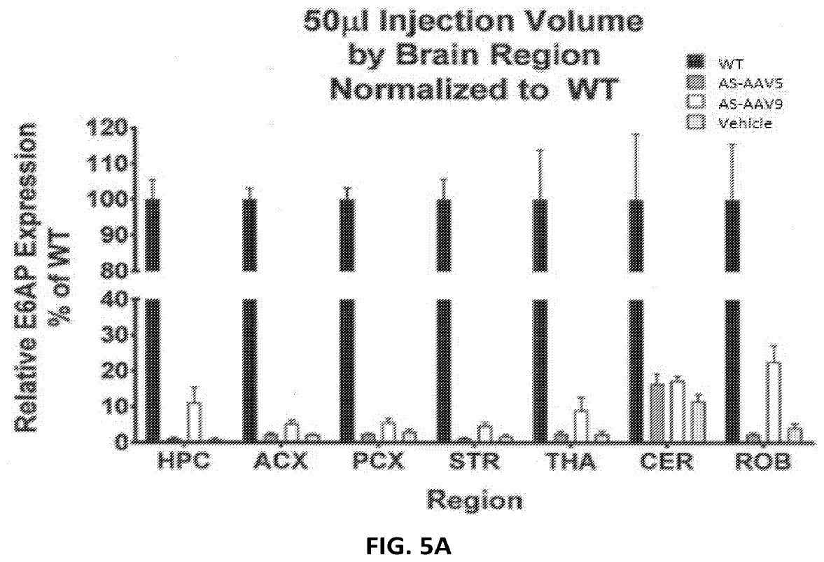

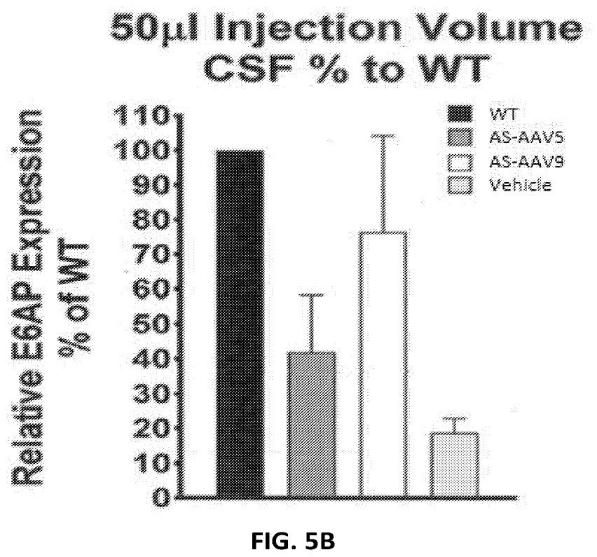

FIG. 5A shows hUBE3A protein distribution in the brains of AS relative to wild type rats from Study 2, as measured in the hippocampus (HPC), anterior cortex (ACX), posterior cortex (PCX), striatum (STR), thalamus (THA), cerebellum (CER), and midbrain and brainstem (ROB). The results show increased hUBE3A protein expression and biodistribution in the brain with the mrAAV9 tyrosine mutated vector compared to the rAAV5 vector.

FIG. 5B shows hUBE3A protein distribution as measured in CSF compared to wild type rats.

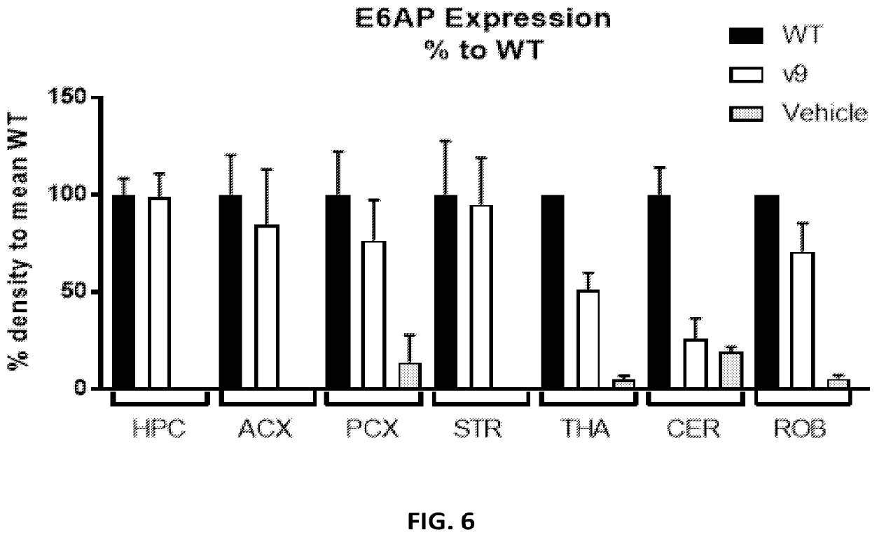

FIG. 6 shows protein expression in the brain of AS mice from Study 3, in which the mice were dosed bilaterally into the lateral ventricle with unilateral doses of 5 μl (1.2×10¹³ vg/ml) of hUBE3a AAV vectors from mAAV9.2 per side. Distribution in the same regions of the brain as illustrated in FIG. 5A was measured. hUBE3A protein expression and biodistribution in the different regions of the brain was found to be at or close to wild type levels.

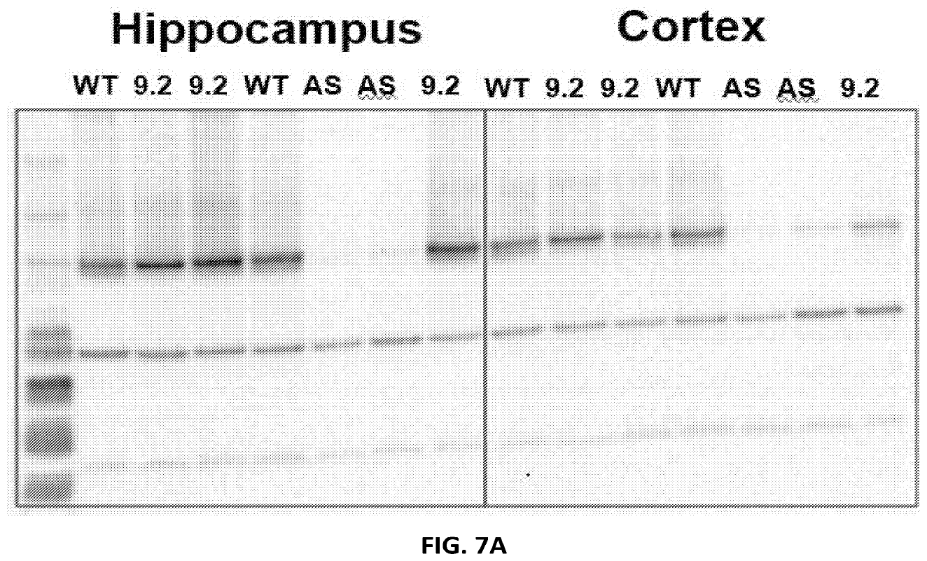

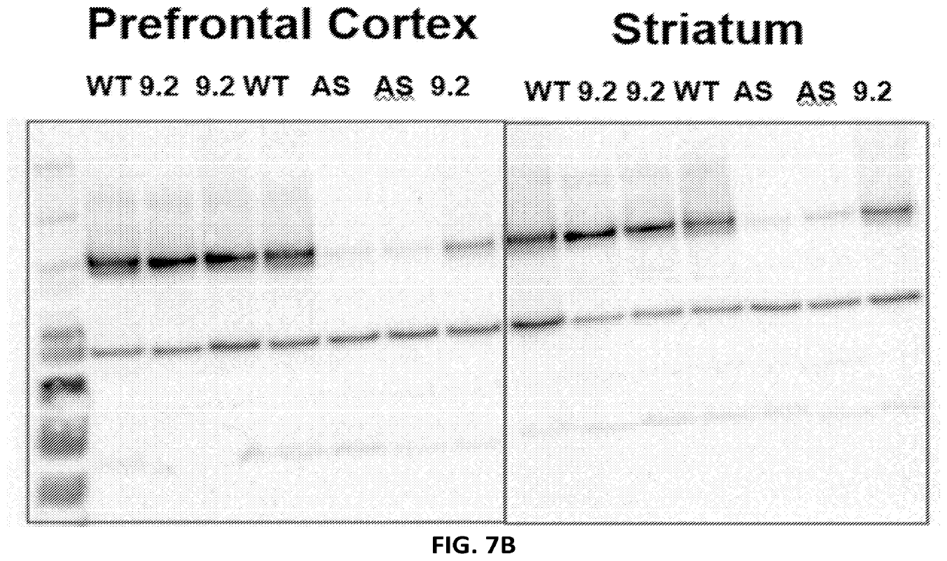

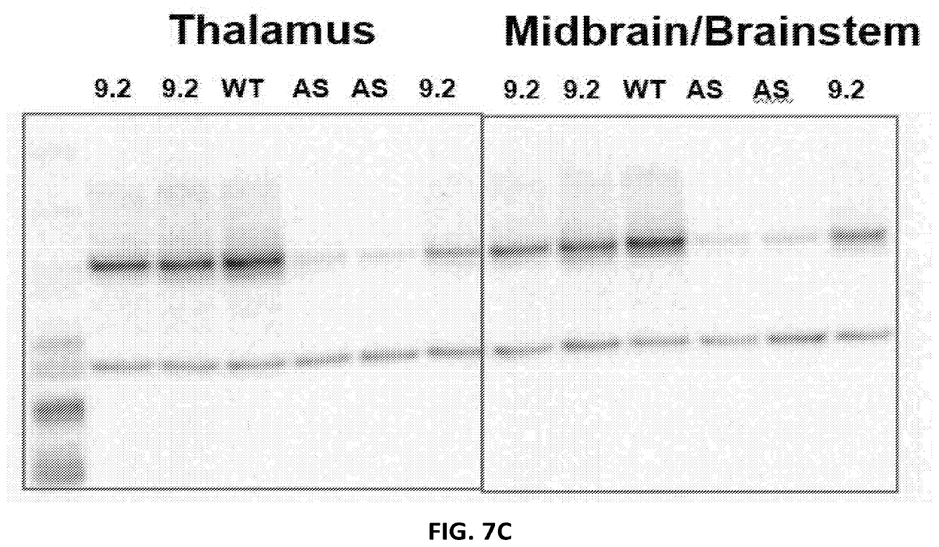

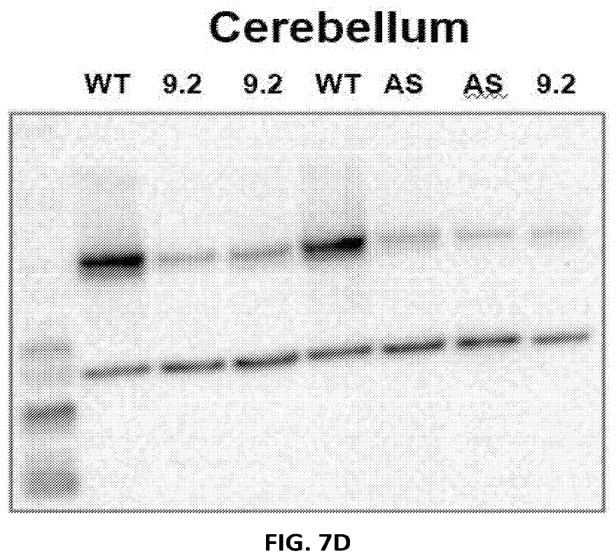

FIG. 7 A-D are Western blots showing hUBE3A protein expression in various parts of the brains of individual AS mice from Study 3. FIG. 7A shows results from the hippocampus and cortex. FIG. 7B shows results from the prefrontal cortex and stratum. FIG. 7C shows results from the thalamus and midbrain/brainstem. FIG. 7D shows results from the cerebellum. All the figures show hUBE3A protein expression and biodistribution in the different regions of the brain at or close to wild-type levels.

### Example 9: Intracerebroventricular AAV Injection of Human UBE3A Recovers Deficits in a Mouse Model for Angelman Syndrome

Maternal UBE3A-deficient mice (UBE3A m−/p+) recapitulate many of the phenotypes seen in the human disorder, including severe motor coordination defects, learning and memory dysfunction, and higher seizure propensity in specific mouse strains. In addition, these mice exhibit a severe defect in hippocampal area CA1 long-term potentiation (LTP) and bidirectional impairments of both LTP and long-term depression (LTD) in the mouse visual cortex. Recently, temporal control over maternal UBE3A expression was reported using a Cre-dependent method of transcriptional control. This model showed that the synaptic plasticity defects could be recovered at any age. However, other behavioral phenotypes were rescued following reinstatement of UBE3A in adolescent mice only. In contrast, recent studies showing motor coordination improvement as well as rescue of the hippocampal plasticity and cognitive defect in the adult AS mouse model following pharmacological intervention suggests that the therapeutic window may not be limited in the mouse or, by extension, in human AS patients.

### AAV Construction

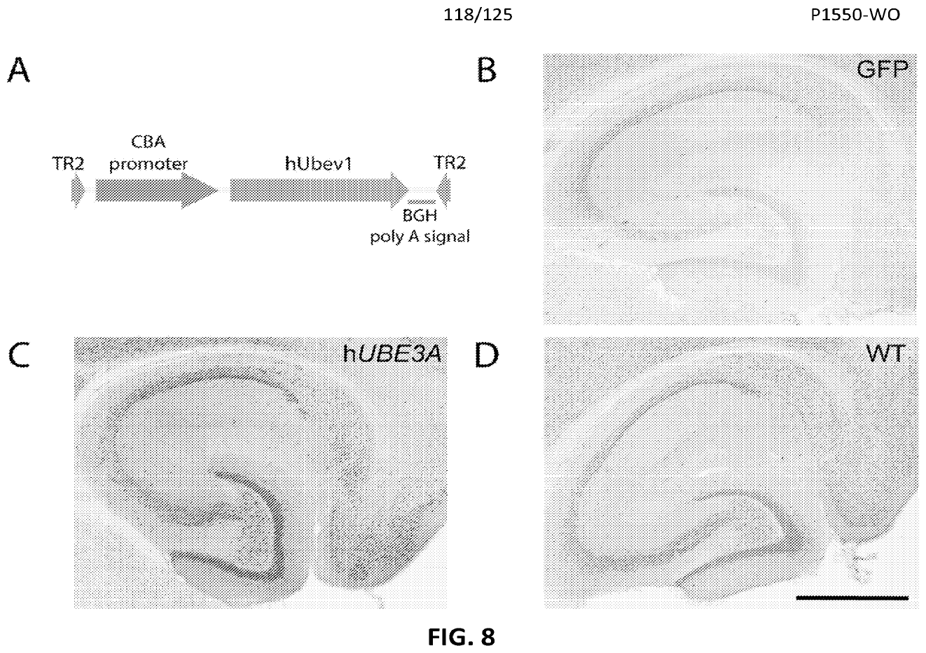

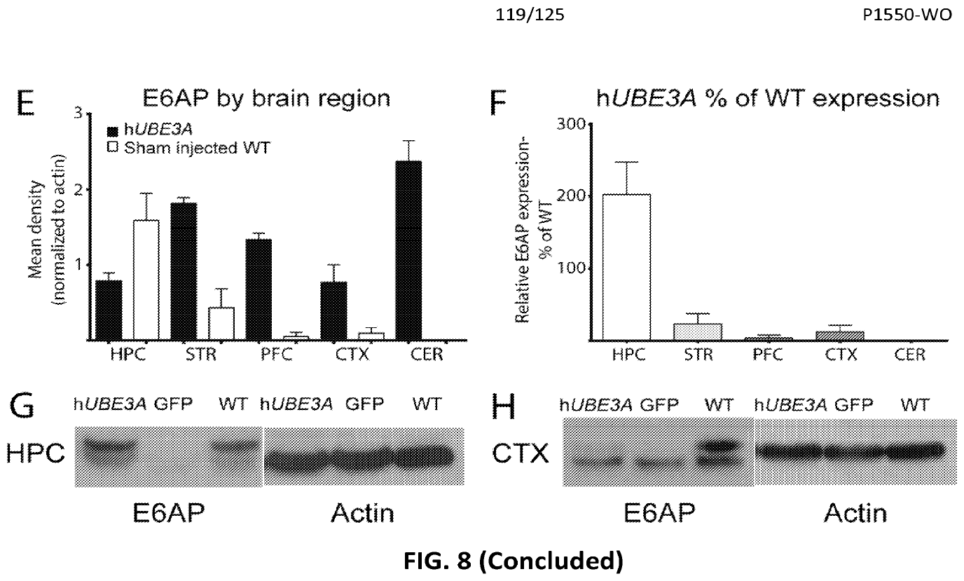

Recombinant AAV serotype 5 (rAAV5) vectors were generated and purified as previously described. rAAV5 expressing human UBE3A isoform 1 protein (GI:19718761) was cloned using PCR from the cDNA clone RC200629 from Origene. hUBE3A was cloned into the pTR12.1-MCSW vector at the Age I and Nhe I cloning sites. This vector contains the AAV2 inverted terminal repeats and the chicken-beta actin-CMV hybrid (CBA) promoter for hUBE3A mRNA transcription (see FIG. 8A). Green Fluorescent Protein (GFP) was also cloned in the same manner and used for control injections. The concentration of rAAV particles was expressed as vector genomes per milliliter (vg/ml). Vector genomes were quantitated using a modified version of the dot plot protocol described by Zolotukhin (Zolotukhin et al. Methods. 2002; 28(2):158-67) using a non-radioactive biotinylated probe for UBE3A generated by PCR. Bound biotinylated probe was detected with IRDye 800CW (Li-Cor Biosciences) and quantitated on the Li-Cor Odyssey.

### Breeding of Animals

Mice with the UBE3A null mutation were described previously (Jiang Y H et al. Neuron. 1998; 21(4):799-811). All experiments were performed on mice obtained through cryopreservation from the Jackson Laboratories (Jackson Labs). Female 129 mice containing the paternal null mutation were bred with wild type C57BL6/J males to produce F1 generation hybrid maternally-deficient AS mice and wild type (WT) littermate controls (purchased from Jackson Laboratories, catalog numbers 00447 and 000664). Animals were kept on a 12h our light/dark cycle and provided food ad libitum. All testing took place during the light cycle.

### Surgical Procedure

Mice were weighed before surgery and anesthetized using isoflurane. Surgery was performed using a stereotaxic apparatus (Digital Mice Stereotaxic Instrument, World Precision Instruments). The cranium was exposed by an incision along the midsagittal plane, and two holes were drilled through the cranium using a dental drill bit (SSW HP-3, SSWhite Burs Inc). A Hamilton microsyringe was lowered, and injections of 3 μl of viral vector in sterile 0.1 M Phosphate Buffered Saline (PBS) at a concentration of ˜5×10¹² vg/ml were dispensed bilaterally into the lateral ventricle (coordinates from bregma; lateral ±1.0 mm; anteroposterior −0.4 mm, vertical, −2.4 mm) using the convection enhanced method described previously (Carty N et al. Convection-enhanced delivery and systemic mannitol increase gene product distribution of AAV vectors 5, 8, and 9 and increase gene product in the adult mouse brain. J Neurosci Methods. 2010; 194(1):144-53). The incision was cleaned and closed with surgical sutures. Sham injected (WT) animals received 3 μl of sterile 0.1 M PBS. AS animals (n=4) injected for testing UBE3A protein activity expressed from rAAV constructs were injected into the hippocampus as previously reported (Daily J L et al. PLoS One. 2011; 6(12): e27221). Mice survived for 4 weeks before analysis of hippocampal tissue.

### Immunohistochemistry

Mice used for immunohistochemistry (IHC) were weighed and overdosed with pentobarbital (200 mg/kg) and transcardially perfused with PBS. Brains were removed and fixed in 4% Paraformaldehyde overnight at 4° C. Brains were placed in 30% sucrose solution before obtaining 25 μm sagittal sections preserved in PBS plus 0.2% sodium azide. Free-floating sections were blocked for 15 minutes (4% Methanol, 4% H₂O₂ in PBS) before permeabilization (Lysine, 1×-Triton, horse serum in PBS) for 30 minutes. Anti-E6AP (MyBioSource, 1:200) or anti-GFP (Abcam, 1:30,000) was applied overnight, then secondary (anti-rabbit biotin 1:3000, Vector Laboratories, Inc; anti-chicken 1:3000, Vector Laboratories, Inc) for 2 hours before applying ABC Peroxidase Staining Kit (Thermo-Fisher) then a nickel chloride enhanced DAB (3,3′-Diaminobenzidine) system. Sections were mounted, dehydrated in Histoclear, and scanned using the Axio Scan Z.1 (Zeiss) slide scanner system.

### Western Blot Analysis

For Western blotting, brain tissue was dissected and homogenized in mammalian protein extraction reagent (M-PER, Pierce) and protease and phosphatase inhibitor cocktail (Sigma; 1× phosphatase inhibitors I and II, 1× complete protease inhibitors, 1× phenylmethylsulfonyl flouride). Protein concentration was standardized to 2 μg/μl after biquinoline acid assay (Pierce) and mixed with equal parts Laemmli buffer. Samples were loaded into a 4-15% gradient gels (Bio-Rad). Transferred PVDF (Immobilion-P) membranes were blocked with 5% non-fat milk and 1× Tris-buffered saline (TBS) for 1 hour before incubating with anti-E6AP (1:2000, MyBioSource) or anti-beta actin (1:5000, Cell Signaling Technology) overnight at 4° C. Anti-rabbit secondary antibody (1:2000; Bethyl Labs) was applied after 3 ten minute rinses with TBS plus Tween-20 for 60 minutes at room temperature. The membranes were washed 3 additional times before exposed by enhanced chemiluminescence method (Thermo Scientific).

### E6AP Ubiquitination Assay

The single system control assay was performed using an E6AP/S5a Ubiquitination Kit (Boston Biochem, K-230). Tissue samples were prepared similar to Western blot samples but standardized to a concentration of 8 μg/μl. Each reaction tube contained water, reaction buffer, E1 enzyme, E2 enzyme, ATP, S5a, E6AP, and ubiquitin to achieve a total volume of 30 μl. For ubiquitination reactions involving lysate, 24 μl of lysate was combined with 3 μl of 5 μM S5a and 3 μl of 500 μM ubiquitin. The ubiquitination reaction was initiated upon the addition of ubiquitin and samples were incubated at 38° C. At specified time points, a 3 μl aliquot was removed from the reaction tube and mixed with 5 μl of 5× loading buffer and 1 μl of 1× dithiothreitol (DTT), terminating the ubiquitination reaction. Samples were snap frozen at −80° C. The designated time points, using a log-based 3-time scale, were 0.11, 0.33, 1, 3, 4.5, 6, 7.5, and 9 hours. Frozen samples were thawed on ice, boiled at 95° C. for 5 minutes, and loaded into hand cast 4-10% polyacrylamide gels. Proteins were separated by SDS-PAGE and transferred onto PVDF blotting membranes (EMD Milipore). The membranes were blocked in 5% non-fat dry milk in 1×TBST (0.1% Tween-20) for 1 hour. Membranes were incubated overnight at 4° C. in primary antibody, washed 3 times for 10 minutes in 1×TBST, and incubated with the corresponding secondary antibody for 1 hour at room temperature. Antibodies used include E6AP (Bethyl Laboratories), Ubiquitin (Cell Signaling Technology), S5a (Boston Biochem), anti-Mouse IgG (Southern Biotech), anti-Rabbit IgG (Southern Biotech), and anti-Goat IgG (Southern Biotech). Primary antibodies were diluted 1:2000 and secondary antibodies were diluted 1:4000 in 2.5% non-fat dry milk in 1×TBST. Membranes were washed 3 times for 10 minutes in 1×TBST and digitally imaged with the Amersham Imager 6000 (GE Healthcare) using ECL Western Blotting Substrate (Thermo Scientific Pierce). Images were analyzed using Image Studio Lite (LICOR). Proteins were quantified by normalizing all proteins of interest to 1:1000 diluted 0-tubulin (Upstate).

Enzymatic activity was calculated by a standard curve of E6AP concentration ranging from 0.25 nM to 10 nM using purified E6AP (Boston Biochem). In triplets, 10 μl of standard curve sample and 10 μl of wild-type lysate from three different animals was vacuum transferred to a nitrocellulose membrane using the Bio Rad Dot Blot Apparatus. The nitrocellulose membrane was blocked in 5% non-fat dry milk in 1×TBST (0.1% Tween-20) for 1 hour. The membrane was incubated overnight at 4° C. in anti-E6AP antibody (Bethyl Laboratories) diluted 1:2000, washed 3 times for 10 minutes in 1×TBST, and incubated with an anti-Rabbit IgG secondary antibody (Southern Biotech) for 1 hour at room temperature. The membrane was washed 3 times for 10 minutes in 1×TBST and digitally imaged with the Amersham Imager 6000 (GE Healthcare) using ECL Western Blotting Substrate (Thermo Scientific Pierce). The captured image was analyzed using Image Studio Lite software (LICOR). The average initial concentration of E6AP in wild-type lysate was determined by comparing densitometry results from each sample to the E6AP standard curve. A time vs. concentration graph was constructed and the initial reaction velocity (v) of the conversion of E6AP to ubiquitinated E6AP was calculated from the slope of the linear portion of the curve. Specific activity was determined by dividing the slope of this line by the amount of total homogenate protein in the tissue lysate samples.

### Behavioral Testing (in Order of Performance)

For behavioral testing, the following numbers of animals were used for each group: 21 AAV5-hUBE3A ICV (for all tests not including elevated plus maze and Rotorod, n=14), 39 AAV5-GFP, 32 sham injected WT. Sex distribution between treatments remained statistically even.

### Hidden Platform Watermaze

Spatial memory was tested with the use of hidden platform watermaze. Mice were trained with 4 sessions per day to find a 10 cm diameter platform located 1 cm below the surface of a 1.2 m diameter pool filled with opaque water. Large cues were placed on the walls and video tracking software (ANY-Maze, Stoelting Instruments) tracked swim speed and latency to reach platform. Mice were placed in the pool in a semi-random order and allowed to search for the platform for a maximum of 60 seconds. If the mouse failed to locate the platform within 60 seconds, the researcher gently guided the mouse to the platform where they remained for 10-15 seconds. Mice were removed from the pool, gently dried, and placed in a cage filled with warm corncob bedding. Inter-trial intervals were 30 minutes and training occurred at the same time for 5 consecutive days. 72 hours after day 5 of training, mice were placed in the pool with the platform lowered beyond escape. Mice remained in the pool for 60 seconds and swim accuracy was recorded.

### General Activity and Anxiety

General activity and anxiety were measured with the open field test. Mice were placed in a 40 cm square opaque-walled chamber with bright lighting conditions and allowed to explore for 15 minutes. Video tracking monitored movement (ANY-Maze, Stoelting Instruments). Anxiety was also tested by the elevated plus maze (EPM) test. The EPM consisted of two well-lit open arms (35 cm) and two well-lit closed arms facing each other with a 4.5 cm common space in between. The EPM was placed 40 cm above the floor and video tracking monitored movement for 5 minutes (ANY-Maze, Stoelting Instruments). Immobility was determined by lack of movement for 2 or more consecutive seconds.

### Motor Coordination

Motor coordination and motor learning were assessed through the accelerating Rotorod (Ugo-Basile). Mice were placed on a 3 cm diameter rod with an initial rotating speed of 4 rpm. Latency to fall was recorded as the rod accelerated up to 40 rpm over 300 seconds. Mice received 4 trials for 2 consecutive days with inter-trial intervals of 30 minutes.

### Marble Burying Assay

Compulsive behaviors and neophobia were assessed using the marble burying test. Mice were placed in a large Plexiglas cage (22×43 cm) with 4 cm deep corncob bedding and 15 black glass marbles (14 mm diameter) placed in an equidistant 3×5 pattern on top of the bedding. Mice explored the cage for 30 minutes under normal lighting conditions. Number of marbles buried greater or equal to ⅔ were recorded as buried. To address potential aversions to novel bedding as reported in AS mice by McCoy et al, mice were introduced to the corncob bedding daily during watermaze testing approximately 4 days prior to testing (McCoy E S et al. J Neurosci. 2017; 37(42):10230-9).

### Extracellular Hippocampal Recordings

Mice were decapitated and brains quickly moved to an ice-cold, high-sucrose cutting solution containing (in mM): 110 sucrose, 60 NaCl, 3 KCl, 28 NaHCO₃, 1.25 NaH₂PO₄, 5 D-glucose, 0.6 ascorbate, 7 MgCl2, and 0.5 CaCl₂). 400 μm horizontal slices were obtained using a Vibratome (Leica VT1200) and hippocampi were dissected into a 50/50 solution of cutting and 95% O₂/5% CO₂ equilibrated Artificial Cerebrospinal Fluid (ACSF) containing (in mM): 125 NaCl, 2.5 KCl, 26 NaHCO₃, 1.25 NaH₂PO₄, 25 D-glucose, 1 MgCl2, and 2 CaCl₂). Slices were then transferred to a 30° C. interface extracellular field recording chamber (AutoMate Scientific) with oxygenated 100% ACSF for at least one hour. Field excitatory postsynaptic potentials (fEPSP) were obtained from the CA1 stratum radiatum using glass micropipettes filled with ACSF and a tip diameter that obtained a 1-4 MQ electrical resistance. Formvar-coated nichrome wires delivered biphasic stimulus pulses (1-15 V; 100 μs duration; 0.05 Hz) in the Schaffer collaterals arising from the CA3 region. pClamp 10 (Molecular Devices) controlled stimulation delivered by a Digidata 1322A interface (Axon Instruments) and a stimulus isolator (A-M Systems). A differential amplifier (A-M Systems) amplified electrical signals filtered at 1 kHz and digitized at 10 kHz. Baseline stimulus intensity was set at a 50% maximum fEPSP response found from an input-output curve (stimulating slices from 0-15 mV at 0.5 mV increments). Paired-pulse facilitation consisted of 2 pulses starting at 20 milliseconds apart with a 20 second inter-trial interval. Subsequent inter-pulse intervals increased by 20 milliseconds for 15 trials. After recording a 20-minute baseline, theta-burst stimulation (tbs) delivered 5 trains of 4 pulse bursts at 200 Hz, with an inter-burst interval of 20 seconds. Recording continued for 60 minutes and slope of fEPSP response change in relation to baseline indicated.

### Statistical Analysis

All data are represented as Mean±SEM. An unpaired Student's t-test or one-way ANOVA with Dunnett's pos-hoc multiple comparisons test was performed and significance was set at p<0.05.

UBE3A Expression after ICV Injection of hUBE3A AAV

Hippocampal-dependent learning and memory defects can be recovered in the adult AS mouse with direct hippocampal injection and normalized mouse UBE3A protein levels (Daily J L et al. PLoS One. 2011; 6(12): e27221). Injection of mice with a murine UBE3A rAAV serotype 9 can rescue both spatial and associative learning and memory, as well as area CA1 LTP. In this set of experiments, the highly homologous human UBE3A (hUBE3A) gene was administered by intracerebroventricular (ICV) injection. Human variant 1 UBE3A gene flanked by AAV2 terminal repeats and the CBA promoter for hUBE3A mRNA transcription was packaged into rAAV serotype 5 capsids (rAAV5) (FIG. 8A). This serotype exhibits attractive biodistribution and cell-tropism characteristics in the CNS and, when injected via ICV, is capable of traversing into the parenchyma and infecting neurons (Davidson B L et al. Proc Natl Acad Sci USA. 2000; 97(7):3428-32). This broad transduction ability of rAAV5 through ependymal cells lining the ventricle is a beneficial mechanism in gene delivery (Bajocchi G et al. Nat Genet. 1993; 3(3):229-34; Ghodsi A et al., Exp Neurol. 1999; 160(1):109-16).

In order to confirm that the human UBE3A gene in rAAV was capable of producing active E6AP protein, ubiquitination activity of the protein examined in injected tissue homogenates. A E6AP ubiquitin ligation assay (Boston Biochem) on homogenates from transduced mouse hippocampal tissue was performed. AS animals were injected with either the corresponding mouse gene, the human gene construct, or a control GFP. As expected, the control AS animals demonstrated little to no UBE3A activity. However, the levels of activity of both the mouse and human E6AP were comparable to the levels found in wild type animals.

Immuno-staining showed that AS animals administered AAV5-hUBE3A by ICV injection express detectable amounts of UBE3A protein (E6AP) in the hippocampus when compared to AAV5-GFP injected AS animals (FIGS. 8B-D). Western blot analysis also shows detectable levels of E6AP expression in the hippocampus, striatum, cortex, and prefrontal cortex of AAV5-hUBE3A ICV injected animals when normalized to actin. AAV5-GFP injected animals expressed no detectable E6AP protein (FIG. 8E). There was an approximately 200% increase in protein expression in the hippocampus of AAV5-hUBE3A ICV injected mice compared to sham injected WT animals (FIG. 8F). Thus, UBE3A AAV administration by ICV injection can significantly increase E6AP expression in the hippocampus without specifically targeting the hippocampus.

Effect of AAV5 hUBE3A ICV Injection on Anxiety, Neophobia, and Compulsive Behaviors in AS Mice

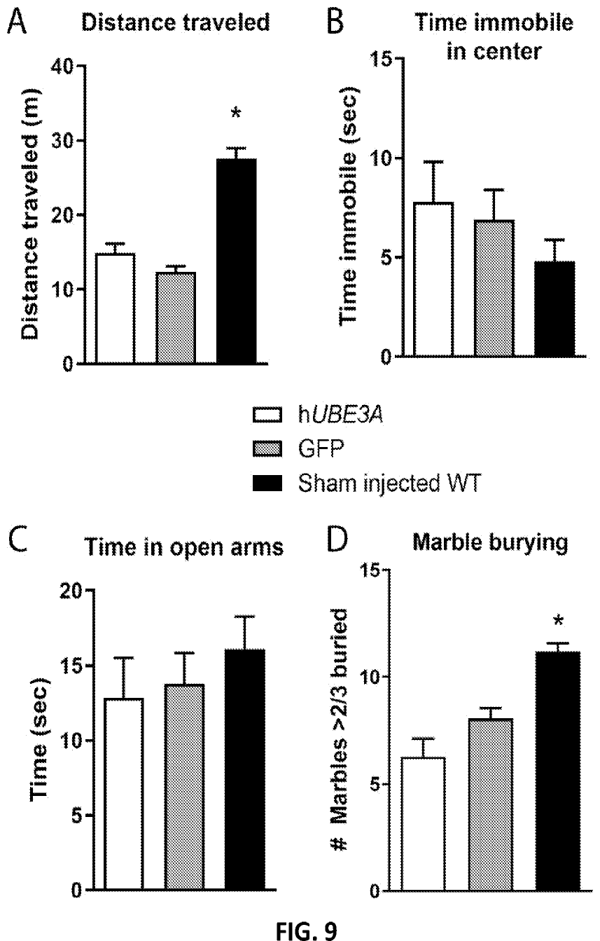

Mice were allowed to explore an open field device for 15 minutes under bright lighting conditions. No differences in overall locomotion were seen between treatments in the AS mice; however, sham injected WT mice did show an increase in distance traveled (FIG. 9A). This difference in activity has been previously shown in AS mice similar to our mice that were bred with a hybrid C57BL6/J x 129Sv/Ev background compared to wild types (Mandel-Brehm et al., Proc Natl Acad Sci USA. 2015; 112(16):5129-34; Sonzogni et al. Mol Autism. 2018; 9:47). There were no statistically significant differences when measuring immobility in the center of the open field apparatus as well as time spent in the well-lit open arms during the elevated plus maze task (FIGS. 9B and 9C). Thus, the increase in activity does not indicate altered anxiety. WT control mice on a C57BL6/J background, as well as the hybrid line, have increased marble burying behaviors compared to AS mice. This difference was also noted in the hybrid strain as seen by lower numbers of marbles buried in AS mice, with no change in AAV treatment (FIG. 9D).

Effect of AAVS hUBE3A ICV Injection on the Motor Coordination Deficits in AS Mice

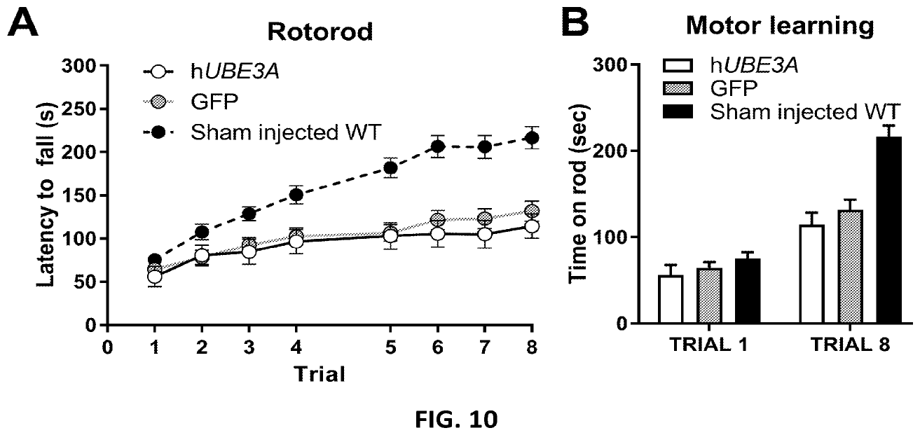

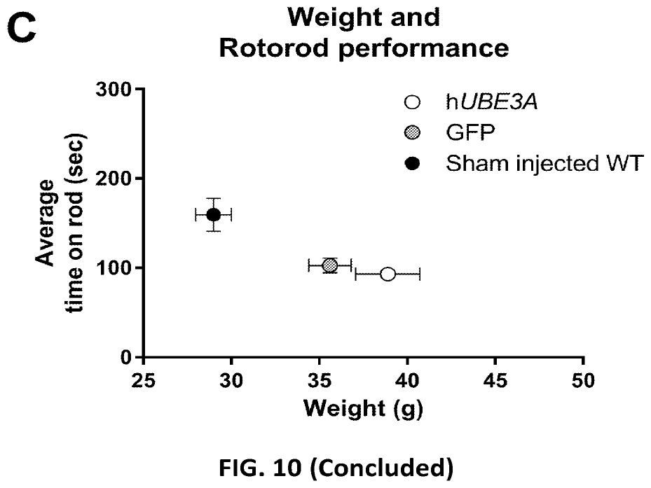

Motor coordination deficits are well established in all strains of AS mice. Mice were tested on a Rotorod apparatus accelerating from 4 to 40 rpm for 4 trials per day for 2 consecutive days. Overall locomotion did not improve with AAV5-hUBE3A treatment (FIG. 10A). All animals showed motor learning improvement from trial 1 to trial 8 (FIG. 10B). However, AS mice are generally heavier than wild type animals, regardless of sex, and the increased weight correlates to decreased Rotorod performance (FIG. 10C). The differences in weight may underlie the persistent motor deficits in AS mice and restoration of UBE3A levels in the brain would not likely alter the mouse weight. Recent studies involving a dietary ketone supplementation in the AS mouse resulted in a normalization of the AS mouse weight compared to wild type controls and Rotorod performance was rescued (Ciarlone et al. Neurobiol Dis. 2016; 96:38-46).

Effect of AAV5 hUBE3A ICV Injection on Spatial Learning Deficits in AS Mice

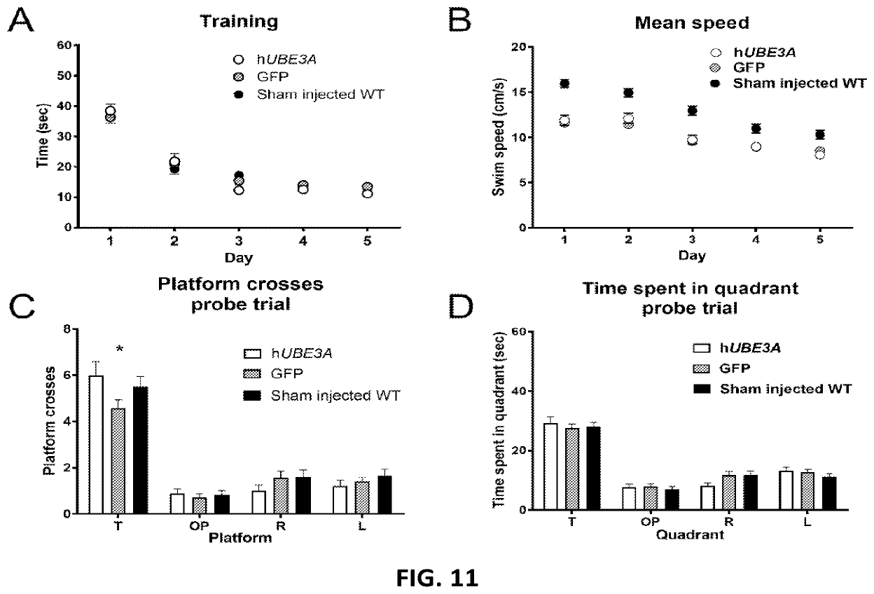

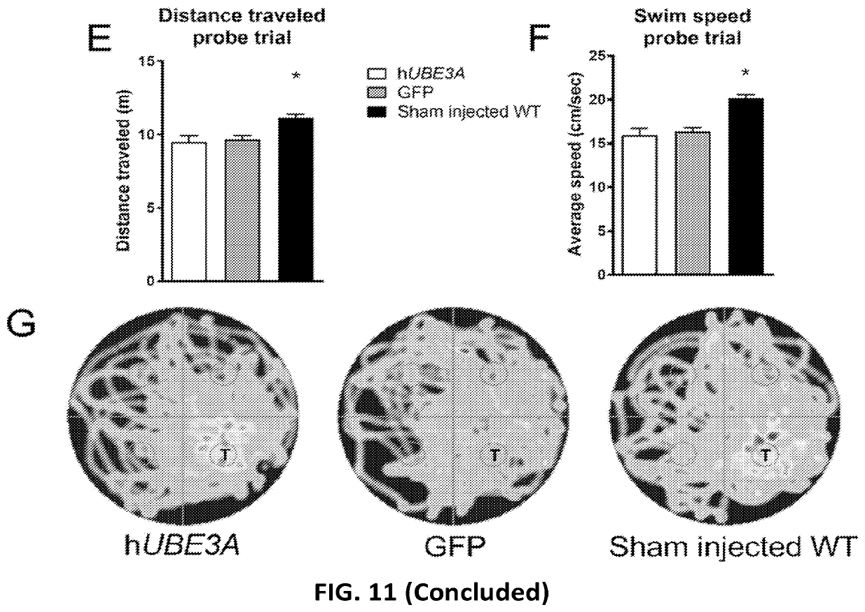

Learning deficiencies in AS mice were evaluated after ICV injection of AAV5-hUBE3A in AS mice. By using the hidden platform watermaze task, mice were trained for 5 days to locate a platform in a pool with large, extra-maze cues. All mice learned the location of the platform over 5 training days (FIG. 11A). During training, AS mice swam slower than sham injected WT mice, but found the platform in the about same amount of time (FIG. 11B). 72 hours after the 5th day of training, the platform was removed, and each mouse was placed in the pool for 60 seconds. AAV5-GFP injected mice did not cross the target platform location as much as AAV5-hUBE3A injected AS mice (FIG. 11C), despite no differences in the time spent in the target quadrant for all groups (FIG. 11D). Both AS groups traveled the same distance and swam at the same speed to each other (FIGS. 11E and 11F). The observation that AAV5-hUBE3A treatment did not recover swim speed but did improve the spatial memory defect indicated that learning and memory rescue was not a result of swim speed changes. These results showed a spatial bias for the target quadrant following hidden platform watermaze training for all groups; however, ICV injection of AAV5-hUBE3A did lead to an improved search strategy for the target platform.

Effect of AAV5 hUBE3A ICV Injection on Synaptic Plasticity Deficits in AS Mice

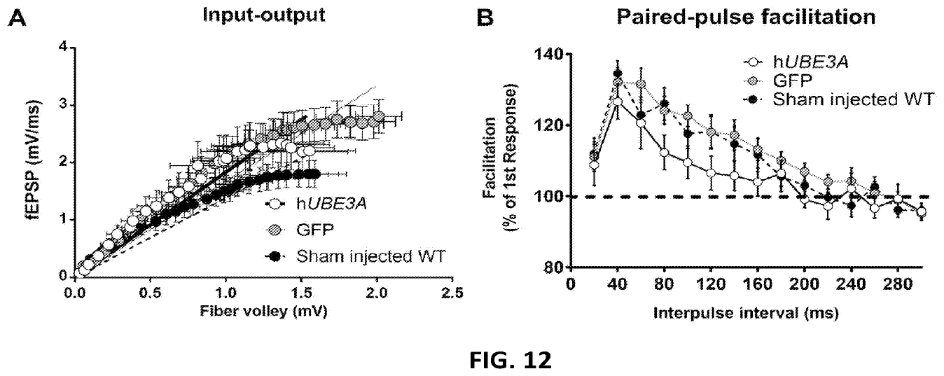

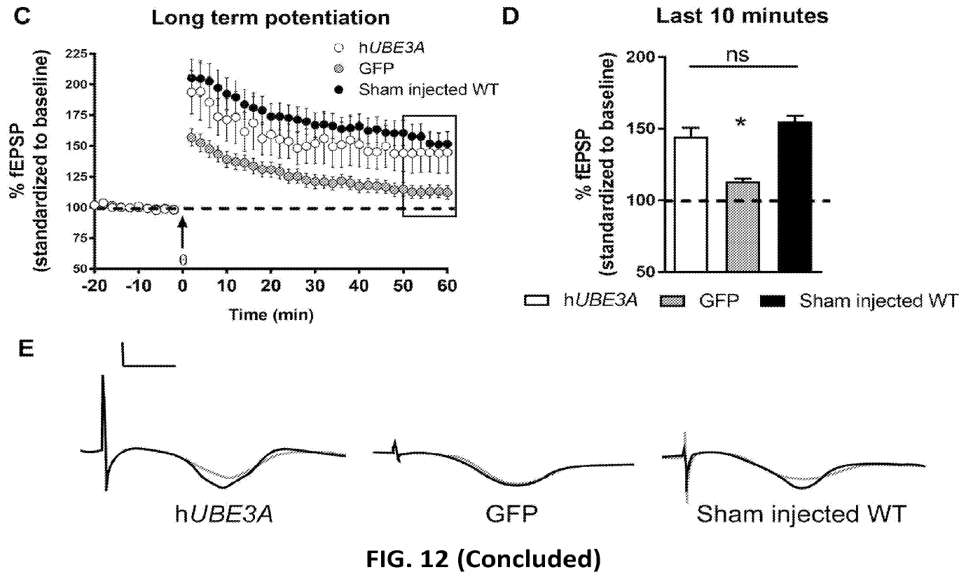

ICV injection of AAV5-hUBE3A is sufficient to recover synaptic plasticity deficits (FIG. 12). All groups had normal synaptic function in response to increasing stimulation (FIG. 12A), indicating that the synaptic plasticity recovery was not due to AAV5-hUBE3A injection-dependent changes in synaptic transmission. No differences in presynaptic responses were observed after pulses presented in close proximity (paired-pulse facilitation, FIG. 12B). Using an extracellular signal-regulated kinase (ERK)-dependent long-term potentiation protocol (theta burst stimulation-tbs), long-term recovery was found in the slopes of field excitatory postsynaptic potentials (fEPSP) (FIG. 12C). By averaging the fEPSPs during the last 10 minutes of recording (50-60 minutes after tbs), there was a significant difference in AAV5-GFP treated AS animals to AAV5-hUBE3A AS and sham injected WT controls (FIG. 12D).

While there has been described and illustrated herein general and specific aspects of the vector and use thereof for treating UBE3A deficiencies, it will be apparent to those skilled in the art that variations and modifications are possible without deviating from the broad spirit and principle of those aspects described herein. It is also to be understood that the following claims are intended to cover all of the generic and specific features of such aspects herein described, and all statements of the scope of such aspects herein described and equivalents thereof, as a matter of language, might be said to fall within.

Unless otherwise defined, all technical and scientific terms used herein have the same meaning as commonly understood by one of ordinary skill in the biological arts. Although any methods and materials similar or equivalent to those described herein can be used in the practice or testing of one or more aspects of the gene therapy described herein, some potential and preferred methods and materials are further described.

All publications mentioned herein are incorporated herein by reference in their entirety to disclose and describe the methods and/or materials in connection with which the publications are cited. It is understood that the present disclosure supersedes any disclosure of an incorporated publication to the extent there is a contradiction.

1. A human UBE3A vector comprising:
a nucleic acid having
i) a 5′ inverted terminal repeat (ITR) sequence;
ii) a promoter downstream of the 5′ ITR sequence;
iii) a UBE3A nucleotide sequence encoding a human UBE3A protein isoform operably linked downstream of the promoter; and,
iv) a 3′ ITR sequence downstream of the UBE3A nucleotide sequence; and
an adeno-associated virus serotype 9 (AAV9) capsid,
wherein the nucleic acid is packaged in the AAV9 capsid, and wherein the nucleic acid does not include a secretion sequence.

2. The vector of claim 1, wherein the 5′ and 3′ ITR sequences are independently selected from the group consisting of adeno-associated virus serotype 1 (AAV1) ITRs, serotype 2 (AAV2) ITRs, serotype 3 (AAV3) ITRs, serotype 4 (AAV4) ITRs, and serotype 9 (AAV9) ITRs.

3. The vector of claim 1, wherein the 5′ and 3′ ITR sequences are both serotype 2 (AAV2) ITRs.

4. The vector of claim 1, wherein the 5′ and/or 3′ ITR sequences comprise the nucleotide sequence of SEQ ID NO: 22.

5. The vector of claim 1, wherein the AAV9 capsid has an amino acid sequence of SEQ ID NO: 32 or SEQ ID NO: 27.

6. The vector of claim 1, wherein the promoter sequence is a cytomegalovirus chicken-beta actin hybrid promoter or human ubiquitin ligase C promoter.

7. The vector of claim 1, wherein the UBE3A nucleotide sequence encodes hUBE3A isoform 1 having the amino acid sequence of SEQ ID NO: 4.

8. The vector of claim 1, wherein the UBE3A nucleotide sequence is SEQ ID NO: 25.

9. A method of delivering to a nerve cell in a brain of a living subject in need thereof comprising, administering a therapeutically effective amount of the human UBE3A vector of claim 1 via intracranial injection to the subject.

10. The method of claim 9, wherein the therapeutically effective amount of the human UBE3A vector is in a range of from about 5×10⁶ viral genomes per gram (vg/g) to about 2.86×10¹² vg/g of brain mass, from about 4×10⁷ vg/g to about 2.86×10¹² vg/g of brain mass, or from about 1×10⁸ to about 2.86×10¹² vg/g of brain mass.

11. The method of claim 9, wherein intracranial administration comprises bilateral injection.

12. The method of claim 9, wherein the administration via intracranial injection comprises intrahippocampal or intracerebroventricular injection.

13. The method of claim 9, wherein the administration is via intracerebroventricular injection (ICV).

14. The method of claim 9, wherein the human UBE3A vector is transduced into at least two of hippocampus, auditory cortex, prefrontal cortex), striatum, thalamus and cerebellum.

15. The method of claim 9, wherein the subject has a UBE3A deficiency.

16. The method of claim 15, wherein the UBE3A deficiency is Angelman Syndrome.

17. The method of claim 16, wherein ICV injection of the human UBE3A vector restores UBE3A expression to wild type levels in at least two of the hippocampus, auditory cortex, prefrontal cortex and striatum.

18. The method of claim 16, wherein the intracerebroventricular injection of the therapeutically effective amount of the human UBE3A vector treats at least one symptom of Angelman Syndrome.

19. The method of claim 18, wherein the at least one symptom of Angelman Syndrome comprises learning and memory deficits.

20. A human UBE3A vector comprising:
a nucleic acid having
i) a 5′ inverted terminal repeat (ITR) sequence;
ii) a promoter downstream of the 5′ ITR sequence;
iii) a UBE3A nucleotide sequence encoding human UBE3A protein isoform 1 having SEQ ID NO: 4 operably linked downstream of the promoter; and
iv) a 3′ ITR sequence downstream of the UBE3A nucleotide sequence; and
an adeno-associated virus stereotype 5 (AAV5) capsid,
wherein the nucleic acid is packaged in the AAV5 capsid, and wherein the nucleic acid does not include a secretion sequence.


SEQUENCE LISTING (46 sequences)

SEQ ID NO: 1 - DNA, 6619 bp, Artificial Sequence
  Plasmid
  misc_feature (1)..(6619): pTR-UphUBE3 plasmid
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatctgaattcgcgatcgcttaattaaggccggccggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctgggtacgtgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacgaagcttaccggtccaccatgaagcgagcagctgcaaagcatctaatagaacgctactaccaccagttaactgagggctgtggaaatgaagcctgcacgaatgagttttgtgcttcctgtccaacttttcttcgtatggataataatgcagcagctattaaagccctcgagctttataagattaatgcaaaactctgtgatcctcatccctccaagaaaggagcaagctcagcttaccttgagaactcgaaaggtgcccccaacaactcctgctctgagataaaaatgaacaagaaaggcgctagaattgattttaaagatgtgacttacttaacagaagagaaggtatatgaaattcttgaattatgtagagaaagagaggattattcccctttaatccgtgttattggaagagttttttctagtgctgaggcattggtacagagcttccggaaagttaaacaacacaccaaggaagaactgaaatctcttcaagcaaaagatgaagacaaagatgaagatgaaaaggaaaaagctgcatgttctgctgctgctatggaagaagactcagaagcatcttcctcaaggataggtgatagctcacagggagacaacaatttgcaaaaattaggccctgatgatgtgtctgtggatattgatgccattagaagggtctacaccagattgctctctaatgaaaaaattgaaactgcctttctcaatgcacttgtatatttgtcacctaacgtggaatgtgacttgacgtatcacaatgtatactctcgagatcctaattatctgaatttgttcattatcgtaatggagaatagaaatctccacagtcctgaatatctggaaatggctttgccattattttgcaaagcgatgagcaagctaccccttgcagcccaaggaaaactgatcagactgtggtctaaatacaatgcagaccagattcggagaatgatggagacatttcagcaacttattacttataaagtcataagcaatgaatttaacagtcgaaatctagtgaatgatgatgatgccattgttgctgcttcgaagtgcttgaaaatggtttactatgcaaatgtagtgggaggggaagtggacacaaatcacaatgaagaagatgatgaagagcccatccctgagtccagcgagctgacacttcaggaacttttgggagaagaaagaagaaacaagaaaggtcctcgagtggaccccctggaaactgaacttggtgttaaaaccctggattgtcgaaaaccacttatcccttttgaagagtttattaatgaaccactgaatgaggttctagaaatggataaagattatacttttttcaaagtagaaacagagaacaaattctcttttatgacatgtccctttatattgaatgctgtcacaaagaatttgggattatattatgacaatagaattcgcatgtacagtgaacgaagaatcactgttctctacagcttagttcaaggacagcagttgaatccatatttgagactcaaagttagacgtgaccatatcatagatgatgcacttgtccggctagagatgatcgctatggaaaatcctgcagacttgaagaagcagttgtatgtggaatttgaaggagaacaaggagttgatgagggaggtgtttccaaagaattttttcagctggttgtggaggaaatcttcaatccagatattggtatgttcacatacgatgaatctacaaaattgttttggtttaatccatcttcttttgaaactgagggtcagtttactctgattggcatagtactgggtctggctatttacaataactgtatactggatgtacattttcccatggttgtctacaggaagctaatggggaaaaaaggaacttttcgtgacttgggagactctcacccagttctatatcagagtttaaaagatttattggagtatgaagggaatgtggaagatgacatgatgatcactttccagatatcacagacagatctttttggtaacccaatgatgtatgatctaaaggaaaatggtgataaaattccaattacaaatgaaaacaggaaggaatttgtcaatctttattctgactacattctcaataaatcagtagaaaaacagttcaaggcttttcggagaggttttcatatggtgaccaatgaatctcccttaaagtacttattcagaccagaagaaattgaattgcttatatgtggaagccggaatctagatttccaagcactagaagaaactacagaatatgacggtggctataccagggactctgttctgattagggagttctgggaaatcgttcattcatttacagatgaacagaaaagactcttcttgcagtttacaacgggcacagacagagcacctgtgggaggactaggaaaattaaagatgattatagccaaaaatggcccagacacagaaaggttacctacatctcatacttgctttaatgtgcttttacttccggaatactcaagcaaagaaaaacttaaagagagattgttgaaggccatcacgtatgccaaaggatttggcatgctgtaagctagccccgggatgcatatcgatgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaacccccccccccccccccctgcaggccagctggcgtaatagcgaagaggcccgcaccgatcgcccttcccaacagttgcgtagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgggctgcagggggggggggggggggg

SEQ ID NO: 2 - DNA, 3744 bp, Artificial Sequence
  Plasmid sequence (ITR-ITR sequence of pTR-UphUbe)
  misc_feature (1)..(3744): ITR-ITR SEQUENCE OF pTR-UphUBE3
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatctgaattcgcgatcgcttaattaaggccggccggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctgggtacgtgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacgaagcttaccggtccaccatgaagcgagcagctgcaaagcatctaatagaacgctactaccaccagttaactgagggctgtggaaatgaagcctgcacgaatgagttttgtgcttcctgtccaacttttcttcgtatggataataatgcagcagctattaaagccctcgagctttataagattaatgcaaaactctgtgatcctcatccctccaagaaaggagcaagctcagcttaccttgagaactcgaaaggtgcccccaacaactcctgctctgagataaaaatgaacaagaaaggcgctagaattgattttaaagatgtgacttacttaacagaagagaaggtatatgaaattcttgaattatgtagagaaagagaggattattcccctttaatccgtgttattggaagagttttttctagtgctgaggcattggtacagagcttccggaaagttaaacaacacaccaaggaagaactgaaatctcttcaagcaaaagatgaagacaaagatgaagatgaaaaggaaaaagctgcatgttctgctgctgctatggaagaagactcagaagcatcttcctcaaggataggtgatagctcacagggagacaacaatttgcaaaaattaggccctgatgatgtgtctgtggatattgatgccattagaagggtctacaccagattgctctctaatgaaaaaattgaaactgcctttctcaatgcacttgtatatttgtcacctaacgtggaatgtgacttgacgtatcacaatgtatactctcgagatcctaattatctgaatttgttcattatcgtaatggagaatagaaatctccacagtcctgaatatctggaaatggctttgccattattttgcaaagcgatgagcaagctaccccttgcagcccaaggaaaactgatcagactgtggtctaaatacaatgcagaccagattcggagaatgatggagacatttcagcaacttattacttataaagtcataagcaatgaatttaacagtcgaaatctagtgaatgatgatgatgccattgttgctgcttcgaagtgcttgaaaatggtttactatgcaaatgtagtgggaggggaagtggacacaaatcacaatgaagaagatgatgaagagcccatccctgagtccagcgagctgacacttcaggaacttttgggagaagaaagaagaaacaagaaaggtcctcgagtggaccccctggaaactgaacttggtgttaaaaccctggattgtcgaaaaccacttatcccttttgaagagtttattaatgaaccactgaatgaggttctagaaatggataaagattatacttttttcaaagtagaaacagagaacaaattctcttttatgacatgtccctttatattgaatgctgtcacaaagaatttgggattatattatgacaatagaattcgcatgtacagtgaacgaagaatcactgttctctacagcttagttcaaggacagcagttgaatccatatttgagactcaaagttagacgtgaccatatcatagatgatgcacttgtccggctagagatgatcgctatggaaaatcctgcagacttgaagaagcagttgtatgtggaatttgaaggagaacaaggagttgatgagggaggtgtttccaaagaattttttcagctggttgtggaggaaatcttcaatccagatattggtatgttcacatacgatgaatctacaaaattgttttggtttaatccatcttcttttgaaactgagggtcagtttactctgattggcatagtactgggtctggctatttacaataactgtatactggatgtacattttcccatggttgtctacaggaagctaatggggaaaaaaggaacttttcgtgacttgggagactctcacccagttctatatcagagtttaaaagatttattggagtatgaagggaatgtggaagatgacatgatgatcactttccagatatcacagacagatctttttggtaacccaatgatgtatgatctaaaggaaaatggtgataaaattccaattacaaatgaaaacaggaaggaatttgtcaatctttattctgactacattctcaataaatcagtagaaaaacagttcaaggcttttcggagaggttttcatatggtgaccaatgaatctcccttaaagtacttattcagaccagaagaaattgaattgcttatatgtggaagccggaatctagatttccaagcactagaagaaactacagaatatgacggtggctataccagggactctgttctgattagggagttctgggaaatcgttcattcatttacagatgaacagaaaagactcttcttgcagtttacaacgggcacagacagagcacctgtgggaggactaggaaaattaaagatgattatagccaaaaatggcccagacacagaaaggttacctacatctcatacttgctttaatgtgcttttacttccggaatactcaagcaaagaaaaacttaaagagagattgttgaaggccatcacgtatgccaaaggatttggcatgctgtaagctagccccgggatgcatatcgatgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaaccc

SEQ ID NO: 3 - DNA, 10893 bp, Homo sapiens
  gene (1)..(10893): UBE3A genomic sequence
  misc_feature (122)..(122): n is a, c, g, or t
  misc_feature (340)..(340): n is a, c, g, or t
  misc_feature (1145)..(1145): n is a, c, g, or t
  misc_feature (1180)..(1180): n is a, c, g, or t
  misc_feature (1257)..(1257): n is a, c, g, or t
  misc_feature (5410)..(5410): n is a, c, g, or t
  misc_feature (7624)..(7624): n is a, c, g, or t
  misc_feature (7644)..(7644): n is a, c, g, or t
  misc_feature (7647)..(7647): n is a, c, g, or t
  misc_feature (7741)..(7741): n is a, c, g, or t
  misc_feature (7836)..(7836): n is a, c, g, or t
ggagtagtttactgagccactaatctaaagtttaatactgtgagtgaataccagtgagtacctttgttaatgtggataaccaatacttggctataggaagttttttagttgtgtgttttatnacacgtatttgactttgtgaataattatggcttataatggcttgtctgttggtatctatgtatagcgtttacagtttcctttaaaaaacatgcattgagttttttaatagtccaacccttaaaataaatgtgttgtatggccacctgatctgaccactttctttcatgttgacatctttaattttaaaactgttttatttagtgcttaaatcttgttnacaaaattgtcttcctaagtaatatgtctacctttttttttggaatatggaatattttgctaactgtttctcaattgcattttacagatcaggagaacctcagtctgacgacattgaagctagccgaatgtaagtgtaacttggttgagactgtggttcttattttgagttgccctagactgctttaaattacgtcacattatttggaaataatttctggttaaaagaaaggaatcatttagcagtaaatgggagataggaacatacctactttttttcctatcagataactctaaacctcggtaacagtttactaggtttctactactagatagataaatgcacacgcctaaattcttagtctttttgcttccctggtagcagttgtagggaaatagggaggttgaggaaagagtttaacagtctcaacgcctaccatatttaaggcatcaagtactatgttatagatacagagatgcgtaataattagttttcaccctacagaaatttatattatactcaagagtgaaagatgcagaagcaaataatttcagtcactgaggtagaatggtatccaaaatacaatagtaacatgaaggagtactggagtaccaggtatgcaataggaatctagtgtagatggcagggaagtaagagtggccaggaaatgctaagttcagtcttgaaatgtgactgggaatcaggcagctatcaactataagtcaaatgtttacaagctgttaaaaatgaaatactgattatgtaaaagaaaaccggattgatgctttaaatagactcattttcntaatgctaatttttaaaatgatagaatcctacaantcttagctgtaaaccttgtgatttttcagctgttgtactaaacaacttaagcacatataccatcagacaagcccccntccccccttttaaaccaaaggaatgtatactctgttaatacagtcagtaagcattgacattctttatcataatatcctagaaaatatttattaactatttcactagtcaggagttgtggtaaatagtgcatctccattttctacttctcatcttcatacacaggttaatcacttcagtgcttgactaacttttgccttgatgatatgttgagctttgtacttgagagctgtactaatcactgtgcttattgtttgaatgtttggtacaggaagcgagcagctgcaaagcatctaatagaacgctactaccaccagttaactgagggctgtggaaatgaagcctgcacgaatgagttttgtgcttcctgtccaacttttcttcgtatggataataatgcagcagctattaaagccctcgagctttataagattaatgcaaaactctgtgatcctcatccctccaagaaaggagcaagctcagcttaccttgagaactcgaaaggtgcccccaacaactcctgctctgagataaaaatgaacaagaaaggcgctagaattgattttaaaggtaagatgttttattttcaattgagaattgttgcctgaaaaccatgtgggagatttaaatgtattagtttttatttgttttttcttctgtgacataaagacattttgatatcgtagaaccaattttttattgtggtaacggacaggaataataactacattttacaggtctaatcattgctaattagaagcagatcatatgccaaaagttcatttgttaatagattgatttgaactttttaaaattcttaggaaaaatgtattaagtggtagtgaatctccaaaactatttaagagctgtattatgattaatcagtacatgacatattggttcatatttataattaaagctatacattaatagatatcttgattataaagaaagtttaaactcatgatcttattaagagttatacattgttgaaagaatgtaaaagcatgggtgaggtcattggtataggtaggtagttcattgaaaaaaataggtaagcattaaattttgtttgctgaatctaagtattagatactttaagagttgtatatcataaatgatattgagcctagaatgtttggctgttttacttttagaactttttgcaacagagtaaacatacatattatgaaaataaatgttctcttttttcctctgattttctagatgtgacttacttaacagaagagaaggtatatgaaattcttgaattatgtagagaaagagaggattattcccctttaatccgtgttattggaagagttttttctagtgctgaggcattggtacagagcttccggaaagttaaacaacacaccaaggaagaactgaaatctcttcaagcaaaagatgaagacaaagatgaagatgaaaaggaaaaagctgcatgttctgctgctgctatggaagaagactcagaagcatcttcctcaaggataggtgatagctcacagggagacaacaatttgcaaaaattaggccctgatgatgtgtctgtggatattgatgccattagaagggtctacaccagattgctctctaatgaaaaaattgaaactgcctttctcaatgcacttgtatatttgtcacctaacgtggaatgtgacttgacgtatcacaatgtatactctcgagatcctaattatctgaatttgttcattatcgtaatggagaatagaaatctccacagtcctgaatatctggaaatggctttgccattattttgcaaagcgatgagcaagctaccccttgcagcccaaggaaaactgatcagactgtggtctaaatacaatgcagaccagattcggagaatgatggagacatttcagcaacttattacttataaagtcataagcaatgaatttaacagtcgaaatctagtgaatgatgatgatgccattgttgctgcttcgaagtgcttgaaaatggtttactatgcaaatgtagtgggaggggaagtggacacaaatcacaatgaagaagatgatgaagagcccatccctgagtccagcgagctgacacttcaggaacttttgggagaagaaagaagaaacaagaaaggtcctcgagtggaccccctggaaactgaacttggtgttaaaaccctggattgtcgaaaaccacttatcccttttgaagagtttattaatgaaccactgaatgaggttctagaaatggataaagattatacttttttcaaagtagaaacagagaacaaattctcttttatgacatgtccctttatattgaatgctgtcacaaagaatttgggattatattatgacaatagaattcgcatgtacagtgaacgaagaatcactgttctctacagcttagttcaaggacagcagttgaatccatatttgagactcaaagttagacgtgaccatatcatagatgatgcacttgtccgggtaagttgggctgctagattaaaaacctaataatggggatatcatgatacagttcagtgaattcattttaaaagtgactgaaaaaaatgataccatatagcataggaacacatggacatttctgatcttatataagtattatacttttgttgttcctgtgcaagtttatagatgtgttctacaaagtatcggttgtattatataatggtcatgctatctttgaaaaagaatgggttttctaaatcttgaaaactaaatccaaagtttctttcattcagaagagaatagagtgttggacaaagaccagaacaagagaaatgtggagatacccaataataagtgtggatgtgcagtcttgaactgggagtaatggtacagtaaaaccataccataaaattataggtagtgtccaaaaaattccatcgtgtaaaattcagagttgcattattgtggacttgaagaagcagttgtatgtgggacggtatcgataagcttgatatcgaattcctgcagcccgggggatccactagtgtggtaattaatactaagtcttactgtgagagaccataaactgctttagtattcagtgtatttttcttaattgaaatatttaacttatgacttagtagatactaagacttaacccttgagtttctattctaataaaggactactaatgaacaattttgaggttagacctctactccattgtttttgctgaaatgatttagctgcttttccatgtcctgtgtagtccagacttaacacacaagtaataaaatcttaattaattgtatgttaatttcataacaaatcagtaaagttagctttttactatgctagtgtctgttttgtgtctgtctttttgattatctttaagactgaatctttgtcttcactggctttttatcagtttgctttctgtttccatttacatacaaaaagtcaaaaatttgtatttgtttcctaatcctactccttgtttttattttgtttttttcctgatactagcaatcatcttcttttcatgtttatcttttcaatcactagctagagatgatcgctatggaaaatcctgcagacttgaagaagcagttgtatgtggaatttgaaggagaacaaggagttgatgagggaggtgtttccaaagaattttttcagctggttgtggaggaaatcttcaatccagatattggtaaatacattagtaatgtgattatggtgtcgtatcatcttttgagttagttatttgtttatcttactttgtaaatattttcagctatgaagagcagcaaaagaaggatttggtatggattacccagaatcacacatcatgactgaatttgtaggttttaggaactgatttgtatcactaatttattcaaattcttttatttcttagaaggaatattctaatgaaggaaattatctctttggtaaactgaattgaaagcactttagaatggtatattggaacagttggagggatttctttgctttttgttgtctaaaaccatcatcaaactcacggttttcctgacctgtgaacttcaaagaacaatggtttgaagagtattgagagactgtctcacaagtatgtcatgctcaaagttcagaaacactagctgatatcacattaattaggtttatttgctataagatttcttggggcttaatatangtagtgttcccccaaactttttgaactccagaactcttttctgccctaacagtagctactcaggagctgaggcaggagaattgtttgaacctaggaggcagaggttgcagtgagctgagatcgtgccactccagcccacccctgggtaacagagcgagactccatctcaaagaaaaaaatgaaaaattgttttcaaaaatagtacgtgtggtacagatataagtaattatatttttataaatgaaacactttggaaatgtagccattttttgtttttttatgtttatttttcagctatgggtggataaagcatgaatataacttttcttatgtgttagtagaaaattagaaagcttgaatttaattaacgtatttttctacccgatgccaccaaattacttactactttattcctttggcttcataaaattacatatcaccattcaccccaatttatagcagatatatgtggacattgttttctcaagtgctaatataatagaaatcaatgttgcatgcctaattacatatattttaaatgttttatatgcataattattttaagtttatatttgtattattcatcagtccttaataaaatacaaaagtaatgtatttttaaaaatcatttcttataggtatgttcacatacgatgaatctacaaaattgttttggtttaatccatcttcttttgaaactgagggtcagtttactctgattggcatagtactgggtctggctatttacaataactgtatactggatgtacattttcccatggttgtctacaggaagctaatggggaaaaaaggaacttttcgtgacttgggagactctcacccagtaagttctttgtcatttttttaattcagtctcttagattttatttaaatgcaaaaatttaatttatgtcaaaattttaaagtttttgtttagaatctttgttgatactcttatcaataagataaaaatgttttaatctgaccgaagtaccagaaacacttaaaaactcaaagggggacatttttatatattgctgtcagcacgaagctttcgtaagattgatttcatagagaagtgtttctaaacattttgtttgtgttttagtgaaatcttaagagataggtaaaaatcagagtagccctggctaagggtcttggtagttacaacgagtgtgcctgctcctaccacccccacccccaccttgagacaccacagaatttctcatagagcacagtgtgaattctattgctaaattggtggtatggggtttctcagcagagaatgggacatcacagtgactgacaatctttcttttataggttggaaactatttgggggactggagggatactgtctacactttttacaatttttattgataagatttttgttgtcttctaagaagagtgatataaattatttgttgtattttgtagttctatggtggcctcaatttaccatttctggttgctaggttctatatcagagtttaaaagatttattggagtatgaagggaatgtggaagatgacatgatgatcactttccagatatcacagacagatctttttggtaacccaatgatgtatgatctaaaggaaaatggtgataaaattccaattacaaatgaaaacaggaaggtaataaatgtttttatgtcacattttgtctcttcattaacactttcaaagcatgtatgcttataatttttaaagaagtatctaatatagtctgtacaaaaaaaaaacaagtaactaagtttatgtaaatgctagagtccacttttctaaatcttggatataagttggtatgaaagcacacagttgggcactaaagccccttttagagaaagaggacatgaagcaggagatagttaatagctaagtgtggttgtagtataaagcaagaagcagggtgtttcttgtattaagctgtaagcaggaacctcatgattaaggtctttatcacagaacaaataaaaattacatttaatttacacatgtatatcctgtttgtgataaaaatacatttctgaaaagtatactttacgtcagatttgggttctattgactaaaatgtgttcatcgggaatgggaataacccagaacataacaagcaaaaaattatgacaaatatatagtatacctttaagaaacatgtttatattgatataattttttgattaaatattatacacactaagggtacaangcacattttccttttatganttngatacagtagtttatgtgtcagtcagatacttccacatttttgctgaactggatacagtaagcagcttaccaaatattctatggtagaaaactnggacttcctggtttgcttaaatcaaatatattgtactctcttaaaacggttggcatttataaatagatggatacatggtttaaatgtgtctgttnacatacctagttgagagaacctaaagaattttctgcgtctccagcatttatattcagttctgtttaatacattatcgaaattgacatttataagtatgacagttttgtgtatatggccttttcatagcttaatattggctgtaacagagaattgtgaaattgtaagaagtagttttctttgtaggtgtaaaattgaatttttaagaatattcttgacagttttatgtatatggccttttcatagcttaatattggctataacagagaattgtgaaattgttaagaagtaggtgtaaaattgaatttttaagaatattcttgaatgtttttttcttggaaaaattaaaaagctatgcagcccaataacttgtgttttgtttgcatagcatattataagaagttcttgtgattaatgttttctacaggaatttgtcaatctttattctgactacattctcaataaatcagtagaaaaacagttcaaggcttttcggagaggttttcatatggtgaccaatgaatctcccttaaagtacttattcagaccagaagaaattgaattgcttatatgtggaagccgggtaagaaagcaggtgtctgcaaaaagtcatgtatcgatttattgtttgtaatgatacagtagtatagcagataactaagacatattttcttgaatttgcagaatctagatttccaagcactagaagaaactacagaatatgacggtggctataccagggactctgttctgattaggtgaggtacttagttcttcagaggaagatttgattcaccaaaggggtgtgtgattttgcttcagacctttatctctaggtactaattcccaaataagcaaactcacaaattgtcatctatatacttagatttgtatttgtaatataatcaccatttttcagagctaatcttgtgatttatttcatgaatgaagtgttgttatatataagtctcatgtaatctcctgcatttggcgtatggattatctagtattcctcactggttagagtatgcttactgctggttagaagataattaaaataaggctaccatgtctgcaatttttcctttcttttgaactctgcatttgtgaactgttacatggcttcccaggatcaagcactttttgagtgaaatggtagtcttttatttaattcttaagataatatgtccagatacatactagtatttccattttacaccctaaaaaactaagccctgaattctcacagaaagatgtagaggttcccagttctatctgcttttaaacaaatgcccttactactctactgtctacttctgtgtactacatcatcgtatgtagttgtttgcatttgggccagttggttggggcaggggtctttttttcttttgtcccttaatctgtatcactttttcctcccaaagttgagttaaaggatgagtagaccaggagaataaaggagaaaggataaataaaatatatacccaaaggcacctggagttaatttttccaaatattcatttcagtctttttcaattcataggattttgtcttttgctcattactgactgcataatgtgattataccatagtttaaatagtcacttcctgttactacacacttgggttttctcaattttttactattgtagtactaatattttactatattgtaatctaatccaaatttttacgtattcagagctgttcaggataaatttgcttggaaatttttaaatcaccagaagtgatactatcctgataattaacttccaagttgtctcttaatatagttttaatgcaaatcataagcttatgttagtaccagtcataatgaatgccaaactgaaaccagtattgtattttttctcattagggagttctgggaaatcgttcattcatttacagatgaacagaaaagactcttcttgcagtttacaacgggcacagacagagcacctgtgggaggactaggaaaattaaagatgattatagccaaaaatggcccagacacagaaaggtaggtaattattaacttgtgactgtatacctaccgaaaaccttgcattcctcgtcacatacatatgaactgtctttatagtttctgagcacattcgtgattttatatacaaatccccaaatcatattagacaattgagaaaatactttgctgtcattgtgtgaggaaacttttaagaaattgccctagttaaaaattattatggggctcacattggtttggaatcaaattagtgtgattcatttacttttttgattcccagcttgttaattgaaagccatataacatgatcatctatttagaatggttacattgaggctcggaagattatcatttgattgtgctagaatcctgttatcaaatcattttcttagtcatattgccagcagtgtttctaataagcatttaagagcacacactttgcagtcttgtaaaacaggtttgagtattttctccaccttagaggaagttacttgacttctcagtgacctaacctctaaagtgcatttactgatgtcctctctgtggttttgttgtggaaagatttagttaaatgaactgtaagaattcagtacctaaaatggtatctgttatgtagtaaaaactcaatggatacagtatcttatcatcgtcactagctttgagtaatttataggataaaggcaacttggtagttacacaacaaaaagtttatgatttgcattaatgtatagtttgcattgcagaccgtctcaactatatacaatctaaaaataggagcatttaattctaagtgtatttcccatgacttacagttttcctgtttttttccccttttctctatttaggttacctacatctcatacttgctttaatgtgcttttacttccggaatactcaagcaaagaaaaacttaaagagagattgttgaaggccatcacgtatgccaaaggatttggcatgctgtaaaacaaaacaaaacaaaataaaacaaaaaaaaggaaggaaaaaaaaagaaaaaatttaaaaaattttaaaaatataacgagggataaatttt

SEQ ID NO: 4 - protein, 852 aa, Homo sapiens
  PEPTIDE (1)..(852): Human UBE3A Isoform 1
MKRAAAKHLIERYYHQLTEGCGNEACTNEFCASCPTFLRMDNNAAAIKALELYKINAKLCDPHPSKKGASSAYLENSKGAPNNSCSEIKMNKKGARIDFKDVTYLTEEKVYEILELCREREDYSPLIRVIGRVFSSAEALVQSFRKVKQHTKEELKSLQAKDEDKDEDEKEKAACSAAAMEEDSEASSSRIGDSSQGDNNLQKLGPDDVSVDIDAIRRVYTRLLSNEKIETAFLNALVYLSPNVECDLTYHNVYSRDPNYLNLFIIVMENRNLHSPEYLEMALPLFCKAMSKLPLAAQGKLIRLWSKYNADQIRRMMETFQQLITYKVISNEFNSRNLVNDDDAIVAASKCLKMVYYANVVGGEVDTNHNEEDDEEPIPESSELTLQELLGEERRNKKGPRVDPLETELGVKTLDCRKPLIPFEEFINEPLNEVLEMDKDYTFFKVETENKFSFMTCPFILNAVTKNLGLYYDNRIRMYSERRITVLYSLVQGQQLNPYLRLKVRRDHIIDDALVRLEMIAMENPADLKKQLYVEFEGEQGVDEGGVSKEFFQLVVEEIFNPDIGMFTYDESTKLFWFNPSSFETEGQFTLIGIVLGLAIYNNCILDVHFPMVVYRKLMGKKGTFRDLGDSHPVLYQSLKDLLEYEGNVEDDMMITFQISQTDLFGNPMMYDLKENGDKIPITNENRKEFVNLYSDYILNKSVEKQFKAFRRGFHMVTNESPLKYLFRPEEIELLICGSRNLDFQALEETTEYDGGYTRDSVLIREFWEIVHSFTDEQKRLFLQFTTGTDRAPVGGLGKLKMIIAKNGPDTERLPTSHTCFNVLLLPEYSSKEKLKERLLKAITYAKGFGML

SEQ ID NO: 5 - DNA, 2559 bp, Homo sapiens
  misc_feature (1)..(2559): UBE3Av1 cDNA
atgaagcgagcagctgcaaagcatctaatagaacgctactaccaccagttaactgagggctgtggaaatgaagcctgcacgaatgagttttgtgcttcctgtccaacttttcttcgtatggataataatgcagcagctattaaagccctcgagctttataagattaatgcaaaactctgtgatcctcatccctccaagaaaggagcaagctcagcttaccttgagaactcgaaaggtgcccccaacaactcctgctctgagataaaaatgaacaagaaaggcgctagaattgattttaaagatgtgacttacttaacagaagagaaggtatatgaaattcttgaattatgtagagaaagagaggattattcccctttaatccgtgttattggaagagttttttctagtgctgaggcattggtacagagcttccggaaagttaaacaacacaccaaggaagaactgaaatctcttcaagcaaaagatgaagacaaagatgaggatgaaaaggaaaaagctgcatgttctgctgctgctatggaagaagactcagaagcatcttcctcaaggataggtgatagctcacagggagacaacaatttgcaaaaattaggccctgatgatgtgtctgtggatattgatgccattagaagggtctacaccagattgctctctaatgaaaaaattgaaactgcctttctcaatgcacttgtatatttgtcacctaacgtggaatgtgacttgacgtatcacaatgtatactctcgagatcctaattatctgaatttgttcattatcgtaatggagaatagaaatctccacagtcctgaatatctggaaatggctttgccattattttgcaaagcgatgagcaagctaccccttgcagcccaaggaaaactgatcagactgtggtctaaatacaatgcagaccagattcggagaatgatggagacatttcagcaacttattacttataaagtcataagcaatgaatttaacagtcgaaatctagtgaatgatgatgatgccattgttgctgcttcgaagtgcttgaaaatggtttactatgcaaatgtagtgggaggggaagtggacacaaatcacaatgaagaagatgatgaagagcccatccctgagtccagcgagctgacacttcaggaacttttgggagaagaaagaagaaacaagaaaggtcctcgagtggaccccctggaaactgaacttggtgttaaaaccctggattgtcgaaaaccacttatcccttttgaagagtttattaatgaaccactgaatgaggttctagaaatggataaagattatacttttttcaaagtagaaacagagaacaaattctcttttatgacatgtccctttatattgaatgctgtcacaaagaatttgggattatattatgacaatagaattcgcatgtacagtgaacgaagaatcactgttctctacagcttagttcaaggacagcagttgaatccatatttgagactcaaagttagacgtgaccatatcatagatgatgcacttgtccggctagagatgatcgctatggaaaatcctgcagacttgaagaagcagttgtatgtggaatttgaaggagaacaaggagttgatgagggaggtgtttccaaagaattttttcagctggttgtggaggaaatcttcaatccagatattggtatgttcacatacgatgaatctacaaaattgttttggtttaatccatcttcttttgaaactgagggtcagtttactctgattggcatagtactgggtctggctatttacaataactgtatactggatgtacattttcccatggttgtctacaggaagctaatggggaaaaaaggaacttttcgtgacttgggagactctcacccagttctatatcagagtttaaaagatttattggagtatgaagggaatgtggaagatgacatgatgatcactttccagatatcacagacagatctttttggtaacccaatgatgtatgatctaaaggaaaatggtgataaaattccaattacaaatgaaaacaggaaggaatttgtcaatctttattctgactacattctcaataaatcagtagaaaaacagttcaaggcttttcggagaggttttcatatggtgaccaatgaatctcccttaaagtacttattcagaccagaagaaattgaattgcttatatgtggaagccggaatctagatttccaagcactagaagaaactacagaatatgacggtggctataccagggactctgttctgattagggagttctgggaaatcgttcattcatttacagatgaacagaaaagactcttcttgcagtttacaacgggcacagacagagcacctgtgggaggactaggaaaattaaagatgattatagccaaaaatggcccagacacagaaaggttacctacatctcatacttgctttaatgtgcttttacttccggaatactcaagcaaagaaaaacttaaagagagattgttgaaggccatcacgtatgccaaaggatttggcatgctgtaa

SEQ ID NO: 6 - DNA, 5276 bp, Homo sapiens
  misc_feature (1)..(5276): UBE3Av2 cDNA
agccagtcctcccgtcttgcgccgcggccgcgagatccgtgtgtctcccaagatggtggcgctgggctcggggtgactacaggagacgacggggccttttcccttcgccaggacccgacacaccaggcttcgctcgctcgcgcacccctccgccgcgtagccatccgccagcgcgggcgcccgccatccgccgcctacttacgcttcacctctgccgacccggcgcgctcggctgcgggcggcggcgcctccttcggctcctcctcggaatagctcgcggcctgtagcccctggcaggagggcccctcagccccccggtgtggacaggcagcggcggctggcgacgaacgccgggatttcggcggccccggcgctccctttcccggcctcgttttccggataaggaagcgcgggtcccgcatgagccccggcggtggcggcagcgaaagagaacgaggcggtggcgggcggaggcggcgggcgagggcgactacgaccagtgaggcggccgccgcagcccaggcgcgggggcgacgacaggttaaaaatctgtaagagcctgattttagaattcaccagctcctcagaagtttggcgaaatatgagttattaagcctacgctcagatcaaggtagcagctagactggtgtgacaacctgtttttaatcagtgactcaaagctgtgatcaccctgatgtcaccgaatggccacagcttgtaaaagagagttacagtggaggtaaaaggagtggcttgcaggatggagaagctgcaccagtgttattggaaatcaggagaacctcagtctgacgacattgaagctagccgaatgaagcgagcagctgcaaagcatctaatagaacgctactaccaccagttaactgagggctgtggaaatgaagcctgcacgaatgagttttgtgcttcctgtccaacttttcttcgtatggataataatgcagcagctattaaagccctcgagctttataagattaatgcaaaactctgtgatcctcatccctccaagaaaggagcaagctcagcttaccttgagaactcgaaaggtgcccccaacaactcctgctctgagataaaaatgaacaagaaaggcgctagaattgattttaaagatgtgacttacttaacagaagagaaggtatatgaaattcttgaattatgtagagaaagagaggattattcccctttaatccgtgttattggaagagttttttctagtgctgaggcattggtacagagcttccggaaagttaaacaacacaccaaggaagaactgaaatctcttcaagcaaaagatgaagacaaagatgaagatgaaaaggaaaaagctgcatgttctgctgctgctatggaagaagactcagaagcatcttcctcaaggataggtgatagctcacagggagacaacaatttgcaaaaattaggccctgatgatgtgtctgtggatattgatgccattagaagggtctacaccagattgctctctaatgaaaaaattgaaactgcctttctcaatgcacttgtatatttgtcacctaacgtggaatgtgacttgacgtatcacaatgtatactctcgagatcctaattatctgaatttgttcattatcgtaatggagaatagaaatctccacagtcctgaatatctggaaatggctttgccattattttgcaaagcgatgagcaagctaccccttgcagcccaaggaaaactgatcagactgtggtctaaatacaatgcagaccagattcggagaatgatggagacatttcagcaacttattacttataaagtcataagcaatgaatttaacagtcgaaatctagtgaatgatgatgatgccattgttgctgcttcgaagtgcttgaaaatggtttactatgcaaatgtagtgggaggggaagtggacacaaatcacaatgaagaagatgatgaagagcccatccctgagtccagcgagctgacacttcaggaacttttgggagaagaaagaagaaacaagaaaggtcctcgagtggaccccctggaaactgaacttggtgttaaaaccctggattgtcgaaaaccacttatcccttttgaagagtttattaatgaaccactgaatgaggttctagaaatggataaagattatacttttttcaaagtagaaacagagaacaaattctcttttatgacatgtccctttatattgaatgctgtcacaaagaatttgggattatattatgacaatagaattcgcatgtacagtgaacgaagaatcactgttctctacagcttagttcaaggacagcagttgaatccatatttgagactcaaagttagacgtgaccatatcatagatgatgcacttgtccggctagagatgatcgctatggaaaatcctgcagacttgaagaagcagttgtatgtggaatttgaaggagaacaaggagttgatgagggaggtgtttccaaagaattttttcagctggttgtggaggaaatcttcaatccagatattggtatgttcacatacgatgaatctacaaaattgttttggtttaatccatcttcttttgaaactgagggtcagtttactctgattggcatagtactgggtctggctatttacaataactgtatactggatgtacattttcccatggttgtctacaggaagctaatggggaaaaaaggaacttttcgtgacttgggagactctcacccagttctatatcagagtttaaaagatttattggagtatgaagggaatgtggaagatgacatgatgatcactttccagatatcacagacagatctttttggtaacccaatgatgtatgatctaaaggaaaatggtgataaaattccaattacaaatgaaaacaggaaggaatttgtcaatctttattctgactacattctcaataaatcagtagaaaaacagttcaaggcttttcggagaggttttcatatggtgaccaatgaatctcccttaaagtacttattcagaccagaagaaattgaattgcttatatgtggaagccggaatctagatttccaagcactagaagaaactacagaatatgacggtggctataccagggactctgttctgattagggagttctgggaaatcgttcattcatttacagatgaacagaaaagactcttcttgcagtttacaacgggcacagacagagcacctgtgggaggactaggaaaattaaagatgattatagccaaaaatggcccagacacagaaaggttacctacatctcatacttgctttaatgtgcttttacttccggaatactcaagcaaagaaaaacttaaagagagattgttgaaggccatcacgtatgccaaaggatttggcatgctgtaaaacaaaacaaaacaaaataaaacaaaaaaaaggaaggaaaaaaaaagaaaaaatttaaaaaattttaaaaatataacgagggataaatttttggtggtgatagtgtcccagtacaaaaaggctgtaagatagtcaaccacagtagtcacctatgtctgtgcctcccttctttattggggacatgtgggctggaacagcagatttcagctacatatatgaacaaatcctttattattattataattatttttttgcgtgaaagtgttacatattctttcacttgtatgtacagagaggtttttctgaatatttattttaagggttaaatcacttttgcttgtgtttattactgcttgaggttgagccttttgagtatttaaaaaatatataccaacagaactactctcccaaggaaaatattgccaccatttgtagaccacgtaaccttcaagtatgtgctacttttttgtccctgtatctaactcaaatcaggaactgtattttttttaatgatttgcttttgaaacttgaagtcttgaaaacagtgtgatgcaattactgctgttctagcccccaaagagttttctgtgcaaaatcttgagaatcaatcaataaagaaagatggaaggaagggagaaattggaatgttttaactgcagccctcagaactttagtaacagcacaacaaattaaaaacaaaaacaactcatgccacagtatgtcgtcttcatgtgtcttgcaatgaactgtttcagtagccaatcctctttcttagtatatgaaaggacagggatttttgttcttgttgttctcgttgttgttttaagtttactggggaaagtgcatttggccaaatgaaatggtagtcaagcctattgcaacaaagttaggaagtttgttgtttgtttattataaacaaaaagcatgtgaaagtgcacttaagatagagtttttattaattacttacttattacctagattttaaatagacaatccaaagtctccccttcgtgttgccatcatcttgttgaatcagccattttatcgaggcacgtgatcagtgttgcaacataatgaaaaagatggctactgtgccttgtgttacttaatcatacagtaagctgacctggaaatgaatgaaactattactcctaagaattacattgtatagccccacagattaaatttaattaattaattcaaaacatgttaaacgttactttcatgtactatggaaaagtacaagtaggtttacattactgatttccagaagtaagtagtttcccctttcctagtcttctgtgtatgtgatgttgttaatttcttttattgcattataaaataaaaggattatgtatttttaactaaggtgagacattgatatatccttttgctacaagctatagctaatgtgctgagcttgtgccttggtgattgattgattgattgactgattgttttaactgattactgtagatcaacctgatgatttgtttgtttgaaattggcaggaaaaatgcagctttcaaatcattggggggagaaaaaggatgtctttcaggattattttaattaatttttttcataattgagacagaactgtttgttatgtaccataatgctaaataaaactgtggcacttttcaccataatttaatttagtggaaaaagaagacaatgctttccatattgtgataaggtaacatggggtttttctgggccagcctttagaacactgttagggtacatacgctaccttgatgaaagggaccttcgtgcaactgtagtcatcttaaaggcttctcatccactgtgcttcttaatgtgtaattaaagtgaggagaaattaaatactctgagggcgttttatataataaattcgtgaaga

SEQ ID NO: 7 - protein, 875 aa, Homo sapiens
  PEPTIDE (1)..(875): UBE3A Isoform 2
MEKLHQCYWKSGEPQSDDIEASRMKRAAAKHLIERYYHQLTEGCGNEACTNEFCASCPTFLRMDNNAAAIKALELYKINAKLCDPHPSKKGASSAYLENSKGAPNNSCSEIKMNKKGARIDFKDVTYLTEEKVYEILELCREREDYSPLIRVIGRVFSSAEALVQSFRKVKQHTKEELKSLQAKDEDKDEDEKEKAACSAAAMEEDSEASSSRIGDSSQGDNNLQKLGPDDVSVDIDAIRRVYTRLLSNEKIETAFLNALVYLSPNVECDLTYHNVYSRDPNYLNLFIIVMENRNLHSPEYLEMALPLFCKAMSKLPLAAQGKLIRLWSKYNADQIRRMMETFQQLITYKVISNEFNSRNLVNDDDAIVAASKCLKMVYYANVVGGEVDTNHNEEDDEEPIPESSELTLQELLGEERRNKKGPRVDPLETELGVKTLDCRKPLIPFEEFINEPLNEVLEMDKDYTFFKVETENKFSFMTCPFILNAVTKNLGLYYDNRIRMYSERRITVLYSLVQGQQLNPYLRLKVRRDHIIDDALVRLEMIAMENPADLKKQLYVEFEGEQGVDEGGVSKEFFQLVVEEIFNPDIGMFTYDESTKLFWFNPSSFETEGQFTLIGIVLGLAIYNNCILDVHFPMVVYRKLMGKKGTFRDLGDSHPVLYQSLKDLLEYEGNVEDDMMITFQISQTDLFGNPMMYDLKENGDKIPITNENRKEFVNLYSDYILNKSVEKQFKAFRRGFHMVTNESPLKYLFRPEEIELLICGSRNLDFQALEETTEYDGGYTRDSVLIREFWEIVHSFTDEQKRLFLQFTTGTDRAPVGGLGKLKMIIAKNGPDTERLPTSHTCFNVLLLPEYSSKEKLKERLLKAITYAKGFGML

SEQ ID NO: 8 - DNA, 2970 bp, Homo sapiens
  misc_feature (1)..(2970): UBE3v3 cDNA
tttttccggataaggaagcgcgggtcccgcatgagccccggcggtggcggcagcgaaagagaacgaggcggtggcgggcggaggcggcgggcgagggcgactacgaccagtgaggcggccgccgcagcccaggcgcgggggcgacgacaggttaaaaatctgtaagagcctgattttagaattcaccagctcctcagaagtttggcgaaatatgagttattaagcctacgctcagatcaaggtagcagctagactggtgtgacaacctgtttttaatcagtgactcaaagctgtgatcaccctgatgtcaccgaatggccacagcttgtaaaagatcaggagaacctcagtctgacgacattgaagctagccgaatgaagcgagcagctgcaaagcatctaatagaacgctactaccaccagttaactgagggctgtggaaatgaagcctgcacgaatgagttttgtgcttcctgtccaacttttcttcgtatggataataatgcagcagctattaaagccctcgagctttataagattaatgcaaaactctgtgatcctcatccctccaagaaaggagcaagctcagcttaccttgagaactcgaaaggtgcccccaacaactcctgctctgagataaaaatgaacaagaaaggcgctagaattgattttaaagatgtgacttacttaacagaagagaaggtatatgaaattcttgaattatgtagagaaagagaggattattcccctttaatccgtgttattggaagagttttttctagtgctgaggcattggtacagagcttccggaaagttaaacaacacaccaaggaagaactgaaatctcttcaagcaaaagatgaagacaaagatgaagatgaaaaggaaaaagctgcatgttctgctgctgctatggaagaagactcagaggcatcttcctcaaggataggtgatagctcacagggagacaacaatttgcaaaaattaggccctgatgatgtgtctgtggatattgatgccattagaagggtctacaccagattgctctctaatgaaaaaattgaaactgcctttctcaatgcacttgtatatttgtcacctaacgtggaatgtgacttgacgtatcacaatgtatactctcgagatcctaattatctgaatttgttcattatcgtaatggagaatagaaatctccacagtcctgaatatctggaaatggctttgccattattttgcaaagcgatgagcaagctaccccttgcagcccaaggaaaactgatcagactgtggtctaaatacaatgcagaccagattcggagaatgatggagacatttcagcaacttattacttataaagtcataagcaatgaatttaacagtcgaaatctagtgaatgatgatgatgccattgttgctgcttcgaagtgcttgaaaatggtttactatgcaaatgtagtgggaggggaagtggacacaaatcacaatgaagaagatgatgaagagcccatccctgagtccagcgagctgacacttcaggaacttttgggagaagaaagaagaaacaagaaaggtcctcgagtggaccccctggaaactgaacttggtgttaaaaccctggattgtcgaaaaccacttatcccttttgaagagtttattaatgaaccactgaatgaggttctagaaatggataaagattatacttttttcaaagtagaaacagagaacaaattctcttttatgacatgtccctttatattgaatgctgtcacaaagaatttgggattatattatgacaatagaattcgcatgtacagtgaacgaagaatcactgttctctacagcttagttcaaggacagcagttgaatccatatttgagactcaaagttagacgtgaccatatcatagatgatgcacttgtccggctagagatgatcgctatggaaaatcctgcagacttgaagaagcagttgtatgtggaatttgaaggagaacaaggagttgatgagggaggtgtttccaaagaattttttcagctggttgtggaggaaatcttcaatccagatattggtatgttcacatacgatgaatctacaaaattgttttggtttaatccatcttcttttgaaactgagggtcagtttactctgattggcatagtactgggtctggctatttacaataactgtatactggatgtacattttcccatggttgtctacaggaagctaatggggaaaaaaggaacttttcgtgacttgggagactctcacccagttctatatcagagtttaaaagatttattggagtatgaagggaatgtggaagatgacatgatgatcactttccagatatcacagacagatctttttggtaacccaatgatgtatgatctaaaggaaaatggtgataaaattccaattacaaatgaaaacaggaaggaatttgtcaatctttattctgactacattctcaataaatcagtagaaaaacagttcaaggcttttcggagaggttttcatatggtgaccaatgaatctcccttaaagtacttattcagaccagaagaaattgaattgcttatatgtggaagccggaatctagatttccaagcactagaagaaactacagaatatgacggtggctataccagggactctgttctgattagggagttctgggaaatcgttcattcatttacagatgaacagaaaagactcttcttgcagtttacaacgggcacagacagagcacctgtgggaggactaggaaaattaaagatgattatagccaaaaatggcccagacacagaaaggttacctacatctcatacttgctttaatgtgcttttacttccggaatactcaagcaaagaaaaacttaaagagagattgttgaaggccatcacgtatgccaaaggatttggcatgctgtaaaacaaaacaaaacaaaataaaacaaaaaaaaggaagg

SEQ ID NO: 9 - protein, 872 aa, Homo sapiens
  PEPTIDE (1)..(872): UBE3A Isoform 3
MATACKRSGEPQSDDIEASRMKRAAAKHLIERYYHQLTEGCGNEACTNEFCASCPTFLRMDNNAAAIKALELYKINAKLCDPHPSKKGASSAYLENSKGAPNNSCSEIKMNKKGARIDFKDVTYLTEEKVYEILELCREREDYSPLIRVIGRVFSSAEALVQSFRKVKQHTKEELKSLQAKDEDKDEDEKEKAACSAAAMEEDSEASSSRIGDSSQGDNNLQKLGPDDVSVDIDAIRRVYTRLLSNEKIETAFLNALVYLSPNVECDLTYHNVYSRDPNYLNLFIIVMENRNLHSPEYLEMALPLFCKAMSKLPLAAQGKLIRLWSKYNADQIRRMMETFQQLITYKVISNEFNSRNLVNDDDAIVAASKCLKMVYYANVVGGEVDTNHNEEDDEEPIPESSELTLQELLGEERRNKKGPRVDPLETELGVKTLDCRKPLIPFEEFINEPLNEVLEMDKDYTFFKVETENKFSFMTCPFILNAVTKNLGLYYDNRIRMYSERRITVLYSLVQGQQLNPYLRLKVRRDHIIDDALVRLEMIAMENPADLKKQLYVEFEGEQGVDEGGVSKEFFQLVVEEIFNPDIGMFTYDESTKLFWFNPSSFETEGQFTLIGIVLGLAIYNNCILDVHFPMVVYRKLMGKKGTFRDLGDSHPVLYQSLKDLLEYEGNVEDDMMITFQISQTDLFGNPMMYDLKENGDKIPITNENRKEFVNLYSDYILNKSVEKQFKAFRRGFHMVTNESPLKYLFRPEEIELLICGSRNLDFQALEETTEYDGGYTRDSVLIREFWEIVHSFTDEQKRLFLQFTTGTDRAPVGGLGKLKMIIAKNGPDTERLPTSHTCFNVLLLPEYSSKEKLKERLLKAITYAKGFGML

SEQ ID NO: 10 - DNA, 2767 bp, Homo sapiens
  misc_feature (1)..(2767): UBE3Av1 cDNA (bottom strand)
attttgttttgttttgttttacagcatgccaaatcctttggcatacgtgatggccttcaacaatctctctttaagtttttctttgcttgagtattccggaagtaaaagcacattaaagcaagtatgagatgtaggtaacctttctgtgtctgggccatttttggctataatcatctttaattttcctagtcctcccacaggtgctctgtctgtgcccgttgtaaactgcaagaagagtcttttctgttcatctgtaaatgaatgaacgatttcccagaactccctaatcagaacagagtccctggtatagccaccgtcatattctgtagtttcttctagtgcttggaaatctagattccggcttccacatataagcaattcaatttcttctggtctgaataagtactttaagggagattcattggtcaccatatgaaaacctctccgaaaagccttgaactgtttttctactgatttattgagaatgtagtcagaataaagattgacaaattccttcctgttttcatttgtaattggaattttatcaccattttcctttagatcatacatcattgggttaccaaaaagatctgtctgtgatatctggaaagtgatcatcatgtcatcttccacattcccttcatactccaataaatcttttaaactctgatatagaactgggtgagagtctcccaagtcacgaaaagttccttttttccccattagcttcctgtagacaaccatgggaaaatgtacatccagtatacagttattgtaaatagccagacccagtactatgccaatcagagtaaactgaccctcagtttcaaaagaagatggattaaaccaaaacaattttgtagattcatcgtatgtgaacataccaatatctggattgaagatttcctccacaaccagctgaaaaaattctttggaaacacctccctcatcaactccttgttctccttcaaattccacatacaactgcttcttcaagtctgcaggattttccatagcgatcatctctagccggacaagtgcatcatctatgatatggtcacgtctaactttgagtctcaaatatggattcaactgctgtccttgaactaagctgtagagaacagtgattcttcgttcactgtacatgcgaattctattgtcataatataatcccaaattctttgtgacagcattcaatataaagggacatgtcataaaagagaatttgttctctgtttctactttgaaaaaagtataatctttatccatttctagaacctcattcagtggttcattaataaactcttcaaaagggataagtggttttcgacaatccagggttttaacaccaagttcagtttccagggggtccactcgaggacctttcttgtttcttctttcttctcccaaaagttcctgaagtgtcagctcgctggactcagggatgggctcttcatcatcttcttcattgtgatttgtgtccacttcccctcccactacatttgcatagtaaaccattttcaagcacttcgaagcagcaacaatggcatcatcatcattcactagatttcgactgttaaattcattgcttatgactttataagtaataagttgctgaaatgtctccatcattctccgaatctggtctgcattgtatttagaccacagtctgatcagttttccttgggctgcaaggggtagcttgctcatcgctttgcaaaataatggcaaagccatttccagatattcaggactgtggagatttctattctccattacgataatgaacaaattcagataattaggatctcgagagtatacattgtgatacgtcaagtcacattccacgttaggtgacaaatatacaagtgcattgagaaaggcagtttcaattttttcattagagagcaatctggtgtagacccttctaatggcatcaatatccacagacacatcatcagggcctaatttttgcaaattgttgtctccctgtgagctatcacctatccttgaggaagatgcttctgagtcttcttccatagcagcagcagaacatgcagctttttccttttcatcctcatctttgtcttcatcttttgcttgaagagatttcagttcttccttggtgtgttgtttaactttccggaagctctgtaccaatgcctcagcactagaaaaaactcttccaataacacggattaaaggggaataatcctctctttctctacataattcaagaatttcatataccttctcttctgttaagtaagtcacatctttaaaatcaattctagcgcctttcttgttcatttttatctcagagcaggagttgttgggggcacctttcgagttctcaaggtaagctgagcttgctcctttcttggagggatgaggatcacagagttttgcattaatcttataaagctcgagggctttaatagctgctgcattattatccatacgaagaaaagttggacaggaagcacaaaactcattcgtgcaggcttcatttccacagccctcagttaactggtggtagtagcgttctattagatgctttgcagctgctcgcttcattcggctagcttcaatgtcgtcagactgaggttctcctgatctggtctccaccagccttgtgggtaagtaccccaagattgcttccaaacttgtatctcccagacagaagagctaaaaattgatgataaccccatgtaccagctgactcatttgtgaaagtgcgaccctccagcatcagatgtcatactgt

SEQ ID NO: 11 - DNA, 5276 bp, Artificial Sequence
  UBE3Av2 cDNA (bottom strand)
  misc_feature (1)..(5276): UBE3A v2 cDNA (bottom strand)
tcttcacgaatttattatataaaacgccctcagagtatttaatttctcctcactttaattacacattaagaagcacagtggatgagaagcctttaagatgactacagttgcacgaaggtccctttcatcaaggtagcgtatgtaccctaacagtgttctaaaggctggcccagaaaaaccccatgttaccttatcacaatatggaaagcattgtcttctttttccactaaattaaattatggtgaaaagtgccacagttttatttagcattatggtacataacaaacagttctgtctcaattatgaaaaaaattaattaaaataatcctgaaagacatcctttttctccccccaatgatttgaaagctgcatttttcctgccaatttcaaacaaacaaatcatcaggttgatctacagtaatcagttaaaacaatcagtcaatcaatcaatcaatcaccaaggcacaagctcagcacattagctatagcttgtagcaaaaggatatatcaatgtctcaccttagttaaaaatacataatccttttattttataatgcaataaaagaaattaacaacatcacatacacagaagactaggaaaggggaaactacttacttctggaaatcagtaatgtaaacctacttgtacttttccatagtacatgaaagtaacgtttaacatgttttgaattaattaattaaatttaatctgtggggctatacaatgtaattcttaggagtaatagtttcattcatttccaggtcagcttactgtatgattaagtaacacaaggcacagtagccatctttttcattatgttgcaacactgatcacgtgcctcgataaaatggctgattcaacaagatgatggcaacacgaaggggagactttggattgtctatttaaaatctaggtaataagtaagtaattaataaaaactctatcttaagtgcactttcacatgctttttgtttataataaacaaacaacaaacttcctaactttgttgcaataggcttgactaccatttcatttggccaaatgcactttccccagtaaacttaaaacaacaacgagaacaacaagaacaaaaatccctgtcctttcatatactaagaaagaggattggctactgaaacagttcattgcaagacacatgaagacgacatactgtggcatgagttgtttttgtttttaatttgttgtgctgttactaaagttctgagggctgcagttaaaacattccaatttctcccttccttccatctttctttattgattgattctcaagattttgcacagaaaactctttgggggctagaacagcagtaattgcatcacactgttttcaagacttcaagtttcaaaagcaaatcattaaaaaaaatacagttcctgatttgagttagatacagggacaaaaaagtagcacatacttgaaggttacgtggtctacaaatggtggcaatattttccttgggagagtagttctgttggtatatattttttaaatactcaaaaggctcaacctcaagcagtaataaacacaagcaaaagtgatttaacccttaaaataaatattcagaaaaacctctctgtacatacaagtgaaagaatatgtaacactttcacgcaaaaaaataattataataataataaaggatttgttcatatatgtagctgaaatctgctgttccagcccacatgtccccaataaagaagggaggcacagacataggtgactactgtggttgactatcttacagcctttttgtactgggacactatcaccaccaaaaatttatccctcgttatatttttaaaattttttaaattttttctttttttttccttccttttttttgttttattttgttttgttttgttttacagcatgccaaatcctttggcatacgtgatggccttcaacaatctctctttaagtttttctttgcttgagtattccggaagtaaaagcacattaaagcaagtatgagatgtaggtaacctttctgtgtctgggccatttttggctataatcatctttaattttcctagtcctcccacaggtgctctgtctgtgcccgttgtaaactgcaagaagagtcttttctgttcatctgtaaatgaatgaacgatttcccagaactccctaatcagaacagagtccctggtatagccaccgtcatattctgtagtttcttctagtgcttggaaatctagattccggcttccacatataagcaattcaatttcttctggtctgaataagtactttaagggagattcattggtcaccatatgaaaacctctccgaaaagccttgaactgtttttctactgatttattgagaatgtagtcagaataaagattgacaaattccttcctgttttcatttgtaattggaattttatcaccattttcctttagatcatacatcattgggttaccaaaaagatctgtctgtgatatctggaaagtgatcatcatgtcatcttccacattcccttcatactccaataaatcttttaaactctgatatagaactgggtgagagtctcccaagtcacgaaaagttccttttttccccattagcttcctgtagacaaccatgggaaaatgtacatccagtatacagttattgtaaatagccagacccagtactatgccaatcagagtaaactgaccctcagtttcaaaagaagatggattaaaccaaaacaattttgtagattcatcgtatgtgaacataccaatatctggattgaagatttcctccacaaccagctgaaaaaattctttggaaacacctccctcatcaactccttgttctccttcaaattccacatacaactgcttcttcaagtctgcaggattttccatagcgatcatctctagccggacaagtgcatcatctatgatatggtcacgtctaactttgagtctcaaatatggattcaactgctgtccttgaactaagctgtagagaacagtgattcttcgttcactgtacatgcgaattctattgtcataatataatcccaaattctttgtgacagcattcaatataaagggacatgtcataaaagagaatttgttctctgtttctactttgaaaaaagtataatctttatccatttctagaacctcattcagtggttcattaataaactcttcaaaagggataagtggttttcgacaatccagggttttaacaccaagttcagtttccagggggtccactcgaggacctttcttgtttcttctttcttctcccaaaagttcctgaagtgtcagctcgctggactcagggatgggctcttcatcatcttcttcattgtgatttgtgtccacttcccctcccactacatttgcatagtaaaccattttcaagcacttcgaagcagcaacaatggcatcatcatcattcactagatttcgactgttaaattcattgcttatgactttataagtaataagttgctgaaatgtctccatcattctccgaatctggtctgcattgtatttagaccacagtctgatcagttttccttgggctgcaaggggtagcttgctcatcgctttgcaaaataatggcaaagccatttccagatattcaggactgtggagatttctattctccattacgataatgaacaaattcagataattaggatctcgagagtatacattgtgatacgtcaagtcacattccacgttaggtgacaaatatacaagtgcattgagaaaggcagtttcaattttttcattagagagcaatctggtgtagacccttctaatggcatcaatatccacagacacatcatcagggcctaatttttgcaaattgttgtctccctgtgagctatcacctatccttgaggaagatgcttctgagtcttcttccatagcagcagcagaacatgcagctttttccttttcatcttcatctttgtcttcatcttttgcttgaagagatttcagttcttccttggtgtgttgtttaactttccggaagctctgtaccaatgcctcagcactagaaaaaactcttccaataacacggattaaaggggaataatcctctctttctctacataattcaagaatttcatataccttctcttctgttaagtaagtcacatctttaaaatcaattctagcgcctttcttgttcatttttatctcagagcaggagttgttgggggcacctttcgagttctcaaggtaagctgagcttgctcctttcttggagggatgaggatcacagagttttgcattaatcttataaagctcgagggctttaatagctgctgcattattatccatacgaagaaaagttggacaggaagcacaaaactcattcgtgcaggcttcatttccacagccctcagttaactggtggtagtagcgttctattagatgctttgcagctgctcgcttcattcggctagcttcaatgtcgtcagactgaggttctcctgatttccaataacactggtgcagcttctccatcctgcaagccactccttttacctccactgtaactctcttttacaagctgtggccattcggtgacatcagggtgatcacagctttgagtcactgattaaaaacaggttgtcacaccagtctagctgctaccttgatctgagcgtaggcttaataactcatatttcgccaaacttctgaggagctggtgaattctaaaatcaggctcttacagatttttaacctgtcgtcgcccccgcgcctgggctgcggcggccgcctcactggtcgtagtcgccctcgcccgccgcctccgcccgccaccgcctcgttctctttcgctgccgccaccgccggggctcatgcgggacccgcgcttccttatccggaaaacgaggccgggaaagggagcgccggggccgccgaaatcccggcgttcgtcgccagccgccgctgcctgtccacaccggggggctgaggggccctcctgccaggggctacaggccgcgagctattccgaggaggagccgaaggaggcgccgccgcccgcagccgagcgcgccgggtcggcagaggtgaagcgtaagtaggcggcggatggcgggcgcccgcgctggcggatggctacgcggcggaggggtgcgcgagcgagcgaagcctggtgtgtcgggtcctggcgaagggaaaaggccccgtcgtctcctgtagtcaccccgagcccagcgccaccatcttgggagacacacggatctcgcggccgcggcgcaagacgggaggactggct

SEQ ID NO: 12 - DNA, 2970 bp, Artificial Sequence
  UBE3Av3 (bottom strand)
  misc_feature (1)..(2970): UBE3Av3 cDNA (bottom strand)
ccttccttttttttgttttattttgttttgttttgttttacagcatgccaaatcctttggcatacgtgatggccttcaacaatctctctttaagtttttctttgcttgagtattccggaagtaaaagcacattaaagcaagtatgagatgtaggtaacctttctgtgtctgggccatttttggctataatcatctttaattttcctagtcctcccacaggtgctctgtctgtgcccgttgtaaactgcaagaagagtcttttctgttcatctgtaaatgaatgaacgatttcccagaactccctaatcagaacagagtccctggtatagccaccgtcatattctgtagtttcttctagtgcttggaaatctagattccggcttccacatataagcaattcaatttcttctggtctgaataagtactttaagggagattcattggtcaccatatgaaaacctctccgaaaagccttgaactgtttttctactgatttattgagaatgtagtcagaataaagattgacaaattccttcctgttttcatttgtaattggaattttatcaccattttcctttagatcatacatcattgggttaccaaaaagatctgtctgtgatatctggaaagtgatcatcatgtcatcttccacattcccttcatactccaataaatcttttaaactctgatatagaactgggtgagagtctcccaagtcacgaaaagttccttttttccccattagcttcctgtagacaaccatgggaaaatgtacatccagtatacagttattgtaaatagccagacccagtactatgccaatcagagtaaactgaccctcagtttcaaaagaagatggattaaaccaaaacaattttgtagattcatcgtatgtgaacataccaatatctggattgaagatttcctccacaaccagctgaaaaaattctttggaaacacctccctcatcaactccttgttctccttcaaattccacatacaactgcttcttcaagtctgcaggattttccatagcgatcatctctagccggacaagtgcatcatctatgatatggtcacgtctaactttgagtctcaaatatggattcaactgctgtccttgaactaagctgtagagaacagtgattcttcgttcactgtacatgcgaattctattgtcataatataatcccaaattctttgtgacagcattcaatataaagggacatgtcataaaagagaatttgttctctgtttctactttgaaaaaagtataatctttatccatttctagaacctcattcagtggttcattaataaactcttcaaaagggataagtggttttcgacaatccagggttttaacaccaagttcagtttccagggggtccactcgaggacctttcttgtttcttctttcttctcccaaaagttcctgaagtgtcagctcgctggactcagggatgggctcttcatcatcttcttcattgtgatttgtgtccacttcccctcccactacatttgcatagtaaaccattttcaagcacttcgaagcagcaacaatggcatcatcatcattcactagatttcgactgttaaattcattgcttatgactttataagtaataagttgctgaaatgtctccatcattctccgaatctggtctgcattgtatttagaccacagtctgatcagttttccttgggctgcaaggggtagcttgctcatcgctttgcaaaataatggcaaagccatttccagatattcaggactgtggagatttctattctccattacgataatgaacaaattcagataattaggatctcgagagtatacattgtgatacgtcaagtcacattccacgttaggtgacaaatatacaagtgcattgagaaaggcagtttcaattttttcattagagagcaatctggtgtagacccttctaatggcatcaatatccacagacacatcatcagggcctaatttttgcaaattgttgtctccctgtgagctatcacctatccttgaggaagatgcctctgagtcttcttccatagcagcagcagaacatgcagctttttccttttcatcttcatctttgtcttcatcttttgcttgaagagatttcagttcttccttggtgtgttgtttaactttccggaagctctgtaccaatgcctcagcactagaaaaaactcttccaataacacggattaaaggggaataatcctctctttctctacataattcaagaatttcatataccttctcttctgttaagtaagtcacatctttaaaatcaattctagcgcctttcttgttcatttttatctcagagcaggagttgttgggggcacctttcgagttctcaaggtaagctgagcttgctcctttcttggagggatgaggatcacagagttttgcattaatcttataaagctcgagggctttaatagctgctgcattattatccatacgaagaaaagttggacaggaagcacaaaactcattcgtgcaggcttcatttccacagccctcagttaactggtggtagtagcgttctattagatgctttgcagctgctcgcttcattcggctagcttcaatgtcgtcagactgaggttctcctgatcttttacaagctgtggccattcggtgacatcagggtgatcacagctttgagtcactgattaaaaacaggttgtcacaccagtctagctgctaccttgatctgagcgtaggcttaataactcatatttcgccaaacttctgaggagctggtgaattctaaaatcaggctcttacagatttttaacctgtcgtcgcccccgcgcctgggctgcggcggccgcctcactggtcgtagtcgccctcgcccgccgcctccgcccgccaccgcctcgttctctttcgctgccgccaccgccggggctcatgcgggacccgcgcttccttatccggaaaaa

SEQ ID NO: 13 - protein, 865 aa, Artificial Sequence
  Consensus amino acid sequence
SGEPQSDDIEASRMKRAAAKHLIERYYHQLTEGCGNEACTNEFCASCPTFLRMDNNAAAIKALELYKINAKLCDPHPSKKGASSAYLENSKGAPNNSCSEIKMNKKGARIDFKDVTYLTEEKVYEILELCREREDYSPLIRVIGRVFSSAEALVQSFRKVKQHTKEELKSLQAKDEDKDEDEKEKAACSAAAMEEDSEASSSRIGDSSQGDNNLQKLGPDDVSVDIDAIRRVYTRLLSNEKIETAFLNALVYLSPNVECDLTYHNVYSRDPNYLNLFIIVMENRNLHSPEYLEMALPLFCKAMSKLPLAAQGKLIRLWSKYNADQIRRMMETFQQLITYKVISNEFNSRNLVNDDDAIVAASKCLKMVYYANVVGGEVDTNHNEEDDEEPIPESSELTLQELLGEERRNKKGPRVDPLETELGVKTLDCRKPLIPFEEFINEPLNEVLEMDKDYTFFKVETENKFSFMTCPFILNAVTKNLGLYYDNRIRMYSERRITVLYSLVQGQQLNPYLRLKVRRDHIIDDALVRLEMIAMENPADLKKQLYVEFEGEQGVDEGGVSKEFFQLVVEEIFNPDIGMFTYDESTKLFWFNPSSFETEGQFTLIGIVLGLAIYNNCILDVHFPMVVYRKLMGKKGTFRDLGDSHPVLYQSLKDLLEYEGNVEDDMMITFQISQTDLFGNPMMYDLKENGDKIPITNENRKEFVNLYSDYILNKSVEKQFKAFRRGFHMVTNESPLKYLFRPEEIELLICGSRNLDFQALEETTEYDGGYTRDSVLIREFWEIVHSFTDEQKRLFLQFTTGTDRAPVGGLGKLKMIIAKNGPDTERLPTSHTCFNVLLLPEYSSKEKLKERLLKAITYAKGFGML

SEQ ID NO: 14 - DNA, 145 bp, adeno-associated virus 1
  misc_feature (1)..(145): AAV1 ITR
ttgcccactccctctctgcgcgctcgctcgctcggtggggcctgcggaccaaaggtccgcagacggcagagctctgctctgccggccccaccgagcgagcgagcgcgcagagagggagtgggcaactccatcactaggggtaatc

SEQ ID NO: 15 - DNA, 145 bp, adeno-associated virus 2
  misc_feature (1)..(145): AAV2 ITR
aggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 16 - DNA, 143 bp, adeno-associated virus 3
  misc_feature (1)..(143): AAV3 ITR
ttggccactccctctatgcgcactcgctcgctcggtggggcctggcgaccaaaggtcgccagacggacgtgctttgcacgtccggccccaccgagcgagcgagtgcgcatagagggagtggccaactccatcactagaggtat

SEQ ID NO: 17 - DNA, 146 bp, adeno-associated virus 4
  misc_feature (1)..(146): AAV4 ITR
atgccggggttctacgagatcgtgctgaaggtgcccagcgacctggacgagcacctgcccggcatttctgactcttttgtgagctgggtggccgagaaggaatgggagctgccgccggattctgacatggacttgaatctgattga

SEQ ID NO: 18 - DNA, 143 bp, adeno-associated virus 5
  misc_feature (1)..(143): AAV5 ITR
ctctcccccctgtcgcgttcgctcgctcgctggctcgtttgggggggtggcagctcaaagagctgccagacgacggccctctggccgtcgcccccccaaacgagccagcgagcgagcgaacgcgacaggggggagagtgccac

SEQ ID NO: 19 - DNA, 145 bp, adeno-associated virus 6
  misc_feature (1)..(145): AAV6 ITR
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcct

SEQ ID NO: 20 - DNA, 145 bp, adeno-associated virus 7
  misc_feature (1)..(145): AAV7 ITR
ttggccactccctctatgcgcgctcgctcgctcggtggggcctgcggaccaaaggtccgcagacggcagagctctgctctgccggccccaccgagcgagcgagcgcgcatagagggagtggccaactccatcactaggggtaccg

SEQ ID NO: 21 - DNA, 146 bp, adeno-associated virus 8
  misc_feature (1)..(146): AAV8 ITR
cagagagggagtggccaactccatcactaggggtagcgcgaagcgcctcccacgctgccgcgtcagcgctgacgtaaattacgtcataggggagtggtcctgtattagctgtcacgtgagtgcttttgcggcattttgcgacacca

SEQ ID NO: 22 - DNA, 61 bp, adeno-associated virus 2
  misc_feature (1)..(61): truncated AAV2 ITR
ctgcgcgctcgctcgctcactgaggccgcccgggcaaagcccgggcgtcgggcgacctttg

SEQ ID NO: 23 - DNA, 6619 bp, Artificial Sequence
  Plasmid sequence
  misc_feature (1)..(6619): pTR-UphUBE3 ITR-ITR sequence (bottom strand)
cccccccccccccccccctgcagcccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctacgcaactgttgggaagggcgatcggtgcgggcctcttcgctattacgccagctggcctgcaggggggggggggggggggttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatctctccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgaggctgatcagcgagctctagtcgacatcgatatgcatcccggggctagcttacagcatgccaaatcctttggcatacgtgatggccttcaacaatctctctttaagtttttctttgcttgagtattccggaagtaaaagcacattaaagcaagtatgagatgtaggtaacctttctgtgtctgggccatttttggctataatcatctttaattttcctagtcctcccacaggtgctctgtctgtgcccgttgtaaactgcaagaagagtcttttctgttcatctgtaaatgaatgaacgatttcccagaactccctaatcagaacagagtccctggtatagccaccgtcatattctgtagtttcttctagtgcttggaaatctagattccggcttccacatataagcaattcaatttcttctggtctgaataagtactttaagggagattcattggtcaccatatgaaaacctctccgaaaagccttgaactgtttttctactgatttattgagaatgtagtcagaataaagattgacaaattccttcctgttttcatttgtaattggaattttatcaccattttcctttagatcatacatcattgggttaccaaaaagatctgtctgtgatatctggaaagtgatcatcatgtcatcttccacattcccttcatactccaataaatcttttaaactctgatatagaactgggtgagagtctcccaagtcacgaaaagttccttttttccccattagcttcctgtagacaaccatgggaaaatgtacatccagtatacagttattgtaaatagccagacccagtactatgccaatcagagtaaactgaccctcagtttcaaaagaagatggattaaaccaaaacaattttgtagattcatcgtatgtgaacataccaatatctggattgaagatttcctccacaaccagctgaaaaaattctttggaaacacctccctcatcaactccttgttctccttcaaattccacatacaactgcttcttcaagtctgcaggattttccatagcgatcatctctagccggacaagtgcatcatctatgatatggtcacgtctaactttgagtctcaaatatggattcaactgctgtccttgaactaagctgtagagaacagtgattcttcgttcactgtacatgcgaattctattgtcataatataatcccaaattctttgtgacagcattcaatataaagggacatgtcataaaagagaatttgttctctgtttctactttgaaaaaagtataatctttatccatttctagaacctcattcagtggttcattaataaactcttcaaaagggataagtggttttcgacaatccagggttttaacaccaagttcagtttccagggggtccactcgaggacctttcttgtttcttctttcttctcccaaaagttcctgaagtgtcagctcgctggactcagggatgggctcttcatcatcttcttcattgtgatttgtgtccacttcccctcccactacatttgcatagtaaaccattttcaagcacttcgaagcagcaacaatggcatcatcatcattcactagatttcgactgttaaattcattgcttatgactttataagtaataagttgctgaaatgtctccatcattctccgaatctggtctgcattgtatttagaccacagtctgatcagttttccttgggctgcaaggggtagcttgctcatcgctttgcaaaataatggcaaagccatttccagatattcaggactgtggagatttctattctccattacgataatgaacaaattcagataattaggatctcgagagtatacattgtgatacgtcaagtcacattccacgttaggtgacaaatatacaagtgcattgagaaaggcagtttcaattttttcattagagagcaatctggtgtagacccttctaatggcatcaatatccacagacacatcatcagggcctaatttttgcaaattgttgtctccctgtgagctatcacctatccttgaggaagatgcttctgagtcttcttccatagcagcagcagaacatgcagctttttccttttcatcttcatctttgtcttcatcttttgcttgaagagatttcagttcttccttggtgtgttgtttaactttccggaagctctgtaccaatgcctcagcactagaaaaaactcttccaataacacggattaaaggggaataatcctctctttctctacataattcaagaatttcatataccttctcttctgttaagtaagtcacatctttaaaatcaattctagcgcctttcttgttcatttttatctcagagcaggagttgttgggggcacctttcgagttctcaaggtaagctgagcttgctcctttcttggagggatgaggatcacagagttttgcattaatcttataaagctcgagggctttaatagctgctgcattattatccatacgaagaaaagttggacaggaagcacaaaactcattcgtgcaggcttcatttccacagccctcagttaactggtggtagtagcgttctattagatgctttgcagctgctcgcttcatggtggaccggtaagcttcgtctaacaaaaaagccaaaaacggccagaatttagcggacaatttactagtctaacactgaaaattacatattgacccaaatgattacatttcaaaaggtgcctaaaaaacttcacaaaacacactcgccaaccccgagcgcacgtacccagcccagcagcccgctactcaccaagtgacgatcacagcgatccacaaacaagaaccgcgacccaaatcccggctgcgacggaactagctgtgccacacccggcgcgtccttatataatcatcggcgttcaccgccccacggagatccctccgcagaatcgccgagaagggactacttttcctcgcctgttccgctctctggaaagaaaaccagtgccctagagtcacccaagtcccgtcctaaaatgtccttctgctgatactggggttctaaggccgagtcttatgagcagcgggccgctgtcctgagcgtccgggcggaaggatcaggacgctcgctgcgcccttcgtctgacgtggcagcgctcgccgtgaggaggggggcgcccgcgggaggcgccaaaacccggcgcggaggccggccggccttaattaagcgatcgcgaattcagatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 24 - DNA, 3744 bp, Artificial Sequence
  Plasmid sequence
  misc_feature (1)..(3744): pTR-UphUBE3 ITR-ITR sequence (bottom strand)
gggttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatctctccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgaggctgatcagcgagctctagtcgacatcgatatgcatcccggggctagcttacagcatgccaaatcctttggcatacgtgatggccttcaacaatctctctttaagtttttctttgcttgagtattccggaagtaaaagcacattaaagcaagtatgagatgtaggtaacctttctgtgtctgggccatttttggctataatcatctttaattttcctagtcctcccacaggtgctctgtctgtgcccgttgtaaactgcaagaagagtcttttctgttcatctgtaaatgaatgaacgatttcccagaactccctaatcagaacagagtccctggtatagccaccgtcatattctgtagtttcttctagtgcttggaaatctagattccggcttccacatataagcaattcaatttcttctggtctgaataagtactttaagggagattcattggtcaccatatgaaaacctctccgaaaagccttgaactgtttttctactgatttattgagaatgtagtcagaataaagattgacaaattccttcctgttttcatttgtaattggaattttatcaccattttcctttagatcatacatcattgggttaccaaaaagatctgtctgtgatatctggaaagtgatcatcatgtcatcttccacattcccttcatactccaataaatcttttaaactctgatatagaactgggtgagagtctcccaagtcacgaaaagttccttttttccccattagcttcctgtagacaaccatgggaaaatgtacatccagtatacagttattgtaaatagccagacccagtactatgccaatcagagtaaactgaccctcagtttcaaaagaagatggattaaaccaaaacaattttgtagattcatcgtatgtgaacataccaatatctggattgaagatttcctccacaaccagctgaaaaaattctttggaaacacctccctcatcaactccttgttctccttcaaattccacatacaactgcttcttcaagtctgcaggattttccatagcgatcatctctagccggacaagtgcatcatctatgatatggtcacgtctaactttgagtctcaaatatggattcaactgctgtccttgaactaagctgtagagaacagtgattcttcgttcactgtacatgcgaattctattgtcataatataatcccaaattctttgtgacagcattcaatataaagggacatgtcataaaagagaatttgttctctgtttctactttgaaaaaagtataatctttatccatttctagaacctcattcagtggttcattaataaactcttcaaaagggataagtggttttcgacaatccagggttttaacaccaagttcagtttccagggggtccactcgaggacctttcttgtttcttctttcttctcccaaaagttcctgaagtgtcagctcgctggactcagggatgggctcttcatcatcttcttcattgtgatttgtgtccacttcccctcccactacatttgcatagtaaaccattttcaagcacttcgaagcagcaacaatggcatcatcatcattcactagatttcgactgttaaattcattgcttatgactttataagtaataagttgctgaaatgtctccatcattctccgaatctggtctgcattgtatttagaccacagtctgatcagttttccttgggctgcaaggggtagcttgctcatcgctttgcaaaataatggcaaagccatttccagatattcaggactgtggagatttctattctccattacgataatgaacaaattcagataattaggatctcgagagtatacattgtgatacgtcaagtcacattccacgttaggtgacaaatatacaagtgcattgagaaaggcagtttcaattttttcattagagagcaatctggtgtagacccttctaatggcatcaatatccacagacacatcatcagggcctaatttttgcaaattgttgtctccctgtgagctatcacctatccttgaggaagatgcttctgagtcttcttccatagcagcagcagaacatgcagctttttccttttcatcttcatctttgtcttcatcttttgcttgaagagatttcagttcttccttggtgtgttgtttaactttccggaagctctgtaccaatgcctcagcactagaaaaaactcttccaataacacggattaaaggggaataatcctctctttctctacataattcaagaatttcatataccttctcttctgttaagtaagtcacatctttaaaatcaattctagcgcctttcttgttcatttttatctcagagcaggagttgttgggggcacctttcgagttctcaaggtaagctgagcttgctcctttcttggagggatgaggatcacagagttttgcattaatcttataaagctcgagggctttaatagctgctgcattattatccatacgaagaaaagttggacaggaagcacaaaactcattcgtgcaggcttcatttccacagccctcagttaactggtggtagtagcgttctattagatgctttgcagctgctcgcttcatggtggaccggtaagcttcgtctaacaaaaaagccaaaaacggccagaatttagcggacaatttactagtctaacactgaaaattacatattgacccaaatgattacatttcaaaaggtgcctaaaaaacttcacaaaacacactcgccaaccccgagcgcacgtacccagcccagcagcccgctactcaccaagtgacgatcacagcgatccacaaacaagaaccgcgacccaaatcccggctgcgacggaactagctgtgccacacccggcgcgtccttatataatcatcggcgttcaccgccccacggagatccctccgcagaatcgccgagaagggactacttttcctcgcctgttccgctctctggaaagaaaaccagtgccctagagtcacccaagtcccgtcctaaaatgtccttctgctgatactggggttctaaggccgagtcttatgagcagcgggccgctgtcctgagcgtccgggcggaaggatcaggacgctcgctgcgcccttcgtctgacgtggcagcgctcgccgtgaggaggggggcgcccgcgggaggcgccaaaacccggcgcggaggccggccggccttaattaagcgatcgcgaattcagatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 25 - DNA, 2559 bp, Homo sapiens; identical to SEQ ID NO: 5
  misc_feature (1)..(2559): UBE3Av1 coding region

SEQ ID NO: 26 - DNA, 2559 bp, Homo sapiens
  misc_feature (1)..(2599): UBE3Av1 cDNA coding strand (bottom strand)
ttacagcatgccaaatcctttggcatacgtgatggccttcaacaatctctctttaagtttttctttgcttgagtattccggaagtaaaagcacattaaagcaagtatgagatgtaggtaacctttctgtgtctgggccatttttggctataatcatctttaattttcctagtcctcccacaggtgctctgtctgtgcccgttgtaaactgcaagaagagtcttttctgttcatctgtaaatgaatgaacgatttcccagaactccctaatcagaacagagtccctggtatagccaccgtcatattctgtagtttcttctagtgcttggaaatctagattccggcttccacatataagcaattcaatttcttctggtctgaataagtactttaagggagattcattggtcaccatatgaaaacctctccgaaaagccttgaactgtttttctactgatttattgagaatgtagtcagaataaagattgacaaattccttcctgttttcatttgtaattggaattttatcaccattttcctttagatcatacatcattgggttaccaaaaagatctgtctgtgatatctggaaagtgatcatcatgtcatcttccacattcccttcatactccaataaatcttttaaactctgatatagaactgggtgagagtctcccaagtcacgaaaagttccttttttccccattagcttcctgtagacaaccatgggaaaatgtacatccagtatacagttattgtaaatagccagacccagtactatgccaatcagagtaaactgaccctcagtttcaaaagaagatggattaaaccaaaacaattttgtagattcatcgtatgtgaacataccaatatctggattgaagatttcctccacaaccagctgaaaaaattctttggaaacacctccctcatcaactccttgttctccttcaaattccacatacaactgcttcttcaagtctgcaggattttccatagcgatcatctctagccggacaagtgcatcatctatgatatggtcacgtctaactttgagtctcaaatatggattcaactgctgtccttgaactaagctgtagagaacagtgattcttcgttcactgtacatgcgaattctattgtcataatataatcccaaattctttgtgacagcattcaatataaagggacatgtcataaaagagaatttgttctctgtttctactttgaaaaaagtataatctttatccatttctagaacctcattcagtggttcattaataaactcttcaaaagggataagtggttttcgacaatccagggttttaacaccaagttcagtttccagggggtccactcgaggacctttcttgtttcttctttcttctcccaaaagttcctgaagtgtcagctcgctggactcagggatgggctcttcatcatcttcttcattgtgatttgtgtccacttcccctcccactacatttgcatagtaaaccattttcaagcacttcgaagcagcaacaatggcatcatcatcattcactagatttcgactgttaaattcattgcttatgactttataagtaataagttgctgaaatgtctccatcattctccgaatctggtctgcattgtatttagaccacagtctgatcagttttccttgggctgcaaggggtagcttgctcatcgctttgcaaaataatggcaaagccatttccagatattcaggactgtggagatttctattctccattacgataatgaacaaattcagataattaggatctcgagagtatacattgtgatacgtcaagtcacattccacgttaggtgacaaatatacaagtgcattgagaaaggcagtttcaattttttcattagagagcaatctggtgtagacccttctaatggcatcaatatccacagacacatcatcagggcctaatttttgcaaattgttgtctccctgtgagctatcacctatccttgaggaagatgcttctgagtcttcttccatagcagcagcagaacatgcagctttttccttttcatcctcatctttgtcttcatcttttgcttgaagagatttcagttcttccttggtgtgttgtttaactttccggaagctctgtaccaatgcctcagcactagaaaaaactcttccaataacacggattaaaggggaataatcctctctttctctacataattcaagaatttcatataccttctcttctgttaagtaagtcacatctttaaaatcaattctagcgcctttcttgttcatttttatctcagagcaggagttgttgggggcacctttcgagttctcaaggtaagctgagcttgctcctttcttggagggatgaggatcacagagttttgcattaatcttataaagctcgagggctttaatagctgctgcattattatccatacgaagaaaagttggacaggaagcacaaaactcattcgtgcaggcttcatttccacagccctcagttaactggtggtagtagcgttctattagatgctttgcagctgctcgcttcat

SEQ ID NO: 27 - protein, 736 aa, adeno-associated virus 9
  PEPTIDE (1)..(660): AAV 9.2 VP1 capsid protein
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYFLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRFLTRNL

SEQ ID NO: 28 - protein, 736 aa, adeno-associated virus 9
  Peptide (1)..(736): wt AAV 9 VPI protein
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 29 - protein, 736 aa, adeno-associated virus 9
  PEPTIDE (1)..(736): AAV9 VP1 protein
  MISC_FEATURE (446)..(446): X= Y or F
  MISC_FEATURE (731)..(731): X= Y or F
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYXLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRXLTRNL

SEQ ID NO: 30 - DNA, 2208 bp, adeno-associated virus 9
  misc_feature (1)..(2208): AAV 9.1 VP1 capsid cDNA
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactttctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctg

SEQ ID NO: 31 - DNA, 2208 bp, adeno-associated virus 9
  misc_feature (1)..(2208): AAV 9.1 cDNA (bottom strand)
cagattacgagtcaggtatctggtgccaatggggcggggttcactatatacaccttcagtattaacagcaaattcaacattattagacttgtaatagttggaagtgtactggatctccgggttccagcgcttgctgttttccttctgcagctcccactcgatctccacgctgacttggccagtagaatactgggtgatgaaagagttcagcttgtccttgttgaaggccgttggaggatccgcaggtacaggtgtgtttttgatgaggatctgaggaggcgggtgcttcattccaaaccctcccatcagcggagaagggtgaaagttgccgtccgtgtgaggaattttggcccaaatgggtccttgcaggtacacatctctgtcctgccaaaccatacccggaagtattccttggttttgaacccagccggtctgcgcctgtgcttgggcactctggtggtttgtggccacttgtccataggactccgttgctaccgggttagtagttttaatttcttcttcgttggttatcatgactttgtccgcatccacgttgtctcttccagttccttgtttgccaaaaattaaagatccagacaaaggaaagaaacggtcctctccttctttgtggctggccatagcaggtccaggattcatcaagctattacgtccattgagagcccaagaagaagctccaggccaagcaaattcgctgttgttgttttgagtcacagtggttgagacacgttgttgtcggtagctgggtccaggtatgtagtttcttccctggacagccatgttgctgggtccggccacactgaattttagcgtttgttgattctgtccagaaccgttaatagtctttgagagaaagtacaagtattggtcgatgagtggattcattagtcggtccaggctttggctgtgagcgtagctgctatggaaaggtacgttctcaaactcgtagctgaactggaagttgttacccgttcttagcatttgcgacgggaaatattccaggcagtaaaaggacgaacgacccacggcctggcttccatcattaagcgtcagatacccgtactgaggaatcatgaaaacgtccgctgggaacggcgggaggcagccctcgtgagccgacccgagcacgtacgggagctgatagtctgagtccgtgaagacctggaccgtgctggtaaggttattggcgatggtcttgactccattgttgtccgtaacctctttgacctgaatgttgaagagcttgaagttgagtcgcttaggccggaatccccagttgttgttgatgagtcgctgccagtcacgtggtgagaagtggcagtggaatctgttgaagtcaaaatacccccagggggtgctgtagccgaagtaggcgttgtcatttgaagatcctccagatgtgctgttggagatttgcttgtagaggtgattgttgtaggtgggcagggcccaggttcgggtgctggtggtgatgactctgtcccccagccattgggaatcgcaatgccaatttcccgaggaactacccactccatcggcaccttcgttattgtctgccactggtgcgccaccacctgaagccattgtaagagatcccacacctgagggggctgcgggaggttctccgattggttgagggtctgggactgactctgtgtcgccagtctgaccgaaattgagtctctttttagcgggctgtgcacccgatttgccaatacccgcggaggagtccggttcctgaggagactgctctacaggcctcttctttccaggagccgtcttagccgcttcctcaaccagaccaagaggttcaagaagcctctttttggcctggaagactgctcgcccgaggttgcccccaaaagacgtatcttctttgagccgctcctggaactcggcgtcggcgtggttgtacttgaggtacgggttgtctccggccttgagctgctggtcgtaggccttgtcgtgctcgagggccgccgcgtctgctgcgttgaccggctcccccttgtcgagtccgttgccgggtccaaggtatttgtaacccggaagcacaagacctcgagcgttgtcttgatgttgttgatttgccttgggttgaggggctccaggtttcaaagcccaccactcgcgaattccttcactaaggttgtcctcgagccaatctggaagataaccatcggcagccat

SEQ ID NO: 32 - protein, 736 aa, adeno-associated virus 9
  peptide (1)..(736): AAV 9.1 VP1 capsid protein
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYFLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 33 - DNA, 2208 bp, adeno-associated virus 9
  misc_feature (1)..(2208): AAV 9.2 VP1 cDNA
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactttctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagattcctgactcgtaatctg

SEQ ID NO: 34 - DNA, 2208 bp, adeno-associated virus 9
  misc_feature (1)..(2208): AAV 9.2 VP1 cDNA (bottom strand)
cagattacgagtcaggaatctggtgccaatggggcggggttcactatatacaccttcagtattaacagcaaattcaacattattagacttgtaatagttggaagtgtactggatctccgggttccagcgcttgctgttttccttctgcagctcccactcgatctccacgctgacttggccagtagaatactgggtgatgaaagagttcagcttgtccttgttgaaggccgttggaggatccgcaggtacaggtgtgtttttgatgaggatctgaggaggcgggtgcttcattccaaaccctcccatcagcggagaagggtgaaagttgccgtccgtgtgaggaattttggcccaaatgggtccttgcaggtacacatctctgtcctgccaaaccatacccggaagtattccttggttttgaacccagccggtctgcgcctgtgcttgggcactctggtggtttgtggccacttgtccataggactccgttgctaccgggttagtagttttaatttcttcttcgttggttatcatgactttgtccgcatccacgttgtctcttccagttccttgtttgccaaaaattaaagatccagacaaaggaaagaaacggtcctctccttctttgtggctggccatagcaggtccaggattcatcaagctattacgtccattgagagcccaagaagaagctccaggccaagcaaattcgctgttgttgttttgagtcacagtggttgagacacgttgttgtcggtagctgggtccaggtatgtagtttcttccctggacagccatgttgctgggtccggccacactgaattttagcgtttgttgattctgtccagaaccgttaatagtctttgagagaaagtacaagtattggtcgatgagtggattcattagtcggtccaggctttggctgtgagcgtagctgctatggaaaggtacgttctcaaactcgtagctgaactggaagttgttacccgttcttagcatttgcgacgggaaatattccaggcagtaaaaggacgaacgacccacggcctggcttccatcattaagcgtcagatacccgtactgaggaatcatgaaaacgtccgctgggaacggcgggaggcagccctcgtgagccgacccgagcacgtacgggagctgatagtctgagtccgtgaagacctggaccgtgctggtaaggttattggcgatggtcttgactccattgttgtccgtaacctctttgacctgaatgttgaagagcttgaagttgagtcgcttaggccggaatccccagttgttgttgatgagtcgctgccagtcacgtggtgagaagtggcagtggaatctgttgaagtcaaaatacccccagggggtgctgtagccgaagtaggcgttgtcatttgaagatcctccagatgtgctgttggagatttgcttgtagaggtgattgttgtaggtgggcagggcccaggttcgggtgctggtggtgatgactctgtcccccagccattgggaatcgcaatgccaatttcccgaggaactacccactccatcggcaccttcgttattgtctgccactggtgcgccaccacctgaagccattgtaagagatcccacacctgagggggctgcgggaggttctccgattggttgagggtctgggactgactctgtgtcgccagtctgaccgaaattgagtctctttttagcgggctgtgcacccgatttgccaatacccgcggaggagtccggttcctgaggagactgctctacaggcctcttctttccaggagccgtcttagccgcttcctcaaccagaccaagaggttcaagaagcctctttttggcctggaagactgctcgcccgaggttgcccccaaaagacgtatcttctttgagccgctcctggaactcggcgtcggcgtggttgtacttgaggtacgggttgtctccggccttgagctgctggtcgtaggccttgtcgtgctcgagggccgccgcgtctgctgcgttgaccggctcccccttgtcgagtccgttgccgggtccaaggtatttgtaacccggaagcacaagacctcgagcgttgtcttgatgttgttgatttgccttgggttgaggggctccaggtttcaaagcccaccactcgcgaattccttcactaaggttgtcctcgagccaatctggaagataaccatcggcagccat

SEQ ID NO: 35 - DNA, 183 bp, Artificial Sequence
  cDNA fragment encoding AZUL domain
  misc_feature (1)..(183): Azul domain
atgaagcgagcagctgcaaagcatctaatagaacgctactaccaccagttaactgagggctgtggaaatgaagcctgcacgaatgagttttgtgcttcctgtccaacttttcttcgtatggataataatgcagcagctattaaagccctcgagctttataagattaatgcaaaactctgtgat

SEQ ID NO: 36 - DNA, 183 bp, Artificial Sequence
  Azul domain cDNA (bottom strand)
  misc_feature (1)..(183): Azul domain (bottom strand)
atcacagagttttgcattaatcttataaagctcgagggctttaatagctgctgcattattatccatacgaagaaaagttggacaggaagcacaaaactcattcgtgcaggcttcatttccacagccctcagttaactggtggtagtagcgttctattagatgctttgcagctgctcgcttcat

SEQ ID NO: 37 - DNA, 20 bp, Artificial Sequence
  primer
taaattctggccgtttttgg

SEQ ID NO: 38 - DNA, 20 bp, Artificial Sequence
  primer
catttccacagccctcagtt

SEQ ID NO: 39 - DNA, 20 bp, Artificial Sequence
  primer
catttccacagccctcagtt

SEQ ID NO: 40 - DNA, 20 bp, Artificial Sequence
  primer
attcgtgcaggcttcatttc

SEQ ID NO: 41 - DNA, 56 bp, Artificial Sequence
  GDNF gene fragment encoding secretion sequence
atgaagttatgggatgtcgtggctgtctgcctggtgctgctccacaccgcgtccgc

SEQ ID NO: 42 - DNA, 72 bp, Artificial Sequence
  Insulin gene fragment encoding secretion sequence
atggccctgtggatgcgcctcctgcccctgctggcgctgctggccctctggggacctgacccagccgcagcc

SEQ ID NO: 43 - DNA, 60 bp, Artificial Sequence
  IgK gene fragment encoding secretion sequence
atggagacagacacactcctgctatgggtactgctgctctgggttccaggttccactggt

SEQ ID NO: 44 - DNA, 3792 bp, Artificial Sequence
  Plasmid sequence (ITR-ITR sequence of pUphUbeKan)
  misc_feature (1)..(3792): ITR-ITR nucleotide sequence of the pUphUbe/kan plasmid
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttaactataacggtcctaaggtagcgagcgatcgcttaattaaggccggccggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctgggtacgtgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacgcctgcaggggcgcgccacgcgtaagcttaccggtccaccatgaagcgagcagctgcaaagcatctaatagaacgctactaccaccagttaactgagggctgtggaaatgaagcctgcacgaatgagttttgtgcttcctgtccaacttttcttcgtatggataataatgcagcagctattaaagccctcgagctttataagattaatgcaaaactctgtgatcctcatccctccaagaaaggagcaagctcagcttaccttgagaactcgaaaggtgcccccaacaactcctgctctgagataaaaatgaacaagaaaggcgctagaattgattttaaagatgtgacttacttaacagaagagaaggtatatgaaattcttgaattatgtagagaaagagaggattattcccctttaatccgtgttattggaagagttttttctagtgctgaggcattggtacagagcttccggaaagttaaacaacacaccaaggaagaactgaaatctcttcaagcaaaagatgaagacaaagatgaagatgaaaaggaaaaagctgcatgttctgctgctgctatggaagaagactcagaagcatcttcctcaaggataggtgatagctcacagggagacaacaatttgcaaaaattaggccctgatgatgtgtctgtggatattgatgccattagaagggtctacaccagattgctctctaatgaaaaaattgaaactgcctttctcaatgcacttgtatatttgtcacctaacgtggaatgtgacttgacgtatcacaatgtatactctcgagatcctaattatctgaatttgttcattatcgtaatggagaatagaaatctccacagtcctgaatatctggaaatggctttgccattattttgcaaagcgatgagcaagctaccccttgcagcccaaggaaaactgatcagactgtggtctaaatacaatgcagaccagattcggagaatgatggagacatttcagcaacttattacttataaagtcataagcaatgaatttaacagtcgaaatctagtgaatgatgatgatgccattgttgctgcttcgaagtgcttgaaaatggtttactatgcaaatgtagtgggaggggaagtggacacaaatcacaatgaagaagatgatgaagagcccatccctgagtccagcgagctgacacttcaggaacttttgggagaagaaagaagaaacaagaaaggtcctcgagtggaccccctggaaactgaacttggtgttaaaaccctggattgtcgaaaaccacttatcccttttgaagagtttattaatgaaccactgaatgaggttctagaaatggataaagattatacttttttcaaagtagaaacagagaacaaattctcttttatgacatgtccctttatattgaatgctgtcacaaagaatttgggattatattatgacaatagaattcgcatgtacagtgaacgaagaatcactgttctctacagcttagttcaaggacagcagttgaatccatatttgagactcaaagttagacgtgaccatatcatagatgatgcacttgtccggctagagatgatcgctatggaaaatcctgcagacttgaagaagcagttgtatgtggaatttgaaggagaacaaggagttgatgagggaggtgtttccaaagaattttttcagctggttgtggaggaaatcttcaatccagatattggtatgttcacatacgatgaatctacaaaattgttttggtttaatccatcttcttttgaaactgagggtcagtttactctgattggcatagtactgggtctggctatttacaataactgtatactggatgtacattttcccatggttgtctacaggaagctaatggggaaaaaaggaacttttcgtgacttgggagactctcacccagttctatatcagagtttaaaagatttattggagtatgaagggaatgtggaagatgacatgatgatcactttccagatatcacagacagatctttttggtaacccaatgatgtatgatctaaaggaaaatggtgataaaattccaattacaaatgaaaacaggaaggaatttgtcaatctttattctgactacattctcaataaatcagtagaaaaacagttcaaggcttttcggagaggttttcatatggtgaccaatgaatctcccttaaagtacttattcagaccagaagaaattgaattgcttatatgtggaagccggaatctagatttccaagcactagaagaaactacagaatatgacggtggctataccagggactctgttctgattagggagttctgggaaatcgttcattcatttacagatgaacagaaaagactcttcttgcagtttacaacgggcacagacagagcacctgtgggaggactaggaaaattaaagatgattatagccaaaaatggcccagacacagaaaggttacctacatctcatacttgctttaatgtgcttttacttccggaatactcaagcaaagaaaaacttaaagagagattgttgaaggccatcacgtatgccaaaggatttggcatgctgtaagctagccccgggatgcatatcgatgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatctaggaaattaccctgttatccctaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 45 - DNA, 3792 bp, Artificial Sequence
  Plasmid sequence
  misc_feature (1)..(3792): ITR-ITR nucleotide sequence of the pUphUbe/kan plasmid (bottom strand)
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttagggataacagggtaatttcctagatctctccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgaggctgatcagcgagctctagtcgacatcgatatgcatcccggggctagcttacagcatgccaaatcctttggcatacgtgatggccttcaacaatctctctttaagtttttctttgcttgagtattccggaagtaaaagcacattaaagcaagtatgagatgtaggtaacctttctgtgtctgggccatttttggctataatcatctttaattttcctagtcctcccacaggtgctctgtctgtgcccgttgtaaactgcaagaagagtcttttctgttcatctgtaaatgaatgaacgatttcccagaactccctaatcagaacagagtccctggtatagccaccgtcatattctgtagtttcttctagtgcttggaaatctagattccggcttccacatataagcaattcaatttcttctggtctgaataagtactttaagggagattcattggtcaccatatgaaaacctctccgaaaagccttgaactgtttttctactgatttattgagaatgtagtcagaataaagattgacaaattccttcctgttttcatttgtaattggaattttatcaccattttcctttagatcatacatcattgggttaccaaaaagatctgtctgtgatatctggaaagtgatcatcatgtcatcttccacattcccttcatactccaataaatcttttaaactctgatatagaactgggtgagagtctcccaagtcacgaaaagttccttttttccccattagcttcctgtagacaaccatgggaaaatgtacatccagtatacagttattgtaaatagccagacccagtactatgccaatcagagtaaactgaccctcagtttcaaaagaagatggattaaaccaaaacaattttgtagattcatcgtatgtgaacataccaatatctggattgaagatttcctccacaaccagctgaaaaaattctttggaaacacctccctcatcaactccttgttctccttcaaattccacatacaactgcttcttcaagtctgcaggattttccatagcgatcatctctagccggacaagtgcatcatctatgatatggtcacgtctaactttgagtctcaaatatggattcaactgctgtccttgaactaagctgtagagaacagtgattcttcgttcactgtacatgcgaattctattgtcataatataatcccaaattctttgtgacagcattcaatataaagggacatgtcataaaagagaatttgttctctgtttctactttgaaaaaagtataatctttatccatttctagaacctcattcagtggttcattaataaactcttcaaaagggataagtggttttcgacaatccagggttttaacaccaagttcagtttccagggggtccactcgaggacctttcttgtttcttctttcttctcccaaaagttcctgaagtgtcagctcgctggactcagggatgggctcttcatcatcttcttcattgtgatttgtgtccacttcccctcccactacatttgcatagtaaaccattttcaagcacttcgaagcagcaacaatggcatcatcatcattcactagatttcgactgttaaattcattgcttatgactttataagtaataagttgctgaaatgtctccatcattctccgaatctggtctgcattgtatttagaccacagtctgatcagttttccttgggctgcaaggggtagcttgctcatcgctttgcaaaataatggcaaagccatttccagatattcaggactgtggagatttctattctccattacgataatgaacaaattcagataattaggatctcgagagtatacattgtgatacgtcaagtcacattccacgttaggtgacaaatatacaagtgcattgagaaaggcagtttcaattttttcattagagagcaatctggtgtagacccttctaatggcatcaatatccacagacacatcatcagggcctaatttttgcaaattgttgtctccctgtgagctatcacctatccttgaggaagatgcttctgagtcttcttccatagcagcagcagaacatgcagctttttccttttcatcttcatctttgtcttcatcttttgcttgaagagatttcagttcttccttggtgtgttgtttaactttccggaagctctgtaccaatgcctcagcactagaaaaaactcttccaataacacggattaaaggggaataatcctctctttctctacataattcaagaatttcatataccttctcttctgttaagtaagtcacatctttaaaatcaattctagcgcctttcttgttcatttttatctcagagcaggagttgttgggggcacctttcgagttctcaaggtaagctgagcttgctcctttcttggagggatgaggatcacagagttttgcattaatcttataaagctcgagggctttaatagctgctgcattattatccatacgaagaaaagttggacaggaagcacaaaactcattcgtgcaggcttcatttccacagccctcagttaactggtggtagtagcgttctattagatgctttgcagctgctcgcttcatggtggaccggtaagcttacgcgtggcgcgcccctgcaggcgtctaacaaaaaagccaaaaacggccagaatttagcggacaatttactagtctaacactgaaaattacatattgacccaaatgattacatttcaaaaggtgcctaaaaaacttcacaaaacacactcgccaaccccgagcgcacgtacccagcccagcagcccgctactcaccaagtgacgatcacagcgatccacaaacaagaaccgcgacccaaatcccggctgcgacggaactagctgtgccacacccggcgcgtccttatataatcatcggcgttcaccgccccacggagatccctccgcagaatcgccgagaagggactacttttcctcgcctgttccgctctctggaaagaaaaccagtgccctagagtcacccaagtcccgtcctaaaatgtccttctgctgatactggggttctaaggccgagtcttatgagcagcgggccgctgtcctgagcgtccgggcggaaggatcaggacgctcgctgcgcccttcgtctgacgtggcagcgctcgccgtgaggaggggggcgcccgcgggaggcgccaaaacccggcgcggaggccggccggccttaattaagcgatcgctcgctaccttaggaccgttatagttaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 46 - protein, 61 aa, Artificial Sequence
  Azul domain peptide
  PEPTIDE (1)..(61): Azul domain peptide
MKRAAAKHLIERYYHQLTEGCGNEACTNEFCASCPTFLRMDNNAAAIKALELYKINAKLCD